(19) United States
(12) Patent Application Publication
(10) Pub. No.: US 2025/0213707 A1
(43) Pub. Date: Jul. 3, 2025

(54) COMPOUND FOR INCREASING EFFICACY OF ONCOLYTIC VIRUSES

(71) Applicant: ABLEVIA BIOTECH GMBH, Wien (AT)

(72) Inventor: OSKAR SMRZKA, Wien (AT)

(21) Appl. No.: 18/849,972

(22) PCT Filed: Mar. 24, 2023
(86) PCT No.: PCT/EP2023/057582

§ 371 Date: Sep. 23, 2024

(30) Foreign Application Priority Data
Mar. 24, 2022 (EP) 22164232.5

(51) Int. Cl. A61K 47/64; A61K 35/768; A61P 35/00

(52) U.S. Cl. CPC A61K 47/643; A61K 35/768; A61K 47/644; A61P 35/00

(57) ABSTRACT

A compound for sequestration of undesirable neutralizing antibodies against oncolytic viruses in a patient, which comprises an inert biopolymer scaffold and at least a first peptide n-mer of the general formula P (—S—P)₍ₙ₋₁₎ and a second peptide n-mer of the general formula P (—S—P)₍ₙ₋₁₎; wherein, independently for each occurrence, P is a peptide with a sequence length of 6-13 amino acids and S is a non-peptide spacer, wherein, independently for each of the peptide n-mers, n is an integer of at least 1, wherein each of the peptide n-mers is bound to the biopolymer scaffold. Independently for each occurrence, P has an amino-acid sequence comprising a sequence fragment with a length of at least six amino acids of a protein sequence of an oncolytic virus. Pharmaceutical compositions comprising the compound and methods.

The field of present invention relates to compounds for increasing efficiency of oncolytic viruses.

Oncolytic viruses, e.g. based on adenovirus (AdV), adeno-associated virus (AAV), measles virus, Herpes simplex virus (HSV), poxvirus, reovirus, Newcastle disease virus (NVD), rhabdovirus, coxsackievirus, lentivirus, alphavirus or flavivirus, are suitable for cancer therapy. Some of them are used in their naturally-occurring form and some of them are used when “armed” with engineered (transgenic) payloads in order to improve their potency (reviewed by Cristi et al, 2022). Numerous studies in tumour models demonstrated substantial tumour regression and prolonged survival rates (Lundstrom, 2018). Several clinical trials confirmed good safety profiles and therapeutic efficacy of oncolytic viruses. Gendicine, based on an oncolytic AdV, was approved for the treatment of head and neck squamous cell carcinoma in China (Zhang et al, 2018). More recently, an oncolytic HSV-1 was approved for the treatment of melanoma in the US and Europe under the name talimogene laherparepvec, “T-Vec” (Conry et al, 2018).

The emerging field of virus-mediated cancer treatments is extensively reviewed by Kontermann et al., 2021. A wide variety of oncolytic viruses for local or systemic application routes is nowadays available (see review by Cook et al., 2021). Further viral vector platforms include vesicular stomatitis virus (VSS), myxoma virus (MYXV), mengovirus, bovine viral diarrhea virus (BVDV), or chimeric constructs. An extensive review of oncolytic vectors is provided by Apolonio et al., 2021.

The oncotoxic effect of oncolytic viruses can be enhanced by inserting various classes of functional genes into the virus genome (as a payload), in order to boost its oncolytic function. This includes the insertion of sequences encoding for e.g. antibodies, bi-specific T cell engagers (BiTEs), bispecific adapter proteins or various forms of fusion proteins, possibly also in combination with chimeric antigen receptor (CAR)-T cell therapies. By these strategies, it is possible to deliver immune checkpoint inhibitors, anti-angiogenic antibodies, or other functional entities such as e.g. cytotoxic fusion proteins to the tumour or to hematologic neoplasms.

However, oncolytic virotherapy encounters resistance mechanisms due to the humoral or cellular immune responses of the host against the oncolytic viral vectors. Bhatt et al., 2021, review the various different types of resistance mechanisms, from interferon signalling to epigenetic- or hypoxia-mediated mechanisms.

Beside the innate immune responses to oncolytic virus vectors (including natural killer cells or interferon responses), the presence of neutralizing antibodies (nAbs) against an oncolytic vector may also lead to the inhibition and (premature) clearance of the vector. Various strategies were proposed to remove or avoid the effect of such nAbs, including PEGylation, encapsulation or polymers for blocking and shielding the viral vector, as e.g. reviewed by de Matos et al, 2020, or Engeland & Ungerechts, 2021.

Despite these efforts, there is still a need for new strategies for improving efficacy of oncolytic viruses.

It is thus an object of the present invention to provide compounds and methods which inhibit oncolytic virus neutralization. Thereby, the efficacy of the oncolytic virus in cancer (or, more generally, anti-neoplastic) therapy is typically increased.

The present invention provides a compound comprising
a biopolymer scaffold and at least
a first peptide n-mer of the general formula:

$$P {( - S - P )}_{( n - 1 )}$$

a second peptide n-mer of the general formula:

$$P {( - S - P )}_{( n - 1 )} .$$

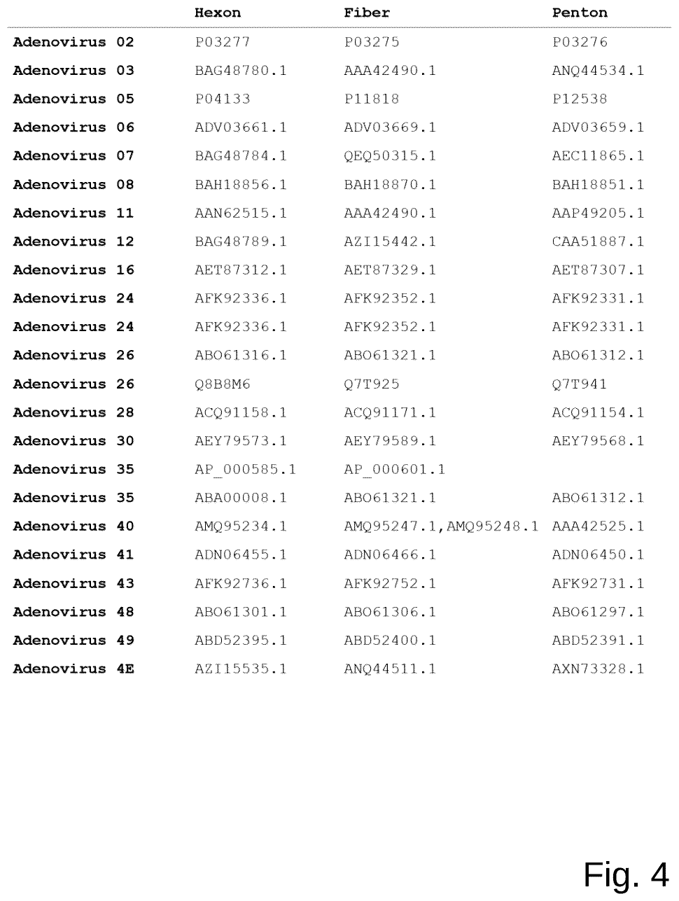

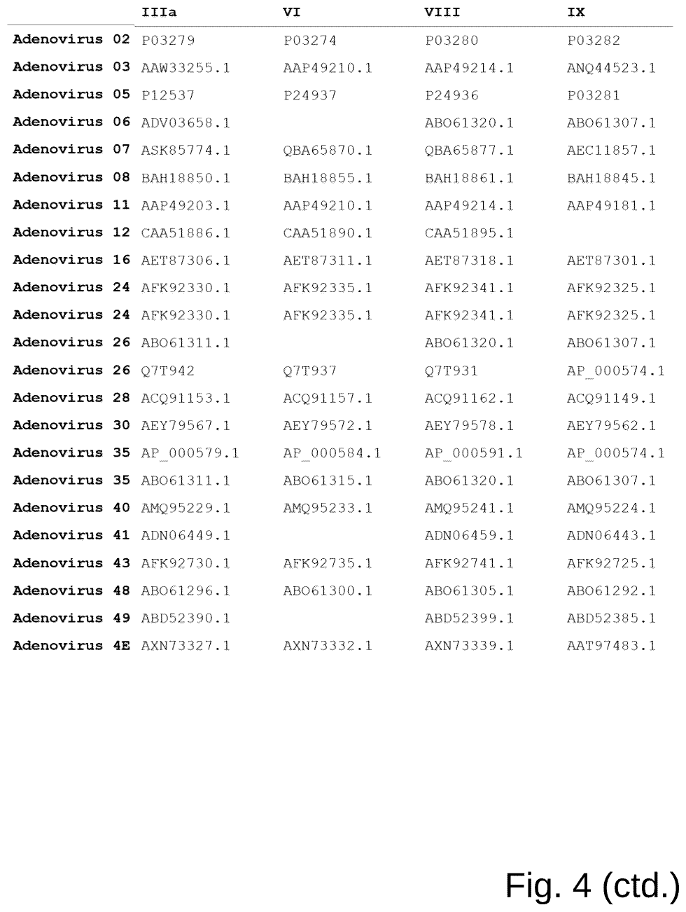

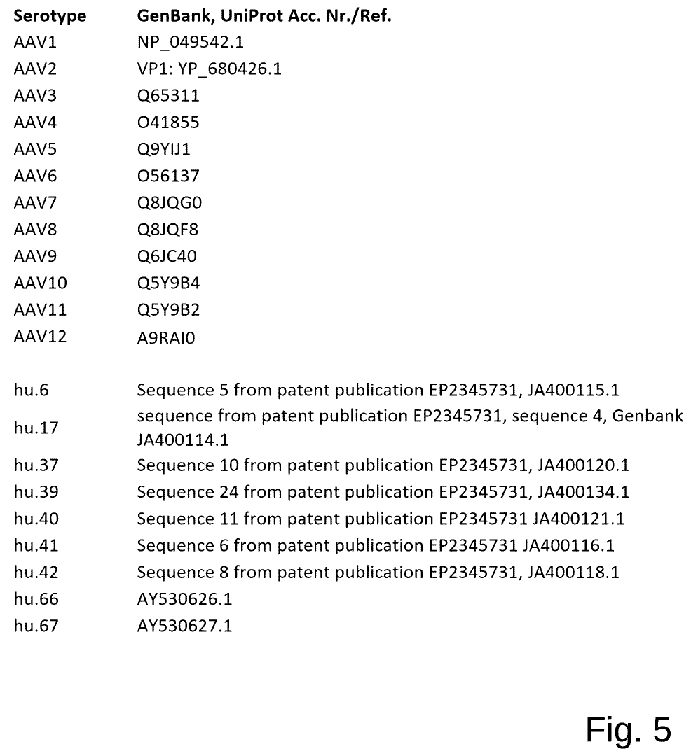

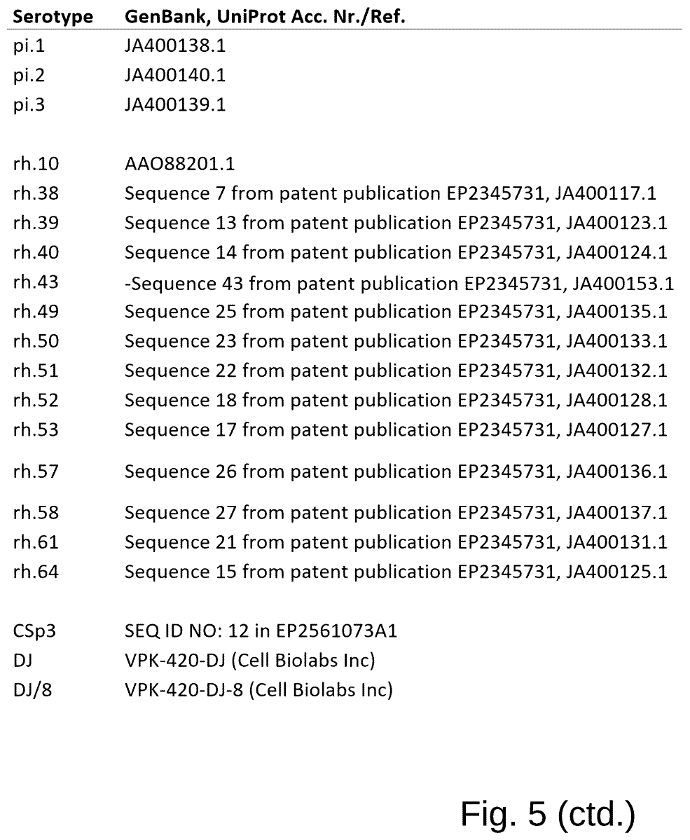

Independently for each occurrence, P is a peptide with a sequence length of 6-13 amino acids, and S is a non-peptide spacer. Independently for each of the peptide n-mers, n is an integer of at least 1, preferably of at least 2, more preferably of at least 3, especially of at least 4. Each of the peptide n-mers is bound to the biopolymer scaffold, preferably via a linker each. Further, independently for each occurrence, P has an amino-acid sequence comprising a sequence fragment with a length of at least six, preferably at least seven, more preferably at least eight, especially at least 9 (or 10, 11, 12 or 13) amino acids of a protein sequence (preferably a capsid protein sequence, nucleocapsid protein sequence, a structural protein sequence, viral envelope- or receptor-binding-protein sequence, tegument protein sequence, viral enzyme sequence, nuclear matrix protein sequence, DNA- or RNA-binding protein sequence, viral packaging protein sequence or viral polymerase sequence, more preferably a capsid protein sequence, nucleocapsid protein sequence, a structural protein sequence, viral envelope- or receptor-binding-protein sequence, or a tegument protein sequence, in particular a capsid protein sequence) of a (non-pathogenic) oncolytic virus (such as AAV or AdV or measles virus), in particular of an AdV hexon protein sequence, an AdV fiber protein sequence, an AdV penton protein sequence, an AdV IIIa protein sequence, an AdV VI protein sequence, an AdV VIII protein sequence or an AdV IX protein sequence or of any one of the capsid protein sequences identified in FIG. 4 and FIG. 5 or of any one of the capsid protein sequences listed in Cearley et al., 2008, or of a capsid protein sequence identified by any one of UniProt P03525, P03526, P03527, P03528, P03529, P0CK31, P11078, P11079, P12418, P12419, P15024, P21270, A1Z0P5, O09800, P03170, P04288, P04289, P04291, P04486, P06477, P06484, P06487, P06491, P08314, P08392, P08543, P10185, P10190, P10191, P10192, P10193, P10200, P10202, P10204, P10205, P10210, P10216, P10218, P10219, P10220, P10225, P10227, P10228, P10229, P10231, P10234, P32888, P68333, P08362, P35971, Q89933, P35974, P35976, P69358, P12576, Q9EMA9 and P03522. Optionally at most three, preferably at most two, most preferably at least one amino acid of the sequence fragment is independently substituted by any other amino acid.

Preferably, at least one occurrence of P is Pₐ and/or at least one occurrence of P is P_{b}. Pₐ is a defined peptide (i.e. a peptide of defined sequence) with a sequence length of 6-13 amino acids, preferably 7-11 amino acids, more preferably 7-9 amino acids. P_{b} is a defined peptide (i.e. a peptide of defined sequence) with a sequence length of 6-13 amino acids, preferably 7-11 amino acids, more preferably 7-9 amino acids.

The present invention also provides a compound comprising
a biopolymer scaffold and at least
a first peptide n-mer which is a peptide dimer of the formula Pₐ—S—Pₐ or Pₐ—S—P_{b}, wherein Pₐ is a defined peptide (i.e. a peptide of defined sequence) with a sequence length of 6-13 amino acids, preferably 7-11 amino acids, more preferably 7-9 amino acids, P_{b} is a defined peptide (i.e. a peptide of defined sequence) with a sequence length of 6-13 amino acids, preferably 7-11 amino acids, more preferably 7-9 amino acids, and S is a non-peptide spacer, wherein the first peptide n-mer is bound to the biopolymer scaffold, preferably via a linker. Pₐ has an amino-acid sequence comprising a sequence fragment with a length of at least six, preferably at least seven, more preferably at least eight, especially at least 9 (or 10, 11, 12 or 13) amino acids of a protein sequence (preferably a capsid protein sequence, nucleocapsid protein sequence, a structural protein sequence, viral envelope- or receptor-binding-protein sequence, tegument protein sequence, viral enzyme sequence, nuclear matrix protein sequence, DNA- or RNA-binding protein sequence, viral packaging protein sequence or viral polymerase sequence, more preferably a capsid protein sequence, nucleocapsid protein sequence, a structural protein sequence, viral envelope- or receptor-binding-protein sequence, or a tegument protein sequence, in particular a capsid protein sequence) of a (non-pathogenic) oncolytic virus, in particular of an AdV hexon protein sequence, an AdV fiber protein sequence, an AdV penton protein sequence, an AdV IIIa protein sequence, an AdV VI protein sequence, an AdV VIII protein sequence or an AdV IX protein sequence or of any one of the capsid protein sequences identified in FIG. 4 and FIG. 5 or of any one of the capsid protein sequences listed in Cearley et al., 2008, or of a capsid protein sequence identified by any one of UniProt P03525, P03526, P03527, P03528, P03529, P0CK31, P11078, P11079, P12418, P12419, P15024, P21270, A1Z0P5, O09800, P03170, P04288, P04289, P04291, P04486, P06477, P06484, P06487, P06491, P08314, P08392, P08543, P10185, P10190, P10191, P10192, P10193, P10200, P10202, P10204, P10205, P10210, P10216, P10218, P10219, P10220, P10225, P10227, P10228, P10229, P10231, P10234, P32888, P68333, P08362, P35971, Q89933, P35974, P35976, P69358, P12576, Q9EMA9 and P03522. Optionally at most three, preferably at most two, most preferably at least one amino acid of the sequence fragment is independently substituted by any other amino acid.

This compound preferably comprises a second peptide n-mer which is a peptide dimer of the formula P_{b}—S—P_{b} or Pₐ—S—P_{b}, wherein the second peptide n-mer is bound to the biopolymer scaffold, preferably via a linker. P_{b} has an amino-acid sequence comprising a sequence fragment with a length of at least six, preferably at least seven, more preferably at least eight, especially at least 9 (or 10, 11, 12 or 13) amino acids of a protein sequence (preferably a capsid protein sequence, nucleocapsid protein sequence, a structural protein sequence, viral envelope- or receptor-binding-protein sequence, tegument protein sequence, viral enzyme sequence, nuclear matrix protein sequence, DNA- or RNA-binding protein sequence, viral packaging protein sequence or viral polymerase sequence, more preferably a capsid protein sequence, nucleocapsid protein sequence, a structural protein sequence, viral envelope- or receptor-binding-protein sequence, or a tegument protein sequence, in particular a capsid protein sequence) of a (non-pathogenic) oncolytic virus, in particular of an AdV hexon protein sequence, an AdV fiber protein sequence, an AdV penton protein sequence, an AdV IIIa protein sequence, an AdV VI protein sequence, an AdV VIII protein sequence or an AdV IX protein sequence or of any one of the capsid protein sequences identified in FIG. 4 and FIG. 5 or of any one of the capsid protein sequences listed in Cearley et al., 2008, or of a capsid protein sequence identified by any one of UniProt P03525, P03526, P03527, P03528, P03529, P0CK31, P11078, P11079, P12418, P12419, P15024, P21270, A1Z0P5, O09800, P03170, P04288, P04289, P04291, P04486, P06477, P06484, P06487, P06491, P08314, P08392, P08543, P10185, P10190, P10191, P10192, P10193, P10200, P10202, P10204, P10205, P10210, P10216, P10218, P10219, P10220, P10225, P10227, P10228, P10229, P10231, P10234, P32888, P68333, P08362, P35971, Q89933, P35974, P35976, P69358, P12576, Q9EMA9 and P03522. Optionally at most three, preferably at most two, most preferably at least one amino acid of the sequence fragment is independently substituted by any other amino acid.

Furthermore, the present invention provides a pharmaceutical composition comprising any one of the aforementioned compounds and at least one pharmaceutically acceptable excipient. Preferably, this pharmaceutical composition is for use in therapy, preferably therapy of a neoplasm such as a benign neoplasm, an in situ neoplasm or a malignant neoplasm, especially therapy of a solid tumor or a hematological malignancy.

In another aspect, the present invention provides a method of (transiently) sequestering (or depleting) one or more antibodies present in an individual, comprising obtaining a pharmaceutical composition as defined herein, the composition being non-immunogenic in the individual, where the one or more antibodies present in the individual are specific for at least one occurrence of P, or for peptide Pₐ and/or peptide P_{b}; and administering the pharmaceutical composition to the individual.

In yet another aspect, the present invention relates to a pharmaceutical composition (i.e. an anti-neoplastic composition), comprising the compound defined herein and further comprising the oncolytic virus (or oncolytic virus-like particle-VLP) and optionally at least one pharmaceutically acceptable excipient. The oncolytic virus typically comprises a peptide fragment with a sequence length of at least six, preferably at least seven, more preferably at least eight, especially at least 9 amino acids. The sequence of at least one occurrence of peptide P, or peptide Pₐ and/or peptide P_{b}, of the compound is at least 70% identical, preferably at least 75% identical, more preferably at least 80% identical, yet more preferably at least 85% identical, even more preferably at least 90% identical, yet even more preferably at least 95% identical, especially completely identical to the sequence of said peptide fragment. Preferably, this pharmaceutical composition is for use in therapy, preferably therapy of a neoplasm such as a benign neoplasm, an in situ neoplasm or an malignant neoplasm, especially therapy of a solid tumor or a hematological malignancy and/or for use in prevention or inhibition of an undesirable (humoral) immune reaction against the oncolytic virus (or VLP).

In even yet another aspect, the present invention provides a method of inhibiting a (undesirable)-especially humoral-immune reaction to a treatment with a anti-neoplastic composition in an individual in need of treatment with the anti-neoplastic composition or of inhibiting neutralization of an oncolytic virus in an anti-neoplastic composition for an individual in need of treatment with the anti-neoplastic composition, comprising obtaining said anti-neoplastic composition; wherein the compound of the anti-neoplastic composition is non-immunogenic in the individual, and administering the anti-neoplastic composition to the individual (preferably systemically or locally, especially systemically).

The neutralizing and inhibitory antibodies against the oncolytic virus also enhance the immune-stimulatory, anti-tumoral activity. Pre-existing inhibitory and at the same time also beneficial antibodies against the oncolytic vector are therefore a double-edged sword.

In other words, on one hand, the immune stimulatory function of oncolytic viruses supports their beneficial effect against cancer cells. On the other hand, the immune stimulatory function may at the same time inhibit oncolytic vector delivery, thereby reducing target access and therapeutic efficacy.

The present invention is suitable for providing a solution in the face of these conflicting goals: In the first phase, nABs are selectively removed by administering the compound of the present invention. This allows for efficient delivery of oncolytic virus vector to the target tissue upon virus administration (especially when the oncolytic virus or VLP is administered systemically—a scenario in which the present invention is especially effective). Since the compound of the present invention will eventually be removed from circulation, the nAb titer will recover after a time window e.g. of few days. The nAbs will then contribute to the required immunostimulatory function of the oncolytic virus.

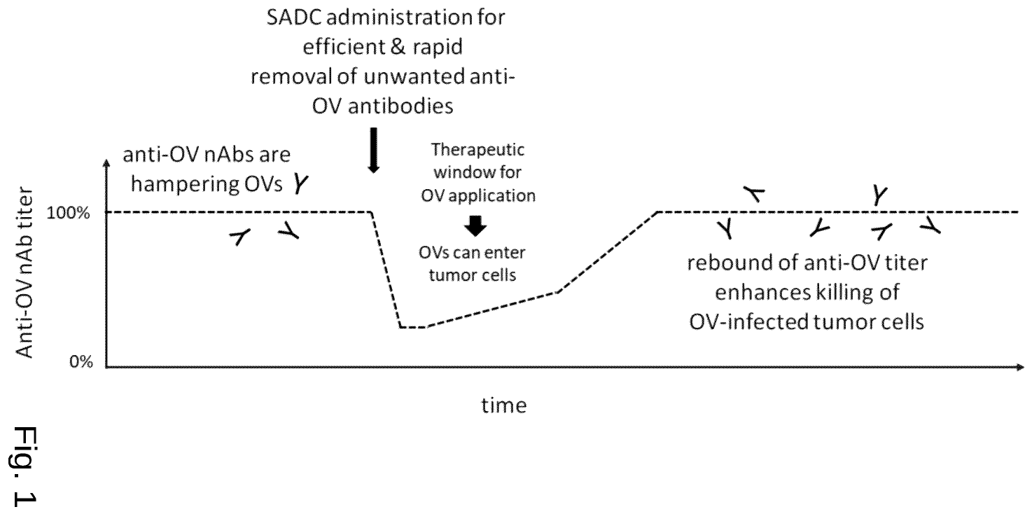

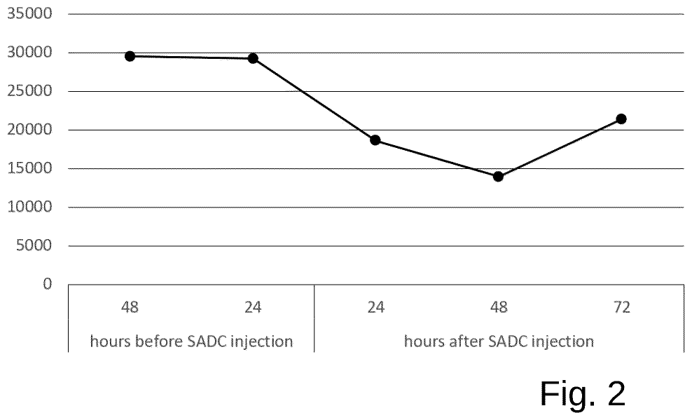

It turned out that the present invention provides a highly suitable transient window for oncolytic virus administration (see e.g. Example 1 and FIGS. 1 and 2). This window is typically diminished when the anti-oncolytic antibody titer recovers. The inventive selective antibody depletion compounds (SADCs, based on the concept generally disclosed first in WO 2020/193486 A1) are therefore well suited for enhancing the effect of locally or systemically applied oncolytic viruses or virus-like particles (VLPs) for targeting solid tumors as well as hematological malignancies.

In addition to their functional utility, polyvalent, peptide-based SADCs with non-immunogenic peptides attached to a non-immunogenic “self”-scaffold protein have another general immunosafety advantage over similar biotherapeutic constructs carrying e.g. DNA or RNA aptamers for sequestering unwanted or harmful antibodies. While it would certainly be possible to manufacture such constructs with a therapeutic effect, in contrast to the present invention, a DNA- or RNA-aptamer based construct would have the inherent risk of acting like a pathogen-associated molecular pattern (PAMP), especially if it would be attached in polyvalent form to a scaffold protein. DNA or RNA aptamer molecules bound to a protein carrier (such as e.g. human albumin or human transferrin) have the inherent risk of triggering the innate immune response via Toll-like receptors (TLRs) and to induce to excess cytokine production with harmful side effects, as reviewed in Bruno, 2018.

The detailed description given below relates to all of the above aspects of the invention unless explicitly excluded.

In general, antibodies are essential components of the humoral immune system, offering protection from infections by foreign organisms including bacteria, viruses, fungi or parasites. However, under certain circumstances-including autoimmune diseases, organ transplantation, blood transfusion or upon administration of biomolecular drugs or gene delivery vectors-antibodies can target the patient's own body (or the foreign tissue or cells or the biomolecular drug or vector just administered), thereby turning into harmful or disease-causing entities. Certain antibodies can also interfere with probes for diagnostic imaging. In the following, such antibodies are generally referred to as “undesired antibodies” or “undesirable antibodies” (as explained above, whether a given antibody is “undesired” may depend on the circumstances; in the case of oncolytic viruses, generally only transient antibody depletion allowing efficient delivery of the virus to the target is preferred).

With few exceptions, selective removal of undesired antibodies has not reached clinical practice. It is presently restricted to very few indications: One of the known techniques for selective antibody removal (although not widely established) is immunoapheresis. In contrast to immunoapheresis (which removes immunoglobulin), selective immunoapheresis involves the filtration of plasma through an extracorporeal, selective antibody-adsorber cartridge that will deplete the undesired antibody based on selective binding to its antigen binding site. Selective immunoapheresis has for instance been used for removing anti-A or anti-B antibodies from the blood prior to ABO-incompatible transplantation or with respect to indications in transfusion medicine (Teschner et al). Selective apheresis was also experimentally applied in other indications, such as neuroimmunological indications (Tetala et al) or myasthenia gravis (Lazaridis et al), but is not yet established in the clinical routine. One reason that selective immunoapheresis is only hesitantly applied is the fact that it is a cost intensive and cumbersome intervention procedure that requires specialized medical care. Moreover, it is not known in the prior art how to deplete undesired antibodies rapidly and efficiently.

Unrelated to apheresis, Morimoto et al. discloses dextran as a generally applicable multivalent scaffold for improving immunoglobulin-binding affinities of peptide and peptidomimetic ligands such as the FLAG peptide. WO 2011/130324 A1 relates to compounds for prevention of cell injury. EP 3 059 244 A1 relates to a C-met protein agonist.

As mentioned, apheresis is applied extracorporeally. By contrast, also several approaches to deplete undesirable antibodies intracorporeally were proposed in the prior art, mostly in connection with certain autoimmune diseases involving autoantibodies or anti-drug antibodies:

Lorentz et al discloses a technique whereby erythrocytes are charged in situ with a tolerogenic payload driving the deletion of antigen-specific T cells. This is supposed to ultimately lead to reduction of the undesired humoral response against a model antigen. A similar approach is proposed in Pishesha et al. In this approach, erythrocytes are loaded ex vivo with a peptide-antigen construct that is covalently bound to the surface and reinjected into the animal model for general immunotolerance induction.

WO 92/13558 A1 relates to conjugates of stable nonimmunogenic polymers and analogs of immunogens that possess the specific B cell binding ability of the immunogen and which, when introduced into individuals, induce humoral anergy to the immunogen. Accordingly, these conjugates are disclosed to be useful for treating antibody-mediated pathologies that are caused by foreign- or self-immunogens. In this connection, see also EP 0 498 658 A2.

Taddeo et al discloses selectively depleting antibody producing plasma cells using anti-CD138 antibody derivatives fused to an ovalbumin model antigen thereby inducing receptor crosslinking and cell suicide in vitro selectively in those cells that express the antibody against the model antigen.

Apitope International NV (Belgium) is presently developing soluble tolerogenic T-cell epitope peptides which may lead to expression of low levels of co-stimulatory molecules from antigen presenting cells inducing tolerance, thereby suppressing antibody response (see e.g. Jansson et al). These products are currently under preclinical and early clinical evaluation, e.g. in multiple sclerosis, Grave's disease, intermediate uveitis, and other autoimmune conditions as well as Factor VIII intolerance.

Similarly, Selecta Biosciences, Inc. (USA) is currently pursuing strategies of tolerance induction by so-called Synthetic Vaccine Particles (SVPs). SVP-Rapamycin is supposed to induce tolerance by preventing undesired antibody production via selectively inducing regulatory T cells (see Mazor et al).

Mingozzi et al discloses decoy adeno-associated virus (AAV) capsids that adsorb antibodies but cannot enter a target cell.

WO 2015/136027 A1 discloses carbohydrate ligands presenting the minimal Human Natural Killer-1 (HNK-1) epitope that bind to anti-MAG (myelin-associated glycoprotein) IgM antibodies, and their use in diagnosis as well as for the treatment of anti-MAG neuropathy. WO 2017/046172 A1 discloses further carbohydrate ligands and moieties, respectively, mimicking glycoepitopes comprised by glycosphingolipids of the nervous system which are bound by anti-glycan antibodies associated with neurological diseases. The document further relates to the use of these carbohydrate ligands/moieties in diagnosis as well as for the treatment of neurological diseases associated with anti-glycan antibodies.

US 2004/0258683 A1 discloses methods for treating systemic lupus erythematosus (SLE) including renal SLE and methods of reducing risk of renal flare in individuals with SLE, and methods of monitoring such treatment. One disclosed method of treating SLE including renal SLE and reducing risk of renal flare in an individual with SLE involves the administration of an effective amount of an agent for reducing the level of anti-double-stranded DNA (dsDNA) antibody, such as a dsDNA epitope as in the form of an epitope-presenting carrier or an epitope-presenting valency platform molecule, to the individual.

US patent no. 5, 637, 454 relates to assays and treatments of autoimmune diseases. Agents used for treatment might include peptides homologous to the identified antigenic, molecular mimicry sequences. It is disclosed that these peptides could be delivered to a patient in order to decrease the amount of circulating antibody with a particular specificity.

US 2007/0026396 A1 relates to peptides directed against antibodies, which cause cold-intolerance, and the use thereof. It is taught that by using the disclosed peptides, in vivo or ex vivo neutralization of undesired autoantibodies is possible. A comparable approach is disclosed in WO 1992/014150 A1 or in WO 1998/030586 A2.

WO 2018/102668 A1 discloses a fusion protein for selective degradation of disease-causing or otherwise undesired antibodies. The fusion protein (termed “Seldeg”) includes a targeting component that specifically binds to a cell surface receptor or other cell surface molecule at near-neutral pH, and an antigen component fused directly or indirectly to the targeting component. Also disclosed is a method of depleting a target antigen-specific antibody from a patient by administering to the patient a Seldeg having an antigen component configured to specifically bind the target antigen-specific antibody.

WO 2015/181393 A1 concerns peptides grafted into sunflower-trypsin-inhibitor-(SFTI-) and cyclotide-based scaffolds. These peptides are disclosed to be effective in autoimmune disease, for instance citrullinated fibrinogen sequences that are grafted into the SFTI scaffold have been shown to block autoantibodies in rheumatoid arthritis and inhibit inflammation and pain. These scaffolds are disclosed to be non-immunogenic.

Erlandsson et al discloses in vivo clearing of idiotypic antibodies with anti-idiotypic antibodies and their derivatives.

Berlin Cures Holding AG (Germany) has proposed an intravenous broad spectrum neutralizer DNA aptamer (see e.g. WO 2016/020377 A1 and WO 2012/000889 A1) for the treatment of dilated cardiomyopathy and other GPCR-autoantibody related diseases that in high dosage is supposed to block autoantibodies by competitive binding to the antigen binding regions of autoantibodies. In general, aptamers did not yet achieve a breakthrough and are still in a preliminary stage of clinical development. The major concerns are still biostability and bioavailability, constraints such as nuclease sensitivity, toxicity, small size and renal clearance. A particular problem with respect to their use as selective antibody antagonists are their propensity to stimulate the innate immune response.

WO 00/33887 A2 discloses methods for reducing circulating levels of antibodies, particularly disease-associated antibodies. The methods entail administering effective amounts of epitope-presenting carriers to an individual. In addition, ex vivo methods for reducing circulating levels of antibodies are disclosed which employ epitope-presenting carriers.

U.S. Pat. No. 6,022,544 A relates to a method for reducing an undesired antibody response in a mammal by administering to the mammal a non-immunogenic construct which is free of high molecular weight immunostimulatory molecules. The construct is disclosed to contain at least two copies of a B cell membrane immunoglobulin receptor epitope bound to a pharmaceutically acceptable non-immunogenic carrier.

WO 2020/193486 A1 relates to a compound for the sequestration of undesirable antibodies in a patient. This document is however silent on oncolysis.

The biopolymer scaffold used in the present invention may be a mammalian biopolymer such as a human biopolymer, a non-human primate biopolymer, a sheep biopolymer, a pig biopolymer, a dog biopolymer or a rodent biopolymer. In particular the biopolymer scaffold is a protein, especially a (non-modified or non-modified with respect to its amino-acid sequence) plasma protein. Preferably, the biopolymer scaffold is a mammalian protein such as a human protein, a non-human primate protein, a sheep protein, a pig protein, a dog protein or a rodent protein. Typically, the biopolymer scaffold is a non-immunogenic and/or non-toxic protein that preferably circulates in the plasma of healthy (human) individuals and can e.g. be efficiently scavenged or recycled by scavenging receptors, such as e.g. present on myeloid cells or on liver sinusoidal endothelial cells (reviewed by Sorensen et al 2015).

WO 2020/193486 A1, in particular examples 1-10 thereof, demonstrates that SADC scaffolds as also used in the present invention are suitable for antibody depletion in a more general context.

According to a particular preference, the biopolymer scaffold is a (preferably human) globulin, preferably selected from the group consisting of immunoglobulins, alpha1-globulins, alpha2-globulins and beta-globulins, in particular immunoglobulin G, haptoglobin and transferrin.

The biopolymer scaffold may also be (preferably human) albumin, hemopexin, alpha-1-antitrypsin, C1 esterase inhibitor, lactoferrin or non-immunogenic (i.e. non-immunogenic in the individual to be treated) fragments of all of the aforementioned proteins, including the globulins.

In another preference, the biopolymer scaffold is an anti-CD163 antibody (i.e. an antibody specific for a CD163 protein) or CD163-binding fragment thereof.

Human CD163 (Cluster of Differentiation 163) is a 130 kDa membrane glycoprotein (formerly called M130) and prototypic class I scavenger receptor with an extracellular portion consisting of nine scavenger receptor cysteine-rich (SRCR) domains that are responsible for ligand binding. CD163 is an endocytic receptor present on macrophages and monocytes, it removes hemoglobin/haptoglobin complexes from the blood but it also plays a role in anti-inflammatory processes and wound healing. Highest expression levels of CD163 are found on tissue macrophages (e.g. Kupffer cells in the liver) and on certain macrophages in spleen and bone marrow. Because of its tissue- and cell-specific expression and entirely unrelated to depletion of undesirable antibodies, CD163 is regarded as a macrophage target for drug delivery of e.g. immunotoxins, liposomes or other therapeutic compound classes (Skytthe et al., 2020).

Monoclonal anti-CD163 antibodies and the SRCR domains they are binding are for instance disclosed in Madsen et al., 2004, in particular FIG. 7. Further anti-CD163 antibodies and fragments thereof are e.g. disclosed in WO 2002/032941 A2 or WO 2011/039510 A2. At least two structurally different binding sites for ligands were mapped by using domain-specific antibodies such as e.g. monoclonal antibody (mAb) EDhu1 (see Madsen et al, 2004). This antibody binds to the third SRCR of CD163 and competes with hemoglobin/haptoglobin binding to CD163. Numerous other antibodies against different domains of CD163 were previously described in the literature, including Mac2-158, KiM8, GHI/61 and RM3/1, targeting SRCR domains 1, 3, 7 and 9, respectively. In addition, conserved bacterial binding sites were mapped and it was demonstrated that certain antibodies were able to inhibit either bacterial binding but not hemoglobin/haptoglobin complex binding and vice versa. This points to different modes of binding and ligand interactions of CD163 (Fabriek et al, 2009; see also citations therein).

Entirely unrelated to depletion of undesirable antibodies, CD163 was proposed as a target for cell-specific drug delivery because of its physiological properties. Tumor-associated macrophages represent one of the main targets where the potential benefit of CD163-targeting is currently explored. Remarkably, numerous tumors and malignancies were shown to correlate with CD163 expression levels, supporting the use of this target for tumor therapy. Other proposed applications include CD163 targeting by anti-drug conjugates (ADCs) in chronic inflammation and neuroinflammation (reviewed in Skytthe et al., 2020). Therefore, CD163-targeting by ADCs notably with dexamethasone or stealth liposome conjugates represents therapeutic principle which is currently studied (Graversen et al., 2012; Etzerodt et al., 2012).

In that context, there are references indicating that anti-CD163 antibodies can be rapidly internalized by endocytosis when applied in vivo. This was shown for example for monoclonal antibody (mAb) Ed-2 (Dijkstra et al., 1985; Graversen et al., 2012) or for mAb Mac2-158/KN2/NRY (Granfeldt et al., 2013). Based on those observations in combination with observations made in the course of the present invention (see in particular example section), anti-CD163 antibodies and CD163-binding turned out to be highly suitable biopolymer scaffolds for depletion/sequestration of undesirable antibodies.

Numerous anti-CD163 antibodies and CD163-binding fragments thereof are known in the art (see e.g. above). These are suitable to be used as a biopolymer scaffold for the present invention. For instance, any anti-CD163 antibody or fragment thereof mentioned herein or in WO 2011/039510 A2 (which is included herein by reference) may be used as a biopolymer scaffold in the invention. Preferably, the biopolymer scaffold of the inventive compound is antibody Mac2-48, Mac2-158, 5C6-FAT, BerMac3, or E10B10 as disclosed in WO 2011/039510, in particular humanised Mac2-48 or Mac2-158 as disclosed in WO 2011/039510 A2.

In a preferred embodiment, the anti-CD163 antibody or CD163-binding fragment thereof comprises a heavy-chain variable (V_{H}) region comprising one or more complementarity-determining region (CDR) sequences selected from the group consisting of SEQ ID NOS: 11-13 of WO 2011/039510 A2.

In addition, or alternatively thereto, in a preferred embodiment, the anti-CD163 antibody or CD163-binding fragment thereof comprises a light-chain variable (V_{L}) region comprising one or more CDR sequences selected from the group consisting of SEQ ID NOs: 14-16 of WO 2011/039510 A2 or selected from the group consisting of SEQ ID NOs: 17-19 of WO 2011/039510 A2.

In a further preferred embodiment, the anti-CD163 antibody or CD163-binding fragment thereof comprises a heavy-chain variable (V_{H}) region comprising or consisting of the amino acid sequence of SEQ ID NO: 20 of WO 2011/039510 A2.

In addition, or alternatively thereto, in a preferred embodiment, the anti-CD163 antibody or CD163-binding fragment thereof comprises a light-chain variable (V_{L}) region comprising or consisting of the amino acid sequence of SEQ ID NO: 21 of WO 2011/039510 A2.

In a further preferred embodiment, the anti-CD163 antibody or CD163-binding fragment thereof comprises a heavy-chain variable (V_{H}) region comprising or consisting of the amino acid sequence of SEQ ID NO: 22 of WO 2011/039510 A2.

In addition, or alternatively thereto, in a preferred embodiment, the anti-CD163 antibody or CD163-binding fragment thereof comprises a light-chain variable (V_{L}) region comprising or consisting of the amino acid sequence of SEQ ID NO: 23 of WO 2011/039510 A2.

In a further preferred embodiment, the anti-CD163 antibody or CD163-binding fragment thereof comprises a heavy-chain variable (V_{H}) region comprising or consisting of the amino acid sequence of SEQ ID NO: 24 of WO 2011/039510 A2.

In addition, or alternatively thereto, in a preferred embodiment, the anti-CD163 antibody or CD163-binding fragment thereof comprises a light-chain variable (V_{L}) region comprising or consisting of the amino acid sequence of SEQ ID NO: 25 of WO 2011/039510 A2.

In the context of the present invention, the anti-CD163 antibody may be a mammalian antibody such as a humanized or human antibody, a non-human primate antibody, a sheep antibody, a pig antibody, a dog antibody or a rodent antibody. In embodiments, the anti-CD163 antibody may monoclonal.

According to a preference, the anti-CD163 antibody is selected from IgG, IgA, IgD, IgE and IgM.

According to a further preference, the CD163-binding fragment is selected from a Fab, a Fab′, a F(ab)2, a Fv, a single-chain antibody, a nanobody and an antigen-binding domain.

CD163 amino acid sequences are for instance disclosed in WO 2011/039510 A2 (which is included here by reference). In the context of the present invention, the anti-CD163 antibody or CD163-binding fragment thereof is preferably specific for a human CD163, especially with the amino acid sequence of any one of SEQ ID NOS: 28-31 of WO 2011/039510 A2.

In a further preferred embodiment, the anti-CD163 antibody or CD163-binding fragment thereof is specific for the extracellular region of CD163 (e.g. for human CD163: amino acids 42-1050 of UniProt Q86VB7, sequence version 2), preferably for an SRCR domain of CD163, more preferably for any one of SRCR domains 1-9 of CD163 (e.g. for human CD163: amino acids 51-152, 159-259, 266-366, 373-473, 478-578, 583-683, 719-819, 824-926 and 929-1029, respectively, of UniProt 086VB7, sequence version 2), even more preferably for any one of SRCR domains 1-3 of CD163 (e.g. for human CD163: amino acids 51-152, 159-259, 266-366, and 373-473, respectively, of UniProt Q86VB7, sequence version 2), especially for SRCR domain 1 of CD163 (in particular with the amino acid sequence of any one of SEQ ID NOS: 1-8 of WO 2011/039510 A2, especially SEQ ID NO: 1 of WO 2011/039510 A2).

In a particular preference, the anti-CD163 antibody or CD163-binding fragment thereof is capable of competing for binding to (preferably human) CD163 with a (preferably human) hemoglobin-haptoglobin complex (e.g. in an ELISA).

In another particular preference, the anti-CD163 antibody or CD163-binding fragment thereof is capable of competing for binding to human CD163 with any of the anti-human CD163 mAbs disclosed herein, in particular Mac2-48 or Mac2-158 as disclosed in WO 2011/039510 A2.

In yet another particular preference, the anti-CD163 antibody or CD163-binding fragment thereof is capable of competing for binding to human CD163 with an antibody having a heavy chain variable (V_{H}) region consisting of the amino acid sequence

(SEQ ID NO: 1)
DVQLQESGPGLVKPSQSLSLTCTVTGYSITSDYAWNWIRQFPGNK
LEWMGYITYSGITNYNPSLKSQISITRDTSKNQFFLQLNSVTTED
TATYYCVSGTYYFDYWGQGTTLTVSS,

and having a light-chain variable (V_{L}) region consisting of the amino acid sequence

(SEQ ID NO: 2)
SVVMTQTPKSLLISIGDRVTITCKASQSVSSDVAWFQQKPGQSPK
PLIYYASNRYTGVPDRFTGSGYGTDFTFTISSVQAEDLAVYFCGQ
DYTSPRTFGGGTKLEIKRA
(e.g. in an ELISA).

Details on competitive binding experiments are known to the person of skilled in the art (e.g. based on ELISA) and are for instance disclosed in WO 2011/039510 A2 (which is included herein by reference).

The epitopes of antibodies E10B10 and Mac2-158 as disclosed in WO 2011/039510 were mapped (see example section). These epitopes are particularly suitable for binding of the anti-CD163 antibody (or CD163-binding fragment thereof) of the inventive compound.

Accordingly, in particularly preferred embodiment, the anti-CD163 antibody or CD163-binding fragment thereof is specific for peptide consisting of 7-25, preferably 8-20, even more preferably 9-15, especially 10-13 amino acids, wherein the peptide comprises the amino acid sequence CSGRVEVKVQEEWGTVCNNGWSMEA (SEQ ID NO; 3) or a 7-24 amino-acid fragment thereof. Preferably, this peptide comprises the amino acid sequence GRVEVKVQEEW (SEQ ID NO: 4), WGTVCNNGWS (SEQ ID NO: 5) or WGTVCNNGW (SEQ ID NO: 6). More preferably, the peptide comprises an amino acid sequence selected from EWGTVCNNGWSME (SEQ ID NO: 7), QEEWGTVCNNGWS (SEQ ID NO: 8), WGTVCNNGWSMEA (SEQ ID NO: 9), EEWGTVCNNGWSM (SEQ ID NO: 10), VQEEWGTVCNNGW (SEQ ID NO: 11), EWGTVCNNGW (SEQ ID NO: 12) and WGTVCNNGWS (SEQ ID NO: 5). Even more preferably, the peptide consists of an amino acid sequence selected from EWGTVCNNGWSME (SEQ ID NO: 7), QEEWGTVCNNGWS (SEQ ID NO: 8), WGTVCNNGWSMEA (SEQ ID NO: 9), EEWGTVCNNGWSM (SEQ ID NO: 10), VQEEWGTVCNNGW (SEQ ID NO: 11), EWGTVCNNGW (SEQ ID NO: 12) and WGTVCNNGWS (SEQ ID NO: 5), optionally with an N-terminal and/or C-terminal cysteine residue.

Accordingly, in another particularly preferred embodiment, the anti-CD163 antibody or CD163-binding fragment thereof is specific for a peptide consisting of 7-25, preferably 8-20, even more preferably 9-15, especially 10-13 amino acids, wherein the peptide comprises the amino acid sequence DHVSCRGNESALWDCKHDGWG (SEQ ID NO: 13) or a 7-20 amino-acid fragment thereof. Preferably, this peptide comprises the amino acid sequence ESALW (SEQ ID NO: 14) or ALW. More preferably, the peptide comprises an amino acid sequence selected from ESALWDC (SEQ ID NO: 15), RGNESALWDC (SEQ ID NO: 16), SCRGNESALW (SEQ ID NO: 17), VSCRGNESALWDC (SEQ ID NO: 18), ALWDCKHDGW (SEQ ID NO: 19), DHVSCRGNESALW (SEQ ID NO: 20), CRGNESALWD (SEQ ID NO: 21), NESALWDCKHDGW (SEQ ID NO: 22) and ESALWDCKHDGWG (SEQ ID NO: 23). Even more preferably, the peptide consists of an amino acid sequence selected from ESALWDC (SEQ ID NO: 15), RGNESALWDC (SEQ ID NO: 16), SCRGNESALW (SEQ ID NO: 17), VSCRGNESALWDC (SEQ ID NO: 18), ALWDCKHDGW (SEQ ID NO: 19), DHVSCRGNESALW (SEQ ID NO: 20), CRGNESALWD (SEQ ID NO: 21), NESALWDCKHDGW (SEQ ID NO: 22) and ESALWDCKHDGWG (SEQ ID NO: 23), optionally with an N-terminal and/or C-terminal cysteine residue,

Accordingly, in another particularly preferred embodiment, the anti-CD163 antibody or CD163-binding fragment thereof is specific for a peptide consisting of 7-25, preferably 8-20, even more preferably 9-15, especially 10-13 amino acids, wherein the peptide comprises the amino acid sequence SSLGGTDKELRLVDGENKCS (SEQ ID NO: 24) or a 7-19 amino-acid fragment thereof. Preferably, this peptide comprises the amino acid sequence SSLGGTDKELR (SEQ ID NO: 25) or SSLGG (SEQ ID NO: 26). More preferably, the peptide comprises an amino acid sequence selected from SSLGGTDKELR (SEQ ID NO: 25), SSLGGTDKEL (SEQ ID NO: 27), SSLGGTDKE (SEQ ID NO: 28), SSLGGTDK (SEQ ID NO: 29), SSLGGTD (SEQ ID NO: 30), SSLGGT (SEQ ID NO: 31) and SSLGG (SEQ ID NO: 26). Even more preferably, the peptide consists of an amino acid sequence selected from SSLGGTDKELR (SEQ ID NO: 25), SSLGGTDKEL (SEQ ID NO: 27), SSLGGTDKE (SEQ ID NO: 28), SSLGGTDK (SEQ ID NO: 29), SSLGGTD (SEQ ID NO: 30), SSLGGT (SEQ ID NO: 31) and SSLGG (SEQ ID NO: 26), optionally with an N-terminal and/or C-terminal cysteine residue.

The peptides (or peptide n-mers) are preferably covalently conjugated (or covalently bound) to the biopolymer scaffold via a (non-immunogenic) linker known in the art such as for example amine-to-sulfhydryl linkers and bifunctional NHS-PEG-maleimide linkers or other linkers known in the art. Alternatively, the peptides (or peptide n-mers) can be bound to the epitope carrier scaffold e.g. by formation of a disulfide bond between the protein and the peptide (which is also referred to as “linker” herein), or using non-covalent assembly techniques, spontaneous isopeptide bond formation or unnatural amino acids for bio-orthogonal chemistry via genetic code expansion techniques (reviewed by Howarth et al 2018 and Lim et al 2016). Covalent and non-covalent bioconjugation strategies suitable for the present invention are also discussed e.g. in Sunasee et al, 2014.

The compound of the present invention may comprise e.g. at least two, preferably between 3 and 40 copies of one or several different peptides (which may be present in different forms of peptide n-mers as disclosed herein). The compound may comprise one type of epitopic peptide (in other words: antibody-binding peptide or paratope-binding peptide), however the diversity of epitopic peptides bound to one biopolymer scaffold molecule can be a mixture of e.g. up to 8 different epitopic peptides.

Typically, since the peptides present in the inventive compound specifically bind to selected undesired antibodies, their sequence is usually selected and optimized such that they provide specific binding in order to guarantee selectivity of undesired antibody depletion from the blood. For this purpose, the peptide sequence of the peptides typically corresponds to the entire epitope sequence or portions of the undesired antibody epitope. The peptides used in the present invention can be further optimized by exchanging one, two or up to four amino-acid positions, allowing e.g. for modulating the binding affinity to the undesired antibody that needs to be depleted. Such single or multiple amino-acid substitution strategies that can provide “mimotopes” with increased binding affinity and are known in the field and were previously developed using phage display strategies or peptide microarrays. In other words, the peptides used in the present invention do not have to be completely identical to the native epitope sequences of the undesired antibodies.

Typically, the peptides used in the compound of the present invention (e.g. peptide P or Pₐ or P_{b}) are composed of one or more of the 20 amino acids commonly present in mammalian proteins. In addition, the amino acid repertoire used in the peptides may be expanded to post-translationally modified amino acids e.g. affecting antigenicity of proteins such as post translational modifications, in particular oxidative post translational modifications (see e.g. Ryan 2014) or modifications to the peptide backbone (see e.g. Müller 2018), or to non-natural amino acids (see e.g. Meister et al 2018). These modifications may also be used in the peptides e.g. to adapt the binding interaction and specificity between the peptide and the variable region of an undesired antibody. In particular, epitopes (and therefore the peptides used in the compound of the present invention) can also contain citrulline as for example in autoimmune diseases. Furthermore, by introducing modifications into the peptide sequence the propensity of binding to an HLA molecule may be reduced, the stability and the physicochemical characteristics may be improved or the affinity to the undesired antibody may be increased.

In many cases, the undesired antibody that is to be depleted is oligo- or polyclonal (e.g. autoantibodies, ADAs or alloantibodies are typically poly- or oligoclonal), implying that undesired (polyclonal) antibody epitope covers a larger epitopic region of a target molecule. To adapt to this situation, the compound of the present invention may comprise a mixture of two or several epitopic peptides (in other words: antibody-binding peptides or paratope-binding peptides), thereby allowing to adapt to the polyclonality or oligoclonality of an undesired antibody.

Such poly-epitopic compounds of the present invention can effectively deplete undesired antibodies and are more often effective than mono-epitopic compounds in case the epitope of the undesired antibody extends to larger amino acid sequence stretches.

It is advantageous if the peptides used for the inventive compound are designed such that they will be specifically recognized by the variable region of the undesired antibodies to be depleted. The sequences of peptides used in the present invention may e.g. be selected by applying fine epitope mapping techniques (i.e. epitope walks, peptide deletion mapping, amino acid substitution scanning using peptide arrays such as described in Carter et al 2004, and Hansen et al 2013) on the undesired antibodies.

The present invention is suitable for (transiently) depleting antibodies against all oncolytic viruses, such as the ones discussed in Zeng et al. 2021. The oncolytic virus may thus be selected e.g. from any one discussed in Zeng et al., 2021.

Wild-type AAVs are typically non-pathogenic and only capable of replicating in the presence of helper viruses. One big advantage of this class of viral vectors is that they maintain long term, sustained gene expression in the host cell, making them ideal for oncolytic gene delivery. Numerous natural subtypes have been isolated showing serological differences and unique tropism in vivo and in vitro. AAV vectors are well suited for targeting different cell types. Importantly, they typically do not integrate into the genome of the host cell (Colella et al, 2017).

AAVs have been extensively studied for oncolytic approaches. For instance, Feiner et al., 2020, discloses tumor targeting by presenting EGFR-binding peptides in the AAV capsid. Kuklik et al., 2021, relates to the development of a bispecific antibody-based platform for retargeting of capsid-modified AAV vectors. Table I of Lundstrom, 2018, contains preclinical examples of anti-neoplastic therapy with oncolytic AAV, among other oncolytic viruses.

To date, many different serotypes and variants are well studied including AAV2, AAV5 or AAV8. Li (Li et al, 2020) provides an extensive review about AAV vectors. New concepts of improving gene expression, tissue specificity, genome stability, combined with capsid engineering can be found in Domenger (Domenger et al, 2019).

Much effort was invested into engineering improved AAV capsid variants to change their biological properties including tropism and safety. However, susceptibility to antibody neutralization by preexisting antibodies of the patient remains a major challenge, see e.g. Costa Verdera et al, 2020.

Kruzik et al, 2019, investigated the prevalence of neutralizing antibodies against various AAV serotypes in different patient cohorts. It was found, for example, that neutralizing antibodies against AAV2 were most abundant at levels up to 74% of the population. Antibodies against AAV8 were found for example in up to 63%. Natural antibodies against AAV5 (up to 59%) and AAV1 (27%) were less abundant. Interestingly, most people tested exhibited antibodies against more than one serotype. A comprehensive review was published by Ronzitti et al, 2020.

Tseng et al, 2014, reviewed epitopes of anti-AAV antibodies found with human serum and monoclonal antibodies. The interaction between preexisting or induced anti-AAV antibodies and virus capsid proteins has mainly been investigated by mutation analysis, by peptide insertions or by peptide scanning and several approaches were tested to develop AAV variants that improve tropism and that escape humoral immune response. These strategies include directed evolution, structure-based approaches, the engineering of chimeric AAV vectors (for example Bennett et al, 2020) or by displaying peptides of the surface of AAV vectors (Borner et al, 2020). Other proposed strategies to avoid the negative impact of neutralizing antibodies include modifying the route of administration (Mimuro et al, 2013), the discovery of new serotypes and variants (Salganik et al, 2015), the reduction of immunogenicity by PEGylation or polymer technologies (Balakrishnan et al, 2019) or the use of capsid decoys intra-(Mingozzi et al, 2013) or extracorporeally (Bertin et al, 2020). Mechanisms of immunogenicity and their clinical impact were extensively reviewed by Monahan et al, 2021.

US 2013/0259885 A1 relates to immunomodulation with peptides containing epitopes recognized by CD4+ natural killer T cells. This is taught to be suitable for increasing efficiency of gene therapy. WO 2005/023848 A2 discloses administration of peptides to patients for increasing efficiency of an adenoviral vector.

WO 2019/018439 A1 relates to the removal of AAV-neutralizing antibodies from a subject by apheresis prior to administering recombinant AAV comprising a heterologous polynucleotide to the subject. Bertin et al, 2020, discloses a similar apheresis approach. Further along similar lines, WO 00/20041 A2 relates to methods of enhancing the effectiveness of therapeutic viral agents by extracorporeal removal of anti-AdV-antibodies with affinity columns based on AdV subunits (i.e. selective apheresis).

Neutralizing antibodies are not only problematic with respect to AAV-based vectors. To date, Adenovirus (AdV) serotype 5 (Ad5), as the prototypic adenoviral vector, was tested in more than 400 clinical trials. The approved oncolytic virus-based product Gendicine is also based on Ad5 (Zhang et al, 2018). Remarkably, up to 80% of the population carries neutralizing antibodies against Ad5 which has a negative impact on the efficacy.

Neutralizing antibodies and epitopes against all types of AdV and other viral vectors for therapy were previously described e.g. in Tian et al, 2018; Wang et al, 2019; Fausther-Bovendo et al, 2014; and Mok et al, 2020). As with AAV vectors, much effort was made to circumvent pre-existing anti vector immunity by engineering and fine mapping of epitopes of adenoviral vectors (Roberts et al, 2006).

According to a preferred embodiment, the oncolytic virus is an AdV vector or an AAV vector, preferably specific for a human host.

In another preferred embodiment, the oncolytic virus a measles virus, a Herpes simplex virus (HSV), a poxvirus such as Vaccinia virus, a reovirus, a Newcastle disease virus (NVD), a rhabdovirus, a coxsackievirus, a lentivirus, an alphavirus such as Semliki Forest virus, a vesicular stomatitis virus (VSS), a myxoma virus (MYXV), a mengovirus, a bovine viral diarrhea virus (BVDV), a chimeric oncolytic virus, a parvovirus, a picornavirus or a flavivirus; preferably specific for a human host.

In another preference, the sequence fragment as used herein comprises an epitope or epitope part (e.g. at least six, especially at least seven or even at least eight amino acids) of an AdV protein (preferably a capsid protein sequence, nucleocapsid protein sequence, a structural protein sequence, viral envelope- or receptor-binding-protein sequence, tegument protein sequence, viral enzyme sequence, nuclear matrix protein sequence, DNA- or RNA-binding protein sequence, viral packaging protein sequence or viral polymerase sequence, more preferably a capsid protein sequence, nucleocapsid protein sequence, a structural protein sequence, viral envelope- or receptor-binding-protein sequence, or a tegument protein sequence, in particular a capsid protein sequence) or of an AAV protein (preferably a capsid protein sequence, nucleocapsid protein sequence, a structural protein sequence, viral envelope- or receptor-binding-protein sequence, tegument protein sequence, viral enzyme sequence, nuclear matrix protein sequence, DNA- or RNA-binding protein sequence, viral packaging protein sequence or viral polymerase sequence, more preferably a capsid protein sequence, nucleocapsid protein sequence, a structural protein sequence, viral envelope- or receptor-binding-protein sequence, or a tegument protein sequence, in particular a capsid protein sequence) (see also Examples), in particular wherein the AAV is one of AAV-8, AAV-9, AAV-6, AAV-2 or AAV-5, or of one of the following viral proteins identified by their UniProt accession code:
A9RAI0, B5SUY7, O41855, O56137, O56139, P03135, P04133, P04882, P08362, P10269, P12538, P69353, Q5Y9B2, Q5Y9B4, Q65311, Q6JC40, Q6VGT5, Q8JQF8, Q8JQG0, Q98654, Q9WBP8, Q9YIJ1,
or of an AdV hexon protein, an AdV fiber protein, an AdV penton protein, an AdV IIIa protein, an AdV VI protein, an AdV VIII protein or an AdV IX protein or of any one of the capsid proteins identified in FIG. 4 and FIG. 5 or of any one of the capsid proteins listed in Cearley et al., 2008, or of a capsid protein sequence identified by any one of UniProt P03525, P03526, P03527, P03528, P03529, P0CK31, P11078, P11079, P12418, P12419, P15024, P21270, A1Z0P5, O09800, P03170, P04288, P04289, P04291, P04486, P06477, P06484, P06487, P06491, P08314, P08392, P08543, P10185, P10190, P10191, P10192, P10193, P10200, P10202, P10204, P10205, P10210, P10216, P10218, P10219, P10220, P10225, P10227, P10228, P10229, P10231, P10234, P32888, P68333, P08362, P35971, Q89933, P35974, P35976, P69358, P12576, Q9EMA9 and P03522.

Particularly suitable epitopes for depleting neutralizing antibodies (against AAV and AdV) were found in epitope screens and screens of human sera (see also Examples). Accordingly, in a preferment, the sequence fragment as used herein comprises a sequence of at least 4 or at least 5 or at least 6, preferably at least 7, more preferably at least 8, even more preferably at least 9, yet even more preferably at least 10 consecutive amino acids selected from:
the group of AdV sequences ETGPPTVPFLTPPF (SEQ ID NO: 32), HDSKLSIATQGPL (SEQ ID NO: 33), LNLRLGQGPLFINSAHNLDINY (SEQ ID NO: 34), VDPMDEPTLLYVLFEVEDVV (SEQ ID NO: 35), MKRARPSEDTENPVYPYD (SEQ ID NO: 36), ISGTVQSAHLIIRFD (SEQ ID NO: 37), LGQGPLFINSAHNLDINYNKGLYLF (SEQ ID NO: 38), SYPFDAQNOLNLRLGQGPLFIN (SEQ ID NO: 39), GDTTPSAYSMSFSWDWSGHNYIN (SEQ ID NO: 40), VLLNNSFLDPEYWNFRN (SEQ ID NO: 41), HNYINEIFATSSYTFSYIA (SEQ ID NO: 42), DEAATALEINLEEEDDDNEDEVDEQAEQQKTH (SEQ ID NO: 43), INLEEEDDDNEDEVDEQAEQ (SEQ ID NO: 44), DNEDEVDEQAEQQKTHVF (SEQ ID NO: 45), EWDEAATALEINLEE (SEQ ID NO: 46), PKVVLYSEDVDIETPDTHISYMP (SEQ ID NO: 47), YIPESYKDRMYSFERNF (SEQ ID NO: 48), DSIGDRTRYFSMW (SEQ ID NO: 49), SYKDRMYSFERNF (SEQ ID NO: 50), and FLVOMLANYNIGYOGFY (SEQ ID NO: 51), or
the group of AAV sequences WONRDVYLQGPIWAKIP (SEQ ID NO: 52), DNTYFGYSTPWGYFDFNRFHC (SEQ ID NO: 53), MANQAKNWLPGPCY (SEQ ID NO: 54), LPYVLGSAHQGCLPPFP (SEQ ID NO: 55), NGSQAVGRSSFYCLEYF (SEQ ID NO: 56), PLIDQYLYYL (SEQ ID NO: 57), EERFFPSNGILIF (SEQ ID NO: 58), ADGVGSSSGNWHC (SEQ ID NO: 59), SEQ ID NOs: 383-1891 (see Table 1)-preferably group III of Table 1, more preferably group II of Table 1, especially group I of Table 1—and SEQ ID NOs: 1892-2063 (see Table 2)-preferably group I of Table 2—and sequences of group II or III of Table 3 (in particular SEQ ID NOs: 2064-2103), more preferably sequences of group I of Table 3,
or the group of sequences of Table 4, in particular the group of sequences identified by SEQ ID NOs: 2104-2190,
or the group of sequences of Table 5.

It is particularly preferred that Pₐ and/or P_{b} or, independently for each occurrence, P comprises a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences consisting of GPPTVPFLTP (SEQ ID NO: 60), ETGPPTVPFLTPP (SEQ ID NO: 61), TGPPTVPFLT (SEQ ID NO: 62), PTVPFLTPPF (SEQ ID NO: 63), HDSKLSIATQGPL (SEQ ID NO: 64), SIATOGP (SEQ ID NO: 65), NLRLGOGPLF (SEQ ID NO: 66), QGPLFINSAH (SEQ ID NO: 67), PLFINSAHNLD (SEQ ID NO: 68), LGQGPLF (SEQ ID NO: 69), LNLRLGQGPL (SEQ ID NO: 70), GQGPLFI (SEQ ID NO: 71), NLRLGQGPLFINS (SEQ ID NO: 72), LFINSAHNLDINY (SEQ ID NO: 73), FINSAHNLDI (SEQ ID NO: 74), LRLGQGPLFI (SEQ ID NO: 75), GPLFINSAHN (SEQ ID NO: 76), DEPTLLYVLFEVF (SEQ ID NO: 77), TLLYVLFEVF (SEQ ID NO: 78), DEPTLLYVLF (SEQ ID NO: 79), TLLYVLFEVEDVV (SEQ ID NO: 80), TLLYVLF (SEQ ID NO: 81), MDEPTLLYVLFEV (SEQ ID NO: 82), EPTLLYVLFE (SEQ ID NO: 83), DPMDEPTLLYVLF (SEQ ID NO: 84), LLYVLFEVED (SEQ ID NO: 85), YVLFEVFDVV (SEQ ID NO: 86), PTLLYVLFEV (SEQ ID NO: 87), PTLLYVLFEVFDV (SEQ ID NO: 88), LYVLFEVEDV (SEQ ID NO: 89), EPTLLYVLFEVED (SEQ ID NO: 90), LYVLFEV (SEQ ID NO: 91), PMDEPTLLYVLFE (SEQ ID NO: 92), LLYVLFE (SEQ ID NO: 93), VDPMDEPTLLYVL (SEQ ID NO: 94), YVLFEVF (SEQ ID NO: 95), PTLLYVL (SEQ ID NO: 96), MKRARPSEDTF (SEQ ID NO: 97), KRARPSEDTF (SEQ ID NO: 98), MKRARPSEDT (SEQ ID NO: 99), MKRARPSEDTEN (SEQ ID NO: 100), ARPSEDTENP (SEQ ID NO: 101), RARPSEDTEN (SEQ ID NO: 102), RPSEDTE (SEQ ID NO: 103), MKRARPSEDTENP (SEQ ID NO: 104), RARPSEDTENPVY (SEQ ID NO: 105), ARPSEDT (SEQ ID NO: 106), EDTENPVYPY (SEQ ID NO: 107), RPSEDTENPVYPY (SEQ ID NO: 108), KRARPSEDTENPV (SEQ ID NO: 109), DTFNPVY (SEQ ID NO: 110), RPSEDTENPV (SEQ ID NO: 111), PSEDTFNPVY (SEQ ID NO: 112), DTENPVYPYD (SEQ ID NO: 113), VQSAHLI IRF (SEQ ID NO: 114), AHLIIRF (SEQ ID NO: 115), SGTVQSAHLIIRF (SEQ ID NO: 116), TVQSAHLIIR (SEQ ID NO: 117), HLIIRFD (SEQ ID NO: 118), SAHLIIR (SEQ ID NO: 119), QSAHLIIRED (SEQ ID NO: 120), ISGTVQSAHLIIR (SEQ ID NO: 121), GTVQSAHLII (SEQ ID NO: 122), GTVQSAHLIIRED (SEQ ID NO: 123), QSAHLII (SEQ ID NO: 124), HNLDINY (SEQ ID NO: 125), LFINSAHNLDINY (SEQ ID NO: 126), NLDINYNKGLYLF (SEQ ID NO: 127), FVSPNG (SEQ ID NO: 128), NYINEIF (SEQ ID NO: 129), NKGLYLF (SEQ ID NO: 130), INYNKGLYLF (SEQ ID NO: 131), NSAHNLDINY (SEQ ID NO: 132), WDWSGHNYINEIF (SEQ ID NO: 133), SGHNYINEIF (SEQ ID NO: 134), LGTGLSF (SEQ ID NO: 135), PFLTPPF (SEQ ID NO: 136), LGQGPLF (SEQ ID NO: 137), NLRLGOGPLF (SEQ ID NO: 138), NOLNLRLGQGPLF (SEQ ID NO: 139), GOGPLFI (SEQ ID NO: 140), QLNLRLGQGPLFI (SEQ ID NO: 141), SYPFDAQNOLNLR (SEQ ID NO: 142), YPFDAQNOLNLRL (SEQ ID NO: 143), LRLGQGPLFI (SEQ ID NO: 144), NQLNLRL (SEQ ID NO: 145), FDAQNOLNLR (SEQ ID NO: 146), QNQLNLR (SEQ ID NO: 147), QGPLFIN (SEQ ID NO: 148), PFDAQNQLNLRLG (SEQ ID NO: 149), DAQNQLNLRL (SEQ ID NO: 150), RLGQGPLFIN (SEQ ID NO: 151), QLNLRLG (SEQ ID NO: 152), FDAQNQLNLRLGQ (SEQ ID NO: 153), LNLRLGQGPLFIN (SEQ ID NO: 154), AQNOLNLRLG (SEQ ID NO: 155), AQNOLNL (SEQ ID NO: 156), LNLRLGQ (SEQ ID NO: 157), SYPFDAQNOL (SEQ ID NO: 158), PFDAQNOLNL (SEQ ID NO: 159), YSMSFSW (SEQ ID NO: 160), TPSAYSMSFSWDW (SEQ ID NO: 161), MSFSWDW (SEQ ID NO: 162), PSAYSMSFSW (SEQ ID NO: 163), DTTPSAYSMSFSW (SEQ ID NO: 164), TTPSAYSMSF (SEQ ID NO: 165), YSMSFSWDWS (SEQ ID NO: 166), TGDTTPSAYSMSF (SEQ ID NO: 167), FSWDWSGHNY (SEQ ID NO: 168), SFSWDWS (SEQ ID NO: 169), SAYSMSF (SEQ ID NO: 170), SFSWDWSGHN (SEQ ID NO: 171), SAYSMSFSWD (SEQ ID NO: 172), SMSFSWD (SEQ ID NO: 173), SWDWSGHNYI (SEQ ID NO: 174), AYSMSFS (SEQ ID NO: 175), SMSFSWDWSGHNY (SEQ ID NO: 176), FSWDWSG (SEQ ID NO: 177), SWDWSGH (SEQ ID NO: 178), FLDPEYWNER (SEQ ID NO: 179), SFLDPEYWNF (SEQ ID NO: 180), PEYWNER (SEQ ID NO: 181), LNNSFLDPEYWNF (SEQ ID NO: 182), NNSFLDPEYWNER (SEQ ID NO: 183), FLDPEYW (SEQ ID NO: 184), DPEYWNF (SEQ ID NO: 185), NNSFLDPEYW (SEQ ID NO: 186), VLLNNSFLDPEYW (SEQ ID NO: 187), EYWNFRN (SEQ ID NO: 188), LNNSELDPEY (SEQ ID NO: 189), LDPEYWNERN (SEQ ID NO: 190), LNNSFLD (SEQ ID NO: 191), NSFLDPEYWN (SEQ ID NO: 192), SSYTFSY (SEQ ID NO: 193), FATSSYTFSY (SEQ ID NO: 194), YINEIFATSSYTF (SEQ ID NO: 195), SYTFSYI (SEQ ID NO: 196), ATSSYTF (SEQ ID NO: 197), EIFATSSYTF (SEQ ID NO: 198), NEIFATSSYTESY (SEQ ID NO: 199), ATSSYTFSYI (SEQ ID NO: 200), HNYINEIFATSSY (SEQ ID NO: 201), IFATSSY (SEQ ID NO: 202), INEIFATSSY (SEQ ID NO: 203), NYINEIFATSSYT (SEQ ID NO: 204), YINEIFA (SEQ ID NO: 205), YTESYIA (SEQ ID NO: 206), EIFATSSYTESYI (SEQ ID NO: 207), ALEINLEEEDDDN (SEQ ID NO: 208), ATALEINLEEEDD (SEQ ID NO: 209), EAATALEINLEEE (SEQ ID NO: 210), LEINLEE (SEQ ID NO: 211), TALEINLEEEDDD (SEQ ID NO: 212), EINLEEE (SEQ ID NO: 213), ALEINLEEED (SEQ ID NO: 214), LEINLEEEDD (SEQ ID NO: 215), TALEINLEEE (SEQ ID NO: 216), DEAATALEINLEE (SEQ ID NO: 217), LEINLEEEDDDNE (SEQ ID NO: 218), AATALEINLEEED (SEQ ID NO: 219), EINLEEEDDD (SEQ ID NO: 220), ATALEINLEE (SEQ ID NO: 221), INLEEEDDDN (SEQ ID NO: 222), NLEEEDDDNE (SEQ ID NO: 223), DEVDEQA (SEQ ID NO: 224), EDDDNEDEVDEQA (SEQ ID NO: 225), DDNEDEVDEQAEQ (SEQ ID NO: 226), EVDEQAE (SEQ ID NO: 227), DNEDEVDEQA (SEQ ID NO: 228), VDEQAEQ (SEQ ID NO: 229), EDEVDEQAEQQKT (SEQ ID NO: 230), EDEVDEQAEQ (SEQ ID NO: 231), DEVDEQAEQQKTH (SEQ ID NO: 232), NEDEVDEQAEQOK (SEQ ID NO: 233), DEVDEQAEQQ (SEQ ID NO: 234), EINLEEEDDDNED (SEQ ID NO: 235), NLEEEDDDNEDEV (SEQ ID NO: 236), INLEEED (SEQ ID NO: 237), LEEEDDDNED (SEQ ID NO: 238), INLEEEDDDNEDE (SEQ ID NO: 239), DDDNEDEVDEQAE (SEQ ID NO: 240), LEEEDDDNEDEVD (SEQ ID NO: 241), DDNEDEVDEQ (SEQ ID NO: 242), EDDDNED (SEQ ID NO: 243), NLEEEDD (SEQ ID NO: 244), DDNEDEV (SEQ ID NO: 245), DDDNEDEVDE (SEQ ID NO: 246), DDDNEDE (SEQ ID NO: 247), EEEDDDNEDE (SEQ ID NO: 248), EEDDDNE (SEQ ID NO: 249), EDDDNEDEVD (SEQ ID NO: 250), EDEVDEQ (SEQ ID NO: 251), EEDDDNEDEVDEQ (SEQ ID NO: 252), EEDDDNEDEV (SEQ ID NO: 253), EEEDDDNEDEVDE (SEQ ID NO: 254), EVDEQAEQOK (SEQ ID NO: 255), DNEDEVDEQAEQQ (SEQ ID NO: 256), VDEQAEQQKT (SEQ ID NO: 257), EVDEQAEQQKTHV (SEQ ID NO: 258), VDEQAEQQKTHVF (SEQ ID NO: 259), ALEINLE (SEQ ID NO: 260), WDEAATALEINLE (SEQ ID NO: 261), AATALEINLE (SEQ ID NO: 262), EWDEAATALEINL (SEQ ID NO: 263), EAATALEINL (SEQ ID NO: 264), LYSEDVDIET (SEQ ID NO: 265), LYSEDVDIETPDT (SEQ ID NO: 266), KVVLYSEDVDIET (SEQ ID NO: 267), IETPDTH (SEQ ID NO: 268), VDIETPDTHI (SEQ ID NO: 269), VLYSEDVDIE (SEQ ID NO: 270), DVDIETPDTHISY (SEQ ID NO: 271), VVLYSEDVDIETP (SEQ ID NO: 272), SEDVDIETPDTHI (SEQ ID NO: 273), ETPDTHI (SEQ ID NO: 274), VLYSEDVDIETPD (SEQ ID NO: 275), DVDIETPDTH (SEQ ID NO: 276), DIETPDTHIS (SEQ ID NO: 277), EDVDIETPDTHIS (SEQ ID NO: 278), IETPDTHISY (SEQ ID NO: 279), YSEDVDIETPDTH (SEQ ID NO: 280), VDIETPDTHISYM (SEQ ID NO: 281), PKVVLYSEDVDIE (SEQ ID NO: 282), DIETPDT (SEQ ID NO: 283), DIETPDTHISYMP (SEQ ID NO: 284), EDVDIETPDT (SEQ ID NO: 285), ETPDTHISYM (SEQ ID NO: 286), IETPDTHISYMP (SEQ ID NO: 287), DRMYSFFRNF (SEQ ID NO: 288), DRMYSFF (SEQ ID NO: 289), YSFERNF (SEQ ID NO: 290), IPESYKDRMYSFF (SEQ ID NO: 291), SYKDRMYSFF (SEQ ID NO: 292), ESYKDRMYSF (SEQ ID NO: 293), KDRMYSF (SEQ ID NO: 294), YIPESYKDRMYSF (SEQ ID NO: 295), PESYKDRMYSFFR (SEQ ID NO: 296), YKDRMYSFFR (SEQ ID NO: 297), TRYFSMW (SEQ ID NO: 298), GDRTRYF (SEQ ID NO: 299), DSIGDRTRYF (SEQ ID NO: 300), DSIGDRTRYFSMW (SEQ ID NO: 301), GDRTRYFSMW (SEQ ID NO: 302), DRMYSFFRNF (SEQ ID NO: 303), SYKDRMYSFERNF (SEQ ID NO: 304), NYNIGYQGFY (SEQ ID NO: 305), ANYNIGYQGF (SEQ ID NO: 306), MLANYNIGYOGFY (SEQ ID NO: 307), IGYQGFY (SEQ ID NO: 308), FLVOMLANYNIGY (SEQ ID NO: 309), NIGYQGF (SEQ ID NO: 310) and QMLANYNIGYQGF (SEQ ID NO: 311), optionally wherein at most three, preferably at most two, most preferably at least one amino acid is independently substituted by any other amino acid.

In another preferred embodiment, Pₐ and/or P_{b} or, independently for each occurrence, P comprises a 6-amino-acid fragment, preferably a 7-amino-acid-fragment, more preferably a 8-amino-acid-fragment, even more preferably a 9-amino-acid fragment, yet even more preferably a 10-amino-acid fragment or even a 11-amino-acid-fragment or yet even a 12-amino-acid-fragment, especially a 13-amino-acid-fragment selected from the group of sequences consisting of SEQ ID NOS: 383-1891 (see Table 1)-preferably group III of Table 1, more preferably group II of Table 1, especially group I of Table 1—and SEQ ID NOs: 1892-2063 (see Table 2)-preferably group I of Table 2—and sequences of group II or III of Table 3 (in particular SEQ ID NOs: 2064-2103), more preferably sequences of group I of Table 3, optionally wherein at most three, preferably at most two, most preferably at least one amino acid is independently substituted by any other amino acid.

In another preferred embodiment, Pₐ and/or P_{b} or, independently for each occurrence, P comprises a 6-amino-acid fragment, preferably a 7-amino-acid-fragment, more preferably a 8-amino-acid-fragment, even more preferably a 9-amino-acid fragment, yet even more preferably a 10-amino-acid fragment or even a 11-amino-acid-fragment or yet even a 12-amino-acid-fragment, especially a 13-amino-acid-fragment selected from the group of sequences of Table 4, in particular the group of sequences identified by SEQ ID NOS: 2104-2190, optionally wherein at most three, preferably at most two, most preferably at least one amino acid is independently substituted by any other amino acid.

In another preferred embodiment, Pₐ and/or P_{b} or, independently for each occurrence, P comprises a 6-amino-acid fragment, preferably a 7-amino-acid-fragment, more preferably a 8-amino-acid-fragment, even more preferably a 9-amino-acid fragment, yet even more preferably a 10-amino-acid fragment or even a 11-amino-acid-fragment or yet even a 12-amino-acid-fragment, especially a 13-amino-acid-fragment selected from the group of sequences of Table 5, optionally wherein at most three, preferably at most two, most preferably at least one amino acid is independently substituted by any other amino acid.

It is further particularly preferred that Pₐ and/or P_{b} or, independently for each occurrence, P comprises a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences consisting of YLOGPIW (SEQ ID NO: 312), VYLOGPI (SEQ ID NO: 313), WONRDVY (SEQ ID NO: 314), DVYLQGP (SEQ ID NO: 315), QNRDVYL (SEQ ID NO: 316), LOGPIWA (SEQ ID NO: 317), RDVYLOG (SEQ ID NO: 318), NRDVYLO (SEQ ID NO: 319), YFGYSTPWGYFDF (SEQ ID NO: 320), FGYSTPWGYF (SEQ ID NO: 321), GYSTPWGYFD (SEQ ID NO: 322), YSTPWGYFDF (SEQ ID NO: 323), NTYFGYSTPWGYF (SEQ ID NO: 324), TPWGYFDFNRFHC (SEQ ID NO: 325), TYFGYSTPWGYFD (SEQ ID NO: 326), DNTYFGYSTPWGY (SEQ ID NO: 327), YFGYSTPWGY (SEQ ID NO: 328), FGYSTPWGYFDEN (SEQ ID NO: 329), NWLPGPC (SEQ ID NO: 330), WLPGPCY (SEQ ID NO: 331), QAKNWLPGPC (SEQ ID NO: 332), AKNWLPGPCY (SEQ ID NO: 333), MANQAKNWLPGPC (SEQ ID NO: 334), QGCLPPF (SEQ ID NO: 335), GCLPPFP (SEQ ID NO: 336), VLGSAHQGCLPPF (SEQ ID NO: 337), LPYVLGSAHOGCL (SEQ ID NO: 338), YVLGSAHOGC (SEQ ID NO: 339), CLPPFPA (SEQ ID NO: 340), SAHQGCLPPF (SEQ ID NO: 341), VLGSAHQGCL (SEQ ID NO: 342), PYVLGSAHQGCLP (SEQ ID NO: 343), GRSSFYC (SEQ ID NO: 344), AVGRSSFYCLEYF (SEQ ID NO: 345), AVGRSSFYCL (SEQ ID NO: 346), QAVGRSSFYCLEY (SEQ ID NO: 347), NGSQAVGRSSFYC (SEQ ID NO: 348), DOYLYYL (SEQ ID NO: 349), PLIDQYLYYL (SEQ ID NO: 350), IDQYLYY (SEQ ID NO: 351), FFPSNGILIF (SEQ ID NO: 352), EERFFPSNGILIF (SEQ ID NO: 353), VGSSSGNWHC (SEQ ID NO: 354) and ADGVGSSSGNWHC (SEQ ID NO: 355), optionally wherein at most three, preferably at most two, most preferably at least one amino acid is independently substituted by any other amino acid.

According to a further preference, Pₐ and/or P_{b} or, independently for each occurrence, P consists of a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in either of the four paragraphs right above, optionally wherein at most three, preferably at most two, most preferably at least one amino acid is independently substituted by any other amino acid, optionally with an N-terminal and/or C-terminal cysteine residue.

In the entire context of the present invention, if a peptide, e.g. Pₐ and/or P_{b} or (independently for each occurrence) peptide P, contains a fragment of at least 4 consecutive amino acids selected from a sequence listed in a row of any one of Tables 1-5 (see below in the Examples section), it is preferred that this fragment is extended (N-terminally or C-terminally) such that the peptide actually contains a longer fragment (e.g. at least 6 or at least 7 or at least 8 or at least 9 or at least 10 or at least 11 or at least 12 or 13 amino acids long) of the source protein given in the same row of the table. In other words, it is preferred that the peptide contains a portion of at least 5 or at least 6 or at least 7 or at least 8 or at least 9 or at least 10 or at least 11 or at least 12 or 13 consecutive amino acids of the viral source protein of the fragment sequence (as given in Tables 1-5).

Further suitable oncolytic viruses (i.e. viruses from which peptide sequences for the present invention may be derived) are disclosed e.g. by Rahman & McFadden 2021, Hromic-Jahjefendic & Lundstrom 2020, and Lawler et al 2017. Therein, lists of oncolytic viruses suitable for the present invention are provided, e.g. DNA viruses such as AdV, Herpes Virus (HSV-1, HSV-2), Parvoviruses (B19PV, H1PV), Poxviruses (VACV, MYXV), Vaccinia Viruses (VACV), as well as RNA viruses such as Alphaviruses (Semiliki Forest virus (SFV), Sindbis virus (SINV), M1), Flavivirus (Zika virus), Paramyxo viruses (Measles virus, Newcastle disease virus (NDV)), Picornaviruses (Coxsackievirus A21, Polio virus, Seneca valley virus (SVV), Reovirus, ECHO-7), Rhabdoviruses (VSV, Maraba virus MG1). Other suitable oncolytic viruses or VLPs are disclosed e.g. in Gao et al., 2021, Jin et al, 2021, Romanenko et al, 2021, Thuenemann et al, 2021, and Kuklik et al., 2021.

Examples for suitable rheovirus sequences include UniProt P03525, P03526, P03527, P03528, P03529, P0CK31, P11078, P11079, P12418, P12419, P15024, and P21270 (reviewed by Müller et al 2020). In other words, the sequence fragment as used herein may comprise an epitope or epitope part (e.g. at least six, especially at least seven or even at least eight amino acids) of said sequences.

Examples for suitable HSV-1 sequences include UniProt sequences A1Z0P5, O09800, P03170, P04288, P04289, P04291, P04486, P06477, P06484, P06487, P06491, P08314, P08392, P08543, P10185, P10190, P10191, P10192, P10193, P10200, P10202, P10204, P10205, P10210, P10216, P10218, P10219, P10220, P10225, P10227, P10228, P10229, P10231, P10234, P32888, and P68333. In other words, the sequence fragment as used herein may comprise an epitope or epitope part (e.g. at least six, especially at least seven or even at least eight amino acids) of said sequences.

Further examples of suitable measles virus sequences include Hemagglutinin H (P08362, P35971), Nucleoprotein N (Q89933), Phosphoprotein P (P35974), Matrix protein M (P35976), Fusion protein F (P69358), Large Protein L (P12576), or unstructured Protein V (Q9EMA9). In other words, the sequence fragment as used herein may comprise an epitope or epitope part (e.g. at least six, especially at least seven or even at least eight amino acids) of said sequences.

An example of a suitable coxsackievirus sequence is Coxsackievirus group B (CVB3) polyprotein-derived protein sequence (see e.g. Liu & Luo, 2021), UniProt sequence A8UMV1. In other words, the sequence fragment as used herein may comprise an epitope or epitope part (e.g. at least six, especially at least seven or even at least eight amino acids) of said sequence.

An example of a suitable VSV sequence is VSV Glycoprotein G (UniProt P03522; see review by Munis (Munis et al, 2020). In other words, the sequence fragment as used herein may comprise an epitope or epitope part (e.g. at least six, especially at least seven or even at least eight amino acids) of said sequence.

Examples of suitable oncolytic poxvirus capsid sequences are disclosed in Torres-Domínguez et al, 2019, and Foloppe et al, 2019. In other words, the sequence fragment as used herein may comprise an epitope or epitope part (e.g. at least six, especially at least seven or even at least eight amino acids) of sequences referenced or disclosed therein.

Examples of suitable vaccinia proteins from which peptides may be derived are disclosed by Resch et al, 2007, and by Condit et al, 2006. In other words, the sequence fragment as used herein may comprise an epitope or epitope part (e.g. at least six, especially at least seven or even at least eight amino acids) of sequences referenced or disclosed therein.

It is highly preferred that the peptides used for the inventive compound do not bind to any HLA Class I or HLA Class II molecule (i.e. of the individual to be treated, e.g. human), in order to prevent presentation and stimulation via a T-cell receptor in vivo and thereby induce an immune reaction. It is generally not desired to involve any suppressive (or stimulatory) T-cell reaction in contrast to antigen-specific immunologic tolerization approaches. Therefore, to avoid T-cell epitope activity as much as possible, the peptides of the compound of the present invention (e.g. peptide P or Pₐ or P_{b}) preferably fulfil one or more of the following characteristics:
To reduce the probability for a peptide used in the compound of the present invention to bind to an HLA Class II or Class I molecule, the peptide (e.g. peptide P or Pₐ or P_{b}) has a preferred length of 4-8 amino acids, although somewhat shorter or longer lengths are still acceptable.
To further reduce the probability that such a peptide binds to an HLA Class II or Class I molecule, it is preferred to test the candidate peptide sequence by HLA binding prediction algorithms such as NetMHCII-2.3 (reviewed by Jensen et al 2018). Preferably, a peptide (e.g. peptide P or Pₐ or P_{b}) used in the compound of the present invention has (predicted) HLA binding (IC50) of at least 500 nM. More preferably, HLA binding (IC50) is more than 1000 nM, especially more than 2000 nM (cf. e.g. Peters et al 2006). In order to decrease the likelihood of HLA Class I binding, NetMHCpan 4.0 may also be applied for prediction (Jurtz et al 2017).
To further reduce the probability that such a peptide binds to an HLA Class I molecule, the NetMHCpan Rank percentile threshhold can be set to a background level of 10% according to Koşaloğlu-Yalçin et al, 2018. Preferably, a peptide (e.g. peptide P or Pₐ or P_{b}) used in the compound of the present invention therefore has a % Rank value of more than 3, preferably more than 5, more preferably more than 10 according to the NetMHCpan algorithm.
To further reduce the probability that such a peptide binds to an HLA Class II molecule, it is beneficial to perform in vitro HLA-binding assays commonly used in the art such as for example refolding assays, iTopia, peptide rescuing assays or array-based peptide binding assays. Alternatively, or in addition thereto, LC-MS based analytics can be used, as e.g. reviewed by Gfeller et al 2016.

For stronger reduction of the titre of the undesired antibodies, it is preferred that the peptides used in the present invention are circularized (see also Example 2). Accordingly, in a preferred embodiment, at least one occurrence of P is a circularized peptide. Preferably at least 10% of all occurrences of P are circularized peptides, more preferably at least 25% of all occurrences of P are circularized peptides, yet more preferably at least 50% of all occurrences of P are circularized peptides, even more preferably at least 75% of all occurrences of P are circularized peptides, yet even more preferably at least 90% of all occurrences of P are circularized peptides or even at least 95% of all occurrences of P are circularized peptides, especially all of the occurrences of P are circularized peptides. Several common techniques are available for circularization of peptides, see e.g. Ong et al 2017. It goes without saying that “circularized peptide” as used herein shall be understood as the peptide itself being circularized, as e.g. disclosed in Ong et al. (and not e.g. grafted on a circular scaffold with a sequence length that is longer than 13 amino acids). Such peptides may also be referred to as cyclopeptides herein.

Further, for stronger reduction of the titre of the undesired antibodies relative to the amount of scaffold used, in a preferred embodiment of the compound of the present invention, independently for each of the peptide n-mers, n is at least 2, more preferably at least 3, especially at least 4. Usually, in order to avoid complexities in the manufacturing process, independently for each of the peptide n-mers, n is less than 10, preferably less than 9, more preferably less than 8, even more preferably less than 7, yet even more preferably less than 6, especially less than 5. To benefit from higher avidity through divalent binding of the undesired antibody, it is highly preferred that, for each of the peptide n-mers, n is 2.

For multivalent binding of the undesired antibodies, it is advantageous that the peptide dimers or n-mers are spaced by a hydrophilic, structurally flexible, immunologically inert, non-toxic and clinically approved spacer such as (hetero-) bifunctional and -trifunctional Polyethylene glycol (PEG) spacers (e.g. NHS-PEG-Maleimide)—a wide range of PEG chains is available and PEG is approved by the FDA. Alternatives to PEG linkers such as immunologically inert and non-toxic synthetic polymers or glycans are also suitable. Accordingly, in the context of the present invention, the spacer (e.g. spacer S) is preferably selected from PEG molecules or glycans. For instance, the spacer such as PEG can be introduced during peptide synthesis. Such spacers (e.g. PEG spacers) may have a molecular weight of e.g. 10000 Dalton. Evidently, within the context of the present invention, the covalent binding of the peptide n-mers to the biopolymer scaffold via a linker each may for example also be achieved by binding of the linker directly to a spacer of the peptide n-mer (instead of, e.g., to a peptide of the peptide n-mer).

Preferably, each of the peptide n-mers is covalently bound to the biopolymer scaffold, preferably via a linker each.

As used herein, the linker may e.g. be selected from disulphide bridges and PEG molecules.

According to a further preferred embodiment of the inventive compound, independently for each occurrence, P is Pₐ or P_{b}.

Furthermore, it is preferred when in the first peptide n-mer, each occurrence of P is Pₐ and, in the second peptide n-mer, each occurrence of P is P_{b}. Alternatively, or in addition thereto, Pₐ and/or P_{b} is circularized.

Divalent binding is particularly suitable to reduce antibody titres. According, in a preferred embodiment,
the first peptide n-mer is Pₐ—S—Pₐ and the second peptide n-mer is Pₐ—S—Pₐ;
the first peptide n-mer is Pₐ—S—Pₐ and the second peptide n-mer is P_{b}—S—P_{b};
the first peptide n-mer is P_{b}—S—P_{b} and the second peptide n-mer is P_{b}—S—P_{b};
the first peptide n-mer is Pₐ—S—P_{b} and the second peptide n-mer is Pₐ—S—P_{b};
the first peptide n-mer is Pₐ—S—P_{b} and the second peptide n-mer is Pₐ—S—Pₐ; or
the first peptide n-mer is Pₐ—S—P_{b} and the second peptide n-mer is P_{b}—S—P_{b}.

For increasing effectivity, in a preferred embodiment the first peptide n-mer is different from the second peptide n-mer. For similar reasons, preferably, the peptide Pₐ is different from the peptide P_{b}, preferably wherein the peptide Pₐ and the peptide P_{b} are two different epitopes of the same antigen or two different epitope parts of the same epitope.

Especially for better targeting of polyclonal antibodies, it is advantageous when the peptide Pₐ and the peptide P_{b} comprise the same amino-acid sequence fragment, wherein the amino-acid sequence fragment has a length of at least 2 amino acids, preferably at least 3 amino acids, more preferably at least 4 amino acids, yet more preferably at least 5 amino acids, even more preferably at least 6 amino acids, yet even more preferably at least 7 amino acids, especially at least 8 amino acids or even at least 9 amino acids.

Further, for stronger reduction of the titre of the undesired antibodies relative to the amount of scaffold used, the compound comprises a plurality of said first peptide n-mer (e.g. up to 10 or 20 or 30) and/or a plurality of said second peptide n-mer (e.g. up to 10 or 20 or 30).

As also illustrated above, it is highly preferred when the compound of the present invention is non-immunogenic in a mammal, preferably in a human, in a non-human primate, in a sheep, in a pig, in a dog or in a rodent.

In the context of the present invention, a non-immunogenic compound preferably is a compound wherein the biopolymer scaffold (if it is a protein) and/or the peptides (of the peptide n-mers) have an IC50 higher than 100 nM, preferably higher than 500 nM, even more preferably higher than 1000 nM, especially higher than 2000 nM, against HLA-DRB1_0101 as predicted by the NetMHCII-2.3 algorithm. The NetMHCII-2.3 algorithm is described in detail in Jensen et al, which is incorporated herein by reference. The algorithm is publicly available under http://www.cbs.dtu.dk/services/NetMHCII-2.3/. Even more preferably, a non-immunogenic compound (or pharmaceutical composition) does not bind to any HLA and/or MHC molecule (e.g. in a mammal, preferably in a human, in a non-human primate, in a sheep, in a pig, in a dog or in a rodent; or of the individual to be treated) in vivo.

According to a further preference, the compound is for (transient) intracorporeal sequestration (or (transient) intracorporeal depletion) of at least one antibody (against the oncolytic virus or neutralizing the oncolytic virus) in an individual, preferably in the bloodstream of the individual and/or for (transient) reduction of the titre of at least one antibody (against the oncolytic virus or neutralizing the oncolytic virus) in the individual, preferably in the bloodstream of the individual.

In another preferred embodiment, the entire sequence, optionally with the exception of an N-terminal and/or C-terminal cysteine, of at least one occurrence of P, preferably of at least 10% of all occurrences of P, more preferably of at least 25% of all occurrences of P, yet more preferably of at least 50% of all occurrences of P, even more preferably of at least 75% of all occurrences of P, yet even more preferably of at least 90% of all occurrences of P or even of at least 95% of all occurrences of P, especially of all of the occurrences of P, is identical to a sequence fragment of a protein, wherein the protein is identified by one of the UniProt accession codes disclosed herein; optionally wherein the sequence fragment comprises at most five, preferably at most four, more preferably at most three, even more preferably at most two, especially at most one amino acid substitutions (e.g. for the purposes mentioned above, such as creating mimotopes).

In another preferred embodiment, the entire sequence, optionally with the exception of an N-terminal and/or C-terminal cysteine, of peptide Pₐ is identical to a sequence fragment of a protein, wherein the protein is identified by one of the UniProt accession codes disclosed herein; optionally wherein said sequence fragment comprises at most five, preferably at most four, more preferably at most three, even more preferably at most two, especially at most one amino acid substitutions (e.g. for the purposes mentioned above, such as creating mimotopes).

In another preferred embodiment, the entire sequence, optionally with the exception of an N-terminal and/or C-terminal cysteine, of peptide P_{b} is identical to a sequence fragment of a protein, wherein the protein is identified by one of the UniProt accession codes disclosed herein; optionally wherein said sequence fragment comprises at most five, preferably at most four, more preferably at most three, even more preferably at most two, especially at most one amino acid substitutions (e.g. for the purposes mentioned above, such as creating mimotopes).

In another preferred embodiment, the entire sequence, optionally with the exception of an N-terminal and/or C-terminal cysteine, of peptide Pₐ is identical to a sequence fragment of a protein and the entire sequence, optionally with the exception of an N-terminal and/or C-terminal cysteine, of peptide P_{b} is identical to the same or another, preferably another, sequence fragment of the same protein, wherein the protein is identified by one of the UniProt accession codes listed herein; optionally wherein said sequence fragment and/or said another sequence fragment comprises at most five, preferably at most four, more preferably at most three, even more preferably at most two, especially at most one amino acid substitutions (e.g. for the purposes mentioned above, such as creating mimotopes).

In an aspect, the present invention relates to a pharmaceutical composition comprising the inventive and at least one pharmaceutically acceptable excipient.

In embodiments, the composition is prepared for intraperitoneal, subcutaneous, intramuscular and/or intravenous administration. In particular, the composition is for repeated administration (since it is typically non-immunogenic).

In a preference, the molar ratio of peptide P or Pₐ or P_{b} to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

In another aspect, the compound of the present invention is for use in therapy, preferably therapy of a neoplasm such as a benign neoplasm, an in situ neoplasm or an malignant neoplasm, especially therapy of a solid tumor or a hematological malignancy. The malignant neoplasm may be any solid tumor, in particular selected from the group of (metastatic) tumors such as brain cancers (e.g. glioma, glioblastoma, cerebellar brain tumors, as reviewed by de Sostoa et al, 2020), breast cancers, colon- and colorectal cancers, esophageal cancers, prostate cancers, bladder cancer, kidney cancers, endometrial cancers, thyroid cancers, gastric cancers, liver cancers, lung cancers, uterine cancers, tracheal cancers, testicular cancers, cervical cancers, head and neck cancers, skin cancers, bone cancers, pancreatic cancers and ovarian cancers. Alternatively, the malignant neoplasm may be a hematological malignancy, in particular selected from the group of leukemias, lymphomas and multiple myelomas such as listed in the reviews by Innao et al, 2020, and Yang et al, 2021. Throughout the entire context of the present invention, the solid tumor may be metastatic. Typically, the solid tumor is malignant.

The individual to be treated (who is preferably human) typically has a neoplasm such as a benign neoplasm, an in situ neoplasm or a malignant neoplasm. The malignant neoplasm may be any solid tumor, in particular as defined in the paragraph above. Alternatively, the malignant neoplasm may be a hematological malignancy, in particular as defined in the paragraph above.

In the course of the present invention, it turned out that the inventive compound provides a highly suitable transient therapeutic window for oncolytic virus administration (see e.g. Example 1 and FIGS. 1 and 2). According to a particular preference, the administration of the compound (or the pharmaceutical composition comprising the compound) is followed by (systemic or local, especially systemic) administration of the oncolytic virus or oncolytic VLP (based on the oncolytic virus) within 96 hours, preferably within 72 hours or even within 60 hours, more preferably within 48 hours, even more preferably within 36 hours, yet even more preferably within 24 hours, especially within 12 hours; preferably wherein the (systemic or local, especially systemic) administration of the oncolytic virus or oncolytic VLP occurs at least 6 hours, preferably at least 12 hours, more preferably at least 24 hours after administration of the pharmaceutical composition. Put differently, the window for (systemic or local, especially systemic) administration of the oncolytic virus or oncolytic VLP is preferably 0-96 hours, more preferably 6-72 hours, even more preferably 12-60 hours, yet even more preferably 24-48 hours post administration of the compound (or the pharmaceutical composition comprising the compound).

In the course of the present invention, it furthermore turned out that the in vivo kinetics of undesirable-antibody lowering by the inventive compound is typically very fast, sometimes followed by a mild rebound of the undesirable antibody. It is thus particularly preferred when the compound (or the pharmaceutical composition comprising the compound) is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window; in particular wherein this window is followed by administration of the anti-neoplastic composition as described herein within 96 hours, preferably within 72 hours or even within 60 hours, more preferably within 48 hours, even more preferably within 36 hours, yet even more preferably within 24 hours, especially within 12 hours; preferably wherein the (systemic or local, especially systemic) administration of the oncolytic virus or oncolytic VLP occurs at least 6 hours, preferably at least 12 hours, more preferably at least 24 hours after administration of the pharmaceutical composition. For instance, the pharmaceutical composition may be administered at −24 hrs and −12 hrs before (systemic or local, especially systemic) administration of the anti-neoplastic composition (oncolytic virus or oncolytic VLP based thereon) at 0 hrs.

According to a particular preference, the compound of the present invention is for use in increasing efficacy of an oncolytic virus or oncolytic VLP based on the oncolytic virus (as defined herein) in an individual, preferably wherein the pharmaceutical composition is administered to the individual prior to or concurrently with (systemic or local, especially systemic) administration of the oncolytic virus or oncolytic VLP based on the oncolytic virus. Alternatively, or in addition thereto, the titer of an antibody specific for the oncolytic virus or oncolytic VLP (and preferably also for the compound) is preferably determined after administration of the pharmaceutical composition and prior to administration of the virus or VLP, especially wherein the virus or VLP is only administered if the titer is below a threshold. Otherwise, administration of the composition may be repeated to further lower the titer.

In embodiments, one or more antibodies are present in the individual which are specific for at least one occurrence of peptide P, or for peptide Pₐ and/or peptide P_{b}, preferably wherein said antibodies are neutralizing antibodies for said oncolytic virus.

It is highly preferred that the composition is non-immunogenic in the individual (e.g. it does not comprise an adjuvant or an immunostimulatory substance that stimulates the innate or the adaptive immune system, e.g. such as an adjuvant or a T-cell epitope).

The composition of the present invention may be administered at a dose of 1-1000 mg, preferably 2-500 mg, more preferably 3-250 mg, even more preferably 4-100 mg, especially 5-50 mg, compound per kg body weight of the individual, preferably wherein the composition is administered repeatedly. Such administration may be intraperitoneally, subcutaneously, intramuscularly or intravenously or systemically or locally.

In an aspect, the present invention relates to a method of (transiently) sequestering (or depleting) one or more antibodies (preferably wherein said antibodies are neutralizing antibodies for said oncolytic virus) present in an individual, comprising
obtaining a pharmaceutical composition as defined herein, wherein the composition is non-immunogenic in the individual and wherein the one or more antibodies present in the individual are specific for at least one occurrence of P, or for peptide Pₐ and/or peptide P_{b}; and
administering (in particular repeatedly administering, e.g. at least two times, preferably at least three times, more preferably at least five times) the pharmaceutical composition to the individual.

In the context of the present invention, the individual (to be treated) may be a human or non-human animal, preferably a non-human primate, a sheep, a pig, a dog or a rodent, in particular a mouse.

Preferably, the biopolymer scaffold is autologous with respect to the individual, preferably wherein the biopolymer scaffold is an autologous protein (i.e. murine albumin is used when the individual is a mouse).

In yet another aspect, the present invention relates to a pharmaceutical composition (i.e. an anti-neoplasticcomposition), comprising the compound defined herein and further comprising the oncolytic virus and optionally at least one pharmaceutically acceptable excipient. The oncolytic virus typically comprises a peptide fragment with a sequence length of at least six, preferably at least seven, more preferably at least eight, especially at least 9 amino acids. The sequence of at least one occurrence of peptide P, or peptide Pₐ and/or peptide P_{b}, of the compound is at least 70% identical, preferably at least 75% identical, more preferably at least 80% identical, yet more preferably at least 85% identical, even more preferably at least 90% identical, yet even more preferably at least 95% identical, especially completely identical to the sequence of said peptide fragment. Preferably, this pharmaceutical composition is for use in therapy of a neoplasm and/or for use in prevention or inhibition of an undesirable immune reaction against the oncolytic virus.

This composition is furthermore preferably non-immunogenic in the individual.

In even yet another aspect, the present invention provides a method of inhibiting a (undesirable)-especially humoral-immune reaction to a treatment with a anti-neoplastic composition in an individual in need of treatment with the anti-neoplastic composition as defined above or of inhibiting neutralization of an oncolytic virus in a anti-neoplastic composition as defined above for an individual in need of treatment with the anti-neoplastic composition, comprising obtaining said anti-neoplastic composition; wherein the compound of the anti-neoplastic composition is non-immunogenic in the individual, and administering (preferably repeatedly administering) the anti-neoplastic composition to the individual (preferably systemically or locally).

In general, screening for peptide mimotopes per se is known in the art, see for instance Shanmugam et al. Mimotope-based compounds of the invention have the following two advantages over compounds based on wild-type epitopes: First, the undesired antibodies, as a rule, have even higher affinities for mimotopes found by screening a peptide library, leading to higher clearance efficiency of the mimotope-based compound. Second, mimotopes further enable avoiding T-cell epitope activity as much as possible (as described hereinabove) in case the wild-type epitope sequence induces such T-cell epitope activity.

In a further aspect, the present invention relates to a peptide, wherein the peptide is defined as disclosed herein for any one of the at least two peptides of the inventive compound, P, Pₐ, or P_{b}.

In certain embodiments, such peptides may be used as probes for the diagnostic typing and analysis of circulating oncolytic virus-neutralizing antibodies. The peptides can e.g. be used as part of a diagnostic vector-neutralizing antibody typing or screening device or kit or procedure, as a companion diagnostic, for patient stratification or for monitoring vector-neutralizing antibody levels prior to, during and/or after anti-neoplastic therapy (with the oncolytic virus).

In a further aspect, the invention relates to a method for detecting and/or quantifying oncolytic virus-neutralizing antibodies in a biological sample comprising the steps of
bringing the sample into contact with the peptide defined as disclosed herein (e.g. for P, Pₐ, or P_{b}) or the compound as disclosed herein, and
detecting the presence and/or concentration of antibodies in the sample.

The skilled person is familiar with methods for detecting and/or quantifying antibodies in biological samples. The method can e.g. be a sandwich assay, preferably an enzyme-linked immunosorbent assay (ELISA), or a surface plasmon resonance (SPR) assay.

In a preference, the peptide (especially at least 10, more preferably at least 100, even more preferably at least 1000, especially at least 10000 different peptides of the invention) or the compound is immobilized on a solid support, preferably an ELISA plate or an SPR chip or a biosensor-based diagnostic device with an electrochemical, fluorescent, magnetic, electronic, gravimetric or optical biotransducer. Alternatively, or in addition thereto, the peptide (especially at least 10, more preferably at least 100, even more preferably at least 1000, especially at least 10000 different peptides of the invention) or the compound may be coupled to a reporter or reporter fragment, such as a reporter fragment suitable for a protein-fragment complementation assay (PCA); see e.g. Li et al, 2019, or Kainulainen et al, 2021.

Preferably, the sample is obtained from a mammal, preferably a human. Preferably the sample is a blood sample, preferably a whole blood, serum, or plasma sample.

According to a preferred embodiment, the mammal (human) has a neoplasm, more preferably wherein the neoplasm is a benign neoplasm, an in situ neoplasm or a malignant neoplasm, even more preferably a solid tumor or a hematological malignancy, especially wherein the mammal has a solid tumor preferably selected from the group of brain cancers, breast cancers, colon cancers, esophageal cancers, prostate cancers, bladder cancer, kidney cancers, endometrial cancers, thyroid cancers, gastric cancers, liver cancers, lung cancers, pancreatic cancers, uterine cancers, tracheal cancers, testicular cancers, cervical cancers, head and neck cancers, skin cancers, bone cancers, pancreatic cancers and ovarian cancers, or a hematological malignancy preferably selected from the group of leukemias, lymphomas and multiple myelomas; in particular wherein the mammal (human) has been selected to undergo therapy with an oncolytic virus or oncolytic VLP or is undergoing therapy with an oncolytic virus or oncolytic VLP.

The invention further relates to the use of a peptide defined as disclosed herein (e.g. for P, Pₐ, or P_{b}) or the compound as disclosed herein in a diagnostic assay, preferably ELISA, preferably as disclosed herein above.

A further aspect of the invention relates to a diagnostic device comprising the peptide defined as disclosed herein (e.g. for P, Pₐ, or P_{b}) or the compound as disclosed herein, preferably immobilized on a solid support. In a preference, the solid support is an ELISA plate or a surface plasmon resonance chip. In another preference, the diagnostic device is a biosensor-based diagnostic device with an electrochemical, fluorescent, magnetic, electronic, gravimetric or optical biotransducer.

In another preferred embodiment, the diagnostic device is a lateral flow assay.

The invention further relates to a diagnostic kit comprising a peptide defined as disclosed herein (e.g. for P, Pₐ, or P_{b}) or the compound as disclosed herein, preferably a diagnostic device as defined herein. Preferably the diagnostic kit further comprises one or more selected from the group of a buffer, a reagent, instructions. Preferably the diagnostic kit is an ELISA kit.

A further aspect relates to an apheresis device comprising the peptide defined as disclosed herein (e.g. for P, Pₐ, or P_{b}). Preferably the peptide is immobilized on a solid carrier. It is especially preferred if the apheresis device comprises at least two, preferably at least three, more preferably at least four different peptides defined as disclosed herein (e.g. for P, Pₐ, or P_{b}). In a preferred embodiment the solid carrier comprises the inventive compound.

Preferably, the solid carrier is capable of being contacted with blood or plasma flow. Preferably, the solid carrier is a sterile and pyrogen-free column.

In the context of the present invention, for improved bioavailability, it is preferred that the inventive compound has a solubility in water at 25° C. of at least 0.1 μg/ml, preferably at least 1 μg/ml, more preferably at least 10 μg/ml, even more preferably at least 100 μg/ml, especially at least 1000 μg/ml.

The term “preventing” or “prevention” as used herein means to stop a disease state or condition from occurring in a patient or subject completely or almost completely or at least to a (preferably significant) extent, especially when the patient or subject or individual is predisposed to such a risk of contracting a disease state or condition.

The pharmaceutical composition of the present invention is preferably provided as a (typically aqueous) solution, (typically aqueous) suspension or (typically aqueous) emulsion. Excipients suitable for the pharmaceutical composition of the present invention are known to the person skilled in the art, upon having read the present specification, for example water (especially water for injection), saline, Ringer's solution, dextrose solution, buffers, Hank solution, vesicle forming compounds (e.g. lipids), fixed oils, ethyl oleate, 5% dextrose in saline, substances that enhance isotonicity and chemical stability, buffers and preservatives. Other suitable excipients include any compound that does not itself induce the production of antibodies in the patient (or individual) that are harmful for the patient (or individual). Examples are well tolerable proteins, polysaccharides, polylactic acids, polyglycolic acid, polymeric amino acids and amino acid copolymers. This pharmaceutical composition can (as a drug) be administered via appropriate procedures known to the skilled person (upon having read the present specification) to a patient or individual in need thereof (i.e. a patient or individual having or having the risk of developing the diseases or conditions mentioned herein). The preferred route of administration of said pharmaceutical composition is parenteral administration, in particular through intraperitoneal, subcutaneous, intramuscular and/or intravenous administration. For parenteral administration, the pharmaceutical composition of the present invention is preferably provided in injectable dosage unit form, e.g. as a solution (typically as an aqueous solution), suspension or emulsion, formulated in conjunction with the above-defined pharmaceutically acceptable excipients. The dosage and method of administration, however, depends on the individual patient or individual to be treated. Said pharmaceutical composition can be administered in any suitable dosage known from other biological dosage regimens or specifically evaluated and optimised for a given individual. For example, the active agent may be present in the pharmaceutical composition in an amount from 1 mg to 10 g, preferably 50 mg to 2 g, in particular 100 mg to 1 g. Usual dosages can also be determined on the basis of kg body weight of the patient, for example preferred dosages are in the range of 0.1 mg to 100 mg/kg body weight, especially 1 to 10 mg/kg body weight (per administration session). The administration may occur e.g. once daily, once every other day, once per week or once every two weeks. As the preferred mode of administration of the inventive pharmaceutical composition is parenteral administration, the pharmaceutical composition according to the present invention is preferably liquid or ready to be dissolved in liquid such sterile, de-ionised or distilled water or sterile isotonic phosphate-buffered saline (PBS). Preferably, 1000 μg (dry-weight) of such a composition comprises or consists of 0.1-990 μg, preferably 1-900 μg, more preferably 10-200 μg compound, and option-ally 1-500 μg, preferably 1-100 μg, more preferably 5-15 μg (buffer) salts (preferably to yield an isotonic buffer in the final volume), and optionally 0.1-999.9 μg, preferably 100-999.9 μg, more preferably 200-999 μg other excipients. Preferably, 100 mg of such a dry composition is dissolved in sterile, de-ionised/distilled water or sterile isotonic phosphate-buffered saline (PBS) to yield a final volume of 0.1-100 ml, preferably 0.5-20 ml, more preferably 1-10 ml.

It is evident to the skilled person that active agents and drugs described herein can also be administered in salt-form (i.e. as a pharmaceutically acceptable salt of the active agent). Accordingly, any mention of an active agent herein shall also include any pharmaceutically acceptable salt forms thereof.

Methods for chemical synthesis of peptides used for the compound of the present invention are well-known in the art. Of course, it is also possible to produce the peptides using recombinant methods. The peptides can be produced in microorganisms such as bacteria, yeast or fungi, in eukaryotic cells such as mammalian or insect cells, or in a recombinant virus vector such as adenovirus, poxvirus, herpesvirus, Simliki forest virus, baculovirus, bacteriophage, sindbis virus or sendai virus. Suitable bacteria for producing the peptides include E. coli, B. subtilis or any other bacterium that is capable of expressing such peptides. Suitable yeast cells for expressing the peptides of the present invention include Saccharomyces cerevisiae, Schizosaccharomyces pombe, Candida, Pichiapastoris or any other yeast capable of expressing peptides. Corresponding means and methods are well known in the art. Also, methods for isolating and purifying recombinantly produced peptides are well known in the art and include e.g. gel filtration, affinity chromatography, ion exchange chromatography etc.

Beneficially, cysteine residues are added to the peptides at the N- and/or C-terminus to facilitate coupling to the biopolymer scaffold, especially.

To facilitate isolation of said peptides, fusion polypeptides may be made wherein the peptides are translationally fused (covalently linked) to a heterologous polypeptide which enables isolation by affinity chromatography. Typical heterologous polypeptides are His-Tag (e.g. His6; 6 histidine residues), GST-Tag (Glutathione-S-transferase) etc. The fusion polypeptide facilitates not only the purification of the peptides but can also prevent the degradation of the peptides during the purification steps. If it is desired to remove the heterologous polypeptide after purification, the fusion polypeptide may comprise a cleavage site at the junction between the peptide and the heterologous polypeptide. The cleavage site may consist of an amino acid sequence that is cleaved with an enzyme specific for the amino acid sequence at the site (e.g. proteases).

The coupling/conjugation chemistry used to link the peptides/peptide n-mers to the biopolymer scaffold (e.g. via heterobifunctional compounds such as GMBS and of course also others as described in “Bioconjugate Techniques”, Greg T. Hermanson) or used to conjugate the spacer to the peptides in the context of the present invention can also be selected from reactions known to the skilled in the art. The biopolymer scaffold itself may be recombinantly produced or obtained from natural sources.

Herein, the term “specific for”—as in “molecule A specific for molecule B”—means that molecule A has a binding preference for molecule B compared to other molecules in an individual's body. Typically, this entails that molecule A (such as an antibody) has a dissociation constant (also called “affinity”) in regard to molecule B (such as the antigen, specifically the binding epitope thereof) that is lower than (i.e. “stronger than”) 1000 nM, preferably lower than 100 nM, more preferably lower than 50 nM, even more preferably lower than 10 nM, especially lower than 5 nM.

Herein, “UniProt” refers to the Universal Protein Resource. UniProt is a comprehensive resource for protein sequence and annotation data. UniProt is a collaboration between the European Bioinformatics Institute (EMBL-EBI), the SIB Swiss Institute of Bioinformatics and the Protein Information Resource (PIR). Across the three institutes more than 100 people are involved through different tasks such as database curation, software development and support. Website: http://www.uniprot.org/

Entries in the UniProt databases are identified by their accession codes (referred to herein e.g. as “UniProt accession code” or briefly as “UniProt” followed by the accession code), usually a code of six alphanumeric letters (e.g. “Q1HVF7”). If not specified otherwise, the accession codes used herein refer to entries in the Protein Knowledgebase (UniProtKB) of UniProt. If not stated otherwise, the UniProt database state for all entries referenced herein is of 23 Feb. 2022 (UniProt/UniProtKB Release 2022_01).

In the context of the present application, sequence variants (designated as “natural variant” in UniProt) are expressly included when referring to a UniProt database entry.

“Percent (%) amino acid sequence identity” or “X % identical” (such as “70% identical”) with respect to a reference polypeptide or protein sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2, Megalign (DNASTAR) or the “needle” pairwise sequence alignment application of the EMBOSS software package. Those skilled in the art can determine appropriate parameters for aligning sequences, including any algorithms needed to achieve maximal alignment over the full length of the sequences being compared. For purposes herein, however, % amino acid sequence identity values are calculated using the sequence alignment of the computer programme “needle” of the EMBOSS software package (publicly available from European Molecular Biology Laboratory; Rice et al., 2000).

The needle programme can be accessed under the web site http://www.ebi.ac.uk/Tools/psa/emboss_needle/ or downloaded for local installation as part of the EMBOSS package from http://emboss.sourceforge.net/. It runs on many widely-used UNIX operating systems, such as Linux.

To align two protein sequences, the needle programme is preferably run with the following parameters:

Commandline: needle-auto-stdout-asequence SEQUENCE_FILE_A -bsequence SEQUENCE_FILE_B-datafile EBLOSUM62-gapopen 10.0-gapextend 0.5-endopen 10.0-endextend 0.5-aformat3 pair-sprotein1-sprotein2 (Align format: pair Report file: stdout)

The % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program needle in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. In cases where “the sequence of A is more than No identical to the entire sequence of B”, Y is the entire sequence length of B (i.e. the entire number of amino acid residues in B). Unless specifically stated otherwise, all % amino acid sequence identity values used herein are obtained as described in the immediately preceding paragraph using the needle computer program.

The present invention further relates to the following embodiments:

Embodiment 1. A compound comprising
a biopolymer scaffold and at least
a first peptide n-mer of the general formula:

P(—S—P)₍ₙ₋₁₎ and
a second peptide n-mer of the general formula:

P(—S—P)₍ₙ₋₁₎;
wherein, independently for each occurrence, P is a peptide with a sequence length of 6-13 amino acids, and S is a non-peptide spacer,
wherein, independently for each of the peptide n-mers, n is an integer of at least 1, preferably of at least 2, more preferably of at least 3, especially of at least 4,
wherein each of the peptide n-mers is bound to the biopolymer scaffold, preferably via a linker each,
wherein, independently for each occurrence, P has an amino-acid sequence comprising a sequence fragment (in particular an epitopic sequence) with a length of at least six (preferably at least 7, more preferably at least 8, especially at least 9) amino acids of a protein sequence (preferably a capsid protein sequence, nucleocapsid protein sequence, a structural protein sequence, viral envelope- or receptor-binding-protein sequence, tegument protein sequence, viral enzyme sequence, nuclear matrix protein sequence, DNA- or RNA-binding protein sequence, viral packaging protein sequence or viral polymerase sequence, more preferably a capsid protein sequence, nucleocapsid protein sequence, a structural protein sequence, viral envelope- or receptor-binding-protein sequence, or a tegument protein sequence, in particular a capsid protein sequence) of an oncolytic virus (in particular of an oncolytic DNA virus or an oncolytic RNA virus or an oncolytic retrovirus), in particular of an AdV hexon protein sequence, an AdV fiber protein sequence, an AdV penton protein sequence, an AdV IIIa protein sequence, an AdV VI protein sequence, an AdV VIII protein sequence or an AdV IX protein sequence or of any one of the capsid protein sequences identified in FIG. 4 and FIG. 5 or of any one of the capsid protein sequences listed in Cearley et al., 2008, or of a capsid protein sequence identified by any one of UniProt P03525, P03526, P03527, P03528, P03529, P0CK31, P11078, P11079, P12418, P12419, P15024, P21270, A1Z0P5, O09800, P03170, P04288, P04289, P04291, P04486, P06477, P06484, P06487, P06491, P08314, P08392, P08543, P10185, P10190, P10191, P10192, P10193, P10200, P10202, P10204, P10205, P10210, P10216, P10218, P10219, P10220, P10225, P10227, P10228, P10229, P10231, P10234, P32888, P68333, P08362, P35971, Q89933, P35974, P35976, P69358, P12576, Q9EMA9 and P03522,
optionally wherein at most three, preferably at most two, most preferably at least one amino acid of the sequence fragment is independently substituted by any other amino acid.

Embodiment 2. The compound of embodiment 1, wherein at least one occurrence of P is a circularized peptide, preferably wherein at least 10% of all occurrences of P are circularized peptides, more preferably wherein at least 25% of all occurrences of P are circularized peptides, yet more preferably wherein at least 50% of all occurrences of P are circularized peptides, even more preferably wherein at least 75% of all occurrences of P are circularized peptides, yet even more preferably wherein at least 90% of all occurrences of P are circularized peptides or even wherein at least 95% of all occurrences of P are circularized peptides, especially wherein all of the occurrences of P are circularized peptides; or wherein at least one occurrence of P is a linear peptide, preferably wherein at least 10% of all occurrences of P are linear peptides, more preferably wherein at least 25% of all occurrences of P are linear peptides, yet more preferably wherein at least 50% of all occurrences of P are linear peptides, even more preferably wherein at least 75% of all occurrences of P are linear peptides, yet even more preferably wherein at least 90% of all occurrences of P are linear peptides or even wherein at least 95% of all occurrences of P are linear peptides, especially wherein all of the occurrences of P are linear peptides.

Embodiment 3. The compound of embodiment 1 or 2, wherein, independently for each of the peptide n-mers, n is at least 2, more preferably at least 3, especially at least 4.

Embodiment 4. The compound of any one of embodiments 1 to 3, wherein, independently for each of the peptide n-mers, n is less than 10, preferably less than 9, more preferably less than 8, even more preferably less than 7, yet even more preferably less than 6, especially less than 5.

Embodiment 5. The compound of any one of embodiments 1 to 4, wherein, for each of the peptide n-mers, n is 2.

Embodiment 6. The compound of any one of embodiments 1 to 5, wherein at least one occurrence of P is Pₐ and/or at least one occurrence of P is P_{b},
wherein Pₐ is a peptide with a sequence length of 6-13 amino acids, preferably 7-11 amino acids, more preferably 7-9 amino acids,
wherein P_{b} is a peptide with a sequence length of 6-13 amino acids, preferably 7-11 amino acids, more preferably 7-9 amino acids;
preferably wherein Pₐ and/or P_{b} comprises an epitope sequence of the protein sequence (preferably a capsid protein sequence, nucleocapsid protein sequence, a structural protein sequence, viral envelope- or receptor-binding-protein sequence, tegument protein sequence, viral enzyme sequence, nuclear matrix protein sequence, DNA- or RNA-binding protein sequence, viral packaging protein sequence or viral polymerase sequence, more preferably a capsid protein sequence, nucleocapsid protein sequence, a structural protein sequence, viral envelope- or receptor-binding-protein sequence, or a tegument protein sequence, in particular a capsid protein sequence) of the oncolytic virus or a mimotope thereof.

Embodiment 7. The compound of any one of embodiments 1 to 6, wherein, independently for each occurrence, P is Pₐ or P_{b}.

Embodiment 8. The compound of any one of embodiments 1 to 7, wherein, in the first peptide n-mer, each occurrence of P is Pₐ and, in the second peptide n-mer, each occurrence of P is P_{b}.

Embodiment 9. The compound of any one of embodiments 1 to 8, wherein
the first peptide n-mer is Pₐ—S—Pₐ and the second peptide n-mer is Pₐ—S—Pₐ; or
the first peptide n-mer is Pₐ—S—Pₐ and the second peptide n-mer is P_{b}—S—P_{b};
the first peptide n-mer is P_{b}—S—P_{b} and the second peptide n-mer is P_{b}—S—P_{b};
the first peptide n-mer is Pₐ—S—P_{b} and the second peptide n-mer is Pₐ—S—P_{b};
the first peptide n-mer is Pₐ—S—P_{b} and the second peptide n-mer is Pₐ—S—Pₐ; or
the first peptide n-mer is Pₐ—S—P_{b} and the second peptide n-mer is P_{b}—S—P_{b}.

Embodiment 10. A compound comprising
a biopolymer scaffold and at least
a first peptide n-mer which is a peptide dimer of the formula

Pₐ—S—Pₐ or Pₐ—S—P_{b},
wherein Pₐ is a peptide with a sequence length of 6-13 amino acids, preferably 7-11 amino acids, more preferably 7-9 amino acids, P_{b} is a peptide with a sequence length of 6-13 amino acids, preferably 7-11 amino acids, more preferably 7-9 amino acids, and S is a non-peptide spacer,
wherein the first peptide n-mer is bound to the biopolymer scaffold, preferably via a linker,
wherein Pₐ has an amino-acid sequence comprising a sequence fragment with a length of at least six, preferably at least seven, more preferably at least eight, especially at least 9 (or 10, 11, 12 or 13) amino acids of a protein sequence (preferably a capsid protein sequence, nucleocapsid protein sequence, a structural protein sequence, viral envelope- or receptor-binding-protein sequence, tegument protein sequence, viral enzyme sequence, nuclear matrix protein sequence, DNA- or RNA-binding protein sequence, viral packaging protein sequence or viral polymerase sequence, more preferably a capsid protein sequence, nucleocapsid protein sequence, a structural protein sequence, viral envelope- or receptor-binding-protein sequence, or a tegument protein sequence, in particular a capsid protein sequence) of a (non-pathogenic) oncolytic virus, in particular of an AdV hexon protein sequence, an AdV fiber protein sequence, an AdV penton protein sequence, an AdV IIIa protein sequence, an AdV VI protein sequence, an AdV VIII protein sequence or an AdV IX protein sequence or of any one of the capsid protein sequences identified in FIG. 4 and FIG. 5 or of any one of the capsid protein sequences listed in Cearley et al., 2008, or of a capsid protein sequence identified by any one of UniProt P03525, P03526, P03527, P03528, P03529, P0CK31, P11078, P11079, P12418, P12419, P15024, P21270, A1Z0P5, O09800, P03170, P04288, P04289, P04291, P04486, P06477, P06484, P06487, P06491, P08314, P08392, P08543, P10185, P10190, P10191, P10192, P10193, P10200, P10202, P10204, P10205, P10210, P10216, P10218, P10219, P10220, P10225, P10227, P10228, P10229, P10231, P10234, P32888, P68333, P08362, P35971, Q89933, P35974, P35976, P69358, P12576, Q9EMA9 and P03522, optionally wherein at most three, preferably at most two, most preferably at least one amino acid of the sequence fragment is independently substituted by any other amino acid.

Embodiment 11. The compound of embodiment 10, further comprising a second peptide n-mer which is a peptide dimer of the formula P_{b}—S—P_{b} or Pₐ—S—P_{b},
wherein the second peptide n-mer is bound to the biopolymer scaffold, preferably via a linker,
wherein P_{b} has an amino-acid sequence comprising a sequence fragment with a length of at least six, preferably at least seven, more preferably at least eight, especially at least 9 (or 10, 11, 12 or 13) amino acids of a protein sequence (preferably a capsid protein sequence, nucleocapsid protein sequence, a structural protein sequence, viral envelope- or receptor-binding-protein sequence, tegument protein sequence, viral enzyme sequence, nuclear matrix protein sequence, DNA- or RNA-binding protein sequence, viral packaging protein sequence or viral polymerase sequence, more preferably a capsid protein sequence, nucleocapsid protein sequence, a structural protein sequence, viral envelope- or receptor-binding-protein sequence, or a tegument protein sequence, in particular a capsid protein sequence) of a (non-pathogenic) oncolytic virus, in particular of an AdV hexon protein sequence, an AdV fiber protein sequence, an AdV penton protein sequence, an AdV IIIa protein sequence, an AdV VI protein sequence, an AdV VIII protein sequence or an AdV IX protein sequence or of any one of the capsid protein sequences identified in FIG. 4 and FIG. 5 or of any one of the capsid protein sequences listed in Cearley et al., 2008, or of a capsid protein sequence identified by any one of UniProt P03525, P03526, P03527, P03528, P03529, P0CK31, P11078, P11079, P12418, P12419, P15024, P21270, A1Z0P5, O09800, P03170, P04288, P04289, P04291, P04486, P06477, P06484, P06487, P06491, P08314, P08392, P08543, P10185, P10190, P10191, P10192, P10193, P10200, P10202, P10204, P10205, P10210, P10216, P10218, P10219, P10220, P10225, P10227, P10228, P10229, P10231, P10234, P32888, P68333, P08362, P35971, Q89933, P35974, P35976, P69358, P12576, Q9EMA9 and P03522, optionally wherein at most three, preferably at most two, most preferably at least one amino acid of the sequence fragment is independently substituted by any other amino acid.

Embodiment 12. The compound of any one of embodiments 1 to 9 and 11, wherein the first peptide n-mer is different from the second peptide n-mer.

Embodiment 13. The compound of any one of embodiments 6 to 12, wherein the peptide Pₐ is different from the peptide P_{b}, preferably wherein the peptide Pₐ and the peptide P_{b} are two different epitopes of the same viral (capsid) antigen or two different epitope parts of the same viral (capsid) epitope.

Embodiment 14. The compound of any one of embodiments 6 to 13, wherein the peptide Pₐ and the peptide P_{b} comprise the same amino-acid sequence fragment, wherein the amino-acid sequence fragment has a length of at least 2 amino acids, preferably at least 3 amino acids, more preferably at least 4 amino acids, yet more preferably at least 5 amino acids, even more preferably at least 6 amino acids, yet even more preferably at least 7 amino acids, especially at least 8 amino acids or even at least 9 amino acids.

Embodiment 15. The compound of any one of embodiments 6 to 14, wherein Pₐ and/or P_{b} is circularized.

Embodiment 16. The compound of any one of embodiments 1 to 15, wherein the compound comprises a plurality of said first peptide n-mer and/or a plurality of said second peptide n-mer.

Embodiment 17. The compound of any one of embodiments 1 to 16, wherein the biopolymer scaffold is a protein, preferably a mammalian protein such as a human protein, a non-human primate protein, a sheep protein, a pig protein, a dog protein or a rodent protein.

Embodiment 18. The compound of embodiment 17, wherein the biopolymer scaffold is a globulin.

Embodiment 19. The compound of embodiment 18, wherein the biopolymer scaffold is selected from the group consisting of immunoglobulins, alpha1-globulins, alpha2-globulins and beta-globulins.

Embodiment 20. The compound of embodiment 19, wherein the biopolymer scaffold is selected from the group consisting of immunoglobulin G, haptoglobin and transferrin.

Embodiment 21. The compound of embodiment 20, wherein the biopolymer scaffold is haptoglobin.

Embodiment 22. The compound of embodiment 17, wherein the biopolymer scaffold is an albumin.

Embodiment 23. The compound of any one of embodiments 1 to 22, wherein the compound is non-immunogenic in a mammal, preferably in a human, in a non-human primate, in a sheep, in a pig, in a dog or in a rodent.

Embodiment 24. The compound of any one of embodiments 1 to 23, wherein the compound is for (transient) intracorporeal sequestration (or intracorporeal depletion) of at least one antibody (against the oncolytic virus or neutralizing the oncolytic virus) in an individual, preferably in the bloodstream of the individual and/or for reduction of the titre of at least one antibody (against the oncolytic virus or neutralizing the oncolytic virus) in the individual, preferably in the bloodstream of the individual.

Embodiment 25. The compound of any one of embodiments 1 to 24, wherein the oncolytic virus is an adenovirus (AdV), an adeno-associated virus (AAV), a measles virus, a Herpes simplex virus (HSV), a poxvirus such as Vaccinia virus, a reovirus, a Newcastle disease virus (NVD), a rhabdovirus, a coxsackievirus, a lentivirus, an alphavirus such as Semliki Forest virus, a vesicular stomatitis virus (VSS), a myxoma virus (MYXV), a mengovirus, a bovine viral diarrhea virus (BVDV), a chimeric oncolytic virus, a picornavirus, a parvovirus or a flavivirus.

Embodiment 26. The compound of any one of embodiments 1 to 25, wherein the entire sequence, optionally with the exception of an N-terminal and/or C-terminal cysteine, of at least one occurrence of P, preferably of at least 10% of all occurrences of P, more preferably of at least 25% of all occurrences of P, yet more preferably of at least 50% of all occurrences of P, even more preferably of at least 75% of all occurrences of P, yet even more preferably of at least 90% of all occurrences of P or even of at least 95% of all occurrences of P, especially of all of the occurrences of P, is identical to a sequence fragment of a protein, wherein the protein is identified by one of the following UniProt accession codes:
A9RAI0, B5SUY7, O41855, O56137, O56139, P03135, P04133, P04882, P08362, P10269, P12538, P69353, Q5Y9B2, Q5Y9B4, Q65311, Q6JC40, Q6VGT5, Q8JQF8, Q8JQG0, Q98654, Q9WBP8, Q9YIJ1, or of an AdV hexon protein, an AdV fiber protein, an AdV penton protein, an AdV IIIa protein, an AdV VI protein, an AdV VIII protein or an AdV IX protein or of any one of the capsid proteins identified in FIG. 4 and FIG. 5 or of any one of the capsid proteins listed in Cearley et al., 2008, or of a capsid protein sequence identified by any one of UniProt P03525, P03526, P03527, P03528, P03529, P0CK31, P11078, P11079, P12418, P12419, P15024, P21270, A1Z0P5, O09800, P03170, P04288, P04289, P04291, P04486, P06477, P06484, P06487, P06491, P08314, P08392, P08543, P10185, P10190, P10191, P10192, P10193, P10200, P10202, P10204, P10205, P10210, P10216, P10218, P10219, P10220, P10225, P10227, P10228, P10229, P10231, P10234, P32888, P68333, P08362, P35971, Q89933, P35974, P35976, P69358, P12576, Q9EMA9 and P03522;
optionally wherein the sequence fragment comprises at most three, even more preferably at most two, especially at most one amino acid substitutions.

Embodiment 27. The compound of any one of embodiments 1 to 26, wherein the entire sequence, optionally with the exception of an N-terminal and/or C-terminal cysteine, of peptide Pₐ is identical to a sequence fragment of a protein, wherein the protein is identified by one of the UniProt accession codes listed in embodiment 26; optionally wherein the sequence fragment comprises at most three, even more preferably at most two, especially at most one amino acid substitutions.

Embodiment 28. The compound of any one of embodiments 1 to 27, wherein the entire sequence, optionally with the exception of an N-terminal and/or C-terminal cysteine, of peptide P_{b} is identical to a sequence fragment of a protein, wherein the protein is identified by one of the UniProt accession codes listed in embodiment 26; optionally wherein the sequence fragment comprises at most three, even more preferably at most two, especially at most one amino acid substitutions.

Embodiment 29. The compound of any one of embodiments 1 to 28, wherein the entire sequence, optionally with the exception of an N-terminal and/or C-terminal cysteine, of peptide Pₐ is identical to a sequence fragment of a protein and the entire sequence, optionally with the exception of an N-terminal and/or C-terminal cysteine, of peptide P_{b} is identical to the same or another, preferably another, sequence fragment of the same protein, wherein the protein is identified by one of the UniProt accession codes listed in embodiment 26;
optionally wherein the sequence fragment comprises at most three, even more preferably at most two, especially at most one amino acid substitutions.

Embodiment 30. The compound of any one of embodiments 1 to 29, wherein said sequence fragment comprises a sequence of at least 4 or at least 5 or at least 6, preferably at least 7, more preferably at least 8, even more preferably at least 9, yet even more preferably at least 10 consecutive amino acids selected from:
the group of AdV sequences ETGPPTVPFLTPPF (SEQ ID NO: 32), HDSKLSIATQGPL (SEQ ID NO: 33), LNLRLGQGPLFINSAHNLDINY (SEQ ID NO: 34), VDPMDEPTLLYVLFEVEDVV (SEQ ID NO: 35), MKRARPSEDTENPVYPYD (SEQ ID NO: 36), ISGTVQSAHLIIRFD (SEQ ID NO: 37), LGQGPLFINSAHNLDINYNKGLYLF (SEQ ID NO: 38), SYPFDAQNQLNLRLGQGPLFIN (SEQ ID NO: 39), GDTTPSAYSMSFSWDWSGHNYIN (SEQ ID NO: 40), VLLNNSFLDPEYWNFRN (SEQ ID NO: 41), HNYINEIFATSSYTESYIA (SEQ ID NO: 42), DEAATALEINLEEEDDDNEDEVDEQAEQQKTH (SEQ ID NO: 43), INLEEEDDDNEDEVDEQAEQ (SEQ ID NO: 44), DNEDEVDEQAEQQKTHVF (SEQ ID NO: 45), EWDEAATALEINLEE (SEQ ID NO: 46), PKVVLYSEDVDIETPDTHISYMP (SEQ ID NO: 47), YIPESYKDRMYSFFRNF (SEQ ID NO: 48), DSIGDRTRYFSMW (SEQ ID NO: 49), SYKDRMYSFERNF (SEQ ID NO: 50), and FLVOMLANYNIGYQGFY (SEQ ID NO: 51), or
the group of AAV sequences WONRDVYLQGPIWAKIP (SEQ ID NO: 52), DNTYFGYSTPWGYFDFNRFHC (SEQ ID NO: 53), MANQAKNWLPGPCY (SEQ ID NO: 54), LPYVLGSAHQGCLPPFP (SEQ ID NO: 55), NGSQAVGRSSFYCLEYF (SEQ ID NO: 56), PLIDQYLYYL (SEQ ID NO: 57), EERFFPSNGILIF (SEQ ID NO: 58) ADGVGSSSGNWHC (SEQ ID NO: 59), SEQ ID NOs: 383-1891 (see Table 1)-preferably group III of Table 1, more preferably group II of Table 1, especially group I of Table 1—and SEQ ID NOs: 1892-2063 (see Table 2)-preferably group I of Table 2—and sequences of group II or III of Table 3 (in particular SEQ ID NOs: 2064-2103), more preferably sequences of group I of Table 3, or the group of sequences of Table 4, in particular the group of sequences identified by SEQ ID NOs: 2104-2190, or the group of sequences of Table 5;
optionally wherein at most three, preferably at most two, most preferably at least one amino acid of the sequence fragment is independently substituted by any other amino acid.

Embodiment 31. The compound of any one of embodiments 1 to 30, wherein, independently for each occurrence, P comprises a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences consisting of GPPTVPFLTP (SEQ ID NO: 60), ETGPPTVPFLIPP (SEQ ID NO: 61), TGPPTVPFLT (SEQ ID NO: 62), PTVPELTPPF (SEQ ID NO: 63), HDSKLSIATQGPL (SEQ ID NO: 64), SIATQGP (SEQ ID NO: 65), NLRLGQGPLF (SEQ ID NO: 66), QGPLFINSAH (SEQ ID NO: 67), PLFINSAHNLD (SEQ ID NO: 68), LGQGPLF (SEQ ID NO: 69), LNLRLGQGPL (SEQ ID NO: 70), GQGPLFI (SEQ ID NO: 71), NLRLGQGPLFINS (SEQ ID NO: 72), LFINSAHNLDINY (SEQ ID NO: 73), FINSAHNLDI (SEQ ID NO: 74), LRLGQGPLFI (SEQ ID NO: 75), GPLFINSAHN (SEQ ID NO: 76), DEPTLLYVLFEVF (SEQ ID NO: 77), TLLYVLFEVE (SEQ ID NO: 78), DEPTLLYVLF (SEQ ID NO: 79), TLLYVLFEVEDVV (SEQ ID NO: 80), TLLYVLF (SEQ ID NO: 81), MDEPTLLYVLFEV (SEQ ID NO: 82), EPTLLYVLFE (SEQ ID NO: 83), DPMDEPTLLYVLF (SEQ ID NO: 84), LLYVLFEVED (SEQ ID NO: 85), YVLFEVEDVV (SEQ ID NO: 86), PTLLYVLFEV (SEQ ID NO: 87), PTLLYVLFEVFDV (SEQ ID NO: 88), LYVLFEVEDV (SEQ ID NO: 89), EPTLLYVLFEVED (SEQ ID NO: 90), LYVLFEV (SEQ ID NO: 91), PMDEPTLLYVLFE (SEQ ID NO: 92), LLYVLFE (SEQ ID NO: 93), VDPMDEPTLLYVL (SEQ ID NO: 94), YVLFEVE (SEQ ID NO: 95), PTLLYVL (SEQ ID NO: 96), MKRARPSEDTF (SEQ ID NO: 97), KRARPSEDTF (SEQ ID NO: 98), MKRARPSEDT (SEQ ID NO: 99), MKRARPSEDTEN (SEQ ID NO: 100), ARPSEDTENP (SEQ ID NO: 101), RARPSEDTEN (SEQ ID NO: 102), RPSEDTE (SEQ ID NO: 103), MKRARPSEDTENP (SEQ ID NO: 104), RARPSEDTENPVY (SEQ ID NO: 105), ARPSEDT (SEQ ID NO: 106), EDTENPVYPY (SEQ ID NO: 107), RPSEDTENPVYPY (SEQ ID NO: 108), KRARPSEDTENPV (SEQ ID NO: 109), DTFNPVY (SEQ ID NO: 110), RPSEDTENPV (SEQ ID NO: 111), PSEDTENPVY (SEQ ID NO: 112), DTENPVYPYD (SEQ ID NO: 113), VOSAHLIIRF (SEQ ID NO: 114), AHLIIRF (SEQ ID NO: 115), SGTVQSAHLIIRF (SEQ ID NO: 116), TVQSAHLIIR (SEQ ID NO: 117), HLIIRFD (SEQ ID NO: 118), SAHLIIR (SEQ ID NO: 119), QSAHLIIRED (SEQ ID NO: 120), ISGTVQSAHLIIR (SEQ ID NO: 121), GTVQSAHLII (SEQ ID NO: 122), GTVQSAHLIIRED (SEQ ID NO: 123), QSAHLII (SEQ ID NO: 124), HNLDINY (SEQ ID NO: 125), LFINSAHNLDINY (SEQ ID NO: 126), NLDINYNKGLYLF (SEQ ID NO: 127), FVSPNG (SEQ ID NO: 128), NYINEIF (SEQ ID NO: 129), NKGLYLF (SEQ ID NO: 130), INYNKGLYLF (SEQ ID NO: 131), NSAHNLDINY (SEQ ID NO: 132), WDWSGHNYINEIF (SEQ ID NO: 133), SGHNYINEIF (SEQ ID NO: 134), LGTGLSF (SEQ ID NO: 135), PFLTPPF (SEQ ID NO: 136), LGQGPLF (SEQ ID NO: 137), NLRLGQGPLF (SEQ ID NO: 138), NQLNLRLGQGPLF (SEQ ID NO: 139), GOGPLFI (SEQ ID NO: 140), QLNLRLGQGPLFI (SEQ ID NO: 141), SYPFDAQNQLNLR (SEQ ID NO: 142), YPFDAQNOLNLRL (SEQ ID NO: 143), LRLGQGPLFI (SEQ ID NO: 144), NOLNLRL (SEQ ID NO: 145), FDAONOLNLR (SEQ ID NO: 146), QNOLNLR (SEQ ID NO: 147), QGPLFIN (SEQ ID NO: 148), PFDAQNOLNLRLG (SEQ ID NO: 149), DAQNQLNLRL (SEQ ID NO: 150), RLGQGPLFIN (SEQ ID NO: 151), QLNLRLG (SEQ ID NO: 152), FDAQNQLNLRLGQ (SEQ ID NO: 153), LNLRLGQGPLFIN (SEQ ID NO: 154), AQNOLNLRLG (SEQ ID NO: 155), AQNQLNL (SEQ ID NO: 156), LNLRLGO (SEQ ID NO: 157), SYPFDAQNQL (SEQ ID NO: 158), PFDAQNOLNL (SEQ ID NO: 159), YSMSFSW (SEQ ID NO: 160), TPSAYSMSFSWDW (SEQ ID NO: 161), MSFSWDW (SEQ ID NO: 162), PSAYSMSFSW (SEQ ID NO: 163), DTTPSAYSMSFSW (SEQ ID NO: 164), TTPSAYSMSF (SEQ ID NO: 165), YSMSFSWDWS (SEQ ID NO: 166), TGDTTPSAYSMSF (SEQ ID NO: 167), FSWDWSGHNY (SEQ ID NO: 168), SFSWDWS (SEQ ID NO: 169), SAYSMSF (SEQ ID NO: 170), SFSWDWSGHN (SEQ ID NO: 171), SAYSMSFSWD (SEQ ID NO: 172), SMSFSWD (SEQ ID NO: 173), SWDWSGHNYI (SEQ ID NO: 174), AYSMSFS (SEQ ID NO: 175), SMSFSWDWSGHNY (SEQ ID NO: 176), FSWDWSG (SEQ ID NO: 177), SWDWSGH (SEQ ID NO: 178), FLDPEYWNER (SEQ ID NO: 179), SELDPEYWNF (SEQ ID NO: 180), PEYWNER (SEQ ID NO: 181), LNNSELDPEYWNF (SEQ ID NO: 182), NNSFLDPEYWNER (SEQ ID NO: 183), FLDPEYW (SEQ ID NO: 184), DPEYWNF (SEQ ID NO: 185), NNSFLDPEYW (SEQ ID NO: 186), VLLNNSFLDPEYW (SEQ ID NO: 187), EYWNFRN (SEQ ID NO: 188), LNNSFLDPEY (SEQ ID NO: 189), LDPEYWNERN (SEQ ID NO: 190), LNNSFLD (SEQ ID NO: 191), NSFLDPEYWN (SEQ ID NO: 192), SSYTESY (SEQ ID NO: 193), FATSSYTESY (SEQ ID NO: 194), YINEIFATSSYTF (SEQ ID NO: 195), SYTESYI (SEQ ID NO: 196), ATSSYTF (SEQ ID NO: 197), EIFATSSYTF (SEQ ID NO: 198), NEIFATSSYTFSY (SEQ ID NO: 199), ATSSYTESYI (SEQ ID NO: 200), HNYINEIFATSSY (SEQ ID NO: 201), IFATSSY (SEQ ID NO: 202), INEIFATSSY (SEQ ID NO: 203), NYINEIFATSSYT (SEQ ID NO: 204), YINEIFA (SEQ ID NO: 205), YTESYIA (SEQ ID NO: 206), EIFATSSYTESYI (SEQ ID NO: 207), ALEINLEEEDDDN (SEQ ID NO: 208), ATALEINLEEEDD (SEQ ID NO: 209), EAATALEINLEEE (SEQ ID NO: 210), LEINLEE (SEQ ID NO: 211), TALEINLEEEDDD (SEQ ID NO: 212), EINLEEE (SEQ ID NO: 213), ALEINLEEED (SEQ ID NO: 214), LEINLEEEDD (SEQ ID NO: 215), TALEINLEEE (SEQ ID NO: 216), DEAATALEINLEE (SEQ ID NO: 217), LEINLEEEDDDNE (SEQ ID NO: 218), AATALEINLEEED (SEQ ID NO: 219), EINLEEEDDD (SEQ ID NO: 220), ATALEINLEE (SEQ ID NO: 221), INLEEEDDDN (SEQ ID NO: 222), NLEEEDDDNE (SEQ ID NO: 223), DEVDEQA (SEQ ID NO: 224), EDDDNEDEVDEQA (SEQ ID NO: 225), DDNEDEVDEQAEQ (SEQ ID NO: 226), EVDEQAE (SEQ ID NO: 227), DNEDEVDEQA (SEQ ID NO: 228), VDEQAEQ (SEQ ID NO: 229), EDEVDEQAEQQKT (SEQ ID NO: 230), EDEVDEQAEQ (SEQ ID NO: 231), DEVDEQAEQQKTH (SEQ ID NO: 232), NEDEVDEQAEQOK (SEQ ID NO: 233), DEVDEQAEQQ (SEQ ID NO: 234), EINLEEEDDDNED (SEQ ID NO: 235), NLEEEDDDNEDEV (SEQ ID NO: 236), INLEEED (SEQ ID NO: 237), LEEEDDDNED (SEQ ID NO: 238), INLEEEDDDNEDE (SEQ ID NO: 239), DDDNEDEVDEQAE (SEQ ID NO: 240), LEEEDDDNEDEVD (SEQ ID NO: 241), DDNEDEVDEQ (SEQ ID NO: 242), EDDDNED (SEQ ID NO: 243), NLEEEDD (SEQ ID NO: 244), DDNEDEV (SEQ ID NO: 245), DDDNEDEVDE (SEQ ID NO: 246), DDDNEDE (SEQ ID NO: 247), (SEQ ID NO: 248), EEDDDNE (SEQ ID NO: 249), EDDDNEDEVD EEEDDDNEDE (SEQ ID NO: 250), EDEVDEQ (SEQ ID NO: 251), EEDDDNEDEVDEQ (SEQ ID NO: 252), EEDDDNEDEV (SEQ ID NO: 253), EEEDDDNEDEVDE (SEQ ID NO: 254), EVDEQAEQOK (SEQ ID NO: 255), DNEDEVDEQAEQQ (SEQ ID NO: 256), VDEQAEQQKT (SEQ ID NO: 257), EVDEQAEQQKTHV (SEQ ID NO: 258), VDEQAEQQKTHVF (SEQ ID NO: 259), ALEINLE (SEQ ID NO: 260), WDEAATALEINLE (SEQ ID NO: 261), A AATALEINLE (SEQ ID NO: 262), EWDEAATALEINL (SEQ ID NO: 263), EAATALEINL (SEQ ID NO: 264), LYSEDVDIET (SEQ ID NO: 265), LYSEDVDIETPDT (SEQ ID NO: 266), KVVLYSEDVDIET (SEQ ID NO: 267), IETPDTH (SEQ ID NO: 268), VDIETPDTHI (SEQ ID NO: 269), VLYSEDVDIE (SEQ ID NO: 270), DVDIETPDTHISY (SEQ ID NO: 271), VVLYSEDVDIETP (SEQ ID NO: 272), SEDVDIETPDTHI (SEQ ID NO: 273), ETPDTHI (SEQ ID NO: 274), VLYSEDVDIETPD (SEQ ID NO: 275), DVDIETPDTH (SEQ ID NO: 276), DIETPDTHIS (SEQ ID NO: 277), EDVDIETPDTHIS (SEQ ID NO: 278), IETPDTHISY (SEQ ID NO: 279), YSEDVDIETPDTH (SEQ ID NO: 280), VDIETPDTHISYM (SEQ ID NO: 281), PKVVLYSEDVDIE (SEQ ID NO: 282), DIETPDT (SEQ ID NO: 283), DIETPDTHISYMP (SEQ ID NO: 284), EDVDIETPDT (SEQ ID NO: 285), ETPDTHISYM (SEQ ID NO: 286), IETPDTHISYMP (SEQ ID NO: 287), DRMYSFERNF (SEQ ID NO: 288), DRMYSFF (SEQ ID NO: 289), YSFERNF (SEQ ID NO: 290), IPESYKDRMYSFF (SEQ ID NO: 291), SYKDRMYSFF (SEQ ID NO: 292), ESYKDRMYSF (SEQ ID NO: 293), KDRMYSF (SEQ ID NO: 294), YIPESYKDRMYSF (SEQ ID NO: 295), PESYKDRMYSFFR (SEQ ID NO: 296), YKDRMYSFER (SEQ ID NO: 297), TRYFSMW (SEQ ID NO: 298), GDRTRYF (SEQ ID NO: 299), DSIGDRTRYF (SEQ ID NO: 300), DSIGDRTRYFSMW (SEQ ID NO: 301), GDRTRYFSMW (SEQ ID NO: 302), DRMYSFERNF (SEQ ID NO: 303), SYKDRMYSFERNF (SEQ ID NO: 304), NYNIGYOGFY (SEQ ID NO: 305), ANYNIGYQGF (SEQ ID NO: 306), MLANYNIGYQGFY (SEQ ID NO: 307), IGYQGFY (SEQ ID NO: 308), FLVQMLANYNIGY (SEQ ID NO: 309), NIGYQGF (SEQ ID NO: 310) and QMLANYNIGYQGF (SEQ ID NO: 311), optionally wherein at most three, preferably at most two, most preferably at least one amino acid is independently substituted by any other amino acid.

Embodiment 32. The compound of any one of embodiments 1 to 30, wherein, independently for each occurrence, P comprises a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences consisting of YLQGPIW (SEQ ID NO: 312), VYLQGPI (SEQ ID NO: 313), WONRDVY (SEQ ID NO: 314), DVYLQGP (SEQ ID NO: 315), QNRDVYL (SEQ ID NO: 316), LOGPIWA (SEQ ID NO: 317), RDVYLQG (SEQ ID NO: 318), NRDVYLQ (SEQ ID NO: 319), YFGYSTPWGYFDF (SEQ ID NO: 320), FGYSTPWGYF (SEQ ID NO: 321), GYSTPWGYFD (SEQ ID NO: 322), YSTPWGYFDF (SEQ ID NO: 323), NTYFGYSTPWGYF (SEQ ID NO: 324), TPWGYFDFNRFHC (SEQ ID NO: 325), TYFGYSTPWGYFD (SEQ ID NO: 326), DNTYFGYSTPWGY (SEQ ID NO: 327), YFGYSTPWGY (SEQ ID NO: 328), FGYSTPWGYFDEN (SEQ ID NO: 329), NWLPGPC (SEQ ID NO: 330), WLPGPCY (SEQ ID NO: 331), QAKNWLPGPC (SEQ ID NO: 332), AKNWLPGPCY (SEQ ID NO: 333), MANQAKNWLPGPC (SEQ ID NO: 334), QGCLPPF (SEQ ID NO: 335), GCLPPFP (SEQ ID NO: 336), VLGSAHQGCLPPF (SEQ ID NO: 337), LPYVLGSAHQGCL (SEQ ID NO: 338), YVLGSAHQGC (SEQ ID NO: 339), CLPPFPA (SEQ ID NO: 340), SAHQGCLPPF (SEQ ID NO: 341), VLGSAHQGCL (SEQ ID NO: 342), PYVLGSAHQGCLP (SEQ ID NO: 343), GRSSFYC (SEQ ID NO: 344), AVGRSSFYCLEYF (SEQ ID NO: 345), AVGRSSFYCL (SEQ ID NO: 346), QAVGRSSFYCLEY (SEQ ID NO: 347), NGSQAVGRSSFYC (SEQ ID NO: 348), DOYLYYL (SEQ ID NO: 349), PLIDQYLYYL (SEQ ID NO: 350), IDQYLYY (SEQ ID NO: 351), FFPSNGILIF (SEQ ID NO: 352), EERFFPSNGILIF (SEQ ID NO: 353), VGSSSGNWHC (SEQ ID NO: 354) and ADGVGSSSGNWHC (SEQ ID NO: 355), optionally wherein at most three, preferably at most two, most preferably at least one amino acid is independently substituted by any other amino acid; or wherein, independently for each occurrence, P comprises a 6-amino-acid fragment, preferably a 7-amino-acid-fragment, more preferably a 8-amino-acid-fragment, even more preferably a 9-amino-acid fragment, yet even more preferably a 10-amino-acid fragment or even a 11-amino-acid-fragment or yet even a 12-amino-acid-fragment, especially a 13-amino-acid-fragment selected from the group of sequences consisting of SEQ ID NOS: 383-1891 (see Table 1)-preferably group III of Table 1, more preferably group II of Table 1, especially group I of Table 1—and SEQ ID NOs: 1892-2063 (see Table 2)-preferably group I of Table 2—and sequences of group II or III of Table 3 (in particular SEQ ID NOs: 2064-2103), more preferably sequences of group I of Table 3, optionally wherein at most three, preferably at most two, most preferably at least one amino acid is independently substituted by any other amino acid; or wherein, independently for each occurrence, P comprises a 6-amino-acid fragment, preferably a 7-amino-acid-fragment, more preferably a 8-amino-acid-fragment, even more preferably a 9-amino-acid fragment, yet even more preferably a 10-amino-acid fragment or even a 11-amino-acid-fragment or yet even a 12-amino-acid-fragment, especially a 13-amino-acid-fragment selected from the group of sequences of Table 4, in particular the group of sequences identified by SEQ ID NOs: 2104-2190, or the group of sequences of Table 5, optionally wherein at most three, preferably at most two, most preferably at least one amino acid is independently substituted by any other amino acid.

Embodiment 33. The compound of any one of embodiments 1 to 32, wherein, independently for each occurrence, P consists of a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in embodiment 31 or selected from the group of sequences set forth in embodiment 32, optionally wherein at most three, preferably at most two, most preferably at least one amino acid is independently substituted by any other amino acid, optionally with an N-terminal and/or C-terminal cysteine residue.

Embodiment 34. The compound of any one of embodiments 1 to 33, wherein each of the peptide n-mers is covalently bound to the biopolymer scaffold, preferably via a linker each.

Embodiment 35. The compound of any one of embodiments 1 to 34, wherein at least one of said linkers is selected from disulphide bridges and PEG molecules.

Embodiment 36. The compound of any one of embodiments 1 to 35, wherein at least one of the spacers S is selected from PEG molecules or glycans.

Embodiment 37. The compound of any one of embodiments 1 to 36, wherein Pₐ comprises a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in embodiment 31, optionally wherein at most three, preferably at most two, most preferably at least one amino acid is independently substituted by any other amino acid.

Embodiment 38. The compound of any one of embodiments 1 to 37, wherein P_{b} comprises a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in embodiment 31, optionally wherein at most three, preferably at most two, most preferably at least one amino acid is independently substituted by any other amino acid.

Embodiment 39. The compound of any one of embodiments 1 to 36, wherein Pₐ comprises a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in embodiment 32, optionally wherein at most three, preferably at most two, most preferably at least one amino acid is independently substituted by any other amino acid.

Embodiment 40. The compound of any one of embodiments 1 to 37, wherein P_{b} comprises a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in embodiment 32, optionally wherein at most three, preferably at most two, most preferably at least one amino acid is independently substituted by any other amino acid.

Embodiment 41. The compound of any one of embodiments 6 to 40, wherein the first peptide n-mer is Pₐ—S—P_{b} and the second peptide n-mer is Pₐ—S—P_{b}.

Embodiment 42. The compound of any one of embodiments 6 to 41, wherein the peptide Pₐ and the peptide P_{b} comprise the same amino-acid sequence fragment, wherein the amino-acid sequence fragment has a length of at least 5 amino acids, even more preferably at least 6 amino acids, yet even more preferably at least 7 amino acids, especially at least 8 amino acids or even at least 9 amino acids.

Embodiment 43. The compound of any one of embodiments 1 to 42, wherein Pₐ consists of a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in embodiment 31, optionally wherein at most three, preferably at most two, most preferably at least one amino acid is independently substituted by any other amino acid, optionally with an N-terminal and/or C-terminal cysteine residue.

Embodiment 44. The compound of any one of embodiments 1 to 43, wherein P_{b} consists of a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in embodiment 31, optionally wherein at most three, preferably at most two, most preferably at least one amino acid is independently substituted by any other amino acid, optionally with an N-terminal and/or C-terminal cysteine residue.

Embodiment 45. The compound of any one of embodiments 1 to 42, wherein Pₐ consists of a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in embodiment 32, optionally wherein at most three, preferably at most two, most preferably at least one amino acid is independently substituted by any other amino acid, optionally with an N-terminal and/or C-terminal cysteine residue.

Embodiment 46. The compound of any one of embodiments 1 to 43, wherein P_{b} consists of a 6-amino-acid fragment, preferably a 7-amino-acid-fragment, more preferably a 8-amino-acid-fragment, even more preferably a 9-amino-acid fragment, yet even more preferably a 10-amino-acid fragment, especially an entire sequence selected from the group of sequences set forth in embodiment 32, optionally wherein at most three, preferably at most two, most preferably at least one amino acid is independently substituted by any other amino acid, optionally with an N-terminal and/or C-terminal cysteine residue

Embodiment 47. The compound of embodiments 1 to 46, wherein the first peptide n-mer is Pₐ—S—P_{b} and the second peptide n-mer is Pₐ—S—P_{b}.

Embodiment 48. The compound of embodiments 1 to 47, wherein the peptide Pₐ and the peptide P_{b} comprise the same amino-acid sequence fragment, wherein the amino-acid sequence fragment has a length of at least 5 amino acids, even more preferably at least 6 amino acids, yet even more preferably at least 7 amino acids, especially at least 8 amino acids or even at least 9 amino acids.

Embodiment 49. The compound of any one of embodiments 1 to 48, wherein the oncolytic virus is non-pathogenic (in the individual to be treated).

Embodiment 50. The compound of any one of embodiments 1 to 49, wherein the biopolymer scaffold is an anti-CD163 antibody (i.e. an antibody specific for a CD163 protein) or CD163-binding fragment thereof.

Embodiment 51. The compound of embodiment 50, wherein the anti-CD163 antibody or CD163-binding fragment thereof is specific for human CD163 and/or is specific for the extracellular region of CD163, preferably for an SRCR domain of CD163, more preferably for any one of SRCR domains 1-9 of CD163, even more preferably for any one of SRCR domains 1-3 of CD163, especially for SRCR domain 1 of CD163.

Embodiment 52. The compound of embodiment 50 or 51, wherein the anti-CD163 antibody or CD163-binding fragment thereof is specific for one of the following peptides:
a peptide consisting of 7-25, preferably 8-20, even more preferably 9-15, especially 10-13 amino acids, wherein the peptide comprises the amino acid sequence CSGRVEVKVQEEWGTVCNNGWSMEA (SEQ ID NO: 3) or a 7-24 amino-acid fragment thereof,
a peptide consisting of 7-25, preferably 8-20, even more preferably 9-15, especially 10-13 amino acids, wherein the peptide comprises the amino acid sequence DHVSCRGNESALWDCKHDGWG (SEQ ID NO: 13) or a 7-20 amino-acid fragment thereof, or
a peptide consisting of 7-25, preferably 8-20, even more preferably 9-15, especially 10-13 amino acids, wherein the peptide comprises the amino acid sequence SSLGGTDKELRLVDGENKCS (SEQ ID NO: 24) or a 7-19 amino-acid fragment thereof.

Embodiment 53. The compound of embodiment 50 or 51, wherein the anti-CD163 antibody or CD163-binding fragment thereof is specific for a peptide comprising the amino acid sequence ESALW (SEQ ID NO: 14) or ALW.

Embodiment 54. The compound of embodiment 50 or 51, wherein the anti-CD163 antibody or CD163-binding fragment thereof is specific for a peptide comprising the amino acid sequence GRVEVKVQEEW (SEQ ID NO: 4), WGTVCNNGWS (SEQ ID NO: 5) or WGTVCNNGW (SEQ ID NO: 6),

Embodiment 55. The compound of embodiment 50 or 51, wherein the anti-CD163 antibody or CD163-binding fragment thereof is specific for a peptide comprising the amino acid sequence SSLGGTDKELR (SEQ ID NO: 25) or SSLGG (SEQ ID NO: 26).

Embodiment 56. The compound of any one of embodiments 1 to 55, wherein the oncolytic virus is AAV1, AAV2, AAV3, AAV5, AAV7 or AAV8.

Embodiment 57. The compound of any one of embodiments 1 to 55, wherein the oncolytic virus is AAV8.

Embodiment 58. The compound of any one of embodiments 1 to 55, wherein the oncolytic virus is Ad5.

Embodiment 59. The compound of any one of embodiments 58, wherein the oncolytic virus is AdHu5.

Embodiment 60. The compound of any one of embodiments 1 to 59, wherein the oncolytic virus is an oncolytic virus specific for a mammal, in particular a human.

Embodiment 61. The compound of any one of embodiments 1 to 60, wherein the biopolymer scaffold is selected from human immunoglobulins and human transferrin.

Embodiment 62. The compound of embodiment any one of embodiments 1 to 61, wherein the biopolymer scaffold is human transferrin.

Embodiment 63. The compound of any one of embodiments 49 to 62, wherein at least one of the at least two peptides is circularized.

Embodiment 64. The compound of any one of embodiments 1 to 63, wherein the compound is non-immunogenic in humans.

Embodiment 65. A pharmaceutical composition comprising the compound of any one of embodiments 1 to 64 and at least one pharmaceutically acceptable excipient.

Embodiment 66. The pharmaceutical composition of embodiment 65, wherein the composition is prepared for intraperitoneal, subcutaneous, intramuscular and/or intravenous administration and/or wherein the composition is for repeated administration.

Embodiment 67. The pharmaceutical composition of any one of embodiments 1 to 66, wherein the molar ratio of peptide P to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

Embodiment 68. The pharmaceutical composition of any one of embodiments 6 to 67, wherein the molar ratio of peptide Pₐ to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

Embodiment 69. The pharmaceutical composition of any one of embodiments 6 to 68, wherein the molar ratio of peptide P_{b} to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

Embodiment 70. The pharmaceutical composition of any one of embodiments 65 to 69 for use in therapy, preferably therapy of a neoplasm such as a benign neoplasm, an in situ neoplasm or an malignant neoplasm, especially therapy of a solid tumor or a hematological malignancy.

Embodiment 71. The pharmaceutical composition for use according to embodiment 70, for use in increasing efficacy of an oncolytic virus or oncolytic VLP in an individual, preferably wherein the pharmaceutical composition is administered to the individual prior to or concurrently with (systemic or local, preferably systemic) administration of the oncolytic virus or oncolytic VLP (based on the oncolytic virus) and/or preferably wherein the titer of an antibody specific for the oncolytic virus or oncolytic VLP (and preferably also for the compound) is determined after administration of the pharmaceutical composition and prior to (systemic or local, preferably systemic) administration of the virus or VLP, especially wherein the virus or VLP is only administered if the titer is below a threshold.

Embodiment 72. The pharmaceutical composition for use according to embodiment 70 or 71, wherein the administration of the pharmaceutical composition is followed by (systemic or local, preferably systemic) administration of the oncolytic virus or oncolytic VLP (based on the oncolytic virus) within 96 hours, preferably within 72 hours or even within 60 hours, more preferably within 48 hours, even more preferably within 36 hours, yet even more preferably within 24 hours, especially within 12 hours; preferably wherein the (systemic or local, preferably systemic) administration of the oncolytic virus or oncolytic VLP occurs at least 6 hours, preferably at least 12 hours, more preferably at least 24 hours after administration of the pharmaceutical composition.

Embodiment 73. The pharmaceutical composition for use according to embodiment 70, for use in increasing efficacy of an oncolytic virus or oncolytic VLP (based on the oncolytic virus) in an individual, preferably wherein the pharmaceutical composition is administered to the individual prior to or concurrently with (systemic or local, preferably systemic) administration of the oncolytic virus or oncolytic VLP (based on the oncolytic virus).

Embodiment 74. The pharmaceutical composition for use according to embodiment 73, wherein the pharmaceutical composition is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window; preferably wherein this window is followed by (systemic or local, preferably systemic) administration of the oncolytic virus or oncolytic VLP (based on the oncolytic virus) within 96 or even within 72 hours, preferably within 60 hours, more preferably within 48 hours, even more preferably within 36 hours, yet even more preferably within 24 hours, especially within 12 hours.

Embodiment 75. The pharmaceutical composition for use according to any one of embodiments 71 to 74, wherein the individual is human.

Embodiment 76. The pharmaceutical composition for use according to any one of embodiments 70 to 75, wherein one or more antibodies are present in the individual which are specific for at least one occurrence of peptide P, or for peptide Pₐ and/or peptide P_{b}, preferably wherein said antibodies are neutralizing antibodies for said oncolytic virus.

Embodiment 77. The pharmaceutical composition for use according to any one of embodiments 70 to 76, wherein the composition is non-immunogenic in the individual.

Embodiment 78. The pharmaceutical composition for use according to any one of embodiments 70 to 77, wherein the composition is administered at a dose of 1-1000 mg, preferably 2-500 mg, more preferably 3-250 mg, even more preferably 4-100 mg, especially 5-50 mg, compound per kg body weight of the individual.

Embodiment 79. The pharmaceutical composition for use according to any one of embodiments 70 to 78, wherein the composition is administered intraperitoneally, subcutaneously, intramuscularly or intravenously.

Embodiment 80. A method of (transiently) sequestering (or depleting) one or more antibodies present in an individual, comprising obtaining a pharmaceutical composition as defined in any one of embodiments 65 to 69, wherein the composition is non-immunogenic in the individual and wherein the one or more antibodies present in the individual are specific for at least one occurrence of P, or for peptide Pₐ and/or peptide P_{b}; and administering the pharmaceutical composition to the individual.

Embodiment 81. The method of embodiment 80, wherein the individual is a human or non-human animal, preferably a non-human primate, a sheep, a pig, a dog or a rodent, in particular a mouse.

Embodiment 82. The method of embodiments 80 or 81, wherein the biopolymer scaffold is autologous with respect to the individual, preferably wherein the biopolymer scaffold is an autologous protein.

Embodiment 83. The method of any one of embodiments 80 to 82, wherein the individual is (systemically or locally, preferably systemically) administered a anti-neoplastic composition comprising an oncolytic virus prior to, concurrent with and/or subsequent to said administering of the pharmaceutical composition.

Embodiment 84. The method of any one of embodiments 80 to 83, wherein the individual is a non-human animal.

Embodiment 85. The method of any one of embodiments 80 to 82, wherein the individual is (systemically or locally, preferably systemically) administered a anti-neoplastic composition comprising an oncolytic virus and wherein the one or more antibodies present in the individual are specific for said oncolytic virus, preferably wherein said administering of the anti-neoplastic composition is prior to, concurrent with and/or subsequent to said administering of the pharmaceutical composition.

Embodiment 86. The method of embodiment 85, wherein the oncolytic virus contains genetic material, preferably recombinant genetic material, and/or a transgenic oncolytic payload.

Embodiment 87. The compound, composition or method of any one of embodiments 1 to 86, wherein the (human) individual has a neoplasm, preferably wherein the neoplasm is a benign neoplasm, an in situ neoplasm or a malignant neoplasm, especially a solid tumor or a hematological malignancy; in particular wherein the individual has a solid tumor preferably selected from the group of brain cancers, breast cancers, colon cancers, esophageal cancers, prostate cancers, bladder cancer, kidney cancers, endometrial cancers, thyroid cancers, gastric cancers, liver cancers, lung cancers, pancreatic cancers, uterine cancers, tracheal cancers, testicular cancers, cervical cancers, head and neck cancers, skin cancers, bone cancers, pancreatic cancers and ovarian cancers, or a hematological malignancy preferably selected from the group of leukemias, lymphomas and multiple myelomas.

Embodiment 88. The method of any one of embodiments 80 to 87, wherein the composition is administered intraperitoneally, subcutaneously, intramuscularly or intravenously.

Embodiment 89. A anti-neoplastic composition, comprising the compound of any one of embodiments 1 to 64 and further comprising the oncolytic virus (typically wherein the oncolytic virus contains genetic material) or oncolytic VLP (based on the oncolytic virus) and optionally at least one pharmaceutically acceptable excipient; preferably wherein the oncolytic virus comprises a peptide fragment with a sequence length of 6-13 amino acids, preferably 7-11 amino acids, more preferably 7-9 amino acids, and wherein the sequence of at least one occurrence of peptide P, or peptide Pₐ and/or peptide P_{b}, of the compound is at least 70% identical, preferably at least 75% identical, more preferably at least 80% identical, yet more preferably at least 85% identical, even more preferably at least 90% identical, yet even more preferably at least 95% identical, especially completely identical to the sequence of said peptide fragment.

Embodiment 90. The anti-neoplastic composition of embodiment 89, wherein the oncolytic virus is an AdV or an AAV or a measles virus, or a Herpes simplex virus (HSV), a poxvirus such as Vaccinia virus, a reovirus, a Newcastle disease virus (NVD), a rhabdovirus, a coxsackievirus, a lentivirus, an alphavirus such as Semliki Forest virus, a vesicular stomatitis virus (VSS), a myxoma virus (MYXV), a mengovirus, a bovine viral diarrhea virus (BVDV), a chimeric oncolytic virus, a picornavirus, a parvovirus, or a flavivirus, or an oncolytic VLP thereof.

Embodiment 91. The anti-neoplastic composition of embodiment 89 or 90, wherein the oncolytic virus is an AdV or an AAV or a measles virus, or an oncolytic VLP thereof.

Embodiment 92. The anti-neoplastic composition of any one of embodiments 89 to 90, wherein the composition is prepared for intravenous administration.

Embodiment 93. The pharmaceutical composition of any one of embodiments 89 to 92, wherein the composition is an aqueous solution.

Embodiment 94. The pharmaceutical composition of any one of embodiments 89 to 93 for use in inhibition of an immune reaction, preferably an antibody-mediated immune reaction, against the active agent.

Embodiment 95. The pharmaceutical composition for use according to embodiment 94, wherein the composition is non-immunogenic in the individual.

Embodiment 96. A method of inhibiting a (humoral) immune reaction to a treatment with an anti-neoplastic composition (comprising the oncolytic virus or oncolytic VLP) in an individual in need of treatment with the anti-neoplastic composition, comprising
obtaining a pharmaceutical composition as defined in any one of embodiments 89 to 95; wherein the compound of the pharmaceutical composition is non-immunogenic in the individual, and
administering the pharmaceutical composition to the individual.

Embodiment 97. The method of embodiment 96, wherein the individual is human.

Embodiment 98. The method of embodiment 96 or 97, wherein the biopolymer scaffold is autologous with respect to the individual, preferably wherein the biopolymer scaffold is an autologous protein.

Embodiment 99. The method of any one of embodiments 96 to 98, wherein the composition is administered intraperitoneally, subcutaneously, intramuscularly or intravenously.

Embodiment 100. A peptide with a sequence length of 6 to 50 amino acids, more preferably 6 to 25 amino acids, even more preferably 6 to 20 amino acids, yet more preferably 6 to 13 amino acids, wherein the peptide comprises a sequence of at least 4 or at least 5 or at least 6, preferably at least 7, more preferably at least 8, even more preferably at least 9, yet even more preferably at least 10 consecutive amino acids selected from:
the group of AdV sequences ETGPPTVPFLTPPF (SEQ ID NO: 32), HDSKLSIATQGPL (SEQ ID NO: 33), LNLRLGQGPLFINSAHNLDINY (SEQ ID NO: 34), VDPMDEPTLLYVLFEVEDVV (SEQ ID NO: 35), MKRARPSEDTENPVYPYD (SEQ ID NO: 36), ISGTVQSAHLIIRED (SEQ ID NO: 37), LGQGPLFINSAHNLDINYNKGLYLF (SEQ ID NO: 38), SYPFDAQNOLNLRLGQGPLFIN (SEQ ID NO: 39), GDTTPSAYSMSFSWDWSGHNYIN (SEQ ID NO: 40), VLLNNSFLDPEYWNFRN (SEQ ID NO: 41), HNYINEIFATSSYTESYIA (SEQ ID NO: 42), DEAATALEINLEEEDDDNEDEVDEQAEQQKTH (SEQ ID NO: 43), INLEEEDDDNEDEVDEQAEQ (SEQ ID NO: 44), DNEDEVDEQAEQQKTHVF (SEQ ID NO: 45), EWDEAATALEINLEE (SEQ ID NO: 46), PKVVLYSEDVDIETPDTHISYMP (SEQ ID NO: 47), YIPESYKDRMYSFERNF (SEQ ID NO: 48), DSIGDRTRYFSMW (SEQ ID NO: 49), SYKDRMYSFFRNE (SEQ ID NO: 50), and FLVOMLANYNIGYQGFY (SEQ ID NO: 51), or
the group of AAV sequences WONRDVYLOGPIWAKIP (SEQ ID NO: 52), DNTYFGYSTPWGYFDFNRFHC (SEQ ID NO: 53), MANQAKNWLPGPCY (SEQ ID NO: 54), LPYVLGSAHQGCLPPFP (SEQ ID NO: 55), NGSQAVGRSSFYCLEYF (SEQ ID NO: 56), PLIDQYLYYL (SEQ ID NO: 57), EERFFPSNGILIF (SEQ ID NO: 58), ADGVGSSSGNWHC (SEQ ID NO: 59), SEQ ID NOs: 383-1891 (see Table 1)-preferably group III of Table 1, more preferably group II of Table 1, especially group I of Table 1—and SEQ ID NOs: 1892-2063 (see Table 2)-preferably group I of Table 2—and sequences of group II or III of Table 3 (in particular SEQ ID NOs: 2064-2103), more preferably sequences of group I of Table 3, or the group of sequences of Table 4, in particular the group of sequences identified by SEQ ID NOs: 2104-2190, or the group of sequences of Table 5,
optionally wherein at most three, preferably at most two, most preferably at least one amino acid of the sequence is independently substituted by any other amino acid;
preferably wherein the peptide is a peptide as defined in embodiment 31, 32 or 33.

Embodiment 101. A method for detecting and/or quantifying oncolytic virus neutralizing antibodies in a biological sample comprising the steps of
bringing the sample into contact with the peptide of embodiment 100 or the compound of any one of embodiments 1 to 64, and
detecting the presence and/or concentration of the antibodies in the sample.

Embodiment 102. The method of embodiment 101, wherein the peptide or the compound is immobilized on a solid support, in particular a biosensor-based diagnostic device with an electrochemical, fluorescent, magnetic, electronic, gravimetric or optical biotransducer and/or wherein the peptide or the compound is coupled to a reporter or reporter fragment, such as a reporter fragment suitable for a PCA.

Embodiment 103. The method of embodiment 101 or 102, wherein the method is a sandwich assay, preferably an enzyme-linked immunosorbent assay (ELISA).

Embodiment 104. The method of any one of embodiments 101 to 103, wherein the sample is obtained from a mammal, preferably a human; preferably wherein the mammal (human) has a neoplasm, more preferably wherein the neoplasm is a benign neoplasm, an in situ neoplasm or a malignant neoplasm, even more preferably a solid tumor or a hematological malignancy, especially wherein the mammal has a solid tumor preferably selected from the group of brain cancers, breast cancers, colon cancers, esophageal cancers, prostate cancers, bladder cancer, kidney cancers, endometrial cancers, thyroid cancers, gastric cancers, liver cancers, lung cancers, pancreatic cancers, uterine cancers, tracheal cancers, testicular cancers, cervical cancers, head and neck cancers, skin cancers, bone cancers, pancreatic cancers and ovarian cancers, or a hematological malignancy preferably selected from the group of leukemias, lymphomas and multiple myelomas; in particular wherein the mammal (human) has been selected to undergo therapy with an oncolytic virus or oncolytic VLP or is undergoing therapy with an oncolytic virus or oncolytic VLP.

Embodiment 105. The method of any one of embodiment 101 to 104, wherein the sample is a blood sample, preferably whole blood, serum, or plasma.

Embodiment 106. Use of the peptide according to embodiment 100 or the compound of any one of embodiments 1 to 64 in an enzyme-linked immunosorbent assay (ELISA), preferably for a method as defined in any one of embodiments 101 to 105.

Embodiment 107. Diagnostic device comprising the peptide according to embodiment 100 or the compound of any one of embodiments 1 to 64 wherein the peptide or the compound is immobilized on a solid support and/or wherein the peptide or the compound is coupled to a reporter or reporter fragment, such as a reporter fragment suitable for a PCA.

Embodiment 108. Diagnostic device according to embodiment 107, wherein the solid support is an ELISA plate or a surface plasmon resonance chip.

Embodiment 109. Diagnostic device according to embodiment 107, wherein the diagnostic device is a lateral flow assay device or a biosensor-based diagnostic device with an electrochemical, fluorescent, magnetic, electronic, gravimetric or optical biotransducer.

Embodiment 110. A diagnostic kit comprising a peptide according to embodiment 100 or the compound of any one of embodiments 1 to 64, preferably diagnostic device according to any one of embodiment 107 to 109, and preferably one or more selected from the group of a buffer, a reagent, instructions.

Embodiment 111. An apheresis device comprising the peptide according to embodiment 100, preferably immobilized on a solid carrier.

Embodiment 112. The apheresis device according to embodiment 111, wherein the solid carrier is capable of being contacted with blood or plasma flow.

Embodiment 113. The apheresis device according to embodiment 111 or 112, wherein the solid carrier comprises the compound according to any one of embodiments 1 to 64.

Embodiment 114. The apheresis device according to any one of embodiment 111 to 113, wherein the solid carrier is a sterile and pyrogen-free column.

Embodiment 115. The apheresis device according to any one of embodiments 111 to 114, wherein the apheresis device comprises at least two, preferably at least three, more preferably at least four different peptides according to embodiment 100.

The present invention is further illustrated by the following figures and examples, without being restricted thereto.

FIG. 1: Schematic illustration of the inventive oncolytic regimen. The antibody lowering effect of SADCs is transient in an organism with pre-existing immunity, allowing for a therapeutic window for oncolytic virus administration. OV: oncolytic virus.

FIG. 2: Transient selective lowering of pre-existing antibody titers. Rapid antibody titer lowering at 24 hrs and 48 hrs after i.v. SADC administration was observed. Titer recovery starts after 48 hrs post SADC administration.

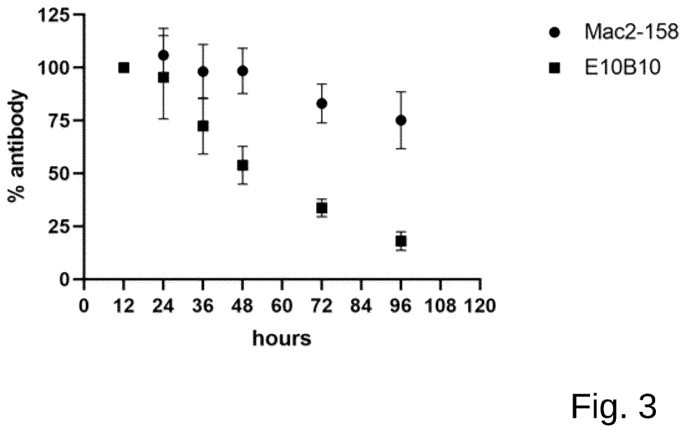

FIG. 3: Blood clearance of an anti-CD163-antibody-based biopolymer scaffold. In a mouse model, mAb E10B10 (specific for murine CD163) is much more rapidly cleared from circulation than mAb Mac2-158 (specific for human CD163 but not for murine CD163, thus serving as negative control in this experiment).

FIG. 4: AdV capsid protein sequences for use in the present invention. Databases accession numbers (in particular UniProt or GenBank accession numbers) are listed.

FIG. 5: AAV capsid protein sequences for use in the present invention. Databases accession numbers (in particular UniProt or GenBank accession numbers are listed), as well as references to sequences in patent publications.

### EXAMPLES

### Example 1: Transient Selective Lowering of Pre-Existing Antibody Titers

An SADC with a transferrin biopolymer scaffold conjugated to model peptides (PAVIMGNWENHWI; SEQ ID NO: 2226) was used to test the antibody-lowering effect in a mouse model (actively immunized using peptide PAVIMGNWENHWI coupled to keyhole limpet hemocyanin (KLH) as immunogen). FIG. 2 shows the binding signal intensities (arbitrary units) against peptide PAVIMGNWENHWI when detecting specific IgG levels by peptide arrays. Rapid titer lowering at 24 hrs and 48 hrs after i.v. SADC administration was observed. Titer recovery starts after 48 hrs post SADC administration. In conclusion, the antibody lowering effect of SADCs is transient in an organism with pre-existing immunity.

As explained above, antibodies against oncolytic viruses support their beneficial effect against cancer cells. However, these antibodies may at the same time inhibit oncolytic virus delivery, thereby reducing target access and therapeutic efficacy. In view of the observations in this example, it is thus especially beneficial to administer the oncolytic virus to a cancer patient in a time window from about 12 hours post SADC administration to about 60 hours post SADC administration (especially in larger mammals such as humans, the window may be extended e.g. up until 96 hours post SADC administration). Typically, the titer of a nAb against oncolytic viruses is determined before administering the oncolytic virus (or VLP) and the oncolytic virus (or VLP) is only administered if the titer is below a certain threshold (e.g. below 50% or 60% or 70% of the original titer). Otherwise, administration of the SADC may be repeated to further lower the titer.

A schematic illustration of the inventive oncolytic regimen is also shown in FIG. 1.

### Example 2: Generation of Mimotope-Based SADCs

mAb 4D2 is a mouse IgG2a mAb targeting the adenovirus fiber epitope peptide (NCBI Reference Sequence: AP 000226.1). It represents a prototype neutralizing antibody that was generated from UV irradiated Ad2 virus (Krasnykh et al, 1998).

Linear and circular peptides derived from wild-type or modified peptide amino acid sequences can be used for the construction of specific SADCs for the selective removal of neutralizing antibodies against viral vectors. In case of a particular epitope, linear peptides or constrained peptides such as cyclopeptides containing portions of an epitope or variants thereof, where for example, one or several amino acids have been substituted or chemically modified in order to improve affinity to an antibody (mimotopes), can be used for constructing SADCs. A peptide screen can be performed with the aim of identifying peptides with optimized affinity to neutralizing antibodies. The flexibility of structural or chemical peptide modification provided a solution to minimize the risk of immunogenicity, in particular of binding of the peptide to HLA and thus the risk of unwanted immune stimulation.

Therefore, wild-type as well as modified linear and circular peptide sequences are derived from an epitope of a viral capsid protein as disclosed herein, e.g. the epitopic sequence LNLRLGQGPLFINSAHNLDINY (SEQ ID NO: 34) of mAb 4D2 found in the course of the present invention (see example further below). Peptides of various length and positions are systematically permutated by amino acid substitutions and synthesized on a peptide array. This allows screening of 60000 circular and linear wild-type and mimotope peptides derived from these sequences. The peptide arrays are incubated with mAb 4D2. This antibody is therefore used to screen the 60000 peptides and 100 circular and 100 linear peptide hits are selected based on their relative binding strength to the antibody. Of these 200 peptides, 51 sequences are identical between the circular and the linear peptide group. All of the best peptides identified have at least one amino acid substitution when aligned to the original sequences, respectively and are therefore regarded as mimotopes. Also, higher binding strengths can be achieved with circularized peptides.

These newly identified peptides, preferentially those with high relative binding values, are used to generate SADCs for increasing efficacy of AdV-based oncolytic viruses.

### Example 3: SADCs to Transiently Reduce Antibodies Against Measles Virus

Three SADCs are provided to reduce anti-measles virus antibodies for oncolytic therapy with measles virus (see also Example 15 for the peptides used):
(a) SADC-a with Mac2-158 (as disclosed in WO 2011/039510 A2) as biopolymer scaffold and at least two peptides with the sequence SITIPYQGSG (SEQ ID NO: 2227) covalently bound to the scaffold,
(b) SADC-b with human transferrin as biopolymer scaffold and at least two peptides with the sequence PEWAPLKDNRI (SEQ ID NO: 2228) covalently bound to the scaffold, and
(c) SADC-c with human albumin as biopolymer scaffold and at least two peptides with the sequence GGHITHSGMEGMG (SEQ ID NO: 2229) covalently bound to the scaffold.

These SADCs are administered intravenously to an individual with cancer who will undergo oncolytic therapy with a measles virus in order to increase efficiency of the oncolytic therapy. 24 hours after administration of the SADCs, the oncolytic measles virus is administered.

### Example 4: In-Vivo Function of Anti-CD163-Antibody-Based SADC Biopolymer Scaffold

Rapid in vivo blood clearance of anti-mouse-CD163 mAb E10B10 (as disclosed in WO 2011/039510 A2). mAb E10B10 was resynthesized with a mouse IgG2a backbone. 50 μg mAb E10B10 and Mac2-158 (human-specific anti-CD163 mAb as disclosed in WO 2011/039510 A2, used as negative control in this example since it does not bind to mouse CD163) were injected i.v. into mice and measured after 12, 24, 36, 48, 72, 96 hours in an ELISA to determine the blood clearance.

mAb E10B10 was much more rapidly cleared from circulation than control mAb Mac2-158 was, as shown in FIG. 3, since E10B10 binds to the mouse CD163 whereas Mac2-158 is human-specific, although both were expressed as mouse IgG2a isotypes for direct comparison.

In conclusion, anti-CD163 antibodies are highly suitable as SADC scaffold because of their clearance profile. SADCs with such scaffolds will rapidly clear undesirable antibodies from circulation.

Detailed methods: 50 μg of biotinylated monoclonal antibodies E10B10 and biotinylated Mac2-158 were injected i.v. into mice and measured after 12, 24, 36, 48, 72, 96 hours to determine the clearance by ELISA: Streptavidin plates were incubated with plasma samples diluted in PBS+0.1% BSA+0.1% Tween20 for 1 h at room temperature (50 μl/well). After washing (3× with PBS+0.1% Tween20), bound biotinylated antibodies were detected with anti-mouse IgG+IgM-HRP antibody at a 1:1000 dilution. After washing, TMB substrate was added and development of the substrate was stopped with TMB Stop Solution. The signal at OD450 nm was read. The EC50 values were calculated by non-linear regression using 4 parametric curve fitting with constrained curves and least squares regression. EC50 values at time-point T12 (this was the first measured time-point after antibody injection) was set at 100%, all other EC50 values were compared to the levels at T12.

### Example 5: Epitope Mapping of Anti-CD163 mAbs

mAb E10B10 provides CD163-mediated, accelerated in vivo clearance from blood in mice (see example 11). The epitope of this antibody was fine mapped using circular peptide arrays, whereby the peptides were derived from mouse CD163. As a result, a peptide cluster that is recognized by mAb E10B10 was identified (see example 13).

The same epitope mapping procedure using circularized peptides was performed with mAb Mac2-158 (as disclosed in WO 2011/039510 A2). Epitope mapping results for mAb Mac2-158 yielded two peptide clusters (see example 13) which allowed further demarcation of CD163 epitope regions that are especially relevant to internalization of ligands and antibodies that bind to the receptor.

These newly characterized epitopes for Mac2-158 and E10B10 thus revealed three preferred binding regions for antibodies against CD163. Based on the fine epitope mapping work, linear or preferentially circular peptides are synthesized and used for the induction, production and selection of polyclonal or monoclonal antibodies or other CD163-binding SADC scaffolds that target CD163.

### Example 6: Epitope Mapping of Anti-CD163 mAbs

Peptides aligned to SRCR domain 1 of human CD163 were selected from the top 20 peptide hits of mAb Mac2-158 circular epitope mapping peptides and the most preferred sequences were selected from two peptide alignment clusters at the N-terminus and at the C-terminus of SRCR-1 of human CD163. As a result, the following sequences (as well as motifs derived therefrom) are highly suitable epitopes anti-CD163 antibodies and fragments thereof used as SADC biopolymer scaffold:

Peptide cluster 1:

| | | |
|---|---|---|
| 04 | ----------------EWGTVCNNGWSME------- | (SEQ ID NO: 7) |
| 07 | -----CSGRVEVKVQEEW------------------ | (SEQ ID NO: 365) |
| 09 | --------------QEEWGTVCNNGWS--------- | (SEQ ID NO: 8) |
| 12 | -----------------WGTVCNNGWSMEA------ | (SEQ ID NO: 9) |
| 14 | ---------------EEWGTVCNNGWSM-------- | (SEQ ID NO: 10) |
| 18 | -------------VQEEWGTVCNNGW---------- | (SEQ ID NO: 11) |
| 19 | ----------------EWGTVCNNGW---------- | (SEQ ID NO: 12) |
| 20 | -----------------WGTVCNNGWS--------- | (SEQ ID NO: 5) |
| huCD163-domain 1-3 | DGENKCSGRVEVKVQEEWGTVCNNGWSMEAVSVICN | (SEQ ID NO: 366) |

Peptide cluster 2:

| | | |
|---|---|---|
| 01 | ------------ESALWDC-------------- | (SEQ ID NO: 15) |
| 02 | ---------RGNESALWDC-------------- | (SEQ ID NO: 16) |
| 03 | -------SCRGNESALW---------------- | (SEQ ID NO: 17) |
| 05 | ------VSCRGNESALWDC-------------- | (SEQ ID NO: 18) |
| 06 | --------------ALWDCKHDGW--------- | (SEQ ID NO. 19) |
| 08 | ----DHVSCRGNESALW---------------- | (SEQ ID NO. 20) |
| 11 | --------CRGNESALWD--------------- | (SEQ ID NO. 21) |
| 13 | -----------NESALWDCKHDGW--------- | (SEQ ID NO. 22) |
| 17 | ------------ESALWDCKHDGWG-------- | (SEQ ID NO. 23) |
| huCD163-domain1-3 | RIWMDHVSCRGNESALWDCKHDGWGKHSNCTHQ | (SEQ ID NO: 367) |

Fine epitope mapping of mAb E10B10 was performed as for Mac2-158. 1068 circular peptides (sized 7, 10 and 13 amino acids) and derived from SRCR-1 to -3 of the mouse CD163 sequence (UniProKB Q2VLH6.2) were screened with mAb E10B10 and the following top binding peptides were obtained (ranked by relative signal strength). The human CD163 sequence was aligned to this cluster of mouse CD163 sequences, revealing another highly suitable epitope:

Peptide cluster 3:

| | | |
|---|---|---|
| 01 | ---------------------VTNAPGEMKKELR--------- | (SEQ ID NO: 368) |
| 02 | ------------------ASAVTNAPGEMKK------------ | (SEQ ID NO: 369) |
| 03 | ---------------------VTNAPGEMKK------------ | (SEQ ID NO: 370) |
| 04 | ---------------------VTNAPGE--------------- | (SEQ ID NO: 371) |
| 05 | ----------------GSASAVTNAPGEM-------------- | (SEQ ID NO: 372) |
| 06 | --------------------AVTNAPGEMKKEL---------- | (SEQ ID NO: 373) |
| 07 | -----------------SASAVTNAPGEMK------------- | (SEQ ID NO: 374) |
| 08 | ---------------SGSASAVTNAPGE--------------- | (SEQ ID NO: 375) |
| 09 | --------------------AVTNAPGEMK------------- | (SEQ ID NO: 376) |
| 10 | -------------------SAVTNAPGEM-------------- | (SEQ ID NO: 377) |
| 11 | ------------------ASAVTNAPGE--------------- | (SEQ ID NO: 378) |
| 12 | -------------------SAVTNAPGEMKKE----------- | (SEQ ID NO: 379) |
| 13 | ----------------------TNAPGEMKKE----------- | (SEQ ID NO: 380) |
| mCD163 (SRCR-1, N-terminus) | VTNAPGEMKKELRLAGGENNCS | (SEQ ID NO: 75) |
| hCD163 (SRCR-1, N-terminus) | SSLGGTDKELRLVDGENKCS | (SEQ ID NO: 24) |

The human homologues of mouse peptides 01-13 from cluster 3 have the following sequences of the N-terminal portion of the mature human CD163 protein (UniProtKB: Q86VB7):

| Cluster 3 | | |
|---|---|---|
| peptides | | |
| (mouse): | human homologues: | |
| 01 | SSLGGTDKELR | (SEQ ID NO: 25) |
| 06 | SSLGGTDKEL | (SEQ ID NO: 27) |
| 12, 13 | SSLGGTDKE | (SEQ ID NO: 28) |
| 02, 03 | SSLGGTDK | (SEQ ID NO: 29) |
| 07, 09 | SSLGGTD | (SEQ ID NO: 30) |
| 05, 10 | SSLGGT | (SEQ ID NO: 31) |
| 04, 08, 11 | SSLGG | (SEQ ID NO: 26) |
| hCD163 | SSLGGTDKELRLVDGENKCS | (SEQ ID NO: 24) |
| (SRCR-1) | | |

These homologue peptides represent further highly suitable epitopes for the anti-CD163 antibody-based biopolymer scaffold.

### Example 7: Epitope Mapping of mAb 4D2 Against AdV

mAb 4D2 is a mouse IgG2a mAb targeting the adenovirus fiber epitope peptide (NCBI Reference Sequence: AP 000226.1). It represents a prototype neutralizing antibody that was generated from UV irradiated Ad2 virus (Krasnykh et al, 1998). In order to obtain cyclic antibody binding peptides from the virus neutralizing epitope, mAb 4D2 was mapped against aligned cyclic peptides derived from the fiber sequence. The sequence at amino acid positions 1 to 581 of NCBI Reference Sequence: AP 000226.1 was used as a starting sequence for designing 7mer, 10mer and 13mer cyclic peptides that were then synthesized and circularized directly on a peptide microarray and subsequently incubated with various concentrations of the antibody. The binding signal of monoclonal antibody 4D2 to the peptides yielded several binding hits that were be aligned against the sequence of the protein and subsequently clustered. The resulting clusters were designated cluster1 (length=14 amino acids), cluster2 (length=13 amino acids) and cluster3 (length=22 amino acids). Below are the aligments of the corresponding new peptides that are able to bind the mAb 4D2 paratope. The number of the peptide names corresponds to the rank of the binding signal of the antibody to the microarray (i.e. peptide 01 binds strongest, 02 second strongest, etc.). A selection of top candidate binding peptides out of the top 50 top binders was aligned against the corresponding protein sequence (first line).

| cluster1 | ETGPPTVPFLTPPF | (SEQ ID NO: 32) |
|---|---|---|
| 08 | --GPPTVPFLTP-- | (SEQ ID NO: 60) |
| 10 | ETGPPTVPFLTPP- | (SEQ ID NO: 61) |
| 21 | -TGPPTVPFLT--- | (SEQ ID NO: 62) |
| 34 | ----PTVPFLTPPF | (SEQ ID NO: 63) |
| cluster2 | HDSKLSIATQGPL | (SEQ ID NO: 64) |
| 03 | HDSKLSIATQGPL | (SEQ ID NO: 64) |
| 11 | -----SIATQGP- | (SEQ ID NO: 65) |
| cluster3 | LNLRLGQGPLFINSAHNLDINY | (SEQ ID NO: 34) |
| 02 | -NLRLGQGPLE----------- | (SEQ ID NO: 66) |
| 12 | ------QGPLFINSAH------ | (SEQ ID NO: 67) |
| 13 | --------PLFINSAHNLD--- | (SEQ ID NO: 68) |
| 15 | ----LGQGPLF----------- | (SEQ ID NO: 69) |
| 17 | LNLRLGQGPL------------ | (SEQ ID NO: 70) |
| 19 | -----GQGPLFI---------- | (SEQ ID NO: 71) |
| 29 | -NLRLGQGPLFINS-------- | (SEQ ID NO: 72) |
| 32 | ---------LFINSAHNLDINY | (SEQ ID NO: 73) |
| 33 | ----------FINSAHNLDI-- | (SEQ ID NO: 74) |
| 37 | --LRLGQGPLFI---------- | (SEQ ID NO: 75) |
| 39 | -------GPLFINSAHN----- | (SEQ ID NO: 76) |

The above peptides/sequences are highly suitable as peptides for SADCs which reduce neutralization of AdV vectors.

### Example 8: Epitope Mapping of Monoclonal Antibody 9C12 Against AdV

Monoclonal antibody 9C12 (alias mAB TC31-9C12·C9-s) was generated by immunizing mice with the hexon protein (Uniprot ID: P04133 which corresponds to GenBank: BAG48782.1). This neutralizing antibody is directed against the hexon protein and the neutralizing activity of this antibody was demonstrated by Varghese (Varghese et al, 2004). In brief, diluted antibody was incubated with GFP-expressing replication-defective Ad vector and subsequently added to Hela cells followed by fluorescence readout. In order to map a region from which paratope binding peptides could be derived, the sequence at amino acid positions 1 to 952 of GenBank: BAG48782.1 was used as a starting sequence for designing cyclic 7mer, 10mer and 13mer peptides that were then synthesized and circularized on a peptide microarray, and subsequently incubated with various concentrations of the antibody. The binding signal of mAb 9C12 to the peptides yielded several candidates that could be aligned and clustered against the protein. An epitopic cluster region of 20 amino acids was identified from which paratope binding peptides can be preferentially derived. Below are the aligments of the corresponding peptide hits from this screen. The number of the peptide names corresponds to the ranked binding signal obtained from the microarray (i.e. peptide 01 binds strongest, 02 second strongest, etc.). Cyclic peptides were selected out of up to 50 top binders in this experiment.

| cluster1 | VDPMDEPTLLYVLFEVEDVV | (SEQ ID NO: 35) |
|---|---|---|
| 01 | ----DEPTLLYVLFEVE--- | (SEQ ID NO: 77) |
| 02 | -------TLLYVLFEVE--- | (SEQ ID NO: 78) |
| 03 | ----DEPTLLYVLF------ | (SEQ ID NO: 79) |
| 04 | -------TLLYVLFEVEDVV | (SEQ ID NO: 80) |
| 05 | -------TLLYVLF------ | (SEQ ID NO: 81) |
| 06 | ---MDEPTLLYVLFEV---- | (SEQ ID NO: 82) |
| 07 | -----EPTLLYVLFE----- | (SEQ ID NO: 83) |
| 08 | -DPMDEPTLLYVLF------ | (SEQ ID NO: 84) |
| 09 | --------LLYVLFEVED-- | (SEQ ID NO: 85) |
| 10 | ----------YVLFEVEDVV | (SEQ ID NO: 86) |
| 11 | ------PTLLYVLFEV---- | (SEQ ID NO: 87) |
| 12 | ------PTLLYVLFEVEDV- | (SEQ ID NO: 88) |
| 13 | ---------LYVLFEVEDV- | (SEQ ID NO: 89) |
| 14 | -----EPTLLYVLFEVED-- | (SEQ ID NO: 90) |
| 15 | ---------LYVLFEV---- | (SEQ ID NO: 91) |
| 16 | --PMDEPTLLYVLFE----- | (SEQ ID NO: 92) |
| 17 | --------LLYVLFE----- | (SEQ ID NO: 93) |
| 18 | VDPMDEPTLLYVL------- | (SEQ ID NO: 94) |
| 19 | -----------YVLFEVF-- | (SEQ ID NO: 95) |
| 20 | ------PTLLYVL------- | (SEQ ID NO: 96) |

The above peptides/sequences are highly suitable as peptides for SADCs which reduce neutralization of AdV vectors.

### Example 9: Epitope Mapping of Polyclonal Antibody Ab6982 Against AdV

Polyclonal antibody ab6982 (Abcam) was generated by immunizing rabbits with purified AdV. It reacts with all capsid proteins of Ad5 including hexon, fiber and penton. It was shown that the antibody neutralizes Ad5 infection in a bioassay at 1000 adenovirus 5 particles/ml, a 50% inactivation of the adenovirus can be achieved at a 1/25,000 dilution of the antibody. In order to identify epitopic regions that could contain peptides for ab6982 paratope binding, the antibody was mapped against the sequences of fiber (NCBI Reference Sequence: AP 000226.1) and hexon protein (GenBank: BAG48782.1). The fiber-sequence at amino acid positions 1 to 581 of (NCBI Reference Sequence: AP 000226.1), and the hexon-sequene (GenBank: BAG48782.1) at amino acid positions 1 to 952 were used as a starting sequence for designing 7mer, 10mer and 13mer cyclic peptides synthesized on a peptid array. The binding signal of this antibody to the array yielded several peptides that were aligned and clustered against the sequence of the protein. The peptide clusters were named cluster1-7 (fiber protein) and clusters8-16 (hexon protein) according to their ranked order of cyclic peptide hits (i.e. cluster contains the strongest binders, cluster2 the second strongest etc.). Below are the aligments of the corresponding peptides that bind to polyclonal antibody ab6982. The number of the peptide names corresponds to the antibody binding signal ranking from the microarray experiment, the numbers of the clusters 1-7 and clusters 8-16 are ranked by the content of top binding peptides, respectively.

| Cluster1 | MKRARPSEDTFNPVYPYD | (SEQ ID NO: 36) |
|---|---|---|
| 001 | MKRARPSEDTF------- | (SEQ ID NO: 97) |
| 002 | -KRARPSEDTF------- | (SEQ ID NO: 98) |
| 003 | MKRARPSEDT-------- | (SEQ ID NO: 99) |
| 005 | MKRARPSEDTFN------ | (SEQ ID NO: 100) |
| 010 | ---ARPSEDTFNP----- | (SEQ ID NO: 101) |
| 019 | --RARPSEDTFN------ | (SEQ ID NO: 102) |
| 024 | ----RPSEDTF------- | (SEQ ID NO: 103) |
| 035 | MKRARPSEDTFNP----- | (SEQ ID NO: 104) |
| 040 | --RARPSEDTFNPVY--- | (SEQ ID NO: 105) |
| 041 | ---ARPSEDT-------- | (SEQ ID NO: 106) |
| 052 | -------EDTFNPVYPY- | (SEQ ID NO: 107) |
| 061 | ----RPSEDTFNPVYPY- | (SEQ ID NO: 108) |
| 129 | -KRARPSEDTFNPV---- | (SEQ ID NO: 109) |
| 130 | --------DTFNPVY--- | (SEQ ID NO: 110) |
| 150 | ----RPSEDTFNPV---- | (SEQ ID NO: 111) |
| 153 | -----PSEDTFNPVY--- | (SEQ ID NO: 112) |
| 163 | --------DTFNPVYPYD | (SEQ ID NO: 113) |
| Cluster2 | ISGTVQSAHLIIRFD | (SEQ ID NO: 37) |
| 004 | ----VQSAHLIIRF- | (SEQ ID NO: 114) |
| 006 | -------AHLIIRF- | (SEQ ID NO: 115) |
| 015 | -SGTVQSAHLIIRF- | (SEQ ID NO: 116) |
| 056 | ---TVQSAHLIIR-- | (SEQ ID NO: 117) |
| 060 | --------HLIIRFD | (SEQ ID NO: 118) |
| 065 | ------SAHLIIR-- | (SEQ ID NO: 119) |
| 076 | -----QSAHLIIRFD | (SEQ ID NO: 120) |
| 085 | ISGTVQSAHLIIR-- | (SEQ ID NO: 121) |
| 118 | --GTVQSAHLII--- | (SEQ ID NO: 122) |
| 123 | --GTVQSARLIIRFD | (SEQ ID NO: 123) |
| 126 | -----QSAHLII--- | (SEQ ID NO: 124) |
| Cluster3 | LGQGPLFINSAHNLDINYNKGLYLF | (SEQ ID NO: 38) |
| 009 | -----------HNLDINY------- | (SEQ ID NO: 125) |
| 011 | -----LFINSAHNLDINY------- | (SEQ ID NO: 126) |
| 012 | ------------NLDINYNKGLYLF | (SEQ ID NO: 127) |
| 013 | -------FVSPNG------------ | (SEQ ID NO: 128) |
| 016 | ----------------NYINEIF-- | (SEQ ID NO: 129) |
| 020 | ------------------NKGLYLF | (SEQ ID NO: 130) |
| 021 | ---------------INYNKGLYLF | (SEQ ID NO: 131) |
| 023 | --------NSAHNLDINY------- | (SEQ ID NO: 132) |
| 032 | ------WDWSGH----NYINEIF-- | (SEQ ID NO: 133) |
| 039 | ---------SGH----NYINEIF-- | (SEQ ID NO: 134) |
| 044 | --LGTGLSF---------------- | (SEQ ID NO: 135) |
| 047 | PELTPPF------------------ | (SEQ ID NO: 136) |
| Cluster4 | SYPFDAQNQLNLRLGQGPLFIN | (SEQ ID NO: 39) |
| 027 | -------------LGQGPLF-- | (SEQ ID NO: 137) |
| 029 | ----------NLRLGQGPLF-- | (SEQ ID NO: 138) |
| 030 | -------NQLNLRLGQGPLF-- | (SEQ ID NO: 139) |
| 058 | --------------GQGPLFI- | (SEQ ID NO: 140) |
| 059 | --------QLNLRLGQGPLFI- | (SEQ ID NO: 141) |
| 062 | SYPFDAQNQLNLR--------- | (SEQ ID NO: 142) |
| 066 | -YPFDAQNQLNLRL-------- | (SEQ ID NO: 143) |
| 070 | -----------LRLGQGPLFI- | (SEQ ID NO: 144) |
| 072 | -------NQLNLRL-------- | (SEQ ID NO: 145) |
| 073 | ---FDAQNQLNLR--------- | (SEQ ID NO: 146) |
| 082 | ------QNQLNLR--------- | (SEQ ID NO: 147) |
| 093 | ---------------QGPLFIN | (SEQ ID NO: 148) |
| 102 | --PFDAQNQLNLRLG------- | (SEQ ID NO: 149) |
| 112 | ----DAQNQLNLRL-------- | (SEQ ID NO: 150) |
| 117 | ------------RLGQGPLFIN | (SEQ ID NO: 151) |
| 136 | --------QLNLRLG------- | (SEQ ID NO: 152) |
| 147 | ---FDAQNQLNLRLGQ------ | (SEQ ID NO: 153) |
| 148 | ---------LNLRLGQGPLFIN | (SEQ ID NO: 154) |
| 169 | -----AQNQLNLRLG------- | (SEQ ID NO: 155) |
| 172 | -----AQNQLNI---------- | (SEQ ID NO: 156) |
| 173 | ---------LNLRLGQ------ | (SEQ ID NO: 157) |
| 178 | SYPFDAQNQL------------ | (SEQ ID NO: 158) |
| 197 | --PFDAQNQLNL---------- | (SEQ ID NO: 159) |
| Cluster5 | GDTTPSAYSMSFSWDWSGHNYIN | (SEQ ID NO: 40) |
| 008 | -------YSMSFSW--------- | (SEQ ID NO: 160) |
| 014 | ---TPSAYSMSFSWDW------- | (SEQ ID NO: 161) |
| 022 | ---------MSFSWDW------- | (SEQ ID NO: 162) |
| 028 | ----PSAYSMSFSW--------- | (SEQ ID NO: 163) |
| 049 | -DTTPSAYSMSFSW--------- | (SEQ ID NO: 164) |
| 078 | --TTPSAYSMSF----------- | (SEQ ID NO: 165) |
| 079 | -------YSMSPSWDWS------ | (SEQ ID NO: 166) |
| 091 | TGDTTPSAYSMSF---------- | (SEQ ID NO: 167) |
| 095 | ------------FSWDWSGHNY- | (SEQ ID NO: 168) |
| 100 | -----------SFSWDWS----- | (SEQ ID NO: 169) |
| 108 | ------SAYSMSF---------- | (SEQ ID NO: 170) |
| 134 | -----------SFSWDWSGHN-- | (SEQ ID NO: 171) |
| 143 | ------SAYSMSFSWD------- | (SEQ ID NO: 172) |
| 144 | ---------SMSFSWD------- | (SEQ ID NO: 173) |
| 149 | -------------SWDWSGHNYI | (SEQ ID NO: 174) |
| 167 | -------AYSMSFS--------- | (SEQ ID NO: 175) |
| 176 | ---------SMSFSWDWSGHNY- | (SEQ ID NO: 176) |
| 186 | ------------FSWDWSG---- | (SEQ ID NO: 177) |
| 193 | -------------SWDWSGH--- | (SEQ ID NO: 178) |
| Cluster6 | VLLNNSFLDPEYWNERN | (SEQ ID NO: 41) |
| 017 | ------FLDPEYWNER- | (SEQ ID NO: 179) |
| 018 | -----SFLDPEYWNF-- | (SEQ ID NO: 180) |
| 031 | ---------PEYWNFR- | (SEQ ID NO: 181) |
| 033 | --LNNSFLDPEYWNE-- | (SEQ ID NO: 182) |
| 034 | ---NNSFLDPEYWNER- | (SEQ ID NO: 183) |
| 050 | ------FLDPEYW---- | (SEQ ID NO: 184) |
| 053 | --------DPEYWNE-- | (SEQ ID NO: 185) |
| 068 | ---NNSFIDPEYW---- | (SEQ ID NO: 186) |
| 088 | VLLNNSELDPEYW---- | (SEQ ID NO: 187) |
| 113 | ----------EYWNFRN | (SEQ ID NO: 188) |
| 114 | --LNNSFLDPEY----- | (SEQ ID NO: 189) |
| 155 | -------LDPEYWNERN | (SEQ ID NO: 190) |
| 180 | --LNNSFLD-------- | (SEQ ID NO: 191) |
| 187 | ----NSFLDPEYWN--- | (SEQ ID NO: 192) |
| Cluster7 | HNYINEIFATSSYTESYIA | (SEQ ID NO: 42) |
| 042 | ----------SSYTFSY-- | (SEQ ID NO: 193) |
| 043 | -------FATSSYTESY-- | (SEQ ID NO: 194) |
| 055 | --YINEIFATSSYTF---- | (SEQ ID NO: 195) |
| 064 | -----------SYTFSYI- | (SEQ ID NO: 196) |
| 080 | --------ATSSYTF---- | (SEQ ID NO: 197) |
| 089 | -----EIFATSSYTF---- | (SEQ ID NO: 198) |
| 092 | ----NEIFATSSYTESY-- | (SEQ ID NO: 199) |
| 097 | --------ATSSYTESYI- | (SEQ ID NO: 200) |
| 099 | HNYINEIFATSSY------ | (SEQ ID NO: 201) |
| 104 | ------IFATSSY------ | (SEQ ID NO: 202) |
| 110 | ---INEIFATSSY------ | (SEQ ID NO: 203) |
| 119 | -NYINEIFATSSYT----- | (SEQ ID NO: 204) |
| 168 | --YINEIFA---------- | (SEQ ID NO: 205) |
| 181 | ------------YTESYIA | (SEQ ID NO: 206) |
| 200 | -----EIFATSSYTESYI- | (SEQ ID NO: 207) |
| Cluster8 | DEAATALEINLEEEDDDNEDEVDEQAEQQKTH | (SEQ ID NO: 43) |
| 01 | -----ALEINLEEEDDDN-------------- | (SEQ ID NO: 208) |
| 02 | ---ATALEINLEEEDD---------------- | (SEQ ID NO: 209) |
| 03 | -EAATALEINLEEE------------------ | (SEQ ID NO: 210) |
| 04 | ------LEINLEE------------------- | (SEQ ID NO: 211) |
| 05 | ----TALEINLEEEDDD--------------- | (SEQ ID NO: 212) |
| 06 | -------EINLEEE------------------ | (SEQ ID NO: 213) |
| 07 | -----ALEINLEEED----------------- | (SEQ ID NO: 214) |
| 08 | ------LEINLEEEDD---------------- | (SEQ ID NO: 215) |
| 09 | ----TALEINLEEE------------------ | (SEQ ID NO: 216) |
| 11 | DEAATALEINLEE------------------- | (SEQ ID NO: 217) |
| 13 | ------LEINLEEEDDDNE | (SEQ ID NO: 218) |
| 14 | --AATALEINLEEED----------------- | (SEQ ID NO: 219) |
| 15 | -------EINLEEEDDD--------------- | (SEQ ID NO: 220) |
| 19 | ---ATALEINLEE------------------- | (SEQ ID NO: 221) |
| 23 | --------INLEEEDDDN-------------- | (SEQ ID NO: 222) |
| 27 | ---------NLEEEDDDNE------------- | (SEQ ID NO: 223) |
| 10 | -------------------DEVDEQA------ | (SEQ ID NO: 224) |
| 12 | -------------EDDDNEDEVDEQA--- | (SEQ ID NO: 225) |
| 16 | ---------------DDNEDEVDEQAEQ---- | (SEQ ID NO: 226) |
| 17 | --------------------EVDEQAE----- | (SEQ ID NO: 227) |
| 18 | ----------------DNEDEVDEQA------ | (SEQ ID NO: 228) |
| 20 | ---------------------VDEQAEQ---- | (SEQ ID NO: 229) |
| 21 | ------------------EDEVDEQAEQQKT- | (SEQ ID NO: 230) |
| 22 | ------------------EDEVDEQAEQ---- | (SEQ ID NO: 231) |
| 24 | -------------------DEVDEQAEQQKTH | (SEQ ID NO: 232) |
| 25 | -----------------NEDEVDEQAEQQK-- | (SEQ ID NO: 233) |
| 26 | -------------------DEVDEQAEQQ--- | (SEQ ID NO: 234) |
| Cluster9 | INLEEEDDDNEDEVDEQAEQ | (SEQ ID NO: 44) |
| 028 | EINLEEEDDDNED------- | (SEQ ID NO: 235) |
| 029 | --NLEEEDDDNEDEV----- | (SEQ ID NO; 236) |
| 032 | -INLEEED------------ | (SEQ ID NO: 237) |
| 034 | ---LEEEDDDNED------- | (SEQ ID NO: 238) |
| 035 | -INLEEEDDDNEDE------ | (SEQ ID NO: 239) |
| 037 | -------DDDNEDEVDEQAE | (SEQ ID NO: 240) |
| 053 | ---LEEEDDDNEDEVD---- | (SEQ ID NO: 241) |
| 057 | --------DDNEDEVDEQ-- | (SEQ ID NO: 242) |
| 063 | ------EDDDNED------- | (SEQ ID NO: 243) |
| 065 | --NLEEEDD----------- | (SEQ ID NO: 244) |
| 078 | --------DDNEDEV----- | (SEQ ID NO: 245) |
| 087 | -------DDDNEDEVDE--- | (SEQ ID NO; 246) |
| 096 | -------DDDNEDE------ | (SEQ ID NO: 247) |
| 097 | ----EEEDDDNEDE------ | (SEQ ID NO: 248) |
| 108 | -----EEDDDNE-------- | (SEQ ID NO: 249) |
| 121 | ------EDDDNEDEVD---- | (SEQ ID NO: 250) |
| 126 | -----------EDEVDEQ--- | (SEQ ID NO: 251) |
| 148 | -----EEDDDNEDEVDEQ--- | (SEQ ID NO: 252) |
| 185 | -----EEDDDNEDEV------ | (SEQ ID NO: 253) |
| 188 | ----EEEDDDNEDEVDE---- | (SEQ ID NO: 254) |
| Cluster10 | DNEDEVDEQAEQQKTHVF | (SEQ ID NO: 45) |
| 030 | ----EVDEQAEQQK---- | (SEQ ID NO: 255) |
| 031 | DNEDEVDEQAEQQ----- | (SEQ ID NO: 256) |
| 036 | -----VDEQAEQQKT--- | (SEQ ID NO: 257) |
| 038 | ----EVDEQAEQQKTHV- | (SEQ ID NO: 258) |
| 041 | -----VDEQAEQQKTHVE | (SEQ ID NO: 259) |
| Cluster11 | EWDEAATALEINLEE | (SEQ ID NO: 46) |
| 033 | --------ALEINLE | (SEQ ID NO: 260) |
| 042 | --WDEAATALEINLE | (SEQ ID NO: 261) |
| 043 | -----AATALEINLE | (SEQ ID NO: 262) |
| 112 | EWDEAATALEINL-- | (SEQ ID NO: 263) |
| 124 | ---EAATALEINL-- | (SEQ ID NO: 264) |
| Cluster12 | PKVVLYSEDVDIETPDTHISYMP | (SEQ ID NO: 47 |
| 039 | ----LYSEDVDIET--------- | (SEQ ID NO: 265) |
| 040 | ----LYSEDVDIETPDT------ | (SEQ ID NO: 266) |
| 044 | -KVVLYSEDVDIET--------- | (SEQ ID NO: 267) |
| 045 | -----------IETPDTH----- | (SEQ ID NO: 268) |
| 046 | ---------VDIETPDTHI---- | (SEQ ID NO: 269) |
| 047 | ---VLYSEDVDIE---------- | (SEQ ID NO: 270) |
| 048 | --------DVDIETPDTHISY-- | (SEQ ID NO: 271) |
| 049 | --VVLYSEDVDIETP-------- | (SEQ ID NO: 272) |
| 050 | ------SEDVDIETPDTHI---- | (SEQ ID NO: 273) |
| 051 | ------------ETPDTHI---- | (SEQ ID NO: 274) |
| 052 | ---VLYSEDVDIETPD------- | (SEQ ID NO: 275) |
| 054 | --------DVDIETPDTH----- | (SEQ ID NO: 276) |
| 055 | ----------DIETPDTHIS--- | (SEQ ID NO; 277) |
| 056 | -------EDVDIETPDTHIS--- | (SEQ ID NO: 278) |
| 058 | -----------IETPDTHISY-- | (SEQ ID NO: 279) |
| 059 | -----YSEDVDIETPDTH----- | (SEQ ID NO; 280) |
| 060 | ---------VDIETPDTHISYM- | (SEQ ID NO: 281) |
| 061 | PKVVLYSEDVDIE---------- | (SEQ ID NO: 282) |
| 062 | ----------DIETPDT------ | (SEQ ID NO: 283) |
| 064 | ----------DIETPDTHISYMP | (SEQ ID NO: 284) |
| 070 | -------EDVDIETPDT------ | (SEQ ID NO: 285) |
| 071 | ------------ETPDTHISYM- | (SEQ ID NO: 286) |
| 159 | -----------IETPDTHISYMP | (SEQ ID NO: 287) |
| Cluster13 | YIPESYKDRMYSFFRNF | (SEQ ID NO: 48) |
| 072 | -------DRMYSFERNF | (SEQ ID NO: 288) |
| 086 | -------DRMYSFF--- | (SEQ ID NO: 289) |
| 104 | ----------YSFFRNF | (SEQ ID NO: 290) |
| 107 | -IPESYKDRMYSFF--- | (SEQ ID NO: 291) |
| 120 | ----SYKDRMYSFF--- | (SEQ ID NO: 292) |
| 127 | ---ESYKDRMYSF---- | (SEQ ID NO: 293) |
| 143 | ------KDRMYSF---- | (SEQ ID NO: 294) |
| 152 | YIPESYKDRMYSF---- | (SEQ ID NO: 295) |
| 153 | --PESYKDRMYSFER-- | (SEQ ID NO: 296) |
| 160 | -----YKDRMYSFER-- | (SEQ ID NO: 297) |
| Cluster14 | DSIGDRTRYFSMW | (SEQ ID NO: 49) |
| 073 | ------TRYFSMW | (SEQ ID NO: 298) |
| 080 | ---GDRTRYF--- | (SEQ ID NO: 299) |
| 095 | DSIGDRTRYF--- | (SEQ ID NO: 300) |
| 100 | DSIGDRTRYFSMW | (SEQ ID NO: 301) |
| 101 | ---GDRTRYFSMW | (SEQ ID NO: 302) |
| Cluster15 | SYKDRMYSFERNF | (SEQ ID NO: 50) |
| 072 | ---DRMYSFERNF | (SEQ ID NO: 303) |
| 099 | SYKDRMYSFERNF | (SEQ ID NO: 304) |
| Cluster16 | FLVQMLANYNIGYQGFY | (SEQ ID NO: 51) |
| 106 | -------NYNIGYQGFY | (SEQ ID NO: 305) |
| 122 | ------ANYNIGYQGF- | (SEQ ID NO: 306) |
| 123 | ----MLANYNIGYQGFY | (SEQ ID NO: 307) |
| 136 | ----------IGYQGFY | (SEQ ID NO: 308) |
| 184 | FLVQMLANYNIGY---- | (SEQ ID NO: 309) |
| 187 | ---------NIGYQGF- | (SEQ ID NO: 310) |
| 190 | ---QMLANYNIGYQGF- | (SEQ ID NO: 311) |

The above peptides/sequences are highly suitable as peptides for SADCs which reduce neutralization of AdV vectors. Importantly, binding of these peptides to the paratope of unwanted antibodies can be even further improved by mutating 1, 2 or 3 amino acids in order to generate mimotopes with improved antibody binding properties.

### Example 10: Epitope Mapping of mAb ADK8 Against AAV

Monoclonal antibody ADK8 was generated by immunizing mice with AAV8 capsids. It is directed against the assembled AAV8 capsid (Sonntag et al, 2011). The neutralizing function of the antibody was previously demonstrated (Gurda et al, 2012). In brief, AAV8 was pre-incubated with ADK8 which lead to a decline in the number of virus particles present in the cytoplasm. Moreover, the binding of AAV8 to the nuclear membrane as well as the nuclear entry were abrogated following neutralization by ADK8. This suggests that ADK8 neutralization might interfere either with the cellular entry and/or the transport to the nucleus. ADK8 also cross-reacts with capsid proteins from other AAV serotypes such as AAV1, AAV3, AAV7 (Mietzsch et al, 2014) and was therefore chosen as an example from which general conclusions about the present invention can be drawn.

As for the other antibodies (see examples above), several clusters were identified, delineating regions from which preferred peptides can be deduced. Most preferably, the peptides aligned below according to their binding strengths can be used for selective antibody depletion and detection as hereinabove.

| Cluster1 | WQNRDVYLQGPIWAKIP | (SEQ ID NO: 52) |
|---|---|---|
| 01 | ------YLQGPIW---- | (SEQ ID NO: 312) |
| 02 | -----VYLQGPI----- | (SEQ ID NO: 313) |
| 03 | WQNRDVY---------- | (SEQ ID NO: 314) |
| 05 | ----DVYLQGP------ | (SEQ ID NO: 315) |
| 08 | -QNRDVYL--------- | (SEQ ID NO: 316) |
| 12 | -------LQGPIWA--- | (SEQ ID NO: 317) |
| 20 | ---RDVYLQG------- | (SEQ ID NO: 318) |
| 50 | --NRDVYLQ-------- | (SEQ ID NO: 319) |
| Cluster2 | DNTYFGYSTPWGYFDFNRFHC | (SEQ ID NO: 53) |
| 07 | ---YFGYSTPWGYFDF----- | (SEQ ID NO: 320) |
| 09 | ----FGYSTPWGYF------- | (SEQ ID NO: 321) |
| 10 | -----GYSTPWGYFD------ | (SEQ ID NO: 322) |
| 11 | ------YSTPWGYFDF----- | (SEQ ID NO: 323) |
| 17 | -NTYFGYSTPWGYF------- | (SEQ ID NO: 324) |
| 18 | --------TPWGYFDFNRFHC | (SEQ ID NO: 325) |
| 23 | --TYFGYSTPWGYFD------ | (SEQ ID NO: 326) |
| 26 | DNTYFGYSTPWGY-------- | (SEQ ID NO: 327) |
| 28 | ---YFGYSTPWGY-------- | (SEQ ID NO: 328) |
| 34 | ----FGYSTPWGYFDFN---- | (SEQ ID NO: 329) |
| Cluster3 | MANQAKNWLPGPCY | (SEQ ID NO: 54) |
| 04 | ------NWLPGPC- | (SEQ ID NO: 330) |
| 15 | -------WIPGPCY | (SEQ ID NO: 331) |
| 16 | --QAKNWLPGPC-- | (SEQ ID NO: 332) |
| 21 | ---AKNWLPGPCY | (SEQ ID NO: 333) |
| 25 | MANQAKNWLPGPC- | (SEQ ID NO: 334) |
| Cluster4 | LPYVLGSAHQGCLPPFP | (SEQ ID NO: 55) |
| 06 | ---------QGCLPPF- | (SEQ ID NO: 335) |
| 13 | ----------GCLPPFP | (SEQ ID NO: 336) |
| 27 | ---VLGSAHQGCLPPF- | (SEQ ID NO: 337) |
| 32 | LPYVLGSAHQGCL---- | (SEQ ID NO: 338) |
| 37 | --YVLGSAHQGC----- | (SEQ ID NO: 339) |
| 38 | ----------CLPPFPA | (SEQ ID NO: 340) |
| 39 | -----SAHQGCLPPF-- | (SEQ ID NO: 341) |
| 45 | --VLGSAHQGCL----- | (SEQ ID NO: 342) |
| 46 | PYVLGSAHQGCLP---- | (SEQ ID NO: 343) |
| Cluster5 | NGSQAVGRSSFYCLEYF | (SEQ ID NO: 56) |
| 14 | ------GRSSFYC---- | (SEQ ID NO: 344) |
| 22 | ----AVGRSSFYCLEYF | (SEQ ID NO: 345) |
| 24 | ----AVGRSSFYCL--- | (SEQ ID NO: 346) |
| 33 | ---QAVGRSSFYCLEY- | (SEQ ID NO: 347) |
| 35 | NGSQAVGRSSFYC---- | (SEQ ID NO: 348) |
| Cluster6 | PLIDQYLYYL | (SEQ ID NO: 57) |
| 19 | ---DQYLYYL | (SEQ ID NO: 349) |
| 29 | PLIDQYLYYL | (SEQ ID NO: 350) |
| 36 | --IDQYLYY- | (SEQ ID NO: 351) |
| Cluster7 | EERFFPSNGILIF | (SEQ ID NO: 58) |
| 31 | ---FFPSNGILIF | (SEQ ID NO: 352) |
| 49 | EERFFPSNGILIF | (SEQ ID NO: 353) |
| Cluster8 | ADGVGSSSGNWHC | (SEQ ID NO: 59) |
| 42 | ---VGSSSGNWHC | (SEQ ID NO: 354) |
| 48 | ADGVGSSSGNWHC | (SEQ ID NO: 355) |

### Example 11: Screen for Anti-AAV Antibodies in Human Sera

2452 linear peptides derived from the sequences of 16 different AAVs and with a sequence length of 15 amino-acids each were synthesized.

Samples obtained from human donors were screened for antibodies against these AAV-derived peptides immobilized on microarrays. To this end, IgG was prepared from blood obtained from the human donors by protein G purification. Each IgG sample was incubated with the peptide microarrays and Ig binding signals were detected by fluorescence. All antibody binding signals to the peptides on the arrays were background subtracted and ranked for each sample and a deduplicated aggregate of the respective top 250 peptide hits for each donor with the corresponding protein sequence of origin (as obtained from UniProt or other sources) was compiled (designated as group IV). Further, the deduplicated aggregate of the respective top 50 peptide hits for each donor was compiled and designated as group III. Further, the deduplicated aggregate of the respective top 25 peptide hits for each donor was compiled and designated as group II. Finally, the deduplicated aggregate of the respective top 10 peptide hits for each donor was compiled and designated as group I.

Detailed results are shown in Table 1 below. Altogether, group I contains 110 distinct peptide hits (assigned to the corresponding AAV vectors in Table 1), group II contains 289 distinct peptide hits, group III contains 428 distinct peptide hits and group IV contains 1271 distinct peptide hits. Evidently, group I is a subset of group II which in turn is a subset of group III which in turn is a subset of group IV. Groups I-IV correspond to the top 4.4%, 10.5%, 17.5% and 51.8%, respectively, of all peptides screened.

Thus, all listed peptides, preferably peptides belonging to group III, even more preferably belonging to group II and most preferably belonging to group I (i.e. to the top 4.4%), provide sequences from which shorter peptide sequences can be derived for antibody depletion according to the present invention. Furthermore, also other peptide sequences (or fragments) from the proteins from which the peptides of Table 1 were derived (preferably from group III, more preferably however from group II, most preferably from group I), are suited to be used for SADCs according to the present invention. In addition, these peptides can also be used as probes for the diagnostic detection of anti-AAV antibodies in biological samples such as human sera.

TABLE 1
This table lists the detailed results of a screen for linear peptides as a
basis for the construction of anti-AAV antibody depleting SADCs according to the
present invention. These peptides are also suitable typing neutralizing
antibodies directed against AAV vectors. If not stated otherwise, the peptides
represent fragments from different AAV VP1 proteins. Source given is either
UniProt ID, GenBank ID, PDB ID or AAV strain name.

| peptide | SEQ ID | | group | group | group | group | |
|---|---|---|---|---|---|---|---|
| # | NO | peptide | I | II | III | IV | source |
| 1 | 383 | LRTGNNFEFSYQFED | X | X | X | X | AAO88201.1 |
| 2 | 384 | TGNNFEFSYQFEDVP | X | X | X | X | AAO88201.1 |
| 3 | 385 | VATEQYGVVADNLQQ | X | X | X | X | AAO88201.1 |
| 4 | 386 | FEFSYFEDVPFHSSY | X | X | X | X | AAV-Rh74 c |
| 5 | 387 | RTGNFEFSYFEDVPF | X | X | X | X | AAV-Rh74 c |
| 6 | 388 | MLRTGNFEFSYFEDV | X | X | X | X | AAV-Rh74 c |
| 7 | 389 | PVPADPPTTFNQAKL | X | X | X | X | AAV-Rh74 c |
| 8 | 390 | TQSTGGTAGTQQLLF | X | X | X | X | AAV-Rh74 c |
| 9 | 391 | EEEIKTTNPVATEQY | X | X | X | X | AAV-Rh74 c |
| 10 | 392 | SSVMLTSEEEIKTTN | X | X | X | X | AAV-Rh74 c |
| 11 | 393 | VDFAVNTEGTYSEPR | X | X | X | X | AAV-Rh74 c |
| 12 | 394 | SVPDPQPIGEPPAGP | X | X | X | X | AAV-Rh74 c |
| 13 | 395 | EEIKTTNPVATEQYG | X | X | X | X | AAV-Rh74 c |
| 14 | 396 | PIGEPPAGPSGLGSG | X | X | X | X | AAV-Rh74 c |
| 15 | 397 | VNTEGTYSEPRPIGT | X | X | X | X | AAV-Rh74 c |
| 16 | 398 | YSSVMLTSEEEIKTT | X | X | X | X | AAV-Rh74 c |
| 17 | 399 | VATNHQSAQTLAVPF | X | X | X | X | ALU85156.1 |
| 18 | 400 | VSTNLQRGNLALGET | X | X | X | X | AOD99651.1 |
| 19 | 401 | ALGETTRPATAAQTQ | X | X | X | X | AOD99656.1 |
| 20 | 402 | TQTTGGTTNTQTLGF | X | X | X | X | pdb3J1QA |
| 21 | 403 | DDEEKFFPQSGVLIF | X | X | X | X | P03135 |
| 22 | 404 | EEEIRTTNPVATEQY | X | X | X | X | P03135 |
| 23 | 405 | PVEPDSSSGTGKAGQ | X | X | X | X | P03135 |
| 24 | 406 | IQYTSNYNKSVNVDF | X | X | X | X | P03135 |
| 25 | 407 | TDEEEIRTTNPVATE | X | X | X | X | P03135 |
| 26 | 408 | VDFTVDTNGVYSEPR | X | X | X | X | P03135 |
| 27 | 409 | VDTNGVYSEPRPIGT | X | X | X | X | P03135 |
| 28 | 410 | TMATGSGAPMADNNE | X | X | X | X | P03135 |
| 29 | 411 | TSTVQVFTDSEYQLP | X | X | X | X | P03135 |
| 30 | 412 | LYYLSRTNTPSGTTT | X | X | X | X | P03135 |
| 31 | 413 | TADVNTQGVLPGMVW | X | X | X | X | P03135 |
| 32 | 414 | GSEKTNVDIEKVMIT | X | X | X | X | P03135 |
| 33 | 415 | TNTMATGSGAPMADN | X | X | X | X | P03135 |
| 34 | 416 | WNPEIQYTSNYNKSV | X | X | X | X | P03135 |
| 35 | 417 | SYTFEDVPFHSSYAH | X | X | X | X | O56137 |
| 36 | 418 | EDVPFHSSYAHSQSL | X | X | X | X | O56137 |
| 37 | 419 | SSTDPATGDVHVMGA | X | X | X | X | O56137 |
| 38 | 420 | ATERFGTVAVNLQSS | X | X | X | X | O56137 |
| 39 | 421 | WLEDNLSEGIREWWD | X | X | X | X | O56137 |
| 40 | 422 | EEEIKATNPVATERF | X | X | X | X | O56137 |
| 41 | 423 | LEDNLSEGIREWWDL | X | X | X | X | O56137 |
| 42 | 424 | DAEFQERLQEDTSFG | X | X | X | X | O56137 |
| 43 | 425 | EWELQKENSKRWNPE | X | X | X | X | O56137 |
| 44 | 426 | SFITQYSTGQVSVEI | X | X | X | X | O56137 |
| 45 | 427 | EIEWELQKENSKRWN | X | X | X | X | O56137 |
| 46 | 428 | ITQYSTGQVSVEIEW | X | X | X | X | O56137 |
| 47 | 429 | TDEEEIKATNPVATE | X | X | X | X | O56137 |
| 48 | 430 | EEGAKTAPGKKRPVE | X | X | X | X | O56137 |
| 49 | 431 | IQVKEVTTNDGVTTI | X | X | X | X | O56137 |
| 50 | 432 | SDSEYQLPYVLGSAH | X | X | X | X | O56137 |
| 51 | 433 | PLGEPPATPAAVGPT | X | X | X | X | O56137 |
| 52 | 434 | SSASTGASNDNHYFG | X | X | X | X | O56137 |
| 53 | 435 | PVDQSPQEPDSSSGV | X | X | X | X | O56139 |
| 54 | 436 | EEAAKTAPGKKRPVD | X | X | X | X | O56139 |
| 55 | 437 | APGKKRPVDQSPQEP | X | X | X | X | O56139 |
| 56 | 438 | ILEPLGLVEEAAKTA | X | X | X | X | O56139 |
| 57 | 439 | TRTVNDQGALPGMVW | X | X | X | X | O56139 |
| 58 | 440 | KRPVDQSPQEPDSSS | X | X | X | X | O56139 |
| 59 | 441 | PLGEPPAAPTSLGSN | X | X | X | X | O56139 |
| 60 | 442 | GKKRPVDQSPQEPDS | X | X | X | X | O56139 |
| 61 | 443 | KTAPGKKRPVDQSPQ | × | X | X | X | O56139 |
| 62 | 444 | SNAELDNVMITDEEE | × | X | X | X | O56139 |
| 63 | 445 | WLEDNLSEGIREWWA | X | X | X | X | O6JC40 |
| 64 | 446 | PVPADPPTAFNKDKL | X | X | X | X | Q6JC40 |
| 65 | 447 | EFENVPFHSSYAHSQ | X | X | X | X | Q6JC40 |
| 66 | 448 | FQERLKEDTSFGGNL | X | X | X | X | Q6JC40 |
| 67 | 449 | PQILIKNTPVPADPP | X | X | X | X | Q6JC40 |
| 68 | 450 | ADAEFQERLKEDTSF | X | X | X | X | Q6JC40 |
| 69 | 451 | EEIKTTNPVATESYG | X | X | X | X | Q6JC40 |
| 70 | 452 | VEFAVNTEGVYSEPR | X | X | X | X | Q6JC40 |
| 71 | 453 | AEFQERLKEDTSFGG | X | X | X | X | Q6JC40 |
| 72 | 454 | EPDSSAGIGKSGAQP | X | X | X | X | Q6JC40 |
| 73 | 455 | LIKNTPVPADPPTAF | X | X | X | X | Q6JC40 |
| 74 | 456 | VMITNEEEIKTTNPV | X | X | X | X | Q6JC40 |
| 75 | 457 | VNTEGVYSEPRPIGT | X | X | X | X | Q6JC40 |
| 76 | 458 | DKLNSFITQYSTGQV | X | X | X | X | Q6JC40 |
| 77 | 459 | VATESYGQVATNHQS | X | X | X | X | Q6JC40 |
| 78 | 460 | DDEERFFPSNGILIF | X | X | X | X | Q8JQF8 |
| 79 | 461 | EEEIKTTNPVATEEY | X | X | X | X | Q8JQF8 |
| 80 | 462 | PVPADPPTTFNQSKL | X | X | X | X | Q8JQF8 |
| 81 | 463 | LIKNTPVPADPPTTF | X | X | X | X | Q8JQF8 |
| 82 | 464 | SSSGNWHCDSTWLGD | X | X | X | X | Q8JQF8 |
| 83 | 465 | TSVDFAVNTEGVYSE | X | X | X | X | Q8JQF8 |
| 84 | 466 | TTTGQNNNSNFAWTA | X | X | X | X | Q8JQF8 |
| 85 | 467 | VDFAVNTEGVYSEPR | X | X | X | X | Q8JQF8 |
| 86 | 468 | VSTTTGQNNNSNFAW | X | X | X | X | Q8JQF8 |
| 87 | 469 | KTAPGKKRPVEPSPQ | X | X | X | X | Q8JQF8 |
| 88 | 470 | TQTTGGTANTQTLGF | X | X | X | X | Q8JQF8 |
| 89 | 471 | VLEPLGLVEEGAKTA | X | X | X | X | Q8JQF8 |
| 90 | 472 | EEVGEGLREFLGLEA | X | X | X | X | Q9YIJ1 |
| 91 | 473 | DAEFQEKLADDTSFG | X | X | X | X | Q9YIJ1 |
| 92 | 474 | SFVDHPPDWLEEVGE | X | X | X | X | Q9YIJ1 |
| 93 | 475 | EWELKKENSKRWNPE | X | X | X | X | Q9YIJ1 |
| 94 | 476 | EMEWELKKENSKRWN | X | X | X | X | Q9YIJ1 |
| 95 | 477 | TNNYNDPQFVDFAPD | X | X | X | X | Q9YIJ1 |
| 96 | 478 | FEEVPFHSSFAPSQN | X | X | X | X | Q9YIJ1 |
| 97 | 479 | QYTNNYNDPQFVDFA | X | X | X | X | Q9YIJ1 |
| 98 | 480 | TSTVQVFTDDDYQLP | X | X | X | X | Q9YIJ1 |
| 99 | 481 | EDSKPSTSSDAEAGP | X | X | X | X | Q9YIJ1 |
| 100 | 482 | EEGAKTAPTGKRIDD | X | X | X | X | Q9YIJ1 |
| 101 | 483 | EVPFHSSFAPSQNLF | X | X | X | X | Q9YIJ1 |
| 102 | 484 | SSFITQYSTGQVTVE | X | X | X | X | Q9YIJ1 |
| 103 | 485 | ELEGASYQVPPQPNG | X | X | X | X | Q9YIJ1 |
| 104 | 486 | ERDVYLQGPIWAKIP | X | X | X | X | Q9YIJ1 |
| 105 | 487 | PQYGYATLNRDNTEN | X | X | X | X | Q9YIJ1 |
| 106 | 488 | QVTVEMEWELKKENS | X | X | X | X | Q9YIJ1 |
| 107 | 489 | VTVQDSTTTIANNLT | X | X | X | X | Q9YIJ1 |
| 108 | 490 | YNEQLEAGDNPYLKY | X | X | X | X | Q9YIJ1 |
| 109 | 491 | YNNHQYREIKSGSVD | X | X | X | X | Q9YIJ1 |
| 110 | 492 | TSSDAEAGPSGSQQL | X | X | X | X | Q9YIJ1 |
| 111 | 493 | FEFSYQFEDVPFHSS | | X | X | X | AAO88201.1 |
| 112 | 494 | NNFEFSYQFEDVPFH | | X | X | X | AAO88201.1 |
| 113 | 495 | YQFEDVPFHSSYAHS | | X | X | X | AAO88201.1 |
| 114 | 496 | QGAGKDNVDYSSVML | | X | X | X | AAO88201.1 |
| 115 | 497 | TPVPADPPTTFSQAK | | X | X | X | AAO88201.1 |
| 116 | 498 | IKTTNPVATEQYGVV | | X | X | X | AAO88201.1 |
| 117 | 499 | GNFEFSYFEDVPFHS | | X | X | X | AAV-Rh74 c |
| 118 | 500 | FSYFEDVPFHSSYAH | | X | X | X | AAV-Rh74 c |
| 119 | 501 | YFEDVPFHSSYAHSQ | | X | X | X | AAV-Rh74 c |
| 120 | 502 | EYFPSQMLRTGNFEF | | X | X | X | AAV-Rh74 c |
| 121 | 503 | EIKTTNPVATEQYGW | | X | X | X | AAV-Rh74 c |
| 122 | 504 | RTQSTGGTAGTQQLL | | X | X | X | AAV-Rh74 c |
| 123 | 505 | DNVDYSSVMLTSEEE | | X | X | X | AAV-Rh74 c |
| 124 | 506 | DEERFFPSSGVLMFG | | X | X | X | AAV-Rh74 c |
| 125 | 507 | TNVDFAVNTEGTYSE | | X | X | X | AAV-Rh74 c |
| 126 | 508 | EIQYTSNYYKSTNVD | | X | X | X | AAV-Rh74 c |
| 127 | 509 | RVSTTLSQNNNSNFA | | X | X | X | AAV-Rh74 c |
| 128 | 510 | SESVPDPQPIGEPPA | | X | X | X | AAV-Rh74 c |
| 129 | 511 | QRVSTTLSQNNNSNF | | X | X | X | AAV-Rh74 c |
| 130 | 512 | VDYSSVMLTSEEEIK | | X | X | X | AAV-Rh74 c |
| 131 | 513 | PQPIGEPPAGPSGLG | | X | X | X | AAV-Rh74 c |
| 132 | 514 | QQRVSTTLSQNNNSN | | X | X | X | AAV-Rh74 c |
| 133 | 515 | VSTTLSQNNNSNFAW | | X | X | X | AAV-Rh74 c |
| 134 | 516 | TTRPATAPQIGTVNS | | X | X | X | AOD99652.1 |
| 135 | 517 | EYGAVAINNQAANLA | | X | X | X | AOD99654.1 |
| 136 | 518 | TTRPATAAQTQVVNN | | X | X | X | AOD99656.1 |
| 137 | 519 | NQSLALGETTRPAST | | X | X | X | AOD99659.1 |
| 138 | 520 | GQMATNNQSLALGET | | X | X | X | AOD99659.1 |
| 139 | 521 | MATNNQSLALGETTR | | X | X | X | AOD99659.1 |
| 140 | 522 | TNNQSLALGETTRPA | | X | X | X | AOD99659.1 |
| 141 | 523 | DSVPDPQPIGEPPAA | | X | X | X | pdb3J1QA |
| 142 | 524 | TGDADSVPDPQPIGE | | X | X | X | pdb3J1QA |
| 143 | 525 | SLTMAAGGGAPMADN | | X | X | X | pdb3J1QA |
| 144 | 526 | TTGGTTNTQTLGFSQ | | X | X | X | pdb3J1QA |
| 145 | 527 | LYYLSRTQTTGGTTN | | X | X | X | pdb3J1QA |
| 146 | 528 | SRTQTTGGTTNTQTL | | X | X | X | pdb3J1QA |
| 147 | 529 | YLSRTQTTGGTTNTQ | | X | X | X | pdb3J1QA |
| 148 | 530 | DGYLPDWLEDTLSEG | | X | X | X | P03135 |
| 149 | 531 | PPFPADVFMVPQYGY | | X | X | X | P03135 |
| 150 | 532 | GEPVNEADAAALEHD | | X | X | X | P03135 |
| 151 | 533 | VNVDFTVDTNGVYSE | | X | X | X | P03135 |
| 152 | 534 | EEKFFPQSGVLIFGK | | X | X | X | P03135 |
| 153 | 535 | PDWLEDTLSEGIRQW | | X | X | X | P03135 |
| 154 | 536 | PVNEADAAALEHDKA | | X | X | X | P03135 |
| 155 | 537 | TNVDIEKVMITDEEE | | X | X | X | P03135 |
| 156 | 538 | VDIEKVMITDEEEIR | | X | X | X | P03135 |
| 157 | 539 | PEIQYTSNYNKSVNV | | X | X | X | P03135 |
| 158 | 540 | VEEPVKTAPGKKRPV | | X | X | X | P03135 |
| 159 | 541 | ASHKDDEEKFFPQSG | | X | X | X | P03135 |
| 160 | 542 | EPVNEADAAALEHDK | | X | X | X | P03135 |
| 161 | 543 | ERHKDDSRGLVLPGY | | X | X | X | P03135 |
| 162 | 544 | GNSSGNWHCDSTWMG | | X | X | X | P03135 |
| 163 | 545 | PVPANPSTTFSAAKF | | X | X | X | P03135 |
| 164 | 546 | AAALEHDKAYDRQLD | | X | X | X | P03135 |
| 165 | 547 | FNGLDKGEPVNEADA | | X | X | X | P03135 |
| 166 | 548 | YTSNYNKSVNVDFTV | | X | X | X | P03135 |
| 167 | 549 | EDTLSEGIRQWWKLK | | X | X | X | P03135 |
| 168 | 550 | EPDSSSGTGKAGQQP | | X | X | X | P03135 |
| 169 | 551 | NNSEYSWTGATKYHL | | X | X | X | P03135 |
| 170 | 552 | SNYNKSVNVDFTVDT | | X | X | X | P03135 |
| 171 | 553 | SSQSGASNDNHYFGY | | X | X | X | P03135 |
| 172 | 554 | KRWNPEIQYTSNYNK | | X | X | X | P03135 |
| 173 | 555 | YLSRTNTPSGTTTQS | | X | X | X | P03135 |
| 174 | 556 | YTFEDVPFHSSYAHS | | X | X | X | O56137 |
| 175 | 557 | FEDVPFHSSYAHSQS | | X | X | X | O56137 |
| 176 | 558 | QSSSTDPATGDVHVM | | X | X | X | O56137 |
| 177 | 559 | PDWLEDNLSEGIREW | | X | X | X | O56137 |
| 178 | 560 | DWLEDNLSEGIREWW | | X | X | X | O56137 |
| 179 | 561 | QYSTGQVSVEIEWEL | | X | X | X | O56137 |
| 180 | 562 | DGYLPDWLEDNLSEG | | X | X | X | O56137 |
| 181 | 563 | EDNLSEGIREWWDLK | | X | X | X | O56137 |
| 182 | 564 | DNHYFGYSTPWGYFD | | X | X | X | O56137 |
| 183 | 565 | PVEQSPQEPDSSSGI | | X | X | X | O56137 |
| 184 | 566 | ADGYLPDWLEDNLSE | | X | X | X | O56137 |
| 185 | 567 | GDSESVPDPQPLGEP | | X | X | X | O56137 |
| 186 | 568 | GEPVNAADAAALEHD | | X | X | X | O56137 |
| 187 | 569 | EDTSFGGNLGRAVFQ | | X | X | X | O56137 |
| 188 | 570 | GCLPPFPADVFMIPQ | | X | X | X | O56137 |
| 189 | 571 | PVPANPPAEFSATKF | | X | X | X | O56137 |
| 190 | 572 | SESVPDPQPLGEPPA | | X | X | X | O56137 |
| 191 | 573 | EFQERLQEDTSFGGN | | X | X | X | O56137 |
| 192 | 574 | DAAALEHDKAYDQQL | | X | X | X | O56137 |
| 193 | 575 | EPVNAADAAALEHDK | | X | X | X | O56137 |
| 194 | 576 | QRVSKTKTDNNNSNF | | X | X | X | O56137 |
| 195 | 577 | DSESVPDPQPLGEPP | | X | X | X | O56137 |
| 196 | 578 | EHDKAYDQQLKAGDN | | X | X | X | O56137 |
| 197 | 579 | LPGMVWQDRDVYLQG | | X | X | X | O56137 |
| 198 | 580 | VSVEIEWELQKENSK | | X | X | X | O56137 |
| 199 | 581 | DSEYQLPYVLGSAHQ | | X | X | X | O56137 |
| 200 | 582 | PQPLGEPPATPAAVG | | X | X | X | O56137 |
| 201 | 583 | VEIEWELQKENSKRW | | X | X | X | O56137 |
| 202 | 584 | EYFPSQMLRTGNNFT | | X | X | X | O56137 |
| 203 | 585 | PLIDQYLYYLNRTQN | | X | X | X | O56137 |
| 204 | 586 | QVSVEIEWELQKENS | | X | X | X | O56137 |
| 205 | 587 | ASNDNHYFGYSTPWG | | X | X | X | O56137 |
| 206 | 588 | EIKATNPVATERFGT | | X | X | X | O56137 |
| 207 | 589 | GSAHQGCLPPFPADV | | X | X | X | O56137 |
| 208 | 590 | SSSGIGKTGQQPAKK | | X | X | X | O56137 |
| 209 | 591 | AAALEHDKAYDQQLK | | X | X | X | O56137 |
| 210 | 592 | NNFQFSYTFEDVPFH | | X | X | X | O56139 |
| 211 | 593 | TGNNFQFSYTFEDVP | | X | X | X | O56139 |
| 212 | 594 | LRTGNNFQFSYTFED | | X | X | X | O56139 |
| 213 | 595 | EADAAALEHDKAYDQ | | X | X | X | O56139 |
| 214 | 596 | PQPLGEPPAAPTSLG | | X | X | X | O56139 |
| 215 | 597 | SSSGVGKSGKQPARK | | X | X | X | O56139 |
| 216 | 598 | DDEEKFFPMHGNLIF | | X | X | X | O56139 |
| 217 | 599 | QRLSKTANDNNNSNF | | X | X | X | O56139 |
| 218 | 600 | TTSGTTNQSRLLFSQ | | X | X | X | O56139 |
| 219 | 601 | SYEFENVPFHSSYAH | | X | X | X | Q6JC40 |
| 220 | 602 | QFSYEFENVPFHSSY | | X | X | X | Q6JC40 |
| 221 | 603 | ERLKEDTSFGGNLGR | | X | X | X | Q6JC40 |
| 222 | 604 | ESVPDPQPIGEPPAA | | X | X | X | Q6JC40 |
| 223 | 605 | NNVEFAVNTEGVYSE | | X | X | X | Q6JC40 |
| 224 | 606 | NEEEIKTTNPVATES | | X | X | X | Q6JC40 |
| 225 | 607 | SLIFGKQGTGRDNVD | | X | X | X | Q6JC40 |
| 226 | 608 | EDNLSEGIREWWALK | | X | X | X | Q6JC40 |
| 227 | 609 | NSFITQYSTGQVSVE | | X | X | X | Q6JC40 |
| 228 | 610 | SSNDNAYFGYSTPWG | | X | X | X | Q6JC40 |
| 229 | 611 | TPVPADPPTAFNKDK | | X | X | X | Q6JC40 |
| 230 | 612 | VEEAAKTAPGKKRPV | | X | X | X | Q6JC40 |
| 231 | 613 | IKNTPVPADPPTAFN | | X | X | X | Q6JC40 |
| 232 | 614 | IKTTNPVATESYGQV | | X | X | X | Q6JC40 |
| 233 | 615 | IQYTSNYYKSNNVEF | | X | X | X | Q6JC40 |
| 234 | 616 | QILIKNTPVPADPPT | | X | X | X | Q6JC40 |
| 235 | 617 | AFNKDKLNSFITQYS | | X | X | X | Q6JC40 |
| 236 | 618 | EYFPSQMLRTGNNFQ | | X | X | X | Q6JC40 |
| 237 | 619 | ITNEEEIKTTNPVAT | | X | X | X | Q6JC40 |
| 238 | 620 | LKEDTSFGGNLGRAV | | X | X | X | Q6JC40 |
| 239 | 621 | VATNHQSAQAQAQTG | | X | X | X | Q6JC40 |
| 240 | 622 | DADKVMITNEEEIKT | | X | X | X | Q6JC40 |
| 241 | 623 | SLTMASGGGAPVADN | | X | X | X | Q6JC40 |
| 242 | 624 | NKDKLNSFITQYSTG | | X | X | X | Q6JC40 |
| 243 | 625 | QPIGEPPAAPSGVGS | | X | X | X | Q6JC40 |
| 244 | 626 | DNADYSDVMLTSEEE | | X | X | X | Q8JQF8 |
| 245 | 627 | TYTFEDVPFHSSYAH | | X | X | X | Q8JQF8 |
| 246 | 628 | ARDNADYSDVMLTSE | | X | X | X | Q8JQF8 |
| 247 | 629 | IGTVNSQGALPGMVW | | X | X | X | Q8JQF8 |
| 248 | 630 | EEYGIVADNLQQQNT | | X | X | X | Q8JQF8 |
| 249 | 631 | QRVSTTTGQNNNSNF | | X | X | X | Q8JQF8 |
| 250 | 632 | DGVGSSSGNWHCDST | | X | X | X | Q8JQF8 |
| 251 | 633 | IKNTPVPADPPTTFN | | X | X | X | Q8JQF8 |
| 252 | 634 | ILIKNTPVPADPPTT | | X | X | X | Q8JQF8 |
| 253 | 635 | STIQVFTDSEYQLPY | | X | X | X | Q8JQF8 |
| 254 | 636 | CYRQQRVSTTTGQNN | | X | X | X | Q8JQF8 |
| 255 | 637 | HDKAYDQQLQAGDNP | | X | X | X | Q8JQF8 |
| 256 | 638 | LYYLSRTQTTGGTAN | | X | X | X | Q8JQF8 |
| 257 | 639 | TFNQSKLNSFITQYS | | X | X | X | Q8JQF8 |
| 258 | 640 | VTQNEGTKTIANNLT | | X | X | X | Q8JQF8 |
| 259 | 641 | QYLYYLSRTQTTGGT | | X | X | X | Q8JQF8 |
| 260 | 642 | SRTQTTGGTANTQTL | | X | X | X | Q8JQF8 |
| 261 | 643 | NTYFGYSTPWGYFDF | | X | X | X | Q8JQF8 |
| 262 | 644 | PVEPSPQRSPDSSTG | | X | X | X | Q8JQF8 |
| 263 | 645 | SVPDPQPLGEPPAAP | | X | X | X | Q8JQF8 |
| 264 | 646 | EPSPQRSPDSSTGIG | | X | X | X | Q8JQF8 |
| 265 | 647 | NNFEFTYNFEEVPFH | | X | X | X | Q9YIJ1 |
| 266 | 648 | PDWLEEVGEGLREFL | | X | X | X | Q9YIJ1 |
| 267 | 649 | TGNNFEFTYNFEEVP | | X | X | X | Q9YIJ1 |
| 268 | 650 | LRTGNNFEFTYNFEE | | X | X | X | Q9YIJ1 |
| 269 | 651 | EPVNRADEVAREHDI | | X | X | X | Q9YIJ1 |
| 270 | 652 | TEEDSKPSTSSDAEA | | X | X | X | Q9YIJ1 |
| 271 | 653 | TQYSTGQVTVEMEWE | | X | X | X | Q9YIJ1 |
| 272 | 654 | ESETQPVNRVAYNVG | | X | X | X | Q9YIJ1 |
| 273 | 655 | NLTSTVQVFTDDDYQ | | X | X | X | Q9YIJ1 |
| 274 | 656 | EIQYTNNYNDPQFVD | | X | X | X | Q9YIJ1 |
| 275 | 657 | PVPGNITSFSDVPVS | | X | X | X | Q9YIJ1 |
| 276 | 658 | TVEMEWELKKENSKR | | X | X | X | Q9YIJ1 |
| 277 | 659 | EFQEKLADDTSFGGN | | X | X | X | Q9YIJ1 |
| 278 | 660 | EQLEAGDNPYLKYNH | | X | X | X | Q9YIJ1 |
| 279 | 661 | NYNDPQFVDFAPDST | | X | X | X | Q9YIJ1 |
| 280 | 662 | SSLGADTMSAGGGGP | | X | X | X | Q9YIJ1 |
| 281 | 663 | SKPSTSSDAEAGPSG | | X | X | X | Q9YIJ1 |
| 282 | 664 | FITQYSTGQVTVEME | | X | X | X | Q9YIJ1 |
| 283 | 665 | EFLGLEAGPPKPKPN | | X | X | X | Q9YIJ1 |
| 284 | 666 | NVGGQMATNNQSSTT | | X | X | X | Q9YIJ1 |
| 285 | 667 | PSKMLRTGNNFEFTY | | X | X | X | Q9YIJ1 |
| 286 | 668 | VLEPFGLVEEGAKTA | | X | X | X | Q9YIJ1 |
| 287 | 669 | AQPASSLGADTMSAG | | X | X | X | Q9YIJ1 |
| 288 | 670 | VQDSTTTIANNLTST | | X | X | X | Q9YIJ1 |
| 289 | 671 | YLEGNMLITSESETQ | | X | X | X | Q9YIJ1 |
| 290 | 672 | NVDFAVNTDGTYSEP | | | X | X | AAO88201.1 |
| 291 | 673 | KDNVDYSSVMLTSEE | | | X | X | AAO88201.1 |
| 292 | 674 | HDDEERFFPSSGVLM | | | X | X | AAV-Rh74 c |
| 293 | 675 | SQMLRTGNFEFSYFE | | | X | X | AAV-Rh74 c |
| 294 | 676 | GDSESVPDPQPIGEP | | | X | X | AAV-Rh74 c |
| 295 | 677 | DNPYLRYHADAEFQE | | | X | X | AAV-Rh74 c |
| 296 | 678 | NTPVPADPPTTFNQA | | | X | X | AAV-Rh74 c |
| 297 | 679 | DSLVNPGVAMATHDD | | | X | X | AAV-Rh74 c |
| 298 | 680 | PLGLVESPVKTAPGK | | | X | X | AAV-Rh74 c |
| 299 | 681 | SRTQSTGGTAGTQQL | | | X | X | AAV-Rh74 c |
| 300 | 682 | TFNQAKLASFITQYS | | | X | X | AAV-Rh74 c |
| 301 | 683 | STTLSQNNNSNFAWT | | | × | X | AAV-Rh74 c |
| 302 | 684 | SSTGIGKKGQQPAKK | | | X | X | AAV-Rh74 c |
| 303 | 685 | KSTNVDFAVNTEGTY | | | X | X | AAV-Rh74 c |
| 304 | 686 | TYSEPRPIGTRYLTR | | | X | X | AAV-Rh74 c |
| 305 | 687 | EYGIVADNLQQQNLA | | | X | X | AOD99652.1 |
| 306 | 688 | RPATAPQIGTVNSQG | | | X | X | AOD99652.1 |
| 307 | 689 | GETTRPATAAQTQVV | | | × | X | AOD99656.1 |
| 308 | 690 | EYGIVSSNLQAANLA | | | × | X | AOD99656.1 |
| 309 | 691 | SNLQAANLALGETTR | | | X | X | AOD99656.1 |
| 310 | 692 | DADSVPDPQPIGEPP | | | X | X | pdb3J1QA |
| 311 | 693 | QGVLPGMVWQDRDVY | | | X | X | P03135 |
| 312 | 694 | NEADAAALEHDKAYD | | | X | X | P03135 |
| 313 | 695 | QGCLPPFPADVFMVP | | | X | X | P03135 |
| 314 | 696 | EHDKAYDRQLDSGDN | | | X | X | P03135 |
| 315 | 697 | PFPADVFMVPQYGYL | | | X | X | P03135 |
| 316 | 698 | EPVKTAPGKKRPVEH | | | X | X | P03135 |
| 317 | 699 | HKDDEEKFFPQSGVL | | | X | X | P03135 |
| 318 | 700 | DKGEPVNEADAAALE | | | X | X | P03135 |
| 319 | 701 | EQYGSVSTNLQRGNR | | | X | X | P03135 |
| 320 | 702 | GPPPPKPAERHKDDS | | | X | X | P03135 |
| 321 | 703 | IEKVMITDEEEIRTT | | | X | X | P03135 |
| 322 | 704 | KSVNVDFTVDTNGVY | | | X | X | P03135 |
| 323 | 705 | RPVEHSPVEPDSSSG | | | X | X | P03135 |
| 324 | 706 | SDIRDQSRNWLPGPC | | | X | X | P03135 |
| 325 | 707 | DNNEGADGVGNSSGN | | | X | X | P03135 |
| 326 | 708 | PVATEQYGSVSTNLQ | | | X | X | P03135 |
| 327 | 709 | QVKEVTQNDGTTTIA | | | X | X | P03135 |
| 328 | 710 | STVQVFTDSEYQLPY | | | X | X | P03135 |
| 329 | 711 | DADSVPDPQPLGQPP | | | X | X | P03135 |
| 330 | 712 | DSGDNPYLKYNHADA | | | X | X | P03135 |
| 331 | 713 | DSLVNPGPAMASHKD | | | X | X | P03135 |
| 332 | 714 | ISSQSGASNDNHYFG | | | X | X | P03135 |
| 333 | 715 | MITDEEEIRTTNPVA | | | X | X | P03135 |
| 334 | 716 | RQWWKLKPGPPPPKP | | | X | X | P03135 |
| 335 | 717 | VKEVTQNDGTTTIAN | | | X | X | P03135 |
| 336 | 718 | VTQNDGTTTIANNLT | | | X | X | P03135 |
| 337 | 719 | ADNNEGADGVGNSSG | | | X | X | P03135 |
| 338 | 720 | PLGLVEEPVKTAPGK | | | X | X | P03135 |
| 339 | 721 | GNNFTFSYTFEDVPF | | | X | X | O56137 |
| 340 | 722 | FSYTFEDVPFHSSYA | | | X | X | O56137 |
| 341 | 723 | TFSYTFEDVPFHSSY | | | X | X | O56137 |
| 342 | 724 | DDKDKFFPMSGVMIF | | | X | X | O56137 |
| 343 | 725 | TFEDVPFHSSYAHSQ | | | X | X | O56137 |
| 344 | 726 | TGDVHVMGALPGMVW | | | X | X | O56137 |
| 345 | 727 | HVMGALPGMVWQDRD | | | X | X | O56137 |
| 346 | 728 | TVAVNLQSSSTDPAT | | | X | X | O56137 |
| 347 | 729 | AVNLQSSSTDPATGD | | | X | X | O56137 |
| 348 | 730 | NHYFGYSTPWGYFDF | | | X | X | O56137 |
| 349 | 731 | GSQAVGRSSFYCLEY | | | X | X | O56137 |
| 350 | 732 | TQYSTGQVSVEIEWE | | | X | X | O56137 |
| 351 | 733 | YLPDWLEDNLSEGIR | | | X | X | O56137 |
| 352 | 734 | SNTALDNVMITDEEE | | | X | X | O56137 |
| 353 | 735 | TPWGYFDFNRFHCHF | | | X | X | O56137 |
| 354 | 736 | YFDFNRFHCHFSPRD | | | X | X | O56137 |
| 355 | 737 | GYLPDWLEDNLSEGI | | | X | X | O56137 |
| 356 | 738 | YSTGQVSVEIEWELQ | | | X | X | O56137 |
| 357 | 739 | SLDRLMNPLIDQYLY | | | X | X | O56137 |
| 358 | 740 | STVQVFSDSEYQLPY | | | X | X | O56137 |
| 359 | 741 | SVPDPQPLGEPPATP | | | X | X | O56137 |
| 360 | 742 | EFSATKFASFITQYS | | | X | X | O56137 |
| 361 | 743 | YSTPWGYFDFNRFHC | | | X | X | O56137 |
| 362 | 744 | AHQGCLPPFPADVFM | | | X | X | O56137 |
| 363 | 745 | ASFITQYSTGQVSVE | | | X | X | O56137 |
| 364 | 746 | EWWDLKPGAPKPKAN | | | X | X | O56137 |
| 365 | 747 | GEPPATPAAVGPTTM | | | X | X | O56137 |
| 366 | 748 | MVWQDRDVYLQGPIW | | | X | X | O56137 |
| 367 | 749 | DKGEPVNAADAAALE | | | X | X | O56137 |
| 368 | 750 | ERLQEDTSFGGNLGR | | | X | X | O56137 |
| 369 | 751 | EYQLPYVLGSAHQGC | | | X | X | O56137 |
| 370 | 752 | FITQYSTGQVSVEIE | | | X | X | O56137 |
| 371 | 753 | MITDEEEIKATNPVA | | | X | X | O56137 |
| 372 | 754 | AADAAALEHDKAYDQ | | | X | X | O56137 |
| 373 | 755 | AVGPTTMASGGGAPM | | | X | X | O56137 |
| 374 | 756 | DSTWLGDRVITTSTR | | | X | X | O56137 |
| 375 | 757 | ATPAAVGPTTMASGG | | | X | X | O56137 |
| 376 | 758 | DNNEGADGVGNASGN | | | X | X | O56137 |
| 377 | 759 | EQSPQEPDSSSGIGK | | | X | X | O56137 |
| 378 | 760 | FNIQVKEVTTNDGVT | | | X | X | O56137 |
| 379 | 761 | PAAVGPTTMASGGGA | | | X | X | O56137 |
| 380 | 762 | SSYAHSQSLDRLMNP | | | X | X | O56137 |
| 381 | 763 | TAPGKKRPVEQSPQE | | | X | X | O56137 |
| 382 | 764 | FHSSYAHSQSLDRLM | | | X | X | O56137 |
| 383 | 765 | FNGLDKGEPVNAADA | | | X | X | O56137 |
| 384 | 766 | HSSYAHSQSLDRLMN | | | X | X | O56137 |
| 385 | 767 | TTSTRTWALPTYNNH | | | X | X | O56137 |
| 386 | 768 | VKEVTTNDGVTTIAN | | | X | X | O56137 |
| 387 | 769 | DGVGNSSGNWHCDSQ | | | X | X | 056139 |
| 388 | 770 | NSSGNWHCDSQWLGD | | | X | X | 056139 |
| 389 | 771 | AKKRILEPLGLVEEA | | | X | X | 056139 |
| 390 | 772 | PVPANPPTTFSPAKF | | | X | X | 056139 |
| 391 | 773 | NNSNFPWTAASKYHL | | | X | X | 056139 |
| 392 | 774 | PDSSSGVGKSGKQPA | | | X | X | 056139 |
| 393 | 775 | QSSNTAPTTRTVNDQ | | | X | X | 056139 |
| 394 | 776 | TVANNLQSSNTAPTT | | | X | X | 056139 |
| 395 | 777 | AELDNVMITDEEEIR | | | X | X | 056139 |
| 396 | 778 | EEKFFPMHGNLIFGK | | | X | X | 056139 |
| 397 | 779 | GNNFQFSYEFENVPF | | | X | X | Q6JC40 |
| 398 | 780 | NVEFAVNTEGVYSEP | | | X | X | Q6JC40 |
| 399 | 781 | MLRTGNNFQFSYEFE | | | X | X | Q6JC40 |
| 400 | 782 | RTGNNFQFSYEFENV | | | X | X | Q6JC40 |
| 401 | 783 | STVQVFTDSDYQLPY | | | X | X | Q6JC40 |
| 402 | 784 | DGSQAVGRSSFYCLE | | | X | X | Q6JC40 |
| 403 | 785 | EFAVNTEGVYSEPRP | | | X | X | Q6JC40 |
| 404 | 786 | EFAWPGASSWALNGR | | | X | X | Q6JC40 |
| 405 | 787 | DTESVPDPQPIGEPP | | | X | X | Q6JC40 |
| 406 | 788 | NNNSEFAWPGASSWA | | | X | X | Q6JC40 |
| 407 | 789 | NTPVPADPPTAFNKD | | | X | X | Q6JC40 |
| 408 | 790 | STTVTQNNNSEFAWP | | | X | X | Q6JC40 |
| 409 | 791 | EIQYTSNYYKSNNVE | | | X | X | Q6JC40 |
| 410 | 792 | ENVPFHSSYAHSQSL | | | X | X | Q6JC40 |
| 411 | 793 | ILIKNTPVPADPPTA | | | X | X | Q6JC40 |
| 412 | 794 | RVSTTVTQNNNSEFA | | | X | X | Q6JC40 |
| 413 | 795 | SDYQLPYVLGSAHEG | | | X | X | Q6JC40 |
| 414 | 796 | GNGLDKGEPVNAADA | | | X | X | Q6JC40 |
| 415 | 797 | KSNNVEFAVNTEGVY | | | X | X | Q6JC40 |
| 416 | 798 | LNSFITQYSTGQVSV | | | X | X | Q6JC40 |
| 417 | 799 | QQTLKFSVAGPSNMA | | | X | X | Q6JC40 |
| 418 | 800 | SSGNWHCDSQWLGDR | | | X | X | Q6JC40 |
| 419 | 801 | ADNNEGADGVGSSSG | | | X | X | Q6JC40 |
| 420 | 802 | ASGGGAPVADNNEGA | | | X | X | Q6JC40 |
| 421 | 803 | IGEPPAAPSGVGSLT | | | X | X | Q6JC40 |
| 422 | 804 | PLGLVEEAAKTAPGK | | | X | X | Q6JC40 |
| 423 | 805 | SAGIGKSGAQPAKKR | | | X | X | Q6JC40 |
| 424 | 806 | ENSKRWNPEIQYTSN | | | X | X | Q6JC40 |
| 425 | 807 | ELQKENSKRWNPEIQ | | | X | X | Q6JC40 |
| 426 | 808 | GNNFQFTYTFEDVPF | | | X | X | Q8JQF8 |
| 427 | 809 | EERFFPSNGILIFGK | | | X | X | Q8JQF8 |
| 428 | 810 | PVATEEYGIVADNLQ | | | X | X | Q8JQF8 |
| 429 | 811 | QFTYTFEDVPFHSSY | | | X | X | Q8JQF8 |
| 430 | 812 | NTPVPADPPTTFNQS | | | X | X | Q8JQF8 |
| 431 | 813 | EIKTTNPVATEEYGI | | | X | X | Q8JQF8 |
| 432 | 814 | GSSSGNWHCDSTWLG | | | X | X | Q8JQF8 |
| 433 | 815 | RTGNNFQFTYTFEDV | | | X | X | Q8JQF8 |
| 434 | 816 | TNPVATEEYGIVADN | | | X | X | Q8JQF8 |
| 435 | 817 | TSEEEIKTTNPVATE | | | X | X | Q8JQF8 |
| 436 | 818 | GPCYRQQRVSTTTGQ | | | X | X | Q8JQF8 |
| 437 | 819 | LPGPCYRQQRVSTTT | | | X | X | Q8JQF8 |
| 438 | 820 | TTGGTANTQTLGFSQ | | | X | X | Q8JQF8 |
| 439 | 821 | KQISNGTSGGATNDN | | | X | X | Q8JQF8 |
| 440 | 822 | KQNAARDNADYSDVM | | | X | X | Q8JQF8 |
| 441 | 823 | NAARDNADYSDVMLT | | | X | X | Q8JQF8 |
| 442 | 824 | SKLNSFITQYSTGQV | | | X | X | Q8JQF8 |
| 443 | 825 | VKEVTQNEGTKTIAN | | | X | X | Q8JQF8 |
| 444 | 826 | VNSQGALPGMVWQNR | | | X | X | Q8JQF8 |
| 445 | 827 | NNSNFAWTAGTKYHL | | | X | X | Q8JQF8 |
| 446 | 828 | QQQNTAPQIGTVNSQ | | | X | X | Q8JQF8 |
| 447 | 829 | YNNHLYKQISNGTSG | | | X | X | Q8JQF8 |
| 448 | 830 | YSDVMLTSEEEIKTT | | | X | X | Q8JQF8 |
| 449 | 831 | DNTYFGYSTPWGYFD | | | X | X | Q8JQF8 |
| 450 | 832 | TYFGYSTPWGYFDFN | | | X | X | Q8JQF8 |
| 451 | 833 | ESVPDPQPLGEPPAA | | | X | X | Q8JQF8 |
| 452 | 834 | STGIGKKGQQPARKR | | | X | X | Q8JQF8 |
| 453 | 835 | FGYSTPWGYFDFNRF | | | X | X | Q9YIJ1 |
| 454 | 836 | YFGYSTPWGYFDFNR | | | X | X | Q9YIJ1 |
| 455 | 837 | NAYFGYSTPWGYFDF | | | X | X | Q9YIJ1 |
| 456 | 838 | HPPDWLEEVGEGLRE | | | X | X | Q9YIJ1 |
| 457 | 839 | TENPTERSSFFCLEY | | | X | X | Q9YIJ1 |
| 458 | 840 | WLEEVGEGLREFLGL | | | X | X | Q9YIJ1 |
| 459 | 841 | YSTGQVTVEMEWELK | | | X | X | Q9YIJ1 |
| 460 | 842 | FVDFAPDSTGEYRTT | | | X | X | Q9YIJ1 |
| 461 | 843 | QEIVPGSVWMERDVY | | | X | X | Q9YIJ1 |
| 462 | 844 | TPWGYFDFNRFHSHW | | | X | X | Q9YIJ1 |
| 463 | 845 | ANAYFGYSTPWGYFD | | | X | X | Q9YIJ1 |
| 464 | 846 | DDDYQLPYVVGNGTE | | | X | X | Q9YIJ1 |
| 465 | 847 | DEVAREHDISYNEQL | | | X | X | Q9YIJ1 |
| 466 | 848 | ELKKENSKRWNPEIQ | | | X | X | Q9YIJ1 |
| 467 | 849 | GNASGDWHCDSTWMG | | | X | X | Q9YIJ1 |
| 468 | 850 | GYNYLGPGNGLDRGE | | | X | X | Q9YIJ1 |
| 469 | 851 | TSFSDVPVSSFITQY | | | X | X | Q9YIJ1 |
| 470 | 852 | ANNLTSTVQVFTDDD | | | X | X | Q9YIJ1 |
| 471 | 853 | DQYLYRFVSTNNTGG | | | X | X | Q9YIJ1 |
| 472 | 854 | EYRTTRPIGTRYLTR | | | X | X | Q9YIJ1 |
| 473 | 855 | IFNIQVKEVTVQDST | | | X | X | Q9YIJ1 |
| 474 | 856 | ENSKRWNPEIQYTNN | | | X | X | Q9YIJ1 |
| 475 | 857 | AGPPKPKPNQQHQDQ | | | X | X | Q9YIJ1 |
| 476 | 858 | PSTSSDAEAGPSGSQ | | | X | X | Q9YIJ1 |
| 477 | 859 | TGQVTVEMEWELKKE | | | X | X | Q9YIJ1 |
| 478 | 860 | VKIFNIQVKEVTVQD | | | X | X | Q9YIJ1 |
| 479 | 861 | DSTTTIANNLTSTVQ | | | X | X | Q9YIJ1 |
| 480 | 862 | ISYNEQLEAGDNPYL | | | X | X | Q9YIJ1 |
| 481 | 863 | LGLEAGPPKPKPNQQ | | | X | X | Q9YIJ1 |
| 482 | 864 | TAPATGTYNLQEIVP | | | X | X | Q9YIJ1 |
| 483 | 865 | TMSAGGGGPLGDNNQ | | | X | X | Q9YIJ1 |
| 484 | 866 | FSYQFEDVPFHSSYA | | | | X | AAO88201.1 |
| 485 | 867 | VLMFGKQGAGKDNVD | | | | X | AAO88201.1 |
| 486 | 868 | DFAVNTDGTYSEPRP | | | | X | AAO88201.1 |
| 487 | 869 | DGTYSEPRPIGTRYL | | | | X | AAO88201.1 |
| 488 | 870 | GAVNSQGALPGMVWQ | | | | X | AAO88201.1 |
| 489 | 871 | QMLRTGNNFEFSYQF | | | | X | AAO88201.1 |
| 490 | 872 | MFGKQGAGKDNVDYS | | | | X | AAO88201.1 |
| 491 | 873 | APIVGAVNSQGALPG | | | | X | AAO88201.1 |
| 492 | 874 | IVGAVNSQGALPGMV | | | | X | AAO88201.1 |
| 493 | 875 | LQQQNAAPIVGAVNS | | | | X | AAO88201.1 |
| 494 | 876 | NTDGTYSEPRPIGTR | | | | X | AAO88201.1 |
| 495 | 877 | STNVDFAVNTDGTYS | | | | X | AAO88201.1 |
| 496 | 878 | KDDEERFFPSSGVLM | | | | X | AAO88201.1 |
| 497 | 879 | THKDDEERFFPSSGV | | | | X | AAO88201.1 |
| 498 | 880 | VNPGVAMATHKDDEE | | | | X | AAO88201.1 |
| 499 | 881 | KNTPVPADPPTTFSQ | | | | X | AAO88201.1 |
| 500 | 882 | MATHKDDEERFFPSS | | | | X | AAO88201.1 |
| 501 | 883 | NPVATEQYGVVADNL | | | | X | AAO88201.1 |
| 502 | 884 | PGVAMATHKDDEERF | | | | X | AAO88201.1 |
| 503 | 885 | RDSLVNPGVAMATHK | | | | X | AAO88201.1 |
| 504 | 886 | TTNPVATEQYGVVAD | | | | X | AAO88201.1 |
| 505 | 887 | VPADPPTTFSQAKLA | | | | X | AAO88201.1 |
| 506 | 888 | YFPSQMLRTGNNFEF | | | | X | AAO88201.1 |
| 507 | 889 | PSQMLRTGNNFEFSY | | | | X | AAO88201.1 |
| 508 | 890 | VLMFGKQGAGDNVDY | | | | X | AAV-Rh74 c |
| 509 | 891 | AGDNPYLRYHADAEF | | | | X | AAV-Rh74 c |
| 510 | 892 | AGDNVDYSSVMLTSE | | | | X | AAV-Rh74 c |
| 511 | 893 | FPSQMLRTGNFEFSY | | | | X | AAV-Rh74 c |
| 512 | 894 | IVGAVSQGALPGMVW | | | | X | AAV-Rh74 c |
| 513 | 895 | NWGFRPKRLNFLFNI | | | | X | AAV-Rh74 c |
| 514 | 896 | PVATEQYGWADNLQQ | | | | X | AAV-Rh74 c |
| 515 | 897 | EQYGWADNLQQQNAA | | | | X | AAV-Rh74 c |
| 516 | 898 | GPFNGLDKGEPVAAD | | | | X | AAV-Rh74 c |
| 517 | 899 | LVNPGVAMATHDDEE | | | | X | AAV-Rh74 c |
| 518 | 900 | LYYLSRTQSTGGTAG | | | | X | AAV-Rh74 c |
| 519 | 901 | NNMSAQAKNWLPGPC | | | | X | AAV-Rh74 c |
| 520 | 902 | YLGPFNGLDKGEPVA | | | | X | AAV-Rh74 c |
| 521 | 903 | ATEQYGWADNLQQQN | | | | X | AAV-Rh74 c |
| 522 | 904 | ATHDDEERFFPSSGV | | | | X | AAV-Rh74 c |
| 523 | 905 | CLEYFPSQMLRTGNF | | | | X | AAV-Rh74 c |
| 524 | 906 | DKGEPVAADAAALEH | | | | X | AAV-Rh74 c |
| 525 | 907 | FLFNIQVKEVTQNEG | | | | X | AAV-Rh74 c |
| 526 | 908 | GEPVAADAAALEHDK | | | | X | AAV-Rh74 c |
| 527 | 909 | LRYHADAEFQERLQE | | | | X | AAV-Rh74 c |
| 528 | 910 | NPGVAMATHDDEERF | | | | X | AAV-Rh74 c |
| 529 | 911 | PVAADAAALEHDKAY | | | | X | AAV-Rh74 c |
| 530 | 912 | QTGDSESVPDPQPIG | | | | X | AAV-Rh74 c |
| 531 | 913 | YHADAEFQERLQEDT | | | | X | AAV-Rh74 c |
| 532 | 914 | DPPTTFNQAKLASFI | | | | X | AAV-Rh74 c |
| 533 | 915 | GFRPKRLNFLFNIQV | | | | X | AAV-Rh74 c |
| 534 | 916 | GPNNMSAQAKNWLPG | | | | X | AAV-Rh74 c |
| 535 | 917 | GVAMATHDDEERFFP | | | | X | AAV-Rh74 c |
| 536 | 918 | KTTNPVATEQYGWAD | | | | X | AAV-Rh74 c |
| 537 | 919 | LEPLGLVESPVKTAP | | | | X | AAV-Rh74 c |
| 538 | 920 | LNFLFNIQVKEVTQN | | | | X | AAV-Rh74 c |
| 539 | 921 | NNNWGFRPKRLNFLF | | | | X | AAV-Rh74 c |
| 540 | 922 | PSSGVLMFGKQGAGD | | | | X | AAV-Rh74 c |
| 541 | 923 | PYLRYHADAEFQERL | | | | X | AAV-Rh74 c |
| 542 | 924 | RVLEPLGLVESPVKT | | | | X | AAV-Rh74 c |
| 543 | 925 | SPVKTAPGKKRPVEP | | | | X | AAV-Rh74 c |
| 544 | 926 | SQAGPNNMSAQAKNW | | | | X | AAV-Rh74 c |
| 545 | 927 | STGGTAGTQQLLFSQ | | | | X | AAV-Rh74 c |
| 546 | 928 | TGGTAGTQQLLFSQA | | | | X | AAV-Rh74 c |
| 547 | 929 | TNPVATEQYGWADNL | | | | X | AAV-Rh74 c |
| 548 | 930 | VESPVKTAPGKKRPV | | | | X | AAV-Rh74 c |
| 549 | 931 | VKTAPGKKRPVEPSP | | | | X | AAV-Rh74 c |
| 550 | 932 | VSQGALPGMVWQNRD | | | | X | AAV-Rh74 c |
| 551 | 933 | YLSRTQSTGGTAGTQ | | | | X | AAV-Rh74 c |
| 552 | 934 | YYLSRTQSTGGTAGT | | | | X | AAV-Rh74 c |
| 553 | 935 | NMSAQAKNWLPGPCY | | | | X | AAV-Rh74 c |
| 554 | 936 | GEPPAGPSGLGSGTM | | | | X | AAV-Rh74 c |
| 555 | 937 | SEEEIKTTNPVATEQ | | | | X | AAV-Rh74 c |
| 556 | 938 | NVDYSSVMLTSEEEI | | | | X | AAV-Rh74 c |
| 557 | 939 | FAVNTEGTYSEPRPI | | | | X | AAV-Rh74 c |
| 558 | 940 | SNYYKSTNVDFAVNT | | | | X | AAV-Rh74 c |
| 559 | 941 | TEGTYSEPRPIGTRY | | | | X | AAV-Rh74 c |
| 560 | 942 | ERFFPSSGVLMFGKQ | | | | X | AAV-Rh74 c |
| 561 | 943 | HLYKQISNGTSGGST | | | | X | AAV-Rh74 c |
| 562 | 944 | IGKKGQQPAKKRLNF | | | | X | AAV-Rh74 c |
| 563 | 945 | MSAQAKNWLPGPCYR | | | | X | AAV-Rh74 c |
| 564 | 946 | PDPQPIGEPPAGPSG | | | | X | AAV-Rh74 c |
| 565 | 947 | QYLYYLSRTQSTGGT | | | | X | AAV-Rh74 c |
| 566 | 948 | SVMLTSEEEIKTTNP | | | | X | AAV-Rh74 c |
| 567 | 949 | TLSQNNNSNFAWTGA | | | | X | AAV-Rh74 c |
| 568 | 950 | YYKSTNVDFAVNTEG | | | | X | AAV-Rh74 c |
| 569 | 951 | AGPSGLGSGTMAAGG | | | | X | AAV-Rh74 c |
| 570 | 952 | AQAKNWLPGPCYRQQ | | | | X | AAV-Rh74 c |
| 571 | 953 | CYRQQRVSTTLSQNN | | | | X | AAV-Rh74 c |
| 572 | 954 | DQYLYYLSRTQSTGG | | | | X | AAV-Rh74 c |
| 573 | 955 | DYSSVMLTSEEEIKT | | | | X | AAV-Rh74 c |
| 574 | 956 | GLGSGTMAAGGGAPM | | | | X | AAV-Rh74 c |
| 575 | 957 | GRDSLVNPGVAMATH | | | | X | AAV-Rh74 c |
| 576 | 958 | IDQYLYYLSRTQSTG | | | | X | AAV-Rh74 c |
| 577 | 959 | IQYTSNYYKSTNVDF | | | | X | AAV-Rh74 c |
| 578 | 960 | KKGQQPAKKRLNFGQ | | | | X | AAV-Rh74 c |
| 579 | 961 | KLASFITQYSTGQVS | | | | X | AAV-Rh74 c |
| 580 | 962 | KYHLNGRDSLVNPGV | | | | X | AAV-Rh74 c |
| 581 | 963 | LNGRDSLVNPGVAMA | | | | X | AAV-Rh74 c |
| 582 | 964 | NPEIQYTSNYYKSTN | | | | X | AAV-Rh74 c |
| 583 | 965 | PEIQYTSNYYKSTNV | | | | X | AAV-Rh74 c |
| 584 | 966 | PLIDQYLYYLSRTQS | | | | X | AAV-Rh74 c |
| 585 | 967 | QAKLASFITQYSTGQ | | | | X | AAV-Rh74 c |
| 586 | 968 | QISNGTSGGSTNDNT | | | | X | AAV-Rh74 c |
| 587 | 969 | QYTSNYYKSTNVDFA | | | | X | AAV-Rh74 c |
| 588 | 970 | RQQRVSTTLSQNNNS | | | | X | AAV-Rh74 c |
| 589 | 971 | SNGTSGGSTNDNTYF | | | | X | AAV-Rh74 c |
| 590 | 972 | SQNNNSNFAWTGATK | | | | X | AAV-Rh74 c |
| 591 | 973 | TGIGKKGQQPAKKRL | | | | X | AAV-Rh74 c |
| 592 | 974 | TSNYYKSTNVDFAVN | | | | X | AAV-Rh74 c |
| 593 | 975 | TTLSQNNNSNFAWTG | | | | X | AAV-Rh74 c |
| 594 | 976 | YKQISNGTSGGSTND | | | | X | AAV-Rh74 c |
| 595 | 977 | YRQQRVSTTLSQNNN | | | | X | AAV-Rh74 c |
| 596 | 978 | NNSNFAWTGATKYHL | | | | X | AAV-Rh74 c |
| 597 | 979 | GSTNDNTYFGYSTPW | | | | X | AAV-Rh74 c |
| 598 | 980 | DNGRGLVLPGYKYLG | | | | X | AAV-Rh74 c |
| 599 | 981 | NNNSNFAWTGATKYH | | | | X | AAV-Rh74 c |
| 600 | 982 | PKPKANQQKQDNGRG | | | | X | AAV-Rh74 c |
| 601 | 983 | QQKQDNGRGLVLPGY | | | | X | AAV-Rh74 c |
| 602 | 984 | SNFAWTGATKYHLNG | | | | X | AAV-Rh74 c |
| 603 | 985 | LAVPFKAQAQTGWVQ | | | | X | ALU85156.1 |
| 604 | 986 | QTLAVPFKAQAQTGW | | | | X | ALU85156.1 |
| 605 | 987 | SRTINGSGQNQQTLK | | | | X | ALU85156.1 |
| 606 | 988 | GETTRPARQAATADV | | | | X | AOD99651.1 |
| 607 | 989 | ALGETTRPARQAATA | | | | X | AOD99651.1 |
| 608 | 990 | GSVSTNLQRGNLALG | | | | X | AOD99651.1 |
| 609 | 991 | QYGSVSTNLQRGNLA | | | | X | AOD99651.1 |
| 610 | 992 | RPARQAATADVNTQG | | | | X | AOD99651.1 |
| 611 | 993 | TTRPARQAATADVNT | | | | X | AOD99651.1 |
| 612 | 994 | DNLQQQNLALGETTR | | | | X | AOD99652.1 |
| 613 | 995 | GETTRPATAPQIGTV | | | | X | AOD99652.1 |
| 614 | 996 | GIVADNLQQQNLALG | | | | X | AOD99652.1 |
| 615 | 997 | VADNLQQQNLALGET | | | | X | AOD99652.1 |
| 616 | 998 | RPATQAQTGLVHNQG | | | | X | AOD99654.1 |
| 617 | 999 | VADNLQQLALGETTR | | | | X | AOD99655.1 |
| 618 | 1000 | GETTRPAANTGPIVG | | | | X | AOD99655.1 |
| 619 | 1001 | RPAANTGPIVGNVNS | | | | X | AOD99655.1 |
| 620 | 1002 | TEQYGVVADNLQQLA | | | | X | AOD99655.1 |
| 621 | 1003 | TTRPAANTGPIVGNV | | | | X | AOD99655.1 |
| 622 | 1004 | VSSNLQAANLALGET | | | | X | AOD99656.1 |
| 623 | 1005 | GIVSSNLQAANLALG | | | | X | AOD99656.1 |
| 624 | 1006 | RPATAAQTQVVNNQG | | | | X | AOD99656.1 |
| 625 | 1007 | GETTRPASTTAPATG | | | | X | AOD99659.1 |
| 626 | 1008 | SLALGETTRPASTTA | | | | X | AOD99659.1 |
| 627 | 1009 | TTRPASTTAPATGTY | | | | X | AOD99659.1 |
| 628 | 1010 | VGGQMATNNQSLALG | | | | X | AOD99659.1 |
| 629 | 1011 | EAAKTAPGKKRPVEH | | | | X | pdb3J1QA |
| 630 | 1012 | APSGVGSLTMAAGGG | | | | X | pdb3J1QA |
| 631 | 1013 | GGTTNTQTLGFSQGG | | | | X | pdb3J1QA |
| 632 | 1014 | VGSLTMAAGGGAPMA | | | | X | pdb3J1QA |
| 633 | 1015 | GADGVGNSSGNWHCD | | | | X | P03135 |
| 634 | 1016 | ADAAALEHDKAYDRQ | | | | X | P03135 |
| 635 | 1017 | CLPPFPADVFMVPQY | | | | X | P03135 |
| 636 | 1018 | DKAYDRQLDSGDNPY | | | | X | P03135 |
| 637 | 1019 | EGADGVGNSSGNWHC | | | | X | P03135 |
| 638 | 1020 | NNEGADGVGNSSGNW | | | | X | P03135 |
| 639 | 1021 | YLPDWLEDTLSEGIR | | | | X | P03135 |
| 640 | 1022 | ALEHDKAYDRQLDSG | | | | X | P03135 |
| 641 | 1023 | DVNTQGVLPGMVWQD | | | | X | P03135 |
| 642 | 1024 | GPAMASHKDDEEKFF | | | | X | P03135 |
| 643 | 1025 | LPPFPADVFMVPQYG | | | | X | P03135 |
| 644 | 1026 | QRVSKTSADNNNSEY | | | | X | P03135 |
| 645 | 1027 | AADGYLPDWLEDTLS | | | | X | P03135 |
| 646 | 1028 | SSGNWHCDSTWMGDR | | | | X | P03135 |
| 647 | 1029 | FPADVFMVPQYGYLT | | | | X | P03135 |
| 648 | 1030 | GPFNGLDKGEPVNEA | | | | X | P03135 |
| 649 | 1031 | GVGNSSGNWHCDSTW | | | | X | P03135 |
| 650 | 1032 | NNLTSTVQVFTDSEY | | | | X | P03135 |
| 651 | 1033 | NPGPAMASHKDDEEK | | | | X | P03135 |
| 652 | 1034 | TAPGKKRPVEHSPVE | | | | X | P03135 |
| 653 | 1035 | VLPGMVWQDRDVYLQ | | | | X | P03135 |
| 654 | 1036 | VSKTSADNNNSEYSW | | | | X | P03135 |
| 655 | 1037 | AMASHKDDEEKFFPQ | | | | X | P03135 |
| 656 | 1038 | ATEQYGSVSTNLQRG | | | | X | P03135 |
| 657 | 1039 | DNNNSEYSWTGATKY | | | | X | P03135 |
| 658 | 1040 | DSVPDPQPLGQPPAA | | | | X | P03135 |
| 659 | 1041 | EKTNVDIEKVMITDE | | | | X | P03135 |
| 660 | 1042 | EVTQNDGTTTIANNL | | | | X | P03135 |
| 661 | 1043 | GNWHCDSTWMGDRVI | | | | X | P03135 |
| 662 | 1044 | KKRPVEHSPVEPDSS | | | | X | P03135 |
| 663 | 1045 | KPGPPPPKPAERHKD | | | | X | P03135 |
| 664 | 1046 | KTSADNNNSEYSWTG | | | | X | P03135 |
| 665 | 1047 | NEGADGVGNSSGNWH | | | | X | P03135 |
| 666 | 1048 | NLTSTVQVFTDSEYQ | | | | X | P03135 |
| 667 | 1049 | NTQGVLPGMVWQDRD | | | | X | P03135 |
| 668 | 1050 | RLNFGQTGDADSVPD | | | | X | P03135 |
| 669 | 1051 | SRLQFSQAGASDIRD | | | | X | P03135 |
| 670 | 1052 | TVQVFTDSEYQLPYV | | | | X | P03135 |
| 671 | 1053 | VQVFTDSEYQLPYVL | | | | X | P03135 |
| 672 | 1054 | WLEDTLSEGIRQWWK | | | | X | P03135 |
| 673 | 1055 | YNKSVNVDFTVDTNG | | | | X | P03135 |
| 674 | 1056 | ADGVGNSSGNWHCDS | | | | X | P03135 |
| 675 | 1057 | ADVFMVPQYGYLTLN | | | | X | P03135 |
| 676 | 1058 | ANNLTSTVQVFTDSE | | | | X | P03135 |
| 677 | 1059 | APSGLGTNTMATGSG | | | | X | P03135 |
| 678 | 1060 | FKLFNIQVKEVTQND | | | | X | P03135 |
| 679 | 1061 | GCLPPFPADVFMVPQ | | | | X | P03135 |
| 680 | 1062 | GIRQWWKLKPGPPPP | | | | X | P03135 |
| 681 | 1063 | GLDKGEPVNEADAAA | | | | X | P03135 |
| 682 | 1064 | KKRVLEPLGLVEEPV | | | | X | P03135 |
| 683 | 1065 | KQGSEKTNVDIEKVM | | | | X | P03135 |
| 684 | 1066 | KVMITDEEEIRTTNP | | | | X | P03135 |
| 685 | 1067 | LTSTVQVFTDSEYQL | | | | X | P03135 |
| 686 | 1068 | MADNNEGADGVGNSS | | | | X | P03135 |
| 687 | 1069 | PARKRLNFGQTGDAD | | | | X | P03135 |
| 688 | 1070 | PPPKPAERHKDDSRG | | | | X | P03135 |
| 689 | 1071 | SADNNNSEYSWTGAT | | | | X | P03135 |
| 690 | 1072 | TLSEGIRQWWKLKPG | | | | X | P03135 |
| 691 | 1073 | TNGVYSEPRPIGTRY | | | | X | P03135 |
| 692 | 1074 | TNTPSGTTTQSRLQF | | | | X | P03135 |
| 693 | 1075 | WMGDRVITTSTRTWA | | | | X | P03135 |
| 694 | 1076 | AATADVNTQGVLPGM | | | | X | P03135 |
| 695 | 1077 | AYDRQLDSGDNPYLK | | | | X | P03135 |
| 696 | 1078 | CDSTWMGDRVITTST | | | | X | P03135 |
| 697 | 1079 | DDSRGLVLPGYKYLG | | | | X | P03135 |
| 698 | 1080 | DPQPLGQPPAAPSGL | | | | X | P03135 |
| 699 | 1081 | EIRTTNPVATEQYGS | | | | X | P03135 |
| 700 | 1082 | FGKQGSEKTNVDIEK | | | | X | P03135 |
| 701 | 1083 | FTVDTNGVYSEPRPI | | | | X | P03135 |
| 702 | 1084 | GASDIRDQSRNWLPG | | | | X | P03135 |
| 703 | 1085 | GLVEEPVKTAPGKKR | | | | X | P03135 |
| 704 | 1086 | GNRQAATADVNTQGV | | | | X | P03135 |
| 705 | 1087 | GQTGDADSVPDPQPL | | | | X | P03135 |
| 706 | 1088 | GRDSLVNPGPAMASH | | | | X | P03135 |
| 707 | 1089 | GSGAPMADNNEGADG | | | | X | P03135 |
| 708 | 1090 | GVLIFGKQGSEKTNV | | | | X | P03135 |
| 709 | 1091 | HSPVEPDSSSGTGKA | | | | X | P03135 |
| 710 | 1092 | IQVKEVTQNDGTTTI | | | | X | P03135 |
| 711 | 1093 | IRDQSRNWLPGPCYR | | | | X | P03135 |
| 712 | 1094 | KEVTQNDGTTTIANN | | | | X | P03135 |
| 713 | 1095 | KGEPVNEADAAALEH | | | | X | P03135 |
| 714 | 1096 | KLFNIQVKEVTQNDG | | | | X | P03135 |
| 715 | 1097 | KLKPGPPPPKPAERH | | | | X | P03135 |
| 716 | 1098 | LEPLGLVEEPVKTAP | | | | X | P03135 |
| 717 | 1099 | LIFGKQGSEKTNVDI | | | | X | P03135 |
| 718 | 1100 | LNFKLFNIQVKEVTQ | | | | X | P03135 |
| 719 | 1101 | NFGQTGDADSVPDPQ | | | | X | P03135 |
| 720 | 1102 | NLQRGNRQAATADVN | | | | X | P03135 |
| 721 | 1103 | NTPVPANPSTTFSAA | | | | X | P03135 |
| 722 | 1104 | PAAPSGLGTNTMATG | | | | X | P03135 |
| 723 | 1105 | PAERHKDDSRGLVLP | | | | X | P03135 |
| 724 | 1106 | PGKKRPVEHSPVEPD | | | | X | P03135 |
| 725 | 1107 | PLIDQYLYYLSRTNT | | | | X | P03135 |
| 726 | 1108 | PMADNNEGADGVGNS | | | | X | P03135 |
| 727 | 1109 | PQILIKNTPVPANPS | | | | X | P03135 |
| 728 | 1110 | QAGASDIRDQSRNWL | | | | X | P03135 |
| 729 | 1111 | QISSQSGASNDNHYF | | | | X | P03135 |
| 730 | 1112 | QRGNRQAATADVNTQ | | | | X | P03135 |
| 731 | 1113 | QSGVLIFGKQGSEKT | | | | X | P03135 |
| 732 | 1114 | RKRLNFGQTGDADSV | | | | X | P03135 |
| 733 | 1115 | RQQRVSKTSADNNNS | | | | X | P03135 |
| 734 | 1116 | RTTNPVATEQYGSVS | | | | X | P03135 |
| 735 | 1117 | RVLEPLGLVEEPVKT | | | | X | P03135 |
| 736 | 1118 | SAAKFASFITQYSTG | | | | X | P03135 |
| 737 | 1119 | SEYSWTGATKYHLNG | | | | X | P03135 |
| 738 | 1120 | SGTTTQSRLQFSQAG | | | | X | P03135 |
| 739 | 1121 | SRGLVLPGYKYLGPF | | | | X | P03135 |
| 740 | 1122 | SSGTGKAGQQPARKR | | | | X | P03135 |
| 741 | 1123 | TFSAAKFASFITQYS | | | | X | P03135 |
| 742 | 1124 | TGDADSVPDPQPLGQ | | | | X | P03135 |
| 743 | 1125 | TNPVATEQYGSVSTN | | | | X | P03135 |
| 744 | 1126 | VFMVPQYGYLTLNNG | | | | X | P03135 |
| 745 | 1127 | VNEADAAALEHDKAY | | | | X | P03135 |
| 746 | 1128 | VPDPQPLGQPPAAPS | | | | X | P03135 |
| 747 | 1129 | WWKLKPGPPPPKPAE | | | | X | P03135 |
| 748 | 1130 | GATKYHLNGRDSLVN | | | | X | P03135 |
| 749 | 1131 | NFTFSYTFEDVPFHS | | | | X | O56137 |
| 750 | 1132 | RTGNNFTFSYTFEDV | | | | X | O56137 |
| 751 | 1133 | EPFGLVEEGAKTAPG | | | | X | O56137 |
| 752 | 1134 | PVATERFGTVAVNLQ | | | | X | O56137 |
| 753 | 1135 | DVHVMGALPGMVWQD | | | | X | O56137 |
| 754 | 1136 | ERFGTVAVNLQSSST | | | | X | O56137 |
| 755 | 1137 | MGGFGLKHPPPQILI | | | | X | O56137 |
| 756 | 1138 | AMASHKDDKDKFFPM | | | | X | O56137 |
| 757 | 1139 | DVPFHSSYAHSQSLD | | | | X | O56137 |
| 758 | 1140 | FGTVAVNLQSSSTDP | | | | X | O56137 |
| 759 | 1141 | FHPSPLMGGFGLKHP | | | | X | O56137 |
| 760 | 1142 | GLKHPPPQILIKNTP | | | | X | O56137 |
| 761 | 1143 | GRAVFQAKKRVLEPF | | | | X | O56137 |
| 762 | 1144 | HPSPLMGGFGLKHPP | | | | X | O56137 |
| 763 | 1145 | KDKFFPMSGVMIFGK | | | | X | O56137 |
| 764 | 1146 | KHPPPQILIKNTPVP | | | | X | O56137 |
| 765 | 1147 | LMGGFGLKHPPPQIL | | | | X | O56137 |
| 766 | 1148 | NLQSSSTDPATGDVH | | | | X | O56137 |
| 767 | 1149 | PSPLMGGFGLKHPPP | | | | X | O56137 |
| 768 | 1150 | TDPATGDVHVMGALP | | | | X | O56137 |
| 769 | 1151 | LPPFPADVFMIPQYG | | | | X | O56137 |
| 770 | 1152 | FDFNRFHCHFSPRDW | | | | X | O56137 |
| 771 | 1153 | PPFPADVFMIPQYGY | | | | X | O56137 |
| 772 | 1154 | HYFGYSTPWGYFDFN | | | | X | O56137 |
| 773 | 1155 | CLPPFPADVFMIPQY | | | | X | O56137 |
| 774 | 1156 | NNLTSTVQVFSDSEY | | | | X | O56137 |
| 775 | 1157 | SQAVGRSSFYCLEYF | | | | X | O56137 |
| 776 | 1158 | DNLSEGIREWWDLKP | | | | X | O56137 |
| 777 | 1159 | LDRLMNPLIDQYLYY | | | | X | O56137 |
| 778 | 1160 | PFPADVFMIPQYGYL | | | | X | O56137 |
| 779 | 1161 | QAVGRSSFYCLEYFP | | | | X | O56137 |
| 780 | 1162 | QGCLPPFPADVFMIP | | | | X | O56137 |
| 781 | 1163 | GADGVGNASGNWHCD | | | | X | O56137 |
| 782 | 1164 | NLSEGIREWWDLKPG | | | | X | O56137 |
| 783 | 1165 | RSSFYCLEYFPSQML | | | | X | O56137 |
| 784 | 1166 | TDGHFHPSPLMGGFG | | | | X | O56137 |
| 785 | 1167 | VDFTVDNNGLYTEPR | | | | X | O56137 |
| 786 | 1168 | AADGYLPDWLEDNLS | | | | X | O56137 |
| 787 | 1169 | ANVDFTVDNNGLYTE | | | | X | O56137 |
| 788 | 1170 | FPADVFMIPQYGYLT | | | | X | O56137 |
| 789 | 1171 | LPDWLEDNLSEGIRE | | | | X | O56137 |
| 790 | 1172 | MAADGYLPDWLEDNL | | | | X | O56137 |
| 791 | 1173 | SNDNHYFGYSTPWGY | | | | X | O56137 |
| 792 | 1174 | VLPGYKYLGPFNGLD | | | | X | O56137 |
| 793 | 1175 | WGYFDFNRFHCHFSP | | | | X | O56137 |
| 794 | 1176 | EVTTNDGVTTIANNL | | | | X | O56137 |
| 795 | 1177 | GNWHCDSTWLGDRVI | | | | X | O56137 |
| 796 | 1178 | NDNHYFGYSTPWGYF | | | | X | O56137 |
| 797 | 1179 | PAEFSATKFASFITQ | | | | X | O56137 |
| 798 | 1180 | PYLRYNHADAEFQER | | | | X | O56137 |
| 799 | 1181 | YNHADAEFQERLQED | | | | X | O56137 |
| 800 | 1182 | DFNRFHCHFSPRDWQ | | | | X | O56137 |
| 801 | 1183 | DNPYLRYNHADAEFQ | | | | X | O56137 |
| 802 | 1184 | DRLMNPLIDQYLYYL | | | | X | O56137 |
| 803 | 1185 | GRSSFYCLEYFPSQM | | | | X | O56137 |
| 804 | 1186 | HQGCLPPFPADVFMI | | | | X | O56137 |
| 805 | 1187 | MGALPGMVWQDRDVY | | | | X | O56137 |
| 806 | 1188 | PHTDGHFHPSPLMGG | | | | X | O56137 |
| 807 | 1189 | QTGDSESVPDPQPLG | | | | X | O56137 |
| 808 | 1190 | SVEIEWELQKENSKR | | | | X | O56137 |
| 809 | 1191 | VGRSSFYCLEYFPSQ | | | | X | O56137 |
| 810 | 1192 | VQVFSDSEYQLPYVL | | | | X | O56137 |
| 811 | 1193 | YKYLGPFNGLDKGEP | | | | X | O56137 |
| 812 | 1194 | DGVGNASGNWHCDST | | | | X | O56137 |
| 813 | 1195 | GDNPYLRYNHADAEF | | | | X | O56137 |
| 814 | 1196 | GGGAPMADNNEGADG | | | | X | O56137 |
| 815 | 1197 | GQVSVEIEWELQKEN | | | | X | O56137 |
| 816 | 1198 | KGEPVNAADAAALEH | | | | X | O56137 |
| 817 | 1199 | LGPFNGLDKGEPVNA | | | | X | O56137 |
| 818 | 1200 | LTSTVQVFSDSEYQL | | | | X | O56137 |
| 819 | 1201 | NASGNWHCDSTWLGD | | | | X | O56137 |
| 820 | 1202 | NNGLYTEPRPIGTRY | | | | X | O56137 |
| 821 | 1203 | PDPQPLGEPPATPAA | | | | X | O56137 |
| 822 | 1204 | QSLDRLMNPLIDQYL | | | | X | O56137 |
| 823 | 1205 | SQSLDRLMNPLIDQY | | | | X | O56137 |
| 824 | 1206 | VEEGAKTAPGKKRPV | | | | X | O56137 |
| 825 | 1207 | ADAEFQERLQEDTSF | | | | X | O56137 |
| 826 | 1208 | ADVFMIPQYGYLTLN | | | | X | O56137 |
| 827 | 1209 | GMVWQDRDVYLQGPI | | | | X | O56137 |
| 828 | 1210 | IDQYLYYLNRTQNQS | | | | X | O56137 |
| 829 | 1211 | KLFNIQVKEVTTNDG | | | | X | O56137 |
| 830 | 1212 | KRPVEQSPQEPDSSS | | | | X | O56137 |
| 831 | 1213 | KTKTDNNNSNFTWTG | | | | X | O56137 |
| 832 | 1214 | KYLGPFNGLDKGEPV | | | | X | O56137 |
| 833 | 1215 | NAADAAALEHDKAYD | | | | X | O56137 |
| 834 | 1216 | NTPVPANPPAEFSAT | | | | X | O56137 |
| 835 | 1217 | PVNAADAAALEHDKA | | | | X | O56137 |
| 836 | 1218 | PWGYFDFNRFHCHFS | | | | X | O56137 |
| 837 | 1219 | QEDTSFGGNLGRAVF | | | | X | O56137 |
| 838 | 1220 | SAHQGCLPPFPADVF | | | | X | O56137 |
| 839 | 1221 | SGGGAPMADNNEGAD | | | | X | O56137 |
| 840 | 1222 | TGQVSVEIEWELQKE | | | | X | O56137 |
| 841 | 1223 | VFSDSEYQLPYVLGS | | | | X | O56137 |
| 842 | 1224 | VSKTKTDNNNSNFTW | | | | X | O56137 |
| 843 | 1225 | VWQDRDVYLQGPIWA | | | | X | O56137 |
| 844 | 1226 | YLGPFNGLDKGEPVN | | | | X | O56137 |
| 845 | 1227 | AALEHDKAYDQQLKA | | | | X | O56137 |
| 846 | 1228 | ADAAALEHDKAYDQQ | | | | X | O56137 |
| 847 | 1229 | AEFQERLQEDTSFGG | | | | X | O56137 |
| 848 | 1230 | AVGRSSFYCLEYFPS | | | | X | O56137 |
| 849 | 1231 | DDGRGLVLPGYKYLG | | | | X | O56137 |
| 850 | 1232 | DNNNSNFTWTGASKY | | | | X | O56137 |
| 851 | 1233 | ESIINPGTAMASHKD | | | | X | O56137 |
| 852 | 1234 | FGQTGDSESVPDPQP | | | | X | O56137 |
| 853 | 1235 | FNRFHCHFSPRDWQR | | | | X | O56137 |
| 854 | 1236 | FQERLQEDTSFGGNL | | | | X | O56137 |
| 855 | 1237 | FRPKRLNFKLFNIQV | | | | X | O56137 |
| 856 | 1238 | FSPRDWQRLINNNWG | | | | X | O56137 |
| 857 | 1239 | FTVDNNGLYTEPRPI | | | | X | O56137 |
| 858 | 1240 | FTWTGASKYNLNGRE | | | | X | O56137 |
| 859 | 1241 | GAPKPKANQQKQDDG | | | | X | O56137 |
| 860 | 1242 | GAPMADNNEGADGVG | | | | X | O56137 |
| 861 | 1243 | GASNTALDNVMITDE | | | | X | O56137 |
| 862 | 1244 | GHFHPSPLMGGFGLK | | | | X | O56137 |
| 863 | 1245 | GLDKGEPVNAADAAA | | | | X | O56137 |
| 864 | 1246 | GRGLVLPGYKYLGPF | | | | X | O56137 |
| 865 | 1247 | GYFDFNRFHCHFSPR | | | | X | O56137 |
| 866 | 1248 | GYKYLGPFNGLDKGE | | | | X | O56137 |
| 867 | 1249 | IEWELQKENSKRWNP | | | | X | O56137 |
| 868 | 1250 | IREWWDLKPGAPKPK | | | | X | O56137 |
| 869 | 1251 | KIPHTDGHFHPSPLM | | | | X | O56137 |
| 870 | 1252 | KKRPVEQSPQEPDSS | | | | X | O56137 |
| 871 | 1253 | KQDDGRGLVLPGYKY | | | | X | O56137 |
| 872 | 1254 | KTDNNNSNFTWTGAS | | | | X | O56137 |
| 873 | 1255 | LDKGEPVNAADAAAL | | | | X | O56137 |
| 874 | 1256 | LEHDKAYDQQLKAGD | | | | X | O56137 |
| 875 | 1257 | LEYFPSQMLRTGNNF | | | | X | O56137 |
| 876 | 1258 | LKPGAPKPKANQQKQ | | | | X | O56137 |
| 877 | 1259 | MASGGGAPMADNNEG | | | | X | O56137 |
| 878 | 1260 | NEGADGVGNASGNWH | | | | X | O56137 |
| 879 | 1261 | NGLDKGEPVNAADAA | | | | X | O56137 |
| 880 | 1262 | NGSQAVGRSSFYCLE | | | | X | O56137 |
| 881 | 1263 | NNSNFTWTGASKYNL | | | | X | O56137 |
| 882 | 1264 | NNWGFRPKRLNFKLF | | | | X | O56137 |
| 883 | 1265 | NPYLRYNHADAEFQE | | | | X | O56137 |
| 884 | 1266 | NVMITDEEEIKATNP | | | | X | O56137 |
| 885 | 1267 | NWGFRPKRLNFKLFN | | | | X | O56137 |
| 886 | 1268 | PADVFMIPQYGYLTL | | | | X | O56137 |
| 887 | 1269 | PDSSSGIGKTGQQPA | | | | X | O56137 |
| 888 | 1270 | PFNGLDKGEPVNAAD | | | | X | O56137 |
| 889 | 1271 | PGKKRPVEQSPQEPD | | | | X | O56137 |
| 890 | 1272 | PGYKYLGPFNGLDKG | | | | X | O56137 |
| 891 | 1273 | PIWAKIPHTDGHFHP | | | | X | O56137 |
| 892 | 1274 | PQILIKNTPVPANPP | | | | X | O56137 |
| 893 | 1275 | QEPDSSSGIGKTGQQ | | | | X | O56137 |
| 894 | 1276 | QISSASTGASNDNHY | | | | X | O56137 |
| 895 | 1277 | QQKQDDGRGLVLPGY | | | | X | O56137 |
| 896 | 1278 | REWWDLKPGAPKPKA | | | | X | O56137 |
| 897 | 1279 | RLMNPLIDQYLYYLN | | | | X | O56137 |
| 898 | 1280 | RLNFKLFNIQVKEVT | | | | X | O56137 |
| 899 | 1281 | SEYQLPYVLGSAHQG | | | | X | O56137 |
| 900 | 1282 | SGNWHCDSTWLGDRV | | | | X | O56137 |
| 901 | 1283 | SPRDWQRLINNNWGF | | | | X | O56137 |
| 902 | 1284 | STGQVSVEIEWELQK | | | | X | O56137 |
| 903 | 1285 | STPWGYFDFNRFHCH | | | | X | O56137 |
| 904 | 1286 | TALDNVMITDEEEIK | | | | X | O56137 |
| 905 | 1287 | TGDSESVPDPQPLGE | | | | X | O56137 |
| 906 | 1288 | VGNASGNWHCDSTWL | | | | X | O56137 |
| 907 | 1289 | WAKIPHTDGHFHPSP | | | | X | O56137 |
| 908 | 1290 | WELQKENSKRWNPEV | | | | X | O56137 |
| 909 | 1291 | YTEPRPIGTRYLTRP | | | | X | O56137 |
| 910 | 1292 | AGMSVQPKNWLPGPC | | | | X | O56137 |
| 911 | 1293 | AHSQSLDRLMNPLID | | | | X | O56137 |
| 912 | 1294 | APGKKRPVEQSPQEP | | | | X | O56137 |
| 913 | 1295 | APKPKANQQKQDDGR | | | | X | O56137 |
| 914 | 1296 | CDSTWLGDRVITTST | | | | X | O56137 |
| 915 | 1297 | CLEYFPSQMLRTGNN | | | | X | O56137 |
| 916 | 1298 | DGRGLVLPGYKYLGP | | | | X | O56137 |
| 917 | 1299 | DKAYDQQLKAGDNPY | | | | X | O56137 |
| 918 | 1300 | DQQLKAGDNPYLRYN | | | | X | O56137 |
| 919 | 1301 | DRDVYLQGPIWAKIP | | | | X | O56137 |
| 920 | 1302 | FPSQMLRTGNNFTFS | | | | X | O56137 |
| 921 | 1303 | GASKYNLNGRESIIN | | | | X | O56137 |
| 922 | 1304 | GDRVITTSTRTWALP | | | | X | O56137 |
| 923 | 1305 | GFRPKRLNFKLFNIQ | | | | X | O56137 |
| 924 | 1306 | GIREWWDLKPGAPKP | | | | X | O56137 |
| 925 | 1307 | GLVEEGAKTAPGKKR | | | | X | O56137 |
| 926 | 1308 | GLVLPGYKYLGPFNG | | | | X | O56137 |
| 927 | 1309 | GLYTEPRPIGTRYLT | | | | X | O56137 |
| 928 | 1310 | GNLGRAVFQAKKRVL | | | | X | O56137 |
| 929 | 1311 | GQTGDSESVPDPQPL | | | | X | O56137 |
| 930 | 1312 | GRESIINPGTAMASH | | | | X | O56137 |
| 931 | 1313 | HADAEFQERLQEDTS | | | | X | O56137 |
| 932 | 1314 | HFSPRDWQRLINNNW | | | | X | O56137 |
| 933 | 1315 | IKNTPVPANPPAEFS | | | | X | O56137 |
| 934 | 1316 | KANQQKQDDGRGLVL | | | | X | O56137 |
| 935 | 1317 | KPGAPKPKANQQKQD | | | | X | O56137 |
| 936 | 1318 | KPKANQQKQDDGRGL | | | | X | O56137 |
| 937 | 1319 | KRLNFGQTGDSESVP | | | | X | O56137 |
| 938 | 1320 | LDNVMITDEEEIKAT | | | | X | O56137 |
| 939 | 1321 | LGRAVFQAKKRVLEP | | | | X | O56137 |
| 940 | 1322 | LNFGQTGDSESVPDP | | | | X | O56137 |
| 941 | 1323 | LNGRESIINPGTAMA | | | | X | O56137 |
| 942 | 1324 | LNNGSQAVGRSSFYC | | | | X | O56137 |
| 943 | 1325 | LPGYKYLGPFNGLDK | | | | X | O56137 |
| 944 | 1326 | LPTYNNHLYKQISSA | | | | X | O56137 |
| 945 | 1327 | LRYNHADAEFQERLQ | | | | X | O56137 |
| 946 | 1328 | LVEEGAKTAPGKKRP | | | | X | O56137 |
| 947 | 1329 | MLRTGNNFTFSYTFE | | | | X | O56137 |
| 948 | 1330 | NFKLFNIQVKEVTTN | | | | X | O56137 |
| 949 | 1331 | NHADAEFQERLQEDT | | | | X | O56137 |
| 950 | 1332 | NLGRAVFQAKKRVLE | | | | X | O56137 |
| 951 | 1333 | PFHSSYAHSQSLDRL | | | | X | O56137 |
| 952 | 1334 | PKRLNFKLFNIQVKE | | | | X | O56137 |
| 953 | 1335 | PPATPAAVGPTTMAS | | | | X | O56137 |
| 954 | 1336 | PPPQILIKNTPVPAN | | | | X | O56137 |
| 955 | 1337 | PQYGYLTLNNGSQAV | | | | X | O56137 |
| 956 | 1338 | PRDWQRLINNNWGFR | | | | X | O56137 |
| 957 | 1339 | PYVLGSAHQGCLPPF | | | | X | O56137 |
| 958 | 1340 | QDDGRGLVLPGYKYL | | | | X | O56137 |
| 959 | 1341 | QDRDVYLQGPIWAKI | | | | X | O56137 |
| 960 | 1342 | QERLQEDTSFGGNLG | | | | X | O56137 |
| 961 | 1343 | QRLINNNWGFRPKRL | | | | X | O56137 |
| 962 | 1344 | QYLYYLNRTQNQSGS | | | | X | O56137 |
| 963 | 1345 | RDVYLQGPIWAKIPH | | | | X | O56137 |
| 964 | 1346 | RDWQRLINNNWGFRP | | | | X | O56137 |
| 965 | 1347 | RGSPAGMSVQPKNWL | | | | X | O56137 |
| 966 | 1348 | RLNFGQTGDSESVPD | | | | X | O56137 |
| 967 | 1349 | RLQEDTSFGGNLGRA | | | | X | O56137 |
| 968 | 1350 | RQQRVSKTKTDNNNS | | | | X | O56137 |
| 969 | 1351 | SEGIREWWDLKPGAP | | | | X | O56137 |
| 970 | 1352 | SKYNLNGRESIINPG | | | | X | O56137 |
| 971 | 1353 | SNFTWTGASKYNLNG | | | | X | O56137 |
| 972 | 1354 | SSFYCLEYFPSQMLR | | | | X | O56137 |
| 973 | 1355 | STRTWALPTYNNHLY | | | | X | O56137 |
| 974 | 1356 | TNPVATERFGTVAVN | | | | X | O56137 |
| 975 | 1357 | TSTRTWALPTYNNHL | | | | X | O56137 |
| 976 | 1358 | TTMASGGGAPMADNN | | | | X | O56137 |
| 977 | 1359 | TWLGDRVITTSTRTW | | | | X | O56137 |
| 978 | 1360 | TYNNHLYKQISSAST | | | | X | O56137 |
| 979 | 1361 | VDNNGLYTEPRPIGT | | | | X | O56137 |
| 980 | 1362 | VFMIPQYGYLTLNNG | | | | X | O56137 |
| 981 | 1363 | VNAADAAALEHDKAY | | | | X | O56137 |
| 982 | 1364 | VPFHSSYAHSQSLDR | | | | X | O56137 |
| 983 | 1365 | VQYTSNYAKSANVDF | | | | X | O56137 |
| 984 | 1366 | WGFRPKRLNFKLFNI | | | | X | O56137 |
| 985 | 1367 | WLGDRVITTSTRTWA | | | | X | O56137 |
| 986 | 1368 | WQDRDVYLQGPIWAK | | | | X | O56137 |
| 987 | 1369 | WQRLINNNWGFRPKR | | | | X | O56137 |
| 988 | 1370 | YAKSANVDFTVDNNG | | | | X | O56137 |
| 989 | 1371 | YGYLTLNNGSQAVGR | | | | X | O56137 |
| 990 | 1372 | YLNRTQNQSGSAQNK | | | | X | O56137 |
| 991 | 1373 | YLRYNHADAEFQERL | | | | X | O56137 |
| 992 | 1374 | YNLNGRESIINPGTA | | | | X | O56137 |
| 993 | 1375 | FQFSYTFEDVPFHSS | | | | X | 056139 |
| 994 | 1376 | QGALPGMVWQDRDVY | | | | X | 056139 |
| 995 | 1377 | NDQGALPGMVWQDRD | | | | X | 056139 |
| 996 | 1378 | EIRTTNPVATEQYGT | | | | X | 056139 |
| 997 | 1379 | EQYGTVANNLQSSNT | | | | X | 056139 |
| 998 | 1380 | PGNGLDKGEPVNEAD | | | | X | 056139 |
| 999 | 1381 | GEPPAAPTSLGSNTM | | | | X | 056139 |
| 1000 | 1382 | GSNTMASGGGAPMAD | | | | X | 056139 |
| 1001 | 1383 | NTMASGGGAPMADNN | | | | X | 056139 |
| 1002 | 1384 | PTTRTVNDQGALPGM | | | | X | 056139 |
| 1003 | 1385 | LGPGNGLDKGEPVNE | | | | X | 056139 |
| 1004 | 1386 | PDPQPLGEPPAAPTS | | | | X | 056139 |
| 1005 | 1387 | PVATEQYGTVANNLQ | | | | X | 056139 |
| 1006 | 1388 | QSMSLQARNWLPGPC | | | | X | 056139 |
| 1007 | 1389 | SGVGKSGKQPARKRL | | | | X | 056139 |
| 1008 | 1390 | TFSPAKFASFITQYS | | | | X | 056139 |
| 1009 | 1391 | TVNDQGALPGMVWQD | | | | X | 056139 |
| 1010 | 1392 | VGNSSGNWHCDSQWL | | | | X | 056139 |
| 1011 | 1393 | EWWALKPGVPQPKAN | | | | X | 056139 |
| 1012 | 1394 | FPWTAASKYHLNGRD | | | | X | 056139 |
| 1013 | 1395 | LINNNWGFRPKKLSF | | | | X | 056139 |
| 1014 | 1396 | MGGFGLKHPPPQIMI | | | | X | 056139 |
| 1015 | 1397 | NPPTTFSPAKFASFI | | | | X | 056139 |
| 1016 | 1398 | NWGFRPKKLSFKLFN | | | | X | 056139 |
| 1017 | 1399 | TNPVATEQYGTVANN | | | | X | 056139 |
| 1018 | 1400 | AAKTAPGKKRPVDQS | | | | X | 056139 |
| 1019 | 1401 | AAPTSLGSNTMASGG | | | | X | 056139 |
| 1020 | 1402 | AASKYHLNGRDSLVN | | | | X | 056139 |
| 1021 | 1403 | ANNLQSSNTAPTTRT | | | | X | 056139 |
| 1022 | 1404 | DQSPQEPDSSSGVGK | | | | X | 056139 |
| 1023 | 1405 | EGIREWWALKPGVPQ | | | | X | 056139 |
| 1024 | 1406 | GKQPARKRLNFGQTG | | | | X | 056139 |
| 1025 | 1407 | GPQSMSLQARNWLPG | | | | X | 056139 |
| 1026 | 1408 | GRAVFQAKKRILEPL | | | | X | 056139 |
| 1027 | 1409 | IKNTPVPANPPTTFS | | | | X | 056139 |
| 1028 | 1410 | IREWWALKPGVPQPK | | | | X | 056139 |
| 1029 | 1411 | KRILEPLGLVEEAAK | | | | X | 056139 |
| 1030 | 1412 | LKPGVPQPKANQQHQ | | | | X | 056139 |
| 1031 | 1413 | NLGRAVFQAKKRILE | | | | X | 056139 |
| 1032 | 1414 | PANPPTTFSPAKFAS | | | | X | 056139 |
| 1033 | 1415 | PQIMIKNTPVPANPP | | | | X | 056139 |
| 1034 | 1416 | QEPDSSSGVGKSGKQ | | | | X | 056139 |
| 1035 | 1417 | SKYHLNGRDSLVNPG | | | | X | 056139 |
| 1036 | 1418 | SLGSNTMASGGGAPM | | | | X | 056139 |
| 1037 | 1419 | SNTAPTTRTVNDQGA | | | | X | 056139 |
| 1038 | 1420 | TAPTTRTVNDQGALP | | | | X | 056139 |
| 1039 | 1421 | HKDDEEKFFPMHGNL | | | | X | 056139 |
| 1040 | 1422 | ASHKDDEEKFFPMHG | | | | X | 056139 |
| 1041 | 1423 | KFFPMHGNLIFGKEG | | | | X | 056139 |
| 1042 | 1424 | NVMITDEEEIRTTNP | | | | X | 056139 |
| 1043 | 1425 | AMASHKDDEEKFFPM | | | | X | 056139 |
| 1044 | 1426 | LDNVMITDEEEIRTT | | | | X | 056139 |
| 1045 | 1427 | FGKEGTTASNAELDN | | | | X | 056139 |
| 1046 | 1428 | KTANDNNNSNFPWTA | | | | X | 056139 |
| 1047 | 1429 | RQQRLSKTANDNNNS | | | | X | 056139 |
| 1048 | 1430 | DNNNSNFPWTAASKY | | | | X | 056139 |
| 1049 | 1431 | FPMHGNLIFGKEGTT | | | | X | 056139 |
| 1050 | 1432 | GNLIFGKEGTTASNA | | | | X | 056139 |
| 1051 | 1433 | KEGTTASNAELDNVM | | | | X | 056139 |
| 1052 | 1434 | LSKTANDNNNSNFPW | | | | X | 056139 |
| 1053 | 1435 | QGTTSGTTNQSRLLF | | | | X | 056139 |
| 1054 | 1436 | TASNAELDNVMITDE | | | | X | 056139 |
| 1055 | 1437 | YLYYLNRTQGTTSGT | | | | X | 056139 |
| 1056 | 1438 | YYLNRTQGTTSGTTN | | | | X | 056139 |
| 1057 | 1439 | QRLINNNWGFRPKKL | | | | X | 056139 |
| 1058 | 1440 | DGNFHPSPLMGGFGM | | | | X | Q6JC40 |
| 1059 | 1441 | GEDRFFPLSGSLIFG | | | | X | Q6JC40 |
| 1060 | 1442 | LEDNLSEGIREWWAL | | | | X | Q6JC40 |
| 1061 | 1443 | NDNAYFGYSTPWGYF | | | | X | Q6JC40 |
| 1062 | 1444 | NFQFSYEFENVPFHS | | | | X | Q6JC40 |
| 1063 | 1445 | NNLTSTVQVFTDSDY | | | | X | Q6JC40 |
| 1064 | 1446 | QGILPGMVWQDRDVY | | | | X | Q6JC40 |
| 1065 | 1447 | EGVYSEPRPIGTRYL | | | | X | Q6JC40 |
| 1066 | 1448 | GILPGMVWQDRDVYL | | | | X | Q6JC40 |
| 1067 | 1449 | NSEFAWPGASSWALN | | | | X | Q6JC40 |
| 1068 | 1450 | PYLKYNHADAEFQER | | | | X | Q6JC40 |
| 1069 | 1451 | KEGEDRFFPLSGSLI | | | | X | Q6JC40 |
| 1070 | 1452 | PGPAMASHKEGEDRF | | | | X | Q6JC40 |
| 1071 | 1453 | QAKKRLLEPLGLVEE | | | | X | Q6JC40 |
| 1072 | 1454 | QNQGILPGMVWQDRD | | | | X | Q6JC40 |
| 1073 | 1455 | EGADGVGSSSGNWHC | | | | X | Q6JC40 |
| 1074 | 1456 | HEGCLPPFPADVFMI | | | | X | Q6JC40 |
| 1075 | 1457 | LVEEAAKTAPGKKRP | | | | X | Q6JC40 |
| 1076 | 1458 | NQGILPGMVWQDRDV | | | | X | Q6JC40 |
| 1077 | 1459 | EVTDNNGVKTIANNL | | | | X | Q6JC40 |
| 1078 | 1460 | GADGVGSSSGNWHCD | | | | X | Q6JC40 |
| 1079 | 1461 | LTSTVQVFTDSDYQL | | | | X | Q6JC40 |
| 1080 | 1462 | NEGADGVGSSSGNWH | | | | X | Q6JC40 |
| 1081 | 1463 | NPVATESYGQVATNH | | | | X | Q6JC40 |
| 1082 | 1464 | NVDADKVMITNEEEI | | | | X | Q6JC40 |
| 1083 | 1465 | PVADNNEGADGVGSS | | | | X | Q6JC40 |
| 1084 | 1466 | SAHEGCLPPFPADVF | | | | X | Q6JC40 |
| 1085 | 1467 | TGDTESVPDPQPIGE | | | | X | Q6JC40 |
| 1086 | 1468 | TMASGGGAPVADNNE | | | | X | Q6JC40 |
| 1087 | 1469 | VEQSPQEPDSSAGIG | | | | X | Q6JC40 |
| 1088 | 1470 | VQVFTDSDYQLPYVL | | | | X | Q6JC40 |
| 1089 | 1471 | EPLGLVEEAAKTAPG | | | | X | Q6JC40 |
| 1090 | 1472 | EPPAAPSGVGSLTMA | | | | X | Q6JC40 |
| 1091 | 1473 | FNKDKLNSFITQYST | | | | X | Q6JC40 |
| 1092 | 1474 | GAPVADNNEGADGVG | | | | X | Q6JC40 |
| 1093 | 1475 | GGGAPVADNNEGADG | | | | X | Q6JC40 |
| 1094 | 1476 | IFGKQGTGRDNVDAD | | | | X | Q6JC40 |
| 1095 | 1477 | ILPGMVWQDRDVYLQ | | | | X | Q6JC40 |
| 1096 | 1478 | MGGFGMKHPPPQILI | | | | X | Q6JC40 |
| 1097 | 1479 | PPQILIKNTPVPADP | | | | X | Q6JC40 |
| 1098 | 1480 | RAVFQAKKRLLEPLG | | | | X | Q6JC40 |
| 1099 | 1481 | RDNVDADKVMITNEE | | | | X | Q6JC40 |
| 1100 | 1482 | SNNVEFAVNTEGVYS | | | | X | Q6JC40 |
| 1101 | 1483 | TEGVYSEPRPIGTRY | | | | X | Q6JC40 |
| 1102 | 1484 | TSGGSSNDNAYFGYS | | | | X | Q6JC40 |
| 1103 | 1485 | TVTQNNNSEFAWPGA | | | | X | Q6JC40 |
| 1104 | 1486 | VLPGYKYLGPGNGLD | | | | X | Q6JC40 |
| 1105 | 1487 | ADPPTAFNKDKLNSF | | | | X | Q6JC40 |
| 1106 | 1488 | ALNGRNSLMNPGPAM | | | | X | Q6JC40 |
| 1107 | 1489 | APSGVGSLTMASGGG | | | | X | Q6JC40 |
| 1108 | 1490 | DNLSEGIREWWALKP | | | | X | Q6JC40 |
| 1109 | 1491 | DPPTAFNKDKLNSFI | | | | X | Q6JC40 |
| 1110 | 1492 | DSQWLGDRVITTSTR | | | | X | Q6JC40 |
| 1111 | 1493 | DSSAGIGKSGAQPAK | | | | X | Q6JC40 |
| 1112 | 1494 | DVFMIPQYGYLTLND | | | | X | Q6JC40 |
| 1113 | 1495 | GIGKSGAQPAKKRLN | | | | X | Q6JC40 |
| 1114 | 1496 | GKQGTGRDNVDADKV | | | | X | Q6JC40 |
| 1115 | 1497 | GNFHPSPLMGGFGMK | | | | X | Q6JC40 |
| 1116 | 1498 | GVGSSSGNWHCDSQW | | | | X | Q6JC40 |
| 1117 | 1499 | ISNSTSGGSSNDNAY | | | | X | Q6JC40 |
| 1118 | 1500 | KLNSFITQYSTGQVS | | | | X | Q6JC40 |
| 1119 | 1501 | KNTPVPADPPTAFNK | | | | X | Q6JC40 |
| 1120 | 1502 | KYLGPGNGLDKGEPV | | | | X | Q6JC40 |
| 1121 | 1503 | LGRAVFQAKKRLLEP | | | | X | Q6JC40 |
| 1122 | 1504 | LGSAHEGCLPPFPAD | | | | X | Q6JC40 |
| 1123 | 1505 | MNPGPAMASHKEGED | | | | X | Q6JC40 |
| 1124 | 1506 | NMAVQGRNYIPGPSY | | | | X | Q6JC40 |
| 1125 | 1507 | NNEGADGVGSSSGNW | | | | X | Q6JC40 |
| 1126 | 1508 | NWHCDSQWLGDRVIT | | | | X | Q6JC40 |
| 1127 | 1509 | PAMASHKEGEDRFFP | | | | X | Q6JC40 |
| 1128 | 1510 | PEIQYTSNYYKSNNV | | | | X | Q6JC40 |
| 1129 | 1511 | QQRVSTTVTQNNNSE | | | | X | Q6JC40 |
| 1130 | 1512 | RLLEPLGLVEEAAKT | | | | X | Q6JC40 |
| 1131 | 1513 | RPVEQSPQEPDSSAG | | | | X | Q6JC40 |
| 1132 | 1514 | TAFNKDKLNSFITQY | | | | X | Q6JC40 |
| 1133 | 1515 | TGWVQNQGILPGMVW | | | | X | Q6JC40 |
| 1134 | 1516 | TQNNNSEFAWPGASS | | | | X | Q6JC40 |
| 1135 | 1517 | VKEVTDNNGVKTIAN | | | | X | Q6JC40 |
| 1136 | 1518 | VPDPQPIGEPPAAPS | | | | X | Q6JC40 |
| 1137 | 1519 | VQNQGILPGMVWQDR | | | | X | Q6JC40 |
| 1138 | 1520 | YFPSQMLRTGNNFQF | | | | X | Q6JC40 |
| 1139 | 1521 | YVLGSAHEGCLPPFP | | | | X | Q6JC40 |
| 1140 | 1522 | ADGVGSSSGNWHCDS | | | | X | Q6JC40 |
| 1141 | 1523 | ANQQHQDNARGLVLP | | | | X | Q6JC40 |
| 1142 | 1524 | AQPAKKRLNFGQTGD | | | | X | Q6JC40 |
| 1143 | 1525 | ARGLVLPGYKYLGPG | | | | X | Q6JC40 |
| 1144 | 1526 | ASSWALNGRNSLMNP | | | | X | Q6JC40 |
| 1145 | 1527 | AVNTEGVYSEPRPIG | | | | X | Q6JC40 |
| 1146 | 1528 | DNNEGADGVGSSSGN | | | | X | Q6JC40 |
| 1147 | 1529 | DPQPIGEPPAAPSGV | | | | X | Q6JC40 |
| 1148 | 1530 | DRFFPLSGSLIFGKQ | | | | X | Q6JC40 |
| 1149 | 1531 | FGMKHPPPQILIKNT | | | | X | Q6JC40 |
| 1150 | 1532 | GLVLPGYKYLGPGNG | | | | X | Q6JC40 |
| 1151 | 1533 | GNLGRAVFQAKKRLL | | | | X | Q6JC40 |
| 1152 | 1534 | GPGNGLDKGEPVNAA | | | | X | Q6JC40 |
| 1153 | 1535 | GQTGDTESVPDPQPI | | | | X | Q6JC40 |
| 1154 | 1536 | GSSSGNWHCDSQWLG | | | | X | Q6JC40 |
| 1155 | 1537 | GWVQNQGILPGMVWQ | | | | X | Q6JC40 |
| 1156 | 1538 | IANNLTSTVQVFTDS | | | | X | Q6JC40 |
| 1157 | 1539 | KKRLLEPLGLVEEAA | | | | X | Q6JC40 |
| 1158 | 1540 | KKRLNFGQTGDTESV | | | | X | Q6JC40 |
| 1159 | 1541 | LEPLGLVEEAAKTAP | | | | X | Q6JC40 |
| 1160 | 1542 | LGLVEEAAKTAPGKK | | | | X | Q6JC40 |
| 1161 | 1543 | LKAGDNPYLKYNHAD | | | | X | Q6JC40 |
| 1162 | 1544 | LMGGFGMKHPPPQIL | | | | X | Q6JC40 |
| 1163 | 1545 | LNDGSQAVGRSSFYC | | | | X | Q6JC40 |
| 1164 | 1546 | LPYVLGSAHEGCLPP | | | | X | Q6JC40 |
| 1165 | 1547 | LSEGIREWWALKPGA | | | | X | Q6JC40 |
| 1166 | 1548 | MKHPPPQILIKNTPV | | | | X | Q6JC40 |
| 1167 | 1549 | NLSEGIREWWALKPG | | | | X | Q6JC40 |
| 1168 | 1550 | NSTSGGSSNDNAYFG | | | | X | Q6JC40 |
| 1169 | 1551 | NTEGVYSEPRPIGTR | | | | X | Q6JC40 |
| 1170 | 1552 | NYYKSNNVEFAVNTE | | | | X | Q6JC40 |
| 1171 | 1553 | PADPPTAFNKDKLNS | | | | X | Q6JC40 |
| 1172 | 1554 | PAKKRLNFGQTGDTE | | | | X | Q6JC40 |
| 1173 | 1555 | PLIDQYLYYLSKTIN | | | | X | Q6JC40 |
| 1174 | 1556 | PLMGGFGMKHPPPQI | | | | X | Q6JC40 |
| 1175 | 1557 | PQEPDSSAGIGKSGA | | | | X | Q6JC40 |
| 1176 | 1558 | PQPKANQQHQDNARG | | | | X | Q6JC40 |
| 1177 | 1559 | PSPLMGGFGMKHPPP | | | | X | Q6JC40 |
| 1178 | 1560 | PSQMLRTGNNFQFSY | | | | X | Q6JC40 |
| 1179 | 1561 | PSYRQQRVSTTVTQN | | | | X | Q6JC40 |
| 1180 | 1562 | QNQQTLKFSVAGPSN | | | | X | Q6JC40 |
| 1181 | 1563 | QTGWVQNQGILPGMV | | | | X | Q6JC40 |
| 1182 | 1564 | QYTSNYYKSNNVEFA | | | | X | Q6JC40 |
| 1183 | 1565 | RLMNPLIDQYLYYLS | | | | X | Q6JC40 |
| 1184 | 1566 | RLNFGQTGDTESVPD | | | | X | Q6JC40 |
| 1185 | 1567 | RNSLMNPGPAMASHK | | | | X | Q6JC40 |
| 1186 | 1568 | SAQAQAQTGWVQNQG | | | | X | Q6JC40 |
| 1187 | 1569 | SGNWHCDSQWLGDRV | | | | X | Q6JC40 |
| 1188 | 1570 | SHKEGEDRFFPLSGS | | | | X | Q6JC40 |
| 1189 | 1571 | SLMNPGPAMASHKEG | | | | X | Q6JC40 |
| 1190 | 1572 | SQMLRTGNNFQFSYE | | | | X | Q6JC40 |
| 1191 | 1573 | TDSDYQLPYVLGSAH | | | | X | Q6JC40 |
| 1192 | 1574 | TESYGQVATNHQSAQ | | | | X | Q6JC40 |
| 1193 | 1575 | TLKFSVAGPSNMAVQ | | | | X | Q6JC40 |
| 1194 | 1576 | TSNYYKSNNVEFAVN | | | | X | Q6JC40 |
| 1195 | 1577 | VFTDSDYQLPYVLGS | | | | X | Q6JC40 |
| 1196 | 1578 | VGSLTMASGGGAPVA | | | | X | Q6JC40 |
| 1197 | 1579 | VPADPPTAFNKDKLN | | | | X | Q6JC40 |
| 1198 | 1580 | WVQNQGILPGMVWQD | | | | X | Q6JC40 |
| 1199 | 1581 | WWALKPGAPQPKANQ | | | | X | Q6JC40 |
| 1200 | 1582 | YDQQLKAGDNPYLKY | | | | X | Q6JC40 |
| 1201 | 1583 | YKYLGPGNGLDKGEP | | | | X | Q6JC40 |
| 1202 | 1584 | YLGPGNGLDKGEPVN | | | | X | Q6JC40 |
| 1203 | 1585 | YRQQRVSTTVTQNNN | | | | X | Q6JC40 |
| 1204 | 1586 | YYKSNNVEFAVNTEG | | | | X | Q6JC40 |
| 1205 | 1587 | TDGNFHPSPLMGGFG | | | | X | Q6JC40 |
| 1206 | 1588 | WELQKENSKRWNPEI | | | | X | Q6JC40 |
| 1207 | 1589 | HTDGNFHPSPLMGGF | | | | X | Q6JC40 |
| 1208 | 1590 | PHTDGNFHPSPLMGG | | | | X | Q6JC40 |
| 1209 | 1591 | AKIPHTDGNFHPSPL | | | | X | Q6JC40 |
| 1210 | 1592 | QKENSKRWNPEIQYT | | | | X | Q6JC40 |
| 1211 | 1593 | GPIWAKIPHTDGNFH | | | | X | Q6JC40 |
| 1212 | 1594 | GVYSEPRPIGTRYLT | | | | X | Q6JC40 |
| 1213 | 1595 | KENSKRWNPEIQYTS | | | | X | Q6JC40 |
| 1214 | 1596 | KIPHTDGNFHPSPLM | | | | X | Q6JC40 |
| 1215 | 1597 | LQKENSKRWNPEIQY | | | | X | Q6JC40 |
| 1216 | 1598 | PIWAKIPHTDGNFHP | | | | X | Q6JC40 |
| 1217 | 1599 | TIQVFTDSEYQLPYV | | | | X | Q8JQF8 |
| 1218 | 1600 | NNLTSTIQVFTDSEY | | | | X | Q8JQF8 |
| 1219 | 1601 | HKDDEERFFPSNGIL | | | | X | Q8JQF8 |
| 1220 | 1602 | SSGNWHCDSTWLGDR | | | | X | Q8JQF8 |
| 1221 | 1603 | ATNDNTYFGYSTPWG | | | | X | Q8JQF8 |
| 1222 | 1604 | NFQFTYTFEDVPFHS | | | | X | Q8JQF8 |
| 1223 | 1605 | TAPGKKRPVEPSPQR | | | | X | Q8JQF8 |
| 1224 | 1606 | AALEHDKAYDQQLQA | | | | X | Q8JQF8 |
| 1225 | 1607 | ADYSDVMLTSEEEIK | | | | X | Q8JQF8 |
| 1226 | 1608 | ATHKDDEERFFPSNG | | | | X | Q8JQF8 |
| 1227 | 1609 | EPPAAPSGVGPNTMA | | | | X | Q8JQF8 |
| 1228 | 1610 | EWWALKPGAPKPKAN | | | | X | Q8JQF8 |
| 1229 | 1611 | FGKQNAARDNADYSD | | | | X | Q8JQF8 |
| 1230 | 1612 | MAAGGGAPMADNNEG | | | | X | Q8JQF8 |
| 1231 | 1613 | PQIGTVNSQGALPGM | | | | X | Q8JQF8 |
| 1232 | 1614 | QVFTDSEYQLPYVLG | | | | X | Q8JQF8 |
| 1233 | 1615 | VGSSSGNWHCDSTWL | | | | X | Q8JQF8 |
| 1234 | 1616 | ANNLTSTIQVFTDSE | | | | X | Q8JQF8 |
| 1235 | 1617 | GIAMATHKDDEERFF | | | | X | Q8JQF8 |
| 1236 | 1618 | GVGSSSGNWHCDSTW | | | | X | Q8JQF8 |
| 1237 | 1619 | IDQYLYYLSRTQTTG | | | | X | Q8JQF8 |
| 1238 | 1620 | IQVFTDSEYQLPYVL | | | | X | Q8JQF8 |
| 1239 | 1621 | RQQRVSTTTGQNNNS | | | | X | Q8JQF8 |
| 1240 | 1622 | TAPQIGTVNSQGALP | | | | X | Q8JQF8 |
| 1241 | 1623 | YDQQLQAGDNPYLRY | | | | X | Q8JQF8 |
| 1242 | 1624 | ATEEYGIVADNLQQQ | | | | X | Q8JQF8 |
| 1243 | 1625 | EGAKTAPGKKRPVEP | | | | X | Q8JQF8 |
| 1244 | 1626 | EVTQNEGTKTIANNL | | | | X | Q8JQF8 |
| 1245 | 1627 | FKLFNIQVKEVTQNE | | | | X | Q8JQF8 |
| 1246 | 1628 | FPSQMLRTGNNFQFT | | | | X | Q8JQF8 |
| 1247 | 1629 | GPNTMANQAKNWLPG | | | | X | Q8JQF8 |
| 1248 | 1630 | LEHDKAYDQQLQAGD | | | | X | Q8JQF8 |
| 1249 | 1631 | LIFGKQNAARDNADY | | | | X | Q8JQF8 |
| 1250 | 1632 | LTSTIQVFTDSEYQL | | | | X | Q8JQF8 |
| 1251 | 1633 | LYKQISNGTSGGATN | | | | X | Q8JQF8 |
| 1252 | 1634 | MANQAKNWLPGPCYR | | | | X | Q8JQF8 |
| 1253 | 1635 | NLTSTIQVFTDSEYQ | | | | X | Q8JQF8 |
| 1254 | 1636 | NPGIAMATHKDDEER | | | | X | Q8JQF8 |
| 1255 | 1637 | NQSKLNSFITQYSTG | | | | X | Q8JQF8 |
| 1256 | 1638 | PNTMAAGGGAPMADN | | | | X | Q8JQF8 |
| 1257 | 1639 | SFKLFNIQVKEVTQN | | | | X | Q8JQF8 |
| 1258 | 1640 | TDSEYQLPYVLGSAH | | | | X | Q8JQF8 |
| 1259 | 1641 | TMAAGGGAPMADNNE | | | | X | Q8JQF8 |
| 1260 | 1642 | AMATHKDDEERFFPS | | | | X | Q8JQF8 |
| 1261 | 1643 | APSGVGPNTMAAGGG | | | | X | Q8JQF8 |
| 1262 | 1644 | DPPTTFNQSKLNSFI | | | | X | Q8JQF8 |
| 1263 | 1645 | DPQPLGEPPAAPSGV | | | | X | Q8JQF8 |
| 1264 | 1646 | EGIREWWALKPGAPK | | | | X | Q8JQF8 |
| 1265 | 1647 | FPSNGILIFGKQNAA | | | | X | Q8JQF8 |
| 1266 | 1648 | FTDSEYQLPYVLGSA | | | | X | Q8JQF8 |
| 1267 | 1649 | GAKTAPGKKRPVEPS | | | | X | Q8JQF8 |
| 1268 | 1650 | GGTANTQTLGFSQGG | | | | X | Q8JQF8 |
| 1269 | 1651 | GILIFGKQNAARDNA | | | | X | Q8JQF8 |
| 1270 | 1652 | GTKTIANNLTSTIQV | | | | X | Q8JQF8 |
| 1271 | 1653 | INNNWGFRPKRLSFK | | | | X | Q8JQF8 |
| 1272 | 1654 | IQYTSNYYKSTSVDF | | | | X | Q8JQF8 |
| 1273 | 1655 | ISNGTSGGATNDNTY | | | | X | Q8JQF8 |
| 1274 | 1656 | KAYDQQLQAGDNPYL | | | | X | Q8JQF8 |
| 1275 | 1657 | KEVTQNEGTKTIANN | | | | X | Q8JQF8 |
| 1276 | 1658 | KLFNIQVKEVTQNEG | | | | X | Q8JQF8 |
| 1277 | 1659 | KSTSVDFAVNTEGVY | | | | X | Q8JQF8 |
| 1278 | 1660 | KTIANNLTSTIQVFT | | | | X | Q8JQF8 |
| 1279 | 1661 | KTTNPVATEEYGIVA | | | | X | Q8JQF8 |
| 1280 | 1662 | LMNPLIDQYLYYLSR | | | | X | Q8JQF8 |
| 1281 | 1663 | LPTYNNHLYKQISNG | | | | X | Q8JQF8 |
| 1282 | 1664 | LTSEEEIKTTNPVAT | | | | X | Q8JQF8 |
| 1283 | 1665 | MADNNEGADGVGSSS | | | | X | Q8JQF8 |
| 1284 | 1666 | NGTSGGATNDNTYFG | | | | X | Q8JQF8 |
| 1285 | 1667 | NHLYKQISNGTSGGA | | | | X | Q8JQF8 |
| 1286 | 1668 | NIQVKEVTQNEGTKT | | | | X | Q8JQF8 |
| 1287 | 1669 | NLQQQNTAPQIGTVN | | | | X | Q8JQF8 |
| 1288 | 1670 | NNHLYKQISNGTSGG | | | | X | Q8JQF8 |
| 1289 | 1671 | NTMANQAKNWLPGPC | | | | X | Q8JQF8 |
| 1290 | 1672 | PEIQYTSNYYKSTSV | | | | X | Q8JQF8 |
| 1291 | 1673 | PGKKRPVEPSPQRSP | | | | X | Q8JQF8 |
| 1292 | 1674 | PTYNNHLYKQISNGT | | | | X | Q8JQF8 |
| 1293 | 1675 | QGGPNTMANQAKNWL | | | | X | Q8JQF8 |
| 1294 | 1676 | QNEGTKTIANNLTST | | | | X | Q8JQF8 |
| 1295 | 1677 | QNNNSNFAWTAGTKY | | | | X | Q8JQF8 |
| 1296 | 1678 | QNTAPQIGTVNSQGA | | | | X | Q8JQF8 |
| 1297 | 1679 | QPLGEPPAAPSGVGP | | | | X | Q8JQF8 |
| 1298 | 1680 | QTLGFSQGGPNTMAN | | | | X | Q8JQF8 |
| 1299 | 1681 | QVKEVTQNEGTKTIA | | | | X | Q8JQF8 |
| 1300 | 1682 | SGVGPNTMAAGGGAP | | | | X | Q8JQF8 |
| 1301 | 1683 | SNFAWTAGTKYHLNG | | | | X | Q8JQF8 |
| 1302 | 1684 | SQGALPGMVWQNRDV | | | | X | Q8JQF8 |
| 1303 | 1685 | TQNEGTKTIANNLTS | | | | X | Q8JQF8 |
| 1304 | 1686 | TSGGATNDNTYFGYS | | | | X | Q8JQF8 |
| 1305 | 1687 | TSTIQVFTDSEYQLP | | | | X | Q8JQF8 |
| 1306 | 1688 | TYNNHLYKQISNGTS | | | | X | Q8JQF8 |
| 1307 | 1689 | VGPNTMAAGGGAPMA | | | | X | Q8JQF8 |
| 1308 | 1690 | VMLTSEEEIKTTNPV | | | | X | Q8JQF8 |
| 1309 | 1691 | WLPGPCYRQQRVSTT | | | | X | Q8JQF8 |
| 1310 | 1692 | YLSRTQTTGGTANTQ | | | | X | Q8JQF8 |
| 1311 | 1693 | YYKSTSVDFAVNTEG | | | | X | Q8JQF8 |
| 1312 | 1694 | DGNFHPSPLMGGFGL | | | | X | Q8JQF8 |
| 1313 | 1695 | NDNTYFGYSTPWGYF | | | | X | Q8JQF8 |
| 1314 | 1696 | GALPGMVWQNRDVYL | | | | X | Q8JQF8 |
| 1315 | 1697 | AGGGAPMADNNEGAD | | | | X | Q8JQF8 |
| 1316 | 1698 | RPVEPSPQRSPDSST | | | | X | Q8JQF8 |
| 1317 | 1699 | MVWQNRDVYLQGPIW | | | | X | Q8JQF8 |
| 1318 | 1700 | TNDNTYFGYSTPWGY | | | | X | Q8JQF8 |
| 1319 | 1701 | KRPVEPSPQRSPDSS | | | | X | Q8JQF8 |
| 1320 | 1702 | LPGMVWQNRDVYLQG | | | | X | Q8JQF8 |
| 1321 | 1703 | VPDPQPLGEPPAAPS | | | | X | Q8JQF8 |
| 1322 | 1704 | VWQNRDVYLQGPIWA | | | | X | Q8JQF8 |
| 1323 | 1705 | AAGGGAPMADNNEGA | | | | X | Q8JQF8 |
| 1324 | 1706 | DSSTGIGKKGQQPAR | | | | X | Q8JQF8 |
| 1325 | 1707 | GKKGQQPARKRLNFG | | | | X | Q8JQF8 |
| 1326 | 1708 | NFHPSPLMGGFGLKH | | | | X | Q8JQF8 |
| 1327 | 1709 | QGALPGMVWQNRDVY | | | | X | Q8JQF8 |
| 1328 | 1710 | QQPARKRLNFGQTGD | | | | X | Q8JQF8 |
| 1329 | 1711 | ALPGMVWQNRDVYLQ | | | | X | Q8JQF8 |
| 1330 | 1712 | GIGKKGQQPARKRLN | | | | X | Q8JQF8 |
| 1331 | 1713 | GMVWQNRDVYLQGPI | | | | X | Q8JQF8 |
| 1332 | 1714 | GNFHPSPLMGGFGLK | | | | X | Q8JQF8 |
| 1333 | 1715 | KKRPVEPSPQRSPDS | | | | X | Q8JQF8 |
| 1334 | 1716 | PDSSTGIGKKGQQPA | | | | X | Q8JQF8 |
| 1335 | 1717 | PGMVWQNRDVYLQGP | | | | X | Q8JQF8 |
| 1336 | 1718 | QRSPDSSTGIGKKGQ | | | | X | Q8JQF8 |
| 1337 | 1719 | VEPSPQRSPDSSTGI | | | | X | Q8JQF8 |
| 1338 | 1720 | QAKKRVLEPLGLVEE | | | | X | Q8JQF8 |
| 1339 | 1721 | EPLGLVEEGAKTAPG | | | | X | Q8JQF8 |
| 1340 | 1722 | RAVFQAKKRVLEPLG | | | | X | Q8JQF8 |
| 1341 | 1723 | AKKRVLEPLGLVEEG | | | | X | Q8JQF8 |
| 1342 | 1724 | FQAKKRVLEPLGLVE | | | | X | Q8JQF8 |
| 1343 | 1725 | PLGLVEEGAKTAPGK | | | | X | Q8JQF8 |
| 1344 | 1726 | RVLEPLGLVEEGAKT | | | | X | Q8JQF8 |
| 1345 | 1727 | GYSTPWGYFDFNRFH | | | | X | Q9YIJ1 |
| 1346 | 1728 | FEFTYNFEEVPFHSS | | | | X | Q9YIJ1 |
| 1347 | 1729 | NPTERSSFFCLEYFP | | | | X | Q9YIJ1 |
| 1348 | 1730 | GDWHCDSTWMGDRVV | | | | X | Q9YIJ1 |
| 1349 | 1731 | TVQVFTDDDYQLPYV | | | | X | Q9YIJ1 |
| 1350 | 1732 | FTYNFEEVPFHSSFA | | | | X | Q9YIJ1 |
| 1351 | 1733 | VDHPPDWLEEVGEGL | | | | X | Q9YIJ1 |
| 1352 | 1734 | RSSFFCLEYFPSKML | | | | X | Q9YIJ1 |
| 1353 | 1735 | TGAHFHPSPAMGGFG | | | | X | Q9YIJ1 |
| 1354 | 1736 | ASGDWHCDSTWMGDR | | | | X | Q9YIJ1 |
| 1355 | 1737 | GVGNASGDWHCDSTW | | | | X | Q9YIJ1 |
| 1356 | 1738 | PQFVDFAPDSTGEYR | | | | X | Q9YIJ1 |
| 1357 | 1739 | RGEPVNRADEVAREH | | | | X | Q9YIJ1 |
| 1358 | 1740 | AYFGYSTPWGYFDFN | | | | X | Q9YIJ1 |
| 1359 | 1741 | DNTENPTERSSFFCL | | | | X | Q9YIJ1 |
| 1360 | 1742 | DYQLPYVVGNGTEGC | | | | X | Q9YIJ1 |
| 1361 | 1743 | PETGAHFHPSPAMGG | | | | X | Q9YIJ1 |
| 1362 | 1744 | RADEVAREHDISYNE | | | | X | Q9YIJ1 |
| 1363 | 1745 | TERSSFFCLEYFPSK | | | | X | Q9YIJ1 |
| 1364 | 1746 | EGLREFLGLEAGPPK | | | | X | Q9YIJ1 |
| 1365 | 1747 | NDPQFVDFAPDSTGE | | | | X | Q9YIJ1 |
| 1366 | 1748 | PAMGGFGLKHPPPMM | | | | X | Q9YIJ1 |
| 1367 | 1749 | VAREHDISYNEQLEA | | | | X | Q9YIJ1 |
| 1368 | 1750 | VNRADEVAREHDISY | | | | X | Q9YIJ1 |
| 1369 | 1751 | YNHADAEFQEKLADD | | | | X | Q9YIJ1 |
| 1370 | 1752 | EPFGLVEEGAKTAPT | | | | X | Q9YIJ1 |
| 1371 | 1753 | FSDVPVSSFITQYST | | | | X | Q9YIJ1 |
| 1372 | 1754 | FTDDDYQLPYVVGNG | | | | X | Q9YIJ1 |
| 1373 | 1755 | HADAEFQEKLADDTS | | | | X | Q9YIJ1 |
| 1374 | 1756 | MGGFGLKHPPPMMLI | | | | X | Q9YIJ1 |
| 1375 | 1757 | NGLDRGEPVNRADEV | | | | X | Q9YIJ1 |
| 1376 | 1758 | NPEIQYTNNYNDPQF | | | | X | Q9YIJ1 |
| 1377 | 1759 | QVFTDDDYQLPYVVG | | | | X | Q9YIJ1 |
| 1378 | 1760 | REHDISYNEQLEAGD | | | | X | Q9YIJ1 |
| 1379 | 1761 | VGEGLREFLGLEAGP | | | | X | Q9YIJ1 |
| 1380 | 1762 | WGYFDFNRFHSHWSP | | | | X | Q9YIJ1 |
| 1381 | 1763 | WSPRDWQRLINNYWG | | | | X | Q9YIJ1 |
| 1382 | 1764 | YFDFNRFHSHWSPRD | | | | X | Q9YIJ1 |
| 1383 | 1765 | ADGVGNASGDWHCDS | | | | X | Q9YIJ1 |
| 1384 | 1766 | ARTEEDSKPSTSSDA | | | | X | Q9YIJ1 |
| 1385 | 1767 | ASVSAFATTNRMELE | | | | X | Q9YIJ1 |
| 1386 | 1768 | DFNRFHSHWSPRDWQ | | | | X | Q9YIJ1 |
| 1387 | 1769 | EGCLPAFPPQVFTLP | | | | X | Q9YIJ1 |
| 1388 | 1770 | ETQPVNRVAYNVGGQ | | | | X | Q9YIJ1 |
| 1389 | 1771 | FHPSPAMGGFGLKHP | | | | X | Q9YIJ1 |
| 1390 | 1772 | GGGGPLGDNNQGADG | | | | X | Q9YIJ1 |
| 1391 | 1773 | GGPLGDNNQGADGVG | | | | X | Q9YIJ1 |
| 1392 | 1774 | IDDHFPKRKKARTEE | | | | X | Q9YIJ1 |
| 1393 | 1775 | LVEEGAKTAPTGKRI | | | | X | Q9YIJ1 |
| 1394 | 1776 | NLQEIVPGSVWMERD | | | | X | Q9YIJ1 |
| 1395 | 1777 | NPYLKYNHADAEFQE | | | | X | Q9YIJ1 |
| 1396 | 1778 | PAFPPQVFTLPQYGY | | | | X | Q9YIJ1 |
| 1397 | 1779 | PASSLGADTMSAGGG | | | | X | Q9YIJ1 |
| 1398 | 1780 | PVSSFITQYSTGQVT | | | | X | Q9YIJ1 |
| 1399 | 1781 | QGADGVGNASGDWHC | | | | X | Q9YIJ1 |
| 1400 | 1782 | TGGVQFNKNLAGRYA | | | | X | Q9YIJ1 |
| 1401 | 1783 | VSAFATTNRMELEGA | | | | X | Q9YIJ1 |
| 1402 | 1784 | YFPSKMLRTGNNFEF | | | | X | Q9YIJ1 |
| 1403 | 1785 | YNFEEVPFHSSFAPS | | | | X | Q9YIJ1 |
| 1404 | 1786 | AFATTNRMELEGASY | | | | X | Q9YIJ1 |
| 1405 | 1787 | AHFHPSPAMGGFGLK | | | | X | Q9YIJ1 |
| 1406 | 1788 | DFAPDSTGEYRTTRP | | | | X | Q9YIJ1 |
| 1407 | 1789 | DGSNANAYFGYSTPW | | | | X | Q9YIJ1 |
| 1408 | 1790 | DNPYLKYNHADAEFQ | | | | X | Q9YIJ1 |
| 1409 | 1791 | DSTGEYRTTRPIGTR | | | | X | Q9YIJ1 |
| 1410 | 1792 | EGNMLITSESETQPV | | | | X | Q9YIJ1 |
| 1411 | 1793 | FVSTNNTGGVQFNKN | | | | X | Q9YIJ1 |
| 1412 | 1794 | GDNPYLKYNHADAEF | | | | X | Q9YIJ1 |
| 1413 | 1795 | GGNLGKAVFQAKKRV | | | | X | Q9YIJ1 |
| 1414 | 1796 | GTEGCLPAFPPQVFT | | | | X | Q9YIJ1 |
| 1415 | 1797 | HDISYNEQLEAGDNP | | | | X | Q9YIJ1 |
| 1416 | 1798 | IVPGSVWMERDVYLQ | | | | X | Q9YIJ1 |
| 1417 | 1799 | KIPETGAHFHPSPAM | | | | X | Q9YIJ1 |
| 1418 | 1800 | KMLRTGNNFEFTYNF | | | | X | Q9YIJ1 |
| 1419 | 1801 | LEAGPPKPKPNQQHQ | | | | X | Q9YIJ1 |
| 1420 | 1802 | LEYFPSKMLRTGNNF | | | | X | Q9YIJ1 |
| 1421 | 1803 | LGPGNGLDRGEPVNR | | | | X | Q9YIJ1 |
| 1422 | 1804 | LPGYNYLGPGNGLDR | | | | X | Q9YIJ1 |
| 1423 | 1805 | LVDQYLYRFVSTNNT | | | | X | Q9YIJ1 |
| 1424 | 1806 | NIQVKEVTVQDSTTT | | | | X | Q9YIJ1 |
| 1425 | 1807 | NITSFSDVPVSSFIT | | | | X | Q9YIJ1 |
| 1426 | 1808 | NNQGADGVGNASGDW | | | | X | Q9YIJ1 |
| 1427 | 1809 | NRASVSAFATTNRME | | | | X | Q9YIJ1 |
| 1428 | 1810 | NTPVPGNITSFSDVP | | | | X | Q9YIJ1 |
| 1429 | 1811 | NYLGPGNGLDRGEPV | | | | X | Q9YIJ1 |
| 1430 | 1812 | PATGTYNLQEIVPGS | | | | X | Q9YIJ1 |
| 1431 | 1813 | PGNGLDRGEPVNRAD | | | | X | Q9YIJ1 |
| 1432 | 1814 | PGNITSFSDVPVSSF | | | | X | Q9YIJ1 |
| 1433 | 1815 | PRDWQRLINNYWGFR | | | | X | Q9YIJ1 |
| 1434 | 1816 | PSPAMGGFGLKHPPP | | | | X | Q9YIJ1 |
| 1435 | 1817 | PYVVGNGTEGCLPAF | | | | X | Q9YIJ1 |
| 1436 | 1818 | QARGLVLPGYNYLGP | | | | X | Q9YIJ1 |
| 1437 | 1819 | QEKLADDTSFGGNLG | | | | X | Q9YIJ1 |
| 1438 | 1820 | QLPYVVGNGTEGCLP | | | | X | Q9YIJ1 |
| 1439 | 1821 | RMELEGASYQVPPQP | | | | X | Q9YIJ1 |
| 1440 | 1822 | SVWMERDVYLQGPIW | | | | X | Q9YIJ1 |
| 1441 | 1823 | TGEYRTTRPIGTRYL | | | | X | Q9YIJ1 |
| 1442 | 1824 | TTTIANNLTSTVQVF | | | | X | Q9YIJ1 |
| 1443 | 1825 | VVGNGTEGCLPAFPP | | | | X | Q9YIJ1 |
| 1444 | 1826 | WMERDVYLQGPIWAK | | | | X | Q9YIJ1 |
| 1445 | 1827 | WMGDRVVTKSTRTWV | | | | X | Q9YIJ1 |
| 1446 | 1828 | YLYRFVSTNNTGGVQ | | | | X | Q9YIJ1 |
| 1447 | 1829 | ADDTSFGGNLGKAVF | | | | X | Q9YIJ1 |
| 1448 | 1830 | AKKRVLEPFGLVEEG | | | | X | Q9YIJ1 |
| 1449 | 1831 | ALENTMIFNSQPANP | | | | X | Q9YIJ1 |
| 1450 | 1832 | APDSTGEYRTTRPIG | | | | X | Q9YIJ1 |
| 1451 | 1833 | APSQNLFKLANPLVD | | | | X | Q9YIJ1 |
| 1452 | 1834 | APTGKRIDDHFPKRK | | | | X | Q9YIJ1 |
| 1453 | 1835 | ASYQVPPQPNGMTNN | | | | X | Q9YIJ1 |
| 1454 | 1836 | ATNNQSSTTAPATGT | | | | X | Q9YIJ1 |
| 1455 | 1837 | DHFPKRKKARTEEDS | | | | X | Q9YIJ1 |
| 1456 | 1838 | DTSFGGNLGKAVFQA | | | | X | Q9YIJ1 |
| 1457 | 1839 | DVPVSSFITQYSTGQ | | | | X | Q9YIJ1 |
| 1458 | 1840 | DWQRLINNYWGFRPR | | | | X | Q9YIJ1 |
| 1459 | 1841 | FPPQVFTLPQYGYAT | | | | X | Q9YIJ1 |
| 1460 | 1842 | FQAKKRVLEPFGLVE | | | | X | Q9YIJ1 |
| 1461 | 1843 | GDNNQGADGVGNASG | | | | X | Q9YIJ1 |
| 1462 | 1844 | GDRVVTKSTRTWVLP | | | | X | Q9YIJ1 |
| 1463 | 1845 | GFRPRSLRVKIFNIQ | | | | X | Q9YIJ1 |
| 1464 | 1846 | GGQMATNNQSSTTAP | | | | X | Q9YIJ1 |
| 1465 | 1847 | GSGVNRASVSAFATT | | | | X | Q9YIJ1 |
| 1466 | 1848 | GSQQLQIPAQPASSL | | | | X | Q9YIJ1 |
| 1467 | 1849 | GVNRASVSAFATTNR | | | | X | Q9YIJ1 |
| 1468 | 1850 | IKSGSVDGSNANAYF | | | | X | Q9YIJ1 |
| 1469 | 1851 | KEVTVQDSTTTIANN | | | | X | Q9YIJ1 |
| 1470 | 1852 | KKENSKRWNPEIQYT | | | | X | Q9YIJ1 |
| 1471 | 1853 | LDRGEPVNRADEVAR | | | | X | Q9YIJ1 |
| 1472 | 1854 | LGADTMSAGGGGPLG | | | | X | Q9YIJ1 |
| 1473 | 1855 | LINNYWGFRPRSLRV | | | | X | Q9YIJ1 |
| 1474 | 1856 | LREFLGLEAGPPKPK | | | | X | Q9YIJ1 |
| 1475 | 1857 | LVLPGYNYLGPGNGL | | | | X | Q9YIJ1 |
| 1476 | 1858 | NLAGRYANTYKNWFP | | | | X | Q9YIJ1 |
| 1477 | 1859 | NLFKLANPLVDQYLY | | | | X | Q9YIJ1 |
| 1478 | 1860 | NNQSSTTAPATGTYN | | | | X | Q9YIJ1 |
| 1479 | 1861 | NNTGGVQFNKNLAGR | | | | X | Q9YIJ1 |
| 1480 | 1862 | NPLVDQYLYRFVSTN | | | | X | Q9YIJ1 |
| 1481 | 1863 | NRDNTENPTERSSFF | | | | X | Q9YIJ1 |
| 1482 | 1864 | PFHSSFAPSQNLFKL | | | | X | Q9YIJ1 |
| 1483 | 1865 | PIWAKIPETGAHFHP | | | | X | Q9YIJ1 |
| 1484 | 1866 | PLGDNNQGADGVGNA | | | | X | Q9YIJ1 |
| 1485 | 1867 | PMGRTQGWNLGSGVN | | | | X | Q9YIJ1 |
| 1486 | 1868 | PMMLIKNTPVPGNIT | | | | X | Q9YIJ1 |
| 1487 | 1869 | PQPNGMTNNLQGSNT | | | | X | Q9YIJ1 |
| 1488 | 1870 | QGPIWAKIPETGAHF | | | | X | Q9YIJ1 |
| 1489 | 1871 | QGSNTYALENTMIFN | | | | X | Q9YIJ1 |
| 1490 | 1872 | QPVNRVAYNVGGQMA | | | | X | Q9YIJ1 |
| 1491 | 1873 | QVKEVTVQDSTTTIA | | | | X | Q9YIJ1 |
| 1492 | 1874 | REIKSGSVDGSNANA | | | | X | Q9YIJ1 |
| 1493 | 1875 | RTTRPIGTRYLTRPL | | | | X | Q9YIJ1 |
| 1494 | 1876 | RWNPEIQYTNNYNDP | | | | X | Q9YIJ1 |
| 1495 | 1877 | RYANTYKNWFPGPMG | | | | X | Q9YIJ1 |
| 1496 | 1878 | SFFCLEYFPSKMLRT | | | | X | Q9YIJ1 |
| 1497 | 1879 | SFGGNLGKAVFQAKK | | | | X | Q9YIJ1 |
| 1498 | 1880 | SNANAYFGYSTPWGY | | | | X | Q9YIJ1 |
| 1499 | 1881 | SNTYALENTMIFNSQ | | | | X | Q9YIJ1 |
| 1500 | 1882 | SQNLFKLANPLVDQY | | | | X | Q9YIJ1 |
| 1501 | 1883 | SVDGSNANAYFGYST | | | | X | Q9YIJ1 |
| 1502 | 1884 | TGTYNLQEIVPGSVW | | | | X | Q9YIJ1 |
| 1503 | 1885 | TLNRDNTENPTERSS | | | | X | Q9YIJ1 |
| 1504 | 1886 | TSESETQPVNRVAYN | | | | X | Q9YIJ1 |
| 1505 | 1887 | TYKNWFPGPMGRTQG | | | | X | Q9YIJ1 |
| 1506 | 1888 | YATLNRDNTENPTER | | | | X | Q9YIJ1 |
| 1507 | 1889 | YGYATLNRDNTENPT | | | | X | Q9YIJ1 |
| 1508 | 1890 | YRFVSTNNTGGVQFN | | | | X | Q9YIJ1 |
| 1509 | 1891 | YSTPWGYFDFNRFHS | | | | X | Q9YIJ1 |

### Example 12: Screen for Anti-AAV Antibodies in Human Sera Based on Cyclic Peptides

More than 1200 cyclic peptides derived from the sequences of human and rhesus monkey AAV sequences and artificial AAV sequences according to Table 2 and with a sequence length of 14 amino-acids each were synthesized.

Samples obtained from human donors were screened for antibodies against these AAV-derived peptides immobilized on microarrays. To this end, IgG was prepared from blood obtained from the human donors by protein G purification. Each IgG sample was incubated with the peptide microarrays and Ig binding signals were detected by fluorescence. All antibody binding signals to the peptides on the arrays were background subtracted and ranked for each sample and a deduplicated aggregate of the respective top 250 peptide hits for each donor with the corresponding protein sequence of origin (as obtained from UniProt or other sources) was compiled (designated as group II). Further, the deduplicated aggregate of the respective top 50 peptide hits for each donor was compiled and designated as group I.

Detailed results are shown in Table 2 below. Altogether, group I contains 47 distinct peptide hits (assigned to the corresponding AAV vectors in Table 2) and group II yielded 172 distinct peptide hits. Evidently, group I is a subset of group II.

Thus, all listed peptides, preferably peptides belonging to group I, provide sequences from which shorter peptide sequences can be derived for antibody depletion according to the present invention. Furthermore, also other peptide sequences (or fragments) from the proteins from which the peptides of Table 2 were derived (preferably from group I), are suited to be used for SADCs according to the present invention. In addition, these peptides can also be used as probes for the diagnostic detection of anti-AAV antibodies in biological samples such as human sera.

TABLE 2
This table lists the detailed results of a screen for
circularized peptides as a basis for the construction of anti-AAV
antibody depleting SADCs according to the present invention. These
peptides are also suitable for typing neutralizing antibodies
directed against AAV vectors. If not stated otherwise, the peptides
represent fragments from different AAV VP1 proteins. Source given
is either UniProt ID, GenBank ID, PDB ID or AAV strain name.

| peptide | SEQ | | group | group | |
|---|---|---|---|---|---|
| # | ID NO | peptide | I | II | source |
| 1 | 1892 | DSQWLGDRVITTST | X | X | AAVLK03.L125I |
| 2 | 1893 | DTNGVYSEPRPIGT | X | X | AAVLK03.L125I |
| 3 | 1894 | STNLQRGNLALGET | X | X | AOD99651.1 |
| 4 | 1895 | ANNLTSTVQIFADS | X | X | A9RAI0 |
| 5 | 1896 | KIFNIQVKEVTTSN | X | X | A9RAI0 |
| 6 | 1897 | GNTSQQQTDRNAFY | X | X | O41855 |
| 7 | 1898 | LEDNLSEGIREWWD | X | X | AAO88201.1 |
| 8 | 1899 | SESVPDPQPIGEPP | X | X | AAO88201.1 |
| 9 | 1900 | LIKNTPVPADPPTT | X | X | AAO88201.1 |
| 10 | 1901 | PQYGYLTLNNGSQA | X | X | AAO88201.1 |
| 11 | 1902 | DEEEIRTTNPVATE | X | X | AAVLK03.L125I |
| 12 | 1903 | NYNKSVNVDFTVDT | X | X | AAVLK03.L125I |
| 13 | 1904 | YHLNGRDSLVNPGP | X | X | AAVLK03.L125I |
| 14 | 1905 | TRPATAPQIGTVNS | X | X | AOD99652.1 |
| 15 | 1906 | GETTRPATAAQTQV | X | X | AOD99656.1 |
| 16 | 1907 | IFNIQVKEVTTSNG | X | X | A9RAI0 |
| 17 | 1908 | SNSQLIFAGPNPSG | X | X | A9RAI0 |
| 18 | 1909 | TTTSSNNLLFTSEE | X | X | A9RAI0 |
| 19 | 1910 | FNIQVKEVTTNDGV | X | X | O56137 |
| 20 | 1911 | GQTGDSESVPDPQP | X | X | AAO88201.1 |
| 21 | 1912 | PQILIKNTPVPADP | X | X | AAO88201.1 |
| 22 | 1913 | QLKAGDNPYLRYNH | X | X | AAO88201.1 |
| 23 | 1914 | SFITQYSTGQVSVE | X | X | AAO88201.1 |
| 24 | 1915 | FGKQGAGRDNVDYS | X | X | AAS99285.1 |
| 25 | 1916 | YYKSTNVDFAVNTE | X | X | AAS99285.1 |
| 26 | 1917 | HYFGYSTPWGYFDF | X | X | AAVLK03.L125I |
| 27 | 1918 | APGKKRPVDQSPQE | X | X | AAVLK03.L125I |
| 28 | 1919 | GKKRPVDQSPQEPD | X | X | AAVLK03.L125I |
| 29 | 1920 | KTAPGKKRPVDQSP | X | X | AAVLK03.L125I |
| 30 | 1921 | PEIQYTSNYNKSVN | X | X | AAVLK03.L125I |
| 31 | 1922 | SESVPDPQPLGEPP | X | X | AAVLK03.L125I |
| 32 | 1923 | TAPGKKRPVDQSPQ | X | X | AAVLK03.L125I |
| 33 | 1924 | YDQQLKAGDNPYLK | X | X | AAVLK03.L125I |
| 34 | 1925 | YLYYLNRTQGTTSG | X | X | AAVLK03.L125I |
| 35 | 1926 | DKAYDRQLDSGDNP | X | X | AAV2i8 |
| 36 | 1927 | GTNTMATGSGAPMA | X | X | AAV2i8 |
| 37 | 1928 | DKAYDQQLQAGDNP | X | X | AAV-Rh74 |
| 38 | 1929 | TESVPDPQPIGEPP | X | X | ALU85156.1 |
| 39 | 1930 | KNTPVPADPPTTES | X | X | AAO88201.1 |
| 40 | 1931 | PVPADPPTTESQAK | X | X | AAO88201.1 |
| 41 | 1932 | DEEEIKATNPVATE | X | X | O56137 |
| 42 | 1933 | DKDKFFPMSGVMIF | X | X | O56137 |
| 43 | 1934 | LQQQNTAPQIGTVN | X | X | Q8JQF8 |
| 44 | 1935 | EEEIKTTNPVATEE | X | X | Q8JQF8 |
| 45 | 1936 | GQNNNSNFAWTAGT | X | X | Q8JQF8 |
| 46 | 1937 | DDEDKFFPMSGVMI | X | X | Q9WBP8 |
| 47 | 1938 | PLVDQYLYRFVSTN | X | X | Q9YIJ1 |
| 48 | 1939 | ADPPTTFSQAKLAS | | X | AAO88201.1 |
| 49 | 1940 | DAAALEHDKAYDQQ | | X | AAO88201.1 |
| 50 | 1941 | DKAYDQQLKAGDNP | | X | AAO88201.1 |
| 51 | 1942 | DSESVPDPQPIGEP | | X | AAO88201.1 |
| 52 | 1943 | DWLEDNLSEGIREW | | X | AAO88201.1 |
| 53 | 1944 | EDNLSEGIREWWDL | | X | AAO88201.1 |
| 54 | 1945 | EEIKTTNPVATEQY | | X | AAO88201.1 |
| 55 | 1946 | ENSKRWNPEIQYTS | | X | AAO88201.1 |
| 56 | 1947 | ESVPDPQPIGEPPA | | X | AAO88201.1 |
| 57 | 1948 | EYQLPYVLGSAHQG | | X | AAO88201.1 |
| 58 | 1949 | FQERLQEDTSFGGN | | X | AAO88201.1 |
| 59 | 1950 | GDSESVPDPQPIGE | | X | AAO88201.1 |
| 60 | 1951 | HSQSLDRLMNPLID | | X | AAO88201.1 |
| 61 | 1952 | KGEPVNAADAAALE | | X | AAO88201.1 |
| 62 | 1953 | KNTPVPADPPTTFS | | X | AAO88201.1 |
| 63 | 1954 | LPYVLGSAHQGCLP | | X | AAO88201.1 |
| 64 | 1955 | LQQQNAAPIVGAVN | | X | AAO88201.1 |
| 65 | 1956 | NAADAAALEHDKAY | | X | AAO88201.1 |
| 66 | 1957 | NPGVAMATHKDDEE | | X | AAO88201.1 |
| 67 | 1958 | PGAPKPKANQQKQD | | X | AAO88201.1 |
| 68 | 1959 | PPQILIKNTPVPAD | | X | AAO88201.1 |
| 69 | 1960 | PRDWQRLINNNWGF | | X | AAO88201.1 |
| 70 | 1961 | PWGYFDFNRFHCHF | | X | AAO88201.1 |
| 71 | 1962 | QLPYVLGSAHQGCL | | X | AAO88201.1 |
| 72 | 1963 | QQRVSTTLSQNNNS | | X | AAO88201.1 |
| 73 | 1964 | SEPRPIGTRYLTRN | | X | AAO88201.1 |
| 74 | 1965 | SGGSTNDNTYFGYS | | X | AAO88201.1 |
| 75 | 1966 | SQSLDRLMNPLIDQ | | X | AAO88201.1 |
| 76 | 1967 | TGDSESVPDPQPIG | | X | AAO88201.1 |
| 77 | 1968 | TIANNLTSTIQVFT | | X | AAO88201.1 |
| 78 | 1969 | TQYSTGQVSVEIEW | | X | AAO88201.1 |
| 79 | 1970 | VTQNEGTKTIANNL | | X | AAO88201.1 |
| 80 | 1971 | WLEDNLSEGIREWW | | X | AAO88201.1 |
| 81 | 1972 | YFGYSTPWGYFDFN | | X | AAO88201.1 |
| 82 | 1973 | LSRTQSTGGTQGTQ | | X | AAS99285.1 |
| 83 | 1974 | TQGTQQLLFSQAGP | | X | AAS99285.1 |
| 84 | 1975 | DAEFQERLKEDTSF | | X | AAVLK03.L125I |
| 85 | 1976 | DDEEKFFPMHGNLI | | X | AAVLK03.L125I |
| 86 | 1977 | DGHFHPSPLMGGFG | | X | AAVLK03.L125I |
| 87 | 1978 | DSESVPDPQPLGEP | | X | AAVLK03.L125I |
| 88 | 1979 | EEEIRTTNPVATEQ | | X | AAVLK03.L125I |
| 89 | 1980 | EEIRTTNPVATEQY | | X | AAVLK03.L125I |
| 90 | 1981 | FQERLKEDTSFGGN | | X | AAVLK03.L125I |
| 91 | 1982 | GADGVGNSSGNWHC | | X | AAVLK03.L125I |
| 92 | 1983 | GDSESVPDPQPLGE | | X | AAVLK03.L125I |
| 93 | 1984 | GNGLDKGEPVNAAD | | X | AAVLK03.L125I |
| 94 | 1985 | KKRPVDQSPQEPDS | | X | AAVLK03.L125I |
| 95 | 1986 | KRPVDQSPQEPDSS | | X | AAVLK03.L125I |
| 96 | 1987 | KSVNVDFTVDTNGV | | X | AAVLK03.L125I |
| 97 | 1988 | LSKTANDNNNSNFP | | X | AAVLK03.L125I |
| 98 | 1989 | MASHKDDEEKFFPM | | X | AAVLK03.L125I |
| 99 | 1990 | NNFQFSYTFEDVPF | | X | AAVLK03.L125I |
| 100 | 1991 | PVDQSPQEPDSSSG | | X | AAVLK03.L125I |
| 101 | 1992 | PVPANPPTTFSPAK | | X | AAVLK03.L125I |
| 102 | 1993 | QQRLSKTANDNNNS | | X | AAVLK03.L125I |
| 103 | 1994 | QSSNTAPTTRTVND | | X | AAVLK03.L125I |
| 104 | 1995 | SKTANDNNNSNFPW | | X | AAVLK03.L125I |
| 105 | 1996 | SNYNKSVNVDFTVD | | X | AAVLK03.L125I |
| 106 | 1997 | TTSGTTNQSRLLFS | | X | AAVLK03.L125I |
| 107 | 1998 | VMITDEEEIRTTNP | | X | AAVLK03.L125I |
| 108 | 1999 | APGKKRPVEHSPVE | | X | AAV2i8 |
| 109 | 2000 | FFPQSGVLIFGKQG | | X | AAV2i8 |
| 110 | 2001 | FGKQGSEKTNVDIE | | X | AAV2i8 |
| 111 | 2002 | HKDDEEKFFPQSGV | | X | AAV2i8 |
| 112 | 2003 | KGEPVNEADAAALE | | X | AAV2i8 |
| 113 | 2004 | NEADAAALEHDKAY | | X | AAV2i8 |
| 114 | 2005 | NPVATEQYGSVSTN | | X | AAV2i8 |
| 115 | 2006 | PQILIKNTPVPANP | | X | AAV2i8 |
| 116 | 2007 | QQRVSKTSADNNNS | | X | AAV2i8 |
| 117 | 2008 | QTGDADSVPDPQPL | | X | AAV2i8 |
| 118 | 2009 | ADPPTTFNQAKLAS | | X | AAV-Rh74 |
| 119 | 2010 | AGDNVDYSSVMLTS | | X | AAV-Rh74 |
| 120 | 2011 | KNTPVPADPPTTFN | | X | AAV-Rh74 |
| 121 | 2012 | KRVLEPLGLVESPV | | X | AAV-Rh74 |
| 122 | 2013 | PYLRYHADAEFQER | | X | AAV-Rh74 |
| 123 | 2014 | EEEIKTTNPVATES | | X | ALU85156.1 |
| 124 | 2015 | EFAWPGASSWALNG | | X | ALU85156.1 |
| 125 | 2016 | KSNNVEFAVNTEGV | | X | ALU85156.1 |
| 126 | 2017 | MNPGPAMASHKEGE | | X | ALU85156.1 |
| 127 | 2018 | PVPADPPTAFNKDK | | X | ALU85156.1 |
| 128 | 2019 | TVQVFTDSDYQLPY | | X | ALU85156.1 |
| 129 | 2020 | NLQAANLALGETTR | | X | AOD99656.1 |
| 130 | 2021 | GGQMATNNQSLALG | | X | AOD99659.1 |
| 131 | 2022 | MATNNQSLALGETT | | X | AOD99659.1 |
| 132 | 2023 | KKRILEPLGLVEEA | | X | AAB95452.1 |
| 133 | 2024 | ADPPTTFNQSKLNS | | X | 3J1Q |
| 134 | 2025 | KSTSVDFAVNTEGV | | X | 3J1Q |
| 135 | 2026 | LQRGNRQAATADVN | | X | 3J1Q |
| 136 | 2027 | PVPADPPTTFNQSK | | X | 3J1Q |
| 137 | 2028 | DDDDRFFPMHGNLI | | X | QLI60567.1 |
| 138 | 2029 | PEPADVFMIPQYGY | | X | AAO88201.1 |
| 139 | 2030 | DIYYQGPIWAKVPH | | X | A9RAI0 |
| 140 | 2031 | FEKVPFHSMYAHSQ | | X | A9RAI0 |
| 141 | 2032 | FSAARINSFLTQYS | | X | A9RAI0 |
| 142 | 2033 | HSQSLDRMMNPLLD | | X | A9RAI0 |
| 143 | 2034 | KKRILEPLGLVEEG | | X | A9RAI0 |
| 144 | 2035 | MVPQYGYCGVVTGK | | X | A9RAI0 |
| 145 | 2036 | NQTDRNAFYCLEYF | | X | A9RAI0 |
| 146 | 2037 | RDTDMFGQIADNNQ | | X | A9RAI0 |
| 147 | 2038 | RDWQRLINNNWGLR | | X | A9RAI0 |
| 148 | 2039 | TVQIFADSTYELPY | | X | A9RAI0 |
| 149 | 2040 | DIYYQGPIWAKIPH | | X | O41855 |
| 150 | 2041 | QIFADSSYELPYVM | | X | O41855 |
| 151 | 2042 | THSTLDGRWSALTP | | X | O41855 |
| 152 | 2043 | TVQIFADSSYELPY | | X | O41855 |
| 153 | 2044 | DKFFPMSGVMIFGK | | X | O56137 |
| 154 | 2045 | EEEIKATNPVATER | | X | O56137 |
| 155 | 2046 | EGADGVGNASGNWH | | X | O56137 |
| 156 | 2047 | LFNIQVKEVTTNDG | | X | O56137 |
| 157 | 2048 | QVKEVTTNDGVTTI | | X | O56137 |
| 158 | 2049 | RVSKTKTDNNNSNF | | X | O56137 |
| 159 | 2050 | SDSEYQLPYVLGSA | | X | O56137 |
| 160 | 2051 | HSQSLDRLMNPLLD | | X | Q5Y9B2 |
| 161 | 2052 | IEMRAAPGGNAVDA | | X | Q5Y9B2 |
| 162 | 2053 | KRLNFEEDTGAGDG | | X | Q5Y9B2 |
| 163 | 2054 | SQSLDRLMNPLLDQ | | X | Q5Y9B2 |
| 164 | 2055 | STGQVAVQIEWEIE | | X | Q5Y9B2 |
| 165 | 2056 | TTSANNLLFTSEEE | | X | Q5Y9B2 |
| 166 | 2057 | TTSGETLNQGNAAT | | X | Q5Y9B2 |
| 167 | 2058 | GESESVPDPQPIGE | | X | Q5Y9B4 |
| 168 | 2059 | GQTGESESVPDPQP | | X | Q5Y9B4 |
| 169 | 2060 | ANPGIAMATHKDDE | | X | Q8JQF8 |
| 170 | 2061 | EGASYQVPPQPNGM | | X | Q9YIJ1 |
| 171 | 2062 | EYRTTRPIGTRYLT | | X | Q9YIJ1 |
| 172 | 2063 | YNLQEIVPGSVWME | | X | Q9YIJ1 |

### Example 13: Further Screen for Anti-AAV Antibodies in Human Sera

By using a cumulative gliding average signal of all sera tested over 4 consecutively aligned peptide signals along the corresponding AAV sequences, 1948 linear peptides were derived from AAV vectors AAV1, AAV2, AAV5, AAV6, AAV8, AAV9 and AAVrh. 10.

Detailed results are shown in Table 3 below. 63 top candidates with the strongest signals were assigned to group I corresponding to 3.2% of all AAV peptides analyzed by gliding average signal along the AAV VP1 sequence. The peptides of group I as well as the 135 peptides with second strongest signals were assigned to group II corresponding to 10.1% of all AAV peptides analyzed. Additional 82 peptides (assigned to group III) were derived from the top 200 ranked peptide signals of the present screen not covered by group I and II. In summary, groups I, II and III thus contain 280 linear peptides suitable (as basis for SADCs) to remove or to detect anti AAV antibodies, in particular antibodies directed against the AAV1, AAV2, AAV5, AAV6, AAV8, AAV9 and AAVrh. 10 VP1 proteins.

TABLE 3
This table provides a separate compilation of suitable
peptides covering stretches along the VP1 sequence of widely used
AAV vectors including AAV1, AAV2, AAV5, AAV6, AAV8, AAV9 and
AAVrh. 10. Source given is either UniProt ID, GenBank ID, PDB ID or
AAV strain name. The asterisk (*) indicates peptide sequences for
which a SEQ ID NO has already been assigned in Table 1 above.

| peptide | SEQ | | group | group | group | |
|---|---|---|---|---|---|---|
| # | ID NO | peptide | I | II | III | source |
| 1 | * | ADPPTAFNKDKLNSF | X | X | | Q6JC40 |
| 2 | 2064 | ADTMSAGGGGPLGDN | X | X | | Q9YIJ1 |
| 3 | * | AEFQERLKEDTSFGG | X | X | | spP03135 |
| 4 | 2065 | AKTAPGKKRPVEPSP | X | X | | Q8JQF8 |
| 5 | * | APTGKRIDDHFPKRK | X | X | | Q9YIJ1 |
| 6 | * | DKLNSFITQYSTGQV | X | X | | Q6JC40 |
| 7 | 2066 | DVYLQGPIWAKIPET | X | X | | Q9YIJ1 |
| 8 | * | EEEIKTTNPVATEEY | X | X | | Q8JQF8 |
| 9 | * | EEEIKTTNPVATEQY | X | X | | AAO88201.1 |
| 10 | * | EEEIRTTNPVATEQY | X | X | | spP03135 |
| 11 | * | EEIKTTNPVATEQYG | X | X | | AAO88201.1 |
| 12 | * | EIKTTNPVATEEYGI | X | X | | Q8JQF8 |
| 13 | * | EIRTTNPVATEQYGS | X | X | | spP03135 |
| 14 | * | ELKKENSKRWNPEIQ | X | X | | Q9YIJ1 |
| 15 | * | EMEWELKKENSKRWN | X | X | | Q9YIJ1 |
| 16 | * | ERDVYLQGPIWAKIP | X | X | | Q9YIJ1 |
| 17 | * | ERLKEDTSFGGNLGR | X | X | | spP03135 |
| 18 | * | EWELKKENSKRWNPE | X | X | | Q9YIJ1 |
| 19 | * | FITQYSTGQVSVEIE | X | X | | spP03135 |
| 20 | * | FITQYSTGQVTVEME | X | X | | Q9YIJ1 |
| 21 | * | FQERLKEDTSFGGNL | X | X | | spP03135 |
| 22 | 2067 | GAKTAPTGKRIDDHF | X | X | | Q9YIJ1 |
| 23 | 2068 | GQVATNHQSAQAQAQ | X | X | | Q6JC40 |
| 24 | * | IKTTNPVATEQYGVV | X | X | | AAO88201.1 |
| 25 | * | IQYTSNYNKSVNVDF | X | X | | spP03135 |
| 26 | * | KKENSKRWNPEIQYT | X | X | | Q9YIJ1 |
| 27 | * | KLNSFITQYSTGQVS | X | X | | Q6JC40 |
| 28 | 2069 | KTAPTGKRIDDHFPK | X | X | | Q9YIJ1 |
| 29 | * | KTTNPVATEEYGIVA | X | X | | Q8JQF8 |
| 30 | * | LKEDTSFGGNLGRAV | X | X | | spP03135 |
| 31 | * | LNSFITQYSTGQVSV | X | X | | Q6JC40 |
| 32 | 2070 | MNPLIDQYLYYLSKT | X | X | | Q6JC40 |
| 33 | 2071 | NHQYREIKSGSVDGS | X | X | | Q9YIJ1 |
| 34 | * | NSFITQYSTGQVSVE | X | X | | Q6JC40 |
| 35 | * | NTEGVYSEPRPIGTR | X | X | | Q6JC40 |
| 36 | * | PADPPTAFNKDKLNS | X | X | | Q6JC40 |
| 37 | * | PEIQYTSNYNKSVNV | X | X | | spP03135 |
| 38 | * | PLIDQYLYYLSKTIN | X | X | | Q6JC40 |
| 39 | 2072 | PTTFNQSKLNSFITQ | X | X | | Q8JQF8 |
| 40 | * | PVPADPPTAFNKDKL | X | X | | Q6JC40 |
| 41 | * | QGPIWAKIPETGAHF | X | X | | Q9YIJ1 |
| 42 | 2073 | QYREIKSGSVDGSNA | X | X | | Q9YIJ1 |
| 43 | * | REIKSGSVDGSNANA | X | X | | Q9YIJ1 |
| 44 | * | RTTNPVATEQYGSVS | X | X | | spP03135 |
| 45 | * | SEEEIKTTNPVATEQ | X | X | | AAO88201.1 |
| 46 | * | SFITQYSTGQVSVEI | X | X | | spP03135 |
| 47 | * | SSFITQYSTGQVTVE | X | X | | Q9YIJ1 |
| 48 | * | SSVMLTSEEEIKTTN | X | X | | AAO88201.1 |
| 49 | * | SSYAHSQSLDRLMNP | X | X | | spP03135 |
| 50 | * | SVMLTSEEEIKTTNP | X | X | | AAO88201.1 |
| 51 | 2074 | SYAHSQSLDRLMNPL | X | X | | spP03135 |
| 52 | * | TMSAGGGGPLGDNNQ | X | X | | Q9YIJ1 |
| 53 | * | TNPVATEEYGIVADN | X | X | | Q8JQF8 |
| 54 | * | TNPVATEQYGSVSTN | X | X | | spP03135 |
| 55 | * | TQTTGGTANTQTLGF | X | X | | Q8JQF8 |
| 56 | * | TTNPVATEQYGVVAD | X | X | | AAO88201.1 |
| 57 | * | VPADPPTAFNKDKLN | X | X | | Q6JC40 |
| 58 | 2075 | VYSEPRPIGTRYLTR | X | X | | spP03135 |
| 59 | * | WNPEIQYTSNYNKSV | X | X | | spP03135 |
| 60 | 2076 | YLQGPIWAKIPETGA | X | X | | Q9YIJ1 |
| 61 | * | YNNHQYREIKSGSVD | X | X | | Q9YIJ1 |
| 62 | * | YSSVMLTSEEEIKTT | X | X | | AAO88201.1 |
| 63 | * | YTSNYNKSVNVDFTV | X | X | | spP03135 |
| 64 | * | ADAEFQERLKEDTSF | | X | | spP03135 |
| 65 | * | AGPPKPKPNQQHQDQ | | X | | Q9YIJ1 |
| 66 | * | AGPSGLGSGTMAAGG | | X | | AAO88201.1 |
| 67 | * | ANNLTSTVQVFTDDD | | X | | Q9YIJ1 |
| 68 | * | CYRQQRVSTTTGQNN | | X | | Q8JQF8 |
| 69 | * | DPPTTFNQSKLNSFI | | X | | Q8JQF8 |
| 70 | 2077 | DSSSGTGKAGQQPAR | | X | | spP03135 |
| 71 | * | DSTTTIANNLTSTVQ | | X | | Q9YIJ1 |
| 72 | * | EDTSFGGNLGRAVFQ | | X | | spP03135 |
| 73 | * | EEGAKTAPGKKRPVE | | X | | Q8JQF8 |
| 74 | * | EEGAKTAPTGKRIDD | | X | | Q9YIJ1 |
| 75 | * | EEIKTTNPVATESYG | | X | | Q6JC40 |
| 76 | * | EFENVPFHSSYAHSQ | | X | | Q6JC40 |
| 77 | * | EGAKTAPGKKRPVEP | | X | | Q8JQF8 |
| 78 | * | EGLREFLGLEAGPPK | | X | | Q9YIJ1 |
| 79 | * | EIQYTSNYYKSTNVD | | X | | AAO88201.1 |
| 80 | * | EKTNVDIEKVMITDE | | X | | spP03135 |
| 81 | * | ELQKENSKRWNPEIQ | | X | | spP03135 |
| 82 | * | ENSKRWNPEIQYTNN | | X | | Q9YIJ1 |
| 83 | * | ENVPFHSSYAHSQSL | | X | | Q6JC40 |
| 84 | * | EPDSSSGTGKAGQQP | | X | | spP03135 |
| 85 | * | EQLEAGDNPYLKYNH | | X | | Q9YIJ1 |
| 86 | * | EWELQKENSKRWNPE | | X | | spP03135 |
| 87 | * | GAKTAPGKKRPVEPS | | X | | Q8JQF8 |
| 88 | * | GEPPAGPSGLGSGTM | | X | | AAO88201.1 |
| 89 | * | GGTANTQTLGFSQGG | | X | | Q8JQF8 |
| 90 | * | GSEKTNVDIEKVMIT | | X | | spP03135 |
| 91 | 2078 | GVVADNLQQQNAAPI | | X | | AAO88201.1 |
| 92 | * | GVYSEPRPIGTRYLT | | X | | spP03135 |
| 93 | 2079 | HSSFAPSQNLFKLAN | | X | | Q9YIJ1 |
| 94 | * | HSSYAHSQSLDRLMN | | X | | spP03135 |
| 95 | * | IKNTPVPADPPTAFN | | X | | Q6JC40 |
| 96 | * | IKTTNPVATESYGQV | | X | | Q6JC40 |
| 97 | * | ILIKNTPVPADPPTA | | X | | Q6JC40 |
| 98 | * | IQYTSNYYKSNNVEF | | X | | Q6JC40 |
| 99 | * | IQYTSNYYKSTNVDF | | X | | AAO88201.1 |
| 100 | * | ITNEEEIKTTNPVAT | | X | | Q6JC40 |
| 101 | * | ITQYSTGQVSVEIEW | | X | | spP03135 |
| 102 | 2080 | KDKLNSFITQYSTGQ | | X | | Q6JC40 |
| 103 | * | KNTPVPADPPTAFNK | | X | | Q6JC40 |
| 104 | * | KRWNPEIQYTSNYNK | | X | | spP03135 |
| 105 | 2081 | KRWNPEIQYTSNYYK | | X | | Q6JC40 |
| 106 | 2082 | LEAGDNPYLKYNHAD | | X | | Q9YIJ1 |
| 107 | * | LEAGPPKPKPNQQHQ | | X | | Q9YIJ1 |
| 108 | * | LGADTMSAGGGGPLG | | X | | Q9YIJ1 |
| 109 | * | LGLEAGPPKPKPNQQ | | X | | Q9YIJ1 |
| 110 | * | LIKNTPVPADPPTAF | | X | | Q6JC40 |
| 111 | 2083 | LKYNHADAEFQEKLA | | X | | Q9YIJ1 |
| 112 | * | LQKENSKRWNPEIQY | | X | | spP03135 |
| 113 | * | LREFLGLEAGPPKPK | | X | | Q9YIJ1 |
| 114 | * | LYYLSRTNTPSGTTT | | X | | spP03135 |
| 115 | * | NEEEIKTTNPVATES | | X | | Q6JC40 |
| 116 | * | NKDKLNSFITQYSTG | | X | | Q6JC40 |
| 117 | * | NNSNFAWTAGTKYHL | | X | | Q8JQF8 |
| 118 | * | NPVATEQYGVVADNL | | X | | AAO88201.1 |
| 119 | * | NPVATESYGQVATNH | | X | | Q6JC40 |
| 120 | 2084 | NSQGALPGMVWQNRD | | X | | Q8JQF8 |
| 121 | * | NTPVPADPPTAFNKD | | X | | Q6JC40 |
| 122 | * | NTPVPADPPTTFNQS | | X | | Q8JQF8 |
| 123 | 2085 | PADPPTTFNQSKLNS | | X | | Q8JQF8 |
| 124 | * | PFHSSFAPSQNLFKL | | X | | Q9YIJ1 |
| 125 | * | PIGEPPAGPSGLGSG | | X | | AAO88201.1 |
| 126 | 2086 | PPAGPSGLGSGTMAA | | X | | AAO88201.1 |
| 127 | * | PQILIKNTPVPADPP | | X | | Q6JC40 |
| 128 | * | PQYGYATLNRDNTEN | | X | | Q9YIJ1 |
| 129 | * | PSTSSDAEAGPSGSQ | | X | | Q9YIJ1 |
| 130 | * | PVATEQYGSVSTNLQ | | X | | spP03135 |
| 131 | * | PVEPDSSSGTGKAGQ | | X | | spP03135 |
| 132 | * | PVPADPPTTFNQSKL | | X | | Q8JQF8 |
| 133 | * | PVPGNITSFSDVPVS | | X | | Q9YIJ1 |
| 134 | 2087 | PYLKYNHADAEFQEK | | X | | Q9YIJ1 |
| 135 | * | QILIKNTPVPADPPT | | X | | Q6JC40 |
| 136 | * | QRVSTTTGQNNNSNF | | X | | Q8JQF8 |
| 137 | 2088 | QSGASNDNHYFGYST | | X | | spP03135 |
| 138 | 2089 | QSTGGTAGTQQLLFS | | X | | AAO88201.1 |
| 139 | * | QVTVEMEWELKKENS | | X | | Q9YIJ1 |
| 140 | 2090 | QYGVVADNLQQQNAA | | X | | AAO88201.1 |
| 141 | * | QYTSNYYKSNNVEFA | | X | | Q6JC40 |
| 142 | * | RQQRVSTTTGQNNNS | | X | | Q8JQF8 |
| 143 | 2091 | RWNPEIQYTSNYYKS | | X | | Q6JC40 |
| 144 | 2092 | SFAPSQNLFKLANPL | | X | | Q9YIJ1 |
| 145 | * | SGNWHCDSTWLGDRV | | X | | Q8JQF8 |
| 146 | 2093 | SKRWNPEIQYTSNYY | | X | | Q6JC40 |
| 147 | * | SNYNKSVNVDFTVDT | | X | | spP03135 |
| 148 | 2094 | SQSGASNDNHYFGYS | | X | | spP03135 |
| 149 | 2095 | SRTNTPSGTTTQSRL | | X | | spP03135 |
| 150 | * | SSGNWHCDSTWLGDR | | X | | Q8JQF8 |
| 151 | * | SSGTGKAGQQPARKR | | X | | spP03135 |
| 152 | * | SSLGADTMSAGGGGP | | X | | Q9YIJ1 |
| 153 | * | SSQSGASNDNHYFGY | | X | | spP03135 |
| 154 | * | SSSGNWHCDSTWLGD | | X | | Q8JQF8 |
| 155 | * | STGGTAGTQQLLFSQ | | X | | AAO88201.1 |
| 156 | * | STTLSQNNNSNFAWT | | X | | AAO88201.1 |
| 157 | 2096 | SYGQVATNHQSAQAQ | | X | | Q6JC40 |
| 158 | 2097 | TANTQTLGFSQGGPN | | X | | Q8JQF8 |
| 159 | * | TEGVYSEPRPIGTRY | | X | | Q6JC40 |
| 160 | 2098 | TEQYGVVADNLQQQN | | X | | AAO88201.1 |
| 161 | * | TESYGQVATNHQSAQ | | X | | Q6JC40 |
| 162 | * | TGGTAGTQQLLFSQA | | X | | AAO88201.1 |
| 163 | 2099 | TGQNNNSNFAWTAGT | | X | | Q8JQF8 |
| 164 | * | TLNRDNTENPTERSS | | X | | Q9YIJ1 |
| 165 | * | TLSQNNNSNFAWTGA | | X | | AAO88201.1 |
| 166 | * | TNGVYSEPRPIGTRY | | X | | spP03135 |
| 167 | * | TNTPSGTTTQSRLQF | | X | | spP03135 |
| 168 | * | TNVDIEKVMITDEEE | | X | | spP03135 |
| 169 | * | TQSTGGTAGTQQLLF | | X | | AAO88201.1 |
| 170 | * | TQYSTGQVSVEIEWE | | X | | spP03135 |
| 171 | * | TQYSTGQVTVEMEWE | | X | | Q9YIJ1 |
| 172 | * | TSNYYKSNNVEFAVN | | X | | Q6JC40 |
| 173 | * | TSSDAEAGPSGSQQL | | X | | Q9YIJ1 |
| 174 | * | TTGGTANTQTLGFSQ | | X | | Q8JQF8 |
| 175 | * | TTLSQNNNSNFAWTG | | X | | AAO88201.1 |
| 176 | 2100 | TTNPVATESYGQVAT | | X | | Q6JC40 |
| 177 | * | TTTGQNNNSNFAWTA | | X | | Q8JQF8 |
| 178 | * | TVEMEWELKKENSKR | | X | | Q9YIJ1 |
| 179 | * | VATEQYGVVADNLQQ | | X | | AAO88201.1 |
| 180 | * | VATESYGQVATNHQS | | X | | Q6JC40 |
| 181 | * | VATNHQSAQAQAQTG | | X | | Q6JC40 |
| 182 | * | VDIEKVMITDEEEIR | | X | | spP03135 |
| 183 | * | VDTNGVYSEPRPIGT | | X | | spP03135 |
| 184 | * | VEIEWELQKENSKRW | | X | | spP03135 |
| 185 | * | VNTEGVYSEPRPIGT | | X | | Q6JC40 |
| 186 | * | VQDSTTTIANNLTST | | X | | Q9YIJ1 |
| 187 | * | VSTTLSQNNNSNFAW | | X | | AAO88201.1 |
| 188 | * | VSTTTGQNNNSNFAW | | X | | Q8JQF8 |
| 189 | * | VTVQDSTTTIANNLT | | X | | Q9YIJ1 |
| 190 | 2101 | WTGATKYHLNGRDSL | | X | | spP03135 |
| 191 | 2102 | YAHSQSLDRLMNPLI | | X | | spP03135 |
| 192 | * | YATLNRDNTENPTER | | X | | Q9YIJ1 |
| 193 | * | YGYATLNRDNTENPT | | X | | Q9YIJ1 |
| 194 | * | YLSRTNTPSGTTTQS | | X | | spP03135 |
| 195 | * | YNKSVNVDFTVDTNG | | X | | spP03135 |
| 196 | * | YNNHLYKQISNGTSG | | X | | Q8JQF8 |
| 197 | * | YSTGQVTVEMEWELK | | X | | Q9YIJ1 |
| 198 | 2103 | YTSNYYKSNNVEFAV | | X | | Q6JC40 |
| 199 | * | ASHKDDEEKFFPQSG | | | X | spP03135 |
| 200 | * | ASNDNHYFGYSTPWG | | | X | spP03135 |
| 201 | * | ATERFGTVAVNLQSS | | | X | O56137 |
| 202 | * | AVNLQSSSTDPATGD | | | X | O56137 |
| 203 | * | DAAALEHDKAYDQQL | | | X | Q6JC40 |
| 204 | * | DAEFQERLQEDTSFG | | | X | Q8JQF8 |
| 205 | * | DDEEKFFPQSGVLIF | | | X | spP03135 |
| 206 | * | EDSKPSTSSDAEAGP | | | X | Q9YIJ1 |
| 207 | * | EDVPFHSSYAHSQSL | | | X | spP03135 |
| 208 | * | EEEIKATNPVATERF | | | X | Q9WBP8 |
| 209 | * | EEVGEGLREFLGLEA | | | X | Q9YIJ1 |
| 210 | * | EEYGIVADNLQQQNT | | | X | Q8JQF8 |
| 211 | * | EFLGLEAGPPKPKPN | | | X | Q9YIJ1 |
| 212 | * | EHDKAYDQQLKAGDN | | | X | Q6JC40 |
| 213 | * | EHDKAYDRQLDSGDN | | | X | spP03135 |
| 214 | * | EIKATNPVATERFGT | | | X | Q9WBP8 |
| 215 | * | EPDSSAGIGKSGAQP | | | X | Q6JC40 |
| 216 | * | EPVNAADAAALEHDK | | | X | Q6JC40 |
| 217 | * | EPVNEADAAALEHDK | | | X | spP03135 |
| 218 | * | ERHKDDSRGLVLPGY | | | X | spP03135 |
| 219 | * | ESVPDPQPIGEPPAA | | | X | Q6JC40 |
| 220 | * | EVPFHSSFAPSQNLF | | | X | Q9YIJ1 |
| 221 | * | FHSSYAHSQSLDRLM | | | X | spP03135 |
| 222 | * | FNGLDKGEPVNAADA | | | X | Q8JQF8 |
| 223 | * | FNGLDKGEPVNEADA | | | X | spP03135 |
| 224 | * | GEPVNAADAAALEHD | | | X | Q6JC40 |
| 225 | * | GEPVNEADAAALEHD | | | X | spP03135 |
| 226 | * | GNGLDKGEPVNAADA | | | X | Q6JC40 |
| 227 | * | GSAHQGCLPPFPADV | | | X | spP03135 |
| 228 | * | GSSSGNWHCDSTWLG | | | X | Q8JQF8 |
| 229 | * | IGTVNSQGALPGMVW | | | X | Q8JQF8 |
| 230 | * | IQVKEVTTNDGVTTI | | | X | Q9WBP8 |
| 231 | * | LIKNTPVPADPPTTF | | | X | Q8JQF8 |
| 232 | * | LRTGNNFEFSYQFED | | | X | AAO88201.1 |
| 233 | * | NEADAAALEHDKAYD | | | X | spP03135 |
| 234 | * | NNNSEFAWPGASSWA | | | X | Q6JC40 |
| 235 | * | NNSEYSWTGATKYHL | | | X | spP03135 |
| 236 | * | NVGGQMATNNQSSTT | | | X | Q9YIJ1 |
| 237 | * | NYNDPQFVDFAPDST | | | X | Q9YIJ1 |
| 238 | * | PLGEPPATPAAVGPT | | | X | Q9WBP8 |
| 239 | * | PQPLGEPPATPAAVG | | | X | Q9WBP8 |
| 240 | * | PSKMLRTGNNFEFTY | | | X | Q9YIJ1 |
| 241 | * | PVATEEYGIVADNLQ | | | X | Q8JQF8 |
| 242 | * | PVEPSPQRSPDSSTG | | | X | Q8JQF8 |
| 243 | * | PVEQSPQEPDSSSGI | | | X | Q9WBP8 |
| 244 | * | PVNEADAAALEHDKA | | | X | spP03135 |
| 245 | * | PVPANPPAEFSATKF | | | X | Q9WBP8 |
| 246 | * | QQRVSTTLSQNNNSN | | | X | AAO88201.1 |
| 247 | * | QRVSKTKTDNNNSNF | | | X | Q9WBP8 |
| 248 | * | QRVSTTLSQNNNSNF | | | X | AAO88201.1 |
| 249 | * | QSSSTDPATGDVHVM | | | X | O56137 |
| 250 | * | QYTNNYNDPQFVDFA | | | X | Q9YIJ1 |
| 251 | * | RVSTTLSQNNNSNFA | | | X | AAO88201.1 |
| 252 | * | SDSEYQLPYVLGSAH | | | X | Q9WBP8 |
| 253 | * | SESVPDPQPIGEPPA | | | X | AAO88201.1 |
| 254 | * | SESVPDPQPLGEPPA | | | X | Q8JQF8 |
| 255 | * | SFVDHPPDWLEEVGE | | | X | Q9YIJ1 |
| 256 | * | SSNDNAYFGYSTPWG | | | X | Q6JC40 |
| 257 | * | SSSGIGKTGQQPAKK | | | X | Q9WBP8 |
| 258 | * | STTVTQNNNSEFAWP | | | X | Q6JC40 |
| 259 | * | SVPDPQPLGEPPAAP | | | X | Q8JQF8 |
| 260 | * | SVPDPQPLGEPPATP | | | X | Q9WBP8 |
| 261 | * | SYEFENVPFHSSYAH | | | X | Q6JC40 |
| 262 | * | SYTFEDVPFHSSYAH | | | X | spP03135 |
| 263 | * | TDEEEIKATNPVATE | | | X | Q9WBP8 |
| 264 | * | TDEEEIRTTNPVATE | | | X | spP03135 |
| 265 | * | TGNNFEFSYQFEDVP | | | X | AAO88201.1 |
| 266 | * | TMATGSGAPMADNNE | | | X | spP03135 |
| 267 | * | TNNYNDPQFVDFAPD | | | X | Q9YIJ1 |
| 268 | * | TNTMATGSGAPMADN | | | X | spP03135 |
| 269 | * | TPVPADPPTAFNKDK | | | X | Q6JC40 |
| 270 | * | TPVPADPPTTFSQAK | | | X | AAO88201.1 |
| 271 | * | TSTVQVFTDSEYQLP | | | X | spP03135 |
| 272 | * | TSVDFAVNTEGVYSE | | | X | Q8JQF8 |
| 273 | * | TVAVNLQSSSTDPAT | | | X | O56137 |
| 274 | * | VDFAVNTEGVYSEPR | | | X | Q8JQF8 |
| 275 | * | VEFAVNTEGVYSEPR | | | X | Q6JC40 |
| 276 | * | VLEPLGLVEEGAKTA | | | X | Q8JQF8 |
| 277 | * | VNVDFTVDTNGVYSE | | | X | spP03135 |
| 278 | * | VSVEIEWELQKENSK | | | X | spP03135 |
| 279 | * | WLEDNLSEGIREWWD | | | X | Q9WBP8 |
| 280 | * | YNEQLEAGDNPYLKY | | | X | Q9YIJ1 |

### Example 14: Further Screen for Anti-Vector Antibodies in Human Sera

Out of the 3285 cyclic peptides derived from Ad5 Hexon, fiber and penton proteins P04133, P11818 and P12538, respectively, and AAV VP1 sequences P03135, Q6JC40, Q8JQF8, Q9WBP8, Q9YIJ1, 056137, AA088201.1, 041855, 056139 and Q8JQG0, the peptides with the top 5% maximal IgG signal strength over the microarray screens were obtained, yielding a total of 164 peptides with top signals from the vector protein sequences screened. The details are shown in Table 4 below.

TABLE 4
This table provides another compilation of viral peptide
sequences suitable as a basis for the present invention.
Specifically, these are suited to be used for SADCs according to
the present invention. Source given is either UniProt ID or GenBank
ID. The asterisk (*) indicates peptide sequences for which a SEQ ID
NO has already been assigned in Table 2 above.

| peptide | SEQ | | |
|---|---|---|---|
| # | ID NO | peptide | source |
| 1 | 2104 | AAEAAAPAAQPEVE | P12538 |
| 2 | 2105 | AAAPAAQPEVEKPQ | P12538 |
| 3 | 2106 | DASEYLSPGLVQFA | P04133 |
| 4 | 2107 | DGLEFGSPNAPNTN | P11818 |
| 5 | 2108 | APVAAALGSPFDAP | P12538 |
| 6 | 2109 | NLEEEDDDNEDEVD | P04133 |
| 7 | 2110 | EEDDDNEDEVDEQA | P04133 |
| 8 | 2111 | DDNEDEVDEQAEQQ | P04133 |
| 9 | 2112 | VLQSSLGNDLRVDG | P04133 |
| 10 | 2113 | PSEDTFNPVYPYDT | P11818 |
| 11 | 2114 | FPVVGAELLPVHSK | P12538 |
| 12 | 2115 | EEIRPTNPVATEEY | Q8JQG0 |
| 13 | 2116 | SGSGAEENSNAAAA | P12538 |
| 14 | 2117 | YEEGPPPSYESVVS | P12538 |
| 15 | 2118 | NAAAAAMQPVEDMN | P12538 |
| 16 | 2119 | AAALGSPFDAPLDP | P12538 |
| 17 | 2120 | NGVLESDIGVKFDT | P12538 |
| 18 | 2121 | AAAMQPVEDMNDHA | P12538 |
| 19 | 2122 | PAQPASSLGADTMS | Q9YIJ1 |
| 20 | 2123 | TVQVFTDDDYQLPY | Q9YIJ1 |
| 21 | 2124 | PVPGNITSFSDVPV | Q9YIJ1 |
| 22 | 2125 | TQYSTGQVTVEMEW | Q9YIJ1 |
| 23 | 2126 | YLGPGNGLDRGEPV | Q9YIJ1 |
| 24 | 2127 | TSESETQPVNRVAY | Q9YIJ1 |
| 25 | 2128 | RARPSEDTFNPVYP | P11818 |
| 26 | 2129 | ATALEINLEEEDDD | P04133 |
| 27 | 2130 | ENSNAAAAAMQPVE | P12538 |
| 28 | 2131 | YLGPFNGLDKGEPV | P03135 |
| 29 | 2132 | NNFEFSYSFEDVPF | Q8JQG0 |
| 30 | 2133 | SFITQYSTGQVTVE | Q9YIJ1 |
| 31 | 2134 | YNKSVNVDFTVDTN | P03135 |
| 32 | 2135 | LGSPFDAPLDPPFV | P12538 |
| 33 | 2136 | PAAQPEVEKPQKKP | P12538 |
| 34 | 2137 | ENSKRWNPEIQYTN | Q9YIJ1 |
| 35 | 2138 | YLVDNKSTDVASLN | P12538 |
| 36 | 2139 | PPMMLIKNTPVPGN | Q9YIJ1 |
| 37 | 2140 | PVPADPPTTFSQAK | AAO88201.1 |
| 38 | 2141 | DTFNPVYPYDTETG | P11818 |
| 39 | 2142 | ITQYSTGQVTVEME | Q9YIJ1 |
| 40 | 2143 | AGNLTSQNVTTVSP | P11818 |
| 41 | 2144 | NNFTFSYTFEDVPF | P03135 |
| 42 | 2145 | WVLPSYNNHQYREI | Q9YIJ1 |
| 43 | 2146 | YLGPGNGLDKGEPV | Q6JC40 |
| 44 | 2147 | FLYSNIALYLPDKL | P04133 |
| 45 | 2148 | NSTGNMGVLAGQAS | P04133 |
| 46 | 2149 | YLKYNHADAEFQER | P03135 |
| 47 | 2150 | LAPKGAPNPCEWDE | P04133 |
| 48 | 2151 | IQYTNNYNDPQFVD | Q9YIJ1 |
| 49 | 2152 | SHGKTAKSNIVSQV | P11818 |
| 50 | 2153 | VSAAPVAAALGSPF | P12538 |
| 51 | 2154 | GNDRLLTPNEFEIK | P04133 |
| 52 | 2155 | LEINLEEEDDDNED | P04133 |
| 53 | 2156 | YLRYNHADAEFQER | Q8JQF8 |
| 54 | 2157 | AAGGAAVEGGQGAD | O41855 |
| 55 | 2158 | VKEVTTSNGETTVA | O41855 |
| 56 | 2159 | EAMLRNDTNDQSFN | P04133 |
| 57 | 2160 | DGHFHPSPLIGGFG | O41855 |
| 58 | 2161 | NMTKDWFLVQMLAN | P04133 |
| 59 | 2162 | VGSGTVAAGGGAPM | Q8JQG0 |
| 60 | 2163 | EQYGTVANNLQSSN | O56139 |
| 61 | 2164 | CLEYFPSKMLRTGN | Q9YIJ1 |
| 62 | 2165 | AHALDMTFEVDPMD | P04133 |
| 63 | 2166 | TMANQAKNWLPGPC | Q8JQF8 |
| 64 | 2167 | KRPVEQSPQEPDSS | Q6JC40 |
| 65 | 2168 | KTANDNNNSNFPWT | O56139 |
| 66 | 2169 | TQNNNSEFAWPGAS | Q6JC40 |
| 67 | 2170 | KNTPVPADPPTAFN | Q6JC40 |
| 68 | 2171 | TVQVFSDSEYQLPY | Q9WBP8 |
| 69 | 2172 | LDKGEPVNAADAAA | Q6JC40 |
| 70 | 2173 | VLEPLGLVEEPVKT | P03135 |
| 71 | 2174 | KLFNIQVKEVTTND | Q9WBP8 |
| 72 | 2175 | LQSSNTAPTTRTVN | 056139 |
| 73 | 2176 | SPPLKKTKSNINLE | P11818 |
| 74 | 2177 | LDKGEPVNEADAAA | P03135 |
| 75 | 2178 | MLRTGNNFQFSYEF | Q6JC40 |
| 76 | 2179 | ARKRLNFGQTGDAD | P03135 |
| 77 | 2180 | NTYNGFSTPWGYFD | O41855 |
| 78 | 2181 | QYGTVANNLQSSNT | O56139 |
| 79 | 2182 | LARPPAPTITTVSE | P12538 |
| 80 | 2183 | FPADVFMIPQYGYL | Q6JC40 |
| 81 | 2184 | STGQVSVEIEWELQ | P03135 |
| 82 | 2185 | LRNDTNDQSFNDYL | P04133 |
| 83 | 2186 | NFQFSYEFENVPFH | Q6JC40 |
| 84 | 2187 | FITQYSTGQVTVEM | Q9YIJ1 |
| 85 | 2188 | QQPARKRLNFGQTG | P03135 |
| 86 | 2189 | HDSKLSIATQGPLT | P11818 |
| 87 | 2190 | NTYFGYSTPWGYFD | Q8JQF8 |
| 88 | * | ANNLTSTVQIFADS | O41855 |
| 89 | * | GNTSQQQTDRNAFY | O41855 |
| 90 | * | DTNGVYSEPRPIGT | P03135 |
| 91 | * | KIFNIQVKEVTTSN | O41855 |
| 92 | * | LIKNTPVPADPPTT | Q8JQF8 |
| 93 | * | DEEEIRTTNPVATE | P03135 |
| 94 | * | DKAYDRQLDSGDNP | P03135 |
| 95 | * | DKDKFFPMSGVMIF | O56137 |
| 96 | * | LQQQNTAPQIGTVN | Q8JQF8 |
| 97 | * | GTNTMATGSGAPMA | P03135 |
| 98 | * | IFNIQVKEVTTSNG | O41855 |
| 99 | * | DKAYDQQLQAGDNP | Q8JQF8 |
| 100 | * | DSQWLGDRVITTST | Q6JC40 |
| 101 | * | DEEEIKATNPVATE | Q9WBP8 |
| 102 | * | SFITQYSTGQVSVE | P03135 |
| 103 | * | PLVDQYLYRFVSTN | Q9YIJ1 |
| 104 | * | GKKRPVDQSPQEPD | O56139 |
| 105 | * | NPVATEQYGSVSTN | P03135 |
| 106 | * | DKAYDQQLKAGDNP | Q6JC40 |
| 107 | * | TAPGKKRPVDQSPQ | O56139 |
| 108 | * | VMITDEEEIRTTNP | P03135 |
| 109 | * | FGKQGSEKTNVDIE | P03135 |
| 110 | * | LQRGNRQAATADVN | P03135 |
| 111 | * | KTAPGKKRPVDQSP | O56139 |
| 112 | * | SESVPDPQPIGEPP | AAO88201.1 |
| 113 | * | LEDNLSEGIREWWD | Q9WBP8 |
| 114 | * | PEIQYTSNYNKSVN | P03135 |
| 115 | * | DKFFPMSGVMIFGK | Q9WBP8 |
| 116 | * | APGKKRPVDQSPQE | O56139 |
| 117 | * | NNFQFSYTFEDVPF | O56139 |
| 118 | * | EFAWPGASSWALNG | Q6JC40 |
| 119 | * | QSSNTAPTTRTVND | O56139 |
| 120 | * | PVPADPPTTFNQSK | Q8JQF8 |
| 121 | * | TQYSTGQVSVEIEW | P03135 |
| 122 | * | SKTANDNNNSNFPW | O56139 |
| 123 | * | TVQIFADSSYELPY | O41855 |
| 124 | * | SESVPDPQPLGEPP | Q8JQF8 |
| 125 | * | TESVPDPQPIGEPP | Q6JC40 |
| 126 | * | KNTPVPADPPTTFS | AAO88201.1 |
| 127 | * | PVPADPPTAFNKDK | Q6JC40 |
| 128 | * | KKRPVDQSPQEPDS | O56139 |
| 129 | * | KNTPVPADPPTTFN | Q8JQF8 |
| 130 | * | PRDWQRLINNNWGF | P03135 |
| 131 | * | LFNIQVKEVTTNDG | Q9WBP8 |
| 132 | * | TTSGTTNQSRLLFS | O56139 |
| 133 | * | LSKTANDNNNSNFP | O56139 |
| 134 | * | ENSKRWNPEIQYTS | P03135 |
| 135 | * | DIYYQGPIWAKIPH | 041855 |
| 136 | * | DDEDKFFPMSGVMI | Q9WBP8 |
| 137 | * | THSTLDGRWSALTP | O41855 |
| 138 | * | GADGVGNSSGNWHC | P03135 |
| 139 | * | TIANNLTSTIQVFT | Q8JQF8 |
| 140 | * | PQILIKNTPVPADP | Q6JC40 |
| 141 | * | QLKAGDNPYLRYNH | Q9WBP8 |
| 142 | * | NYNKSVNVDFTVDT | P03135 |
| 143 | * | EEEIKTTNPVATEE | Q8JQF8 |
| 144 | * | KGEPVNEADAAALE | P03135 |
| 145 | * | KGEPVNAADAAALE | Q6JC40 |
| 146 | * | DGHFHPSPLMGGFG | P03135 |
| 147 | * | HYFGYSTPWGYFDF | P03135 |
| 148 | * | EEIKTTNPVATEQY | AAO88201.1 |
| 149 | * | FNIQVKEVTTNDGV | Q9WBP8 |
| 150 | * | PWGYFDFNRFHCHF | P03135 |
| 151 | * | LQQQNAAPIVGAVN | AAO88201.1 |
| 152 | * | DWLEDNLSEGIREW | Q6JC40 |
| 153 | * | WLEDNLSEGIREWW | Q6JC40 |
| 154 | * | DSESVPDPQPIGEP | AAO88201.1 |
| 155 | * | KRPVDQSPQEPDSS | O56139 |
| 156 | * | HSQSLDRLMNPLID | P03135 |
| 157 | * | FEKVPFHSMYAHSQ | O41855 |
| 158 | * | YDQQLKAGDNPYLK | Q6JC40 |
| 159 | * | EDNLSEGIREWWDL | Q9WBP8 |
| 160 | * | QVKEVTTNDGVTTI | Q9WBP8 |
| 161 | * | PQYGYLTLNNGSQA | P03135 |
| 162 | * | EEEIKTTNPVATES | Q6JC40 |
| 163 | * | EGADGVGNASGNWH | Q9WBP8 |
| 164 | * | DSESVPDPQPLGEP | Q8JQF8 |

Thus, all listed peptides, preferably peptides belonging to the Top 10% Hits, provide sequences from which shorter peptide sequences can be derived for (transient) antibody depletion with the compound according to the present invention. In addition, these peptides and fragments thereof can also be used as probes for the diagnostic detection of anti-AAV or anti-AdV antibodies in biological samples such as human sera.

### Example 15: Identification of Epitopes for the Transient Depletion of Anti-Measles Virus Antibodies

Three structurally especially exposed regions of Measles Virus hemagglutinin glycoprotein H were selected, spanning amino-acid residues 290-335, 365-416 and 571-610 (numbering based on UniProt sequence entry P08362). Based on this selection, 140 cyclic 16mer peptides were synthesized starting from each single amino-acid position out of these three regions.

Samples obtained from human donors were screened for antibodies against these measles virus-derived peptides immobilized on microarrays. To this end, IgG was prepared from blood obtained from the human donors by protein G purification. Each IgG sample was incubated with the peptide microarrays and Ig binding signals were detected by fluorescence

The Top 25% IgG binding hits derived from the plasma samples screened, are listed in Table 5 below. The peptides belonging to the Top 10% hits and the corresponding starting amino-acid positions within UniProt sequence entry P08362 are specifically indicated.

TABLE 5

| peptide | SEQ | starting pos. | | |
|---|---|---|---|---|
| # | ID NO | in P08362 | peptide | top 10% |
| 1 | 2191 | 290 | ALGELKLAALCHGEDS | |
| 2 | 2192 | 299 | LCHGEDSITIPYQGSG | |
| 3 | 2193 | 302 | GEDSITIPYQGSGKGV | X |
| 4 | 2194 | 303 | EDSITIPYQGSGKGVS | X |
| 5 | 2195 | 304 | DSITIPYQGSGKGVSF | X |
| 6 | 2196 | 307 | TIPYQGSGKGVSFQLV | |
| 7 | 2197 | 308 | IPYQGSGKGVSFQLVK | |
| 8 | 2198 | 309 | PYQGSGKGVSFQLVKL | X |
| 9 | 2199 | 310 | YQGSGKGVSFQLVKLG | |
| 10 | 2200 | 311 | QGSGKGVSFQLVKLGV | |
| 11 | 2201 | 313 | SGKGVSFQLVKLGVWK | X |
| 12 | 2202 | 314 | GKGVSFQLVKLGVWKS | X |
| 13 | 2203 | 366 | AVPTTRTDDKLRMETC | |
| 14 | 2204 | 367 | VPTTRTDDKLRMETCF | X |
| 15 | 2205 | 368 | PTTRTDDKLRMETCFQ | |
| 16 | 2206 | 373 | DDKLRMETCFQQACKG | |
| 17 | 2207 | 374 | DKLRMETCFQQACKGK | X |
| 18 | 2208 | 375 | KLRMETCFQQACKGKI | X |
| 19 | 2209 | 376 | LRMETCFQQACKGKIQ | |
| 20 | 2210 | 388 | GKIQALCENPEWAPLK | |
| 21 | 2211 | 390 | IQALCENPEWAPLKDN | |
| 22 | 2212 | 391 | QALCENPEWAPLKDNR | |
| 23 | 2213 | 392 | ALCENPEWAPLKDNRI | |
| 24 | 2214 | 393 | LCENPEWAPLKDNRIP | X |
| 25 | 2215 | 394 | CENPEWAPLKDNRIPS | |
| 26 | 2216 | 396 | NPEWAPLKDNRIPSYG | |
| 27 | 2217 | 397 | PEWAPLKDNRIPSYGV | |
| 28 | 2218 | 398 | EWAPLKDNRIPSYGVL | X |
| 29 | 2219 | 401 | PLKDNRIPSYGVLSVD | X |
| 30 | 2220 | 574 | DQKLWCRHFCVLADSE | X |
| 31 | 2221 | 581 | HFCVLADSESGGHITH | |
| 32 | 2222 | 585 | LADSESGGHITHSGME | |
| 33 | 2223 | 587 | DSESGGHITHSGMEGM | X |
| 34 | 2224 | 588 | SESGGHITHSGMEGMG | X |
| 35 | 2225 | 589 | ESGGHITHSGMEGMGV | |

Thus, all listed peptides, preferably peptides belonging to the Top 10% hits, provide sequences from which shorter peptide sequences can be derived for (transient) antibody depletion with the compound according to the present invention. In addition, these peptides and fragments thereof can also be used as probes for the diagnostic detection of anti-measles virus antibodies in biological samples such as human sera.

### Non-Patent References

Apolonio, Jonathan Santos, et al. “Oncolytic virus therapy in cancer: A current review.” World Journal of Virology 10.5 (2021): 229.

Balakrishnan, Balaji, and Ernest David. “Biopolymers augment viral vectors based gene delivery.” Journal of biosciences 44.4 (2019): 1-8.

Bennett, Antonette, et al. “Structure comparison of the chimeric AAV2. 7m8 vector with parental AAV2.” Journal of structural biology 209.2 (2020): 107433.

Bertin, Berangere, et al. “Capsid-specific removal of circulating antibodies to adeno-associated virus vectors.” Scientific reports 10.1 (2020): 1-11.

Bhatt, Darshak K., Roger Chammas, and Toos Daemen. “Resistance mechanisms influencing oncolytic virotherapy, a systematic analysis.” Vaccines 9.10 (2021): 1166.

Börner, Kathleen, et al. “Pre-arrayed pan-AAV peptide display libraries for rapid single-round screening.” Molecular Therapy 28.4 (2020): 1016-1032.

Bruno, John G. “Potential inherent stimulation of the innate immune system by nucleic acid aptamers and possible corrective approaches.” Pharmaceuticals 11.3 (2018): 62.

Carter, John Mark, and Larry Loomis-Price. “B cell epitope mapping using synthetic peptides.” Current protocols in immunology 60.1 (2004): 9-4.

Cearley, Cassia N., et al. “Expanded repertoire of AAV vector serotypes mediate unique patterns of transduction in mouse brain.” Molecular therapy 16.10 (2008): 1710-1718.

Colella, Pasqualina, Giuseppe Ronzitti, and Federico Mingozzi. “Emerging issues in AAV-mediated in vivo gene therapy.” Molecular Therapy-Methods & Clinical Development 8 (2018): 87-104.

Condit, Richard C., Nissin Moussatche, and Paula Traktman. “In a nutshell: structure and assembly of the vaccinia virion.” Advances in virus research 66 (2006): 31-124.

Cook, Joselle, et al. “Oncolytic virotherapy-Forging its place in the immunomodulatory paradigm for Multiple Myeloma.” Cancer Treatment and Research Communications 29 (2021): 100473.

Conry, Robert M., et al. “Talimogene laherparepvec: First in class oncolytic virotherapy.” Human vaccines & immunotherapeutics 14.4 (2018): 839-846.

Cristi, Francisca, et al. “Genetic modifications that expand oncolytic virus potency.” Frontiers in Molecular Biosciences 9 (2022): 25.

De Matos, Ana Lemos, Lina S. Franco, and Grant McFadden. “Oncolytic viruses and the immune system: the dynamic duo.” Molecular Therapy-Methods & Clinical Development 17 (2020): 349-358.

De Sostoa, Jana, Valérie Dutoit, and Denis Migliorini. “Oncolytic viruses as a platform for the treatment of malignant brain tumors.” International journal of molecular sciences 21.20 (2020): 7449.

Dijkstra, C. D., et al. “The heterogeneity of mononuclear phagocytes in lymphoid organs: distinct macrophage subpopulations in rat recognized by monoclonal antibodies ED1, ED2 and ED3.” Microenvironments in the Lymphoid System. Springer, Boston, MA, 1985. 409-419.

Domenger, Claire, and Dirk Grimm. “Next-generation AAV vectors-do not judge a virus (only) by its cover.” Human molecular genetics 28.R1 (2019): R3-R14.

Elliott, Serra E., et al. “A pre-eclampsia-associated Epstein-Barr virus antibody cross-reacts with placental GPR50.” Clinical Immunology 168 (2016): 64-71.

Engeland, Christine E., and Guy Ungerechts. “Measles virus as an oncolytic immunotherapy.” Cancers 13.3 (2021): 544.

Erlandsson, Ann, et al. “In vivo clearing of idiotypic antibodies with antiidiotypic antibodies and their derivatives.” Molecular immunology 43.6 (2006): 599-606.

Etzerodt, Anders, et al. “Efficient intracellular drug-targeting of macrophages using stealth liposomes directed to the hemoglobin scavenger receptor CD163.” Journal of controlled release 160.1 (2012): 72-80.

Fabriek, Babs O., et al. “The macrophage scavenger receptor CD163 functions as an innate immune sensor for bacteria.” Blood 113.4 (2009): 887-892.

Fausther-Bovendo, Hugues, and Gary P. Kobinger. “Pre-existing immunity against Ad vectors: humoral, cellular, and innate response, what's important?.” Human vaccines & immunotherapeutics 10.10 (2014): 2875-2884.

Feiner, Rebecca C., et al. “EGFR-binding peptides: From computational design towards tumor-targeting of adeno-associated virus capsids.” International journal of molecular sciences 21.24 (2020): 9535.

Foloppe, Johann, et al. “The enhanced tumor specificity of TG6002, an armed oncolytic vaccinia virus deleted in two genes involved in nucleotide metabolism.” Molecular Therapy-Oncolytics 14 (2019): 1-14.

Gao, Yang, et al. “The application of oncolytic viruses in cancer therapy.” Biotechnology Letters 43.10 (2021): 1945-1954.

Garces, Jorge Carlos, et al. “Antibody-mediated rejection: a review.” The Ochsner Journal 17.1 (2017): 46.

Gazarian, Karlen, et al. “Mimotope peptides selected from phage display combinatorial library by serum antibodies of pigs experimentally infected with Taenia solium as leads to developing diagnostic antigens for human neurocysticercosis.” Peptides 38.2 (2012): 381-388.

Gfeller, David, et al. “Current tools for predicting cancer-specific T cell immunity.” Oncoimmunology 5.7 (2016): e1177691.

Granfeldt, Asger, et al. “Targeting dexamethasone to macrophages in a porcine endotoxemic model.” Critical Care Medicine 41.11 (2013): e309-e318.

Graversen, Jonas H., et al. “Targeting the hemoglobin scavenger receptor CD163 in macrophages highly increases the anti-inflammatory potency of dexamethasone.” Molecular Therapy 20.8 (2012): 1550-1558.

Gurda, Brittney L., et al. “Mapping a neutralizing epitope onto the capsid of adeno-associated virus serotype 8.” Journal of virology 86.15 (2012): 7739-7751.

Hansen, Lajla Bruntse, Soren Buus, and Claus Schafer-Nielsen. “Identification and mapping of linear antibody epitopes in human serum albumin using high-density peptide arrays.” PLOS One 8.7 (2013): e68902.

Homma, Masayuki, et al. “A Novel Fusion Protein, AChR-Fc, Ameliorates Myasthenia Gravis by Neutralizing Antiacetylcholine Receptor Antibodies and Suppressing Acetylcholine Receptor-Reactive B Cells.” Neurotherapeutics 14.1 (2017): 191-198.

Howard Jr, James F. “Myasthenia gravis: the role of complement at the neuromuscular junction.” Annals of the New York Academy of Sciences 1412.1 (2018): 113-128.

Howarth, M., & Brune, K. D. (2018). New routes and opportunities for modular construction of particulate vaccines: stick, click and glue. Frontiers in immunology, 9, 1432.

Hromic-Jahjefendic, Altijana, and Kenneth Lundstrom. “Viral vector-based melanoma gene therapy.” Biomedicines 8.3 (2020): 60.

Innao, Vanessa, et al. “Oncolytic viruses and hematological malignancies: a new class of immunotherapy drugs.” Current Oncology 28.1 (2020): 159-183.

Jansson, Liselotte, et al. “Immunotherapy With Apitopes Blocks the Immune Response to TSH Receptor in HLA-DR Transgenic Mice.” Endocrinology 159.9 (2018): 3446-3457.

Jensen, Kamilla Kjærgaard, et al. “Improved methods for predicting peptide binding affinity to MHC class II molecules.” Immunology 154.3 (2018): 394-406.

Jin, Shengye, et al. “Oncolytic viruses for triple negative breast cancer and beyond.” Biomarker Research 9 (2021): 1-16.

Jurtz, Vanessa, et al. “NetMHCpan-4.0: improved peptide-MHC class I interaction predictions integrating eluted ligand and peptide binding affinity data.” The Journal of Immunology 199.9 (2017): 3360-3368.

Kainulainen, Markus H., et al. “High-throughput quantitation of SARS-COV-2 antibodies in a single-dilution homogeneous assay.” Scientific reports 11.1 (2021): 1-9.

Kontermann, Roland E., Guy Ungerechts, and Dirk M. Nettelbeck. “Viro-antibody therapy: engineering oncolytic viruses for genetic delivery of diverse antibody-based biotherapeutics.” MAbs. Vol. 13. No. 1. Taylor & Francis, 2021.

Koşaloğlu-Yalçin, Zeynep, et al. “Predicting T cell recognition of MHC class I restricted neoepitopes.” Oncoimmunology 7.11 (2018): e1492508.

Krasnykh, Victor, et al. “Characterization of an adenovirus vector containing a heterologous peptide epitope in the HI loop of the fiber knob.” Journal of virology 72.3 (1998): 1844-1852.

Kruzik, Anita, et al. “Prevalence of anti-adeno-associated virus immune responses in international cohorts of healthy donors.” Molecular Therapy-Methods & Clinical Development 14 (2019): 126-133.

Kuklik, Juliane, et al. “Development of a Bispecific Antibody-Based Platform for Retargeting of Capsid Modified AAV Vectors.” International journal of molecular sciences 22.15 (2021): 8355.

Lawler, Sean E., et al. “Oncolytic viruses in cancer treatment: a review.” JAMA oncology 3.6 (2017): 841-849.

Lazaridis, Konstantinos, et al. “Specific removal of autoantibodies by extracorporeal immunoadsorption ameliorates experimental autoimmune myasthenia gravis.” Journal of neuroimmunology 312 (2017): 24-30.

Leung, Nicki Y H, et al. “Screening and identification of mimotopes of the major shrimp allergen tropomyosin using one-bead-one-compound peptide libraries.” Cellular & molecular immunology 14.3 (2017): 308-318.

Li, Chengwen, and R. Jude Samulski. “Engineering adeno-associated virus vectors for gene therapy.” Nature Reviews Genetics 21.4 (2020): 255-272.

Li, Peipei, Li Wang, and Li-jun Di. “Applications of protein fragment complementation assays for analyzing biomolecular interactions and biochemical networks in living cells.” Journal of proteome research 18.8 (2019): 2987-2998.

Lim, Sung In, and Inchan Kwon. “Bioconjugation of therapeutic proteins and enzymes using the expanded set of genetically encoded amino acids.” Critical reviews in biotechnology 36.5 (2016): 803-815.

Lin, Chia-Hao, et al. “Identification of a major epitope by anti-interferon-y autoantibodies in patients with mycobacterial disease.” Nature medicine 22.9 (2016): 994.

Liu, Huitao, and Honglin Luo. “Development of group B coxsackievirus as an oncolytic virus: opportunities and challenges.” Viruses 13.6 (2021): 1082.

Lorentz, Kristen M., et al. “Engineered binding to erythrocytes induces immunological tolerance to E. coli asparaginase.” Science advances 1.6 (2015): e1500112.

Lundstrom, Kenneth. “New frontiers in oncolytic viruses: optimizing and selecting for virus strains with improved efficacy.” Biologics: targets & therapy 12 (2018): 43.

Luo, Jie, et al. “Main immunogenic region structure promotes binding of conformation-dependent myasthenia gravis autoantibodies, nicotinic acetylcholine receptor conformation maturation, and agonist sensitivity.” Journal of Neuroscience 29.44 (2009): 13898-13908.

Luo, Jie, and Jon Lindstrom. “AChR-specific immunosuppressive therapy of myasthenia gravis.” Biochemical pharmacology 97.4 (2015): 609-619.

Madsen, Mette, et al. “Molecular Characterization of the Haptoglobin. Hemoglobin Receptor CD163 ligand binding properties of the scavenger receptor cysteine-rich domain region.” Journal of Biological Chemistry 279.49 (2004): 51561-51567.

Majowicz, Anna, et al. “Seroprevalence of pre-existing NABs against AAV1, 2, 5, 6 and 8 in the South African Hemophilia B patient population.” (2019): 3353-3353.

Manno, Catherine S., et al. “Successful transduction of liver in hemophilia by AAV-Factor IX and limitations imposed by the host immune response.” Nature medicine 12.3 (2006): 342-347.

Mazor, Ronit, et al. “Tolerogenic nanoparticles restore the antitumor activity of recombinant immunotoxins by mitigating immunogenicity.” Proceedings of the National Academy of Sciences 115.4 (2018): E733-E742.

Meister, Daniel, S. Maryamdokht Taimoory, and John F. Trant. “Unnatural amino acids improve affinity and modulate immunogenicity: Developing peptides to treat MHC type II autoimmune disorders.” Peptide Science 111.1 (2019): e24058.

Mietzsch, Mario, et al. “OneBac: platform for scalable and high-titer production of adeno-associated virus serotype 1-12 vectors for gene therapy.” Human gene therapy 25.3 (2014): 212-222.

Mimuro, Jun, et al. “Minimizing the inhibitory effect of neutralizing antibody for efficient gene expression in the liver with adeno-associated virus 8 vectors.” Molecular Therapy 21.2 (2013): 318-323.

Mingozzi, Federico, et al. “Overcoming preexisting humoral immunity to AAV using capsid decoys.” Science translational medicine 5.194 (2013): 194ra92-194ra92.

Mingozzi, Federico, and Katherine A. High. “Overcoming the host immune response to adeno-associated virus gene delivery vectors: the race between clearance, tolerance, neutralization, and escape.” Annual review of virology 4 (2017): 511-534.

Mok, Darren Z L, and Kuan Rong Chan. “The effects of pre-existing antibodies on live-attenuated viral vaccines.” Viruses 12.5 (2020): 520.

Monahan, Paul E., et al. “Emerging Immunogenicity and Genotoxicity Considerations of Adeno-Associated Virus Vector Gene Therapy for Hemophilia.” Journal of Clinical Medicine 10.11 (2021): 2471.

Morimoto et. al., Bioconjugate Chemistry 25 (8) (2014): 1479-1491

Moussa, Ehab M., et al. “Immunogenicity of therapeutic protein aggregates.” Journal of pharmaceutical sciences 105.2 (2016): 417-430.

Müller, Manuel M. “Post-translational modifications of protein backbones: unique functions, mechanisms, and challenges.” Biochemistry 57.2 (2017): 177-185.

Müller, Louise, et al. “Past, present and future of oncolytic reovirus.” Cancers 12.11 (2020): 3219.

Munis, Altar M., Emma M. Bentley, and Yasuhiro Takeuchi. “A tool with many applications: vesicular stomatitis virus in research and medicine.” Expert Opinion on Biological Therapy 20.10 (2020): 1187-1201.

Siang Ong, Yong, et al. “Recent advances in synthesis and identification of cyclic peptides for bioapplications.” Current topics in medicinal chemistry 17.20 (2017): 2302-2318.

Peters, Bjoern, et al. “A community resource benchmarking predictions of peptide binding to MHC-I molecules.” PLOS computational biology 2.6 (2006): e65.

Pishesha, Novalia, et al. “Engineered erythrocytes covalently linked to antigenic peptides can protect against autoimmune disease.” Proceedings of the National Academy of Sciences (2017): 201701746.

Rahman, Masmudur M., and Grant McFadden. “Oncolytic viruses: newest frontier for cancer immunotherapy.” Cancers 13.21 (2021): 5452.

Resch, Wolfgang, et al. “Protein composition of the vaccinia virus mature virion.” Virology 358.1 (2007): 233-247.

Rey et al., Clinical Immunology 96 (3) (2000): 269-279

Rice, Peter, Ian Longden, and Alan Bleasby. “EMBOSS: the European molecular biology open software suite.” Trends in genetics 16.6 (2000): 276-277.

Roberts, Diane M., et al. “Hexon-chimaeric adenovirus serotype 5 vectors circumvent pre-existing anti-vector immunity.” Nature 441.7090 (2006): 239-243.

Romanenko, Margarita, et al. “Adenovirus type 6: Subtle structural distinctions from adenovirus type 5 result in essential differences in properties and perspectives for gene therapy.” Pharmaceutics 13.10 (2021): 1641.

Ronzitti, Giuseppe, David-Alexandre Gross, and Federico Mingozzi. “Human immune responses to adeno-associated virus (AAV) vectors.” Frontiers in immunology 11 (2020): 670.

Ruff, Robert L., and Robert P. Lisak. “Nature and action of antibodies in myasthenia gravis.” Neurologic clinics 36.2 (2018): 275-291.

Rummler, Silke, et al. “Current techniques for ABO-incompatible living donor liver transplantation.” World journal of transplantation 6.3 (2016): 548.

Runcie, Karie, et al. “Bi-specific and tri-specific antibodies—the next big thing in solid tumor therapeutics.” Molecular Medicine 24.1 (2018): 50.

Ryan, Brent J., Ahuva Nissim, and Paul G. Winyard. “Oxidative post-translational modifications and their involvement in the pathogenesis of autoimmune diseases.” Redox biology 2 (2014): 715-724.

Salganik, Max, Matthew L. Hirsch, and Richard Jude Samulski. “Adeno-associated virus as a mammalian DNA vector.” Mobile DNA III (2015): 827-849.

Shanmugam, Arulkumaran, et al. “Identification of PSA peptide mimotopes using phage display peptide library.” Peptides 32.6 (2011): 1097-1102.

Skytthe, Maria K., Jonas Heilskov Graversen, and Søren K. Moestrup. “Targeting of CD163+Macrophages in Inflammatory and Malignant Diseases.” International Journal of Molecular Sciences 21.15 (2020): 5497.

Sonntag, F., et al. “The assembly-activating protein promotes capsid assembly of different adeno-associated virus serotypes.” Journal of virology 85.23 (2011): 12686-12697.

Sørensen, Karen Kristine, et al. “Liver sinusoidal endothelial cells.” Comprehensive Physiology 5.4 (2011): 1751-1774.

Spiess, Christoph, Qianting Zhai, and Paul J. Carter. “Alternative molecular formats and therapeutic applications for bispecific antibodies.” Molecular immunology 67.2 (2015): 95-106.

Sunasee, Rajesh, and Ravin Narain. “Covalent and noncovalent bioconjugation strategies.” Chemistry of Bioconjugates: Synthesis, Characterization, and Biomedical Applications (2014): 1-75.

Taddeo, Adriano, et al. “Selection and depletion of plasma cells based on the specificity of the secreted antibody.” European journal of immunology 45.1 (2015): 317-319.

Teschner, Sven, et al. “ABO-incompatible kidney transplantation using regenerative selective immunoglobulin adsorption.” Journal of clinical apheresis 27.2 (2012): 51-60.

Tetala, Kishore K R, et al. “Selective depletion of neuropathy-related antibodies from human serum by monolithic affinity columns containing ganglioside mimics.” Journal of medicinal chemistry 54.10 (2011): 3500-3505.

Tian, Xingui, et al. “Broadly neutralizing monoclonal antibodies against human adenovirus types 55, 14p, 7, and 11 generated with recombinant type 11 fiber knob.” Emerging microbes & infections 7.1 (2018): 1-12.

Torres-Domínguez, Lino E., and Grant McFadden. “Poxvirus oncolytic virotherapy.” Expert opinion on biological therapy 19.6 (2019): 561-573.

Tseng, Yu-Shan, and Mavis Agbandje-McKenna. “Mapping the AAV capsid host antibody response toward the development of second generation gene delivery vectors.” Frontiers in immunology 5 (2014): 9.

Thuenemann, Eva C., et al. “Bluetongue virus particles as nanoreactors for enzyme delivery and cancer therapy.” Molecular pharmaceutics 18.3 (2021): 1150-1156.

Varghese, Robin, et al. “Postentry neutralization of adenovirus type 5 by an antihexon antibody.” Journal of virology 78.22 (2004): 12320-12332.

Verdera, Helena Costa, Klaudia Kuranda, and Federico Mingozzi. “AAV vector immunogenicity in humans: a long journey to successful gene transfer.” Molecular Therapy 28.3 (2020): 723-746.

Vincent, Angela, et al. “Serological and experimental studies in different forms of myasthenia gravis.” Annals of the New York Academy of Sciences 1413.1 (2018): 143-153.

Wallukat, Gerd, et al. “Patients with preeclampsia develop agonistic autoantibodies against the angiotensin AT 1 receptor.” The Journal of clinical investigation 103.7 (1999): 945-952.

Wang, Rong, et al. “A Murine Monoclonal Antibody With Potent Neutralization Ability Against Human Adenovirus 7.” Frontiers in cellular and infection microbiology 9 (2019): 417.

Yang, Chen, et al. “Oncolytic viruses as a promising therapeutic strategy for hematological malignancies.” Biomedicine & Pharmacotherapy 139 (2021): 111573.

Zeng, Jiayi, et al. “Oncolytic viro-immunotherapy: an emerging option in the treatment of gliomas.” Frontiers in immunology 12 (2021): 721830.

Zhang, Wei-Wei, et al. “The first approved gene therapy product for cancer Ad-p53 (Gendicine): 12 years in the clinic.” Human gene therapy 29.2 (2018): 160-179.

Zhou, Cissy C., et al. “Angiotensin receptor agonistic autoantibodies induce pre-eclampsia in pregnant mice.” Nature medicine 14.8 (2008): 855.

Zhu, Feng-Cai, et al. “Safety, tolerability, and immunogenicity of a recombinant adenovirus type-5 vectored COVID-19 vaccine: a dose-escalation, open-label, non-randomised, first-in-human trial.” The Lancet 395.10240 (2020): 1845-1854.

1. A method for increasing efficacy of an oncolytic virus or oncolytic virus-like particle (VLP) based thereon by transient selective antibody depletion, the method comprising the following steps:
(i) selecting an individual having a neoplasm for therapy with the oncolytic virus or oncolytic VLP based thereon,
(ii) administering a pharmaceutical composition to the individual,
wherein the pharmaceutical composition comprises at least one pharmaceutically acceptable excipient and a compound comprising
a biopolymer scaffold, wherein the biopolymer scaffold is a protein which is non-immunogenic in the individual, and at least
a first peptide n-mer of the general formula:
$$P {( - S - P )}_{( n - 1 )}$$
a second peptide n-mer of the general formula:
$$P {( - S - P )}_{( n - 1 )} [ [ ; ] ] ,$$
wherein, independently for each occurrence, P is a peptide with a sequence length of 6-13 amino acids, and S is a non-peptide spacer,
wherein, independently for each of the peptide n-mers, n is an integer of at least 1,
wherein each of the peptide n-mers is bound to the biopolymer scaffold,
wherein, independently for each occurrence, P has an amino-acid sequence comprising a sequence fragment with a length of at least six amino acids of a protein sequence of the oncolytic virus,
optionally wherein at most three amino acids of the sequence fragment are independently substituted by any other amino acid; and
(iii) administering the oncolytic virus or oncolytic VLP based thereon to the individual within 96 hours of performing step (ii);
wherein one or more antibodies against the oncolytic virus are present in the individual which are specific for at least one occurrence of peptide P.

2. The method of claim 1, wherein the oncolytic virus is a measles virus, a Herpes simplex virus (HSV), a poxvirus such as Vaccinia virus, a reovirus, a Newcastle disease virus (NVD), a rhabdovirus, a coxsackievirus, a lentivirus, an alphavirus, a vesicular stomatitis virus (VSS), a myxoma virus (MYXV), a mengovirus, a bovine viral diarrhea virus (BVDV), a chimeric oncolytic virus, a picornavirus, a parvovirus, an adenovirus (AdV), an adeno-associated virus (AAV) or a flavivirus.

3.-15. (canceled)

16. The method of claim 1, wherein at least one occurrence of P is a circularized peptide.

17. The method of claim 1, wherein, independently for each occurrence, P is Pₐ or P_{b},
wherein Pₐ has an amino-acid sequence comprising a first sequence fragment with a length of at least six amino acids of a protein sequence of an oncolytic virus, optionally wherein at most three amino acids of the sequence fragment are independently substituted by any other amino acid,
wherein P_{b} has an amino-acid sequence comprising a second sequence fragment with a length of at least six amino acids of a protein sequence of an oncolytic virus, optionally wherein at most three amino acids of the sequence fragment are independently substituted by any other amino acid; and wherein
the first peptide n-mer is Pₐ—S—Pₐ and the second peptide n-mer is Pₐ—S—Pₐ,
the first peptide n-mer is Pₐ—S—Pₐ and the second peptide n-mer is P_{b}—S—P_{b},
the first peptide n-mer is P_{b}—S—P_{b} and the second peptide n-mer is P_{b}—S—P_{b},
the first peptide n-mer is Pₐ—S—P_{b} and the second peptide n-mer is Pₐ—S—P_{b},
the first peptide n-mer is Pₐ—S—P_{b} and the second peptide n-mer is Pₐ—S—Pₐ, or
the first peptide n-mer is Pₐ—S—P_{b} and the second peptide n-mer is P_{b}—S—P_{b}.

18. The method of claim 17, wherein the peptide Pₐ and the peptide P_{b} are two different epitopes of the same viral antigen or two different epitope parts of the same viral epitope.

19. The method of claim 1, wherein the biopolymer scaffold is selected from the group consisting of albumins and globulins.

20. The method of claim 19, wherein the biopolymer scaffold is transferrin.

21. The method of claim 20, wherein the biopolymer scaffold is human transferrin.

22. The method of claim 19, wherein the biopolymer scaffold is albumin.

23. The method of claim 22, wherein the biopolymer scaffold is human albumin.

24. The method of claim 1, wherein said protein sequence of the oncolytic virus is a capsid protein sequence, a nucleocapsid protein sequence, a structural protein sequence, viral envelope- or receptor-binding-protein sequence, or a tegument protein sequence.

25. The method of claim 24, wherein said protein sequence is a capsid protein sequence.

26. The method of claim 1, wherein the pharmaceutical composition is non-immunogenic in the individual.

27. The method of claim 1, wherein the neoplasm is a solid tumor or a hematological malignancy.

28. The method of claim 1, wherein step (ii) is performed prior to or concurrently with step (iii).

29. The method of claim 1, wherein step (ii) is performed at least once prior to step (iii).

30. The method of claim 1, wherein step (ii) is performed at least twice prior to step (iii).

31. The method of claim 1, wherein at least one of said administering of step (ii) and step (iii) is systemic.

32. An anti-neoplastic composition, comprising a compound and further comprising an oncolytic virus or oncolytic VLP based thereon, and at least one pharmaceutically acceptable excipient;
wherein the compound comprises
a biopolymer scaffold, wherein the biopolymer scaffold is a protein which is non-immunogenic in the individual, and at least
a first peptide n-mer of the general formula:
$$P {( - S - P )}_{( n - 1 )}$$
a second peptide n-mer of the general formula:
$$P {( - S - P )}_{( n - 1 )} ,$$
wherein, independently for each occurrence, P is a peptide with a sequence length of 6-13 amino acids, and S is a non-peptide spacer,
wherein, independently for each of the peptide n-mers, n is an integer of at least 1,
wherein each of the peptide n-mers is bound to the biopolymer scaffold,
wherein, independently for each occurrence, P has an amino-acid sequence comprising a sequence fragment with a length of at least six amino acids of a protein sequence of the oncolytic virus, optionally wherein at most three amino acids of the sequence fragment are independently substituted by any other amino acid.

33. A method of inhibiting an immune reaction to a treatment with an oncolytic virus or oncolytic VLP based thereon present in an anti-neoplastic composition in an individual in need of treatment with the anti-neoplastic composition, comprising
obtaining the anti-neoplastic composition as defined in claim 32; wherein the compound of the anti-neoplastic composition is non-immunogenic in the individual, and
administering the anti-neoplastic composition to the individual, wherein the compound transiently reduces the titer of one or more antibodies against the oncolytic virus or VLP based thereon in the individual.
